(19) United States
(12) Patent Application Publication
(10) Pub. No.: US 2022/0177524 A1
(43) Pub. Date: Jun. 9, 2022

(54) NEF-CONTAINING T CELLS AND METHODS OF PRODUCING THEREOF

(71) Applicant: NANJING LEGEND BIOTECH CO., LTD., Nanjing, Jiangsu (CN)

(72) Inventors: Xiaohu FAN, Edmonton, Alberta (CA); Yuncheng ZHAO, Nanjing, Jiangsu (CN); Dawei YU, Hefei, Anhui (CN); Wujinan ZHI, Hengyang, Hunan (CN); Chenchen WANG, Xuzhou, Jiangsu (CN); Qiuchuan ZHUANG, Nanjing, Jiangsu (CN); Pingyan WANG, Fengyang, Anhui (CN); Xuanxuan GUO, Suzhou, Jiangsu (CN)

(21) Appl. No.: 17/262,787

(22) PCT Filed: Jul. 26, 2019
(86) PCT No.: PCT/CN2019/097969

§ 371 Date: Jan. 25, 2021

(30) Foreign Application Priority Data
Jul. 26, 2018 (CN) PCT/CN2018/097235

(51) Int. Cl. C07K 14/005; C12N 5/0783; A61K 35/17; C07K 14/725

(52) U.S. Cl. CPC C07K 14/005; C12N 5/0636; C12N 2740/16022; C07K 14/7051; C12N 2740/15022; A61K 35/17

(57) ABSTRACT

Provided are a method of producing a modified T cell comprising introducing into a precursor T cell a first nucleic acid encoding a Nef protein, wherein the Nef protein upon expression results in down-modulation of the endogenous T cell receptor (TCR) in the modified T cell, wherein the modified T cell furthermore expresses a functional exogenous receptor, such as an engineered TCR (e.g., chimeric TCR), T cell antigen coupler (TAC), TAC-like chimeric receptor, or a chimeric antigen receptor (CAR), the modified cell obtained by the method and the pharmaceutical composition comprising the modified T cell. Also provided is a non-naturally occurring Nef protein comprising one or more mutations.

### CROSS REFERENCE TO RELATED APPLICATIONS

This application is a national stage application under 35 U.S.C. § 371 of International Patent Application No. PCT/CN201.9/097969, filed internationally on Jul. 26, 2019, which claims priority benefit of International Patent Application No. PCT/CN2018/097235, filed Jul. 26, 2018, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422001700SEQLIST.TXT, date recorded: Jan. 19, 2021, size: 126 KB).

### FIELD OF THE PRESENT APPLICATION

The present application relates to a method of producing a modified cell with down-modulated endogenous T cell receptor (TCR). The present application also provides a method of producing a modified T cell with down-modulated endogenous TCR, further expressing an exogenous receptor, such as an engineered TCR or a chimeric antigen receptor (CAR). Further provided are modified T cells produced by the methods described herein, pharmaceutical compositions, kits, and methods of treatment thereof.

### BACKGROUND OF THE PRESENT APPLICATION

Chimeric antigen receptor (CAR)-T cell therapy utilizes genetically modified T cells carrying an engineered receptor specifically recognizing a target tumor antigen to direct T cells to tumor site. It has shown promising results in treating hematological cancer and multiple myeloma (MM). Nevertheless, due to individual differences, autologous CAR-T or TCR-T therapy (using patient's own T cells) presents significant challenges in manufacturing and standardization, with extremely expensive cost for manufacturing and treatment. Furthermore, cancer patients usually have lower immune function, with lymphocytes having reduced number, lower immune activity, and hard to expand in vitro.

Universal allogeneic CAR-T or TCR-T therapy is considered as an ideal model, with T cells derived from healthy donors. However, the key challenge is how to effectively eliminate graft-versus-host disease (GvHD) during treatment due to histoincompatibility. TCR is a cell surface receptor involved in T cell activation in response to antigen presentation. 95% of T cells in human have TCR consisting of an alpha (α) chain and a beta (β) chain, TCRα and TCRβ chains combine to form a heterodimer and associate with CD3 subunits to form a TCR complex present on the cell surface. GvHD happens when donor's T cells recognize non-self major histocompatibility complex (MHC) molecules via TCR and perceive host (transplant recipient) tissues as antigenically foreign and attack them. In order to eliminate endogenous TCR from donor T cells thereby preventing GvHD, people have been using gene editing technologies such as Zinc Finger Nuclease (ZFN), transcription activator-like effector nucleases (TALEN), and Clustered Regularly Interspaced Short Palindromic Repeats (CRISPR)-CRISPR associated (Cas) (CRISPR/Cas) for endogenous TCRα or TCRβ gene knockout (KO), then enriching TCR-negative T cells for allogeneic CAR-T or TCR-T production. However, TCR deletion may lead to impaired CD3 downstream signal transduction pathway, and affect T cell expansion.

The disclosures of all publications, patents, patent applications and published patent applications referred to herein are hereby incorporated herein by reference in their entirety.

### BRIEF SUMMARY OF THE PRESENT APPLICATION

The present application provides a method of producing a modified T cell expressing a Nef protein (e.g., wt Nef, or mutant Nef such as mutant SIV Nef), which down-modulates endogenous TCR. The present application also provides a method of producing a modified T cell expressing a Nef protein (e.g., wt Nef, or mutant Nef such as mutant SIV Nef) and an exogenous receptor, such as engineered TCR (e.g., traditional engineered TCR, chimeric TCR (cTCR)), T cell antigen coupler (TAC), TAC-like chimeric receptor, or CAR (e.g., antibody-based CAR, ligand/receptor-based CAR, or ACTR). Modified T cells produced by the methods described herein, pharmaceutical compositions, kits, and methods of treatment thereof are also provided.

In some embodiments, there is provided a method of producing a modified T cell, comprising: introducing into a precursor T cell a first nucleic acid encoding a Nef protein (e.g., wt Nef, or mutant Nef such as mutant SIV Net), wherein the Nef protein upon expression results in down-modulation of the endogenous T cell receptor (TCR) in the modified T cell. In some embodiments, the down-modulation comprises down-regulating cell surface expression of endogenous TCR by at least about 50%. In some embodiments, the modified T cell expressing Nef comprises a modified endogenous TCR locus.

The Nef protein described herein in some embodiments is selected from the group consisting of SIV Nef, HIV1 Nef, HIV2 Nef, and Nef homologous protein. In some embodiments, the Nef protein is a wildtype Nef. In some embodiments, the Nef protein is a mutant Nef, such as a mutant Nef comprising the amino acid sequence of any one of SEQ ID NOs: 18-22. In some embodiments, the Nef protein is a mutant SIV Nef comprising one or more mutations at any of amino acid residues listed in Table 11. In some embodiments, the Nef protein is a mutant SIV Nef comprising one of more mutations at amino acid residues at any of: (i) aa 2-4, an 8-10, aa 11-13, aa 38-40, aa 44-46, an 47-49, aa 50-52, aa 53-55, aa 56-58, aa 59-61, aa 62-64, an 65-67, aa 98-100, an 107-109, aa, 110-112, an 137-139, aa 152-154, aa 164-166, an 167-169, aa 170-172, aa 173-175, aa 176-178, aa 178-179, 179-181aa, aa 182-184, aa 185-187, aa 188-190, an 191-193, an 194-196, aa 203-205, aa 206-208, aa 212-214, aa 215-217, an 218-220, aa 221-223, an 8-13, an 44-67, aa 107-112, an 164-196, aa 203-208, or an 212-223; (ii) an 2-4, an 44-46, an 56-58, an 59-61, aa 62-64, aa 65-67, aa 98-100, aa 107-109, aa 137-139, aa 152-154, aa 164-166, aa 167-169, an 176-178, aa 178-179, aa 179-181, aa 185-187, aa 188-190, an 194-196, an 203-205, an 44-67, aa 164-169, an 176-181, an 185-190; (iii) an 2-4, an 56-58, aa 59-61, an 62-64, aa 65-67, aa 107-109, aa 137-139, an 152-154, aa 164-166, an 167-169, an 170-172, aa 173-175, an 176-178, 178-179aa, an 179-181, an 182-184, aa 185-187, an 188-190, an 194-196, an 203-205, an 56-67, or an 164-190; or (iv) an 2-4, an 56-58, an 59-61, an 62-64, an 65-67, aa 107-109, an 137-139, an 152-154, an 164-166, an 167-169, aa 176-178, an 178-179, an 179-181, an 185-187, an 188-190, an 194-196, an 203-205, aa 56-67, aa 164-169, aa 176-181, or aa 185-190; wherein the amino acid residue position corresponds to that of wildtype SIV Nef. In some embodiments, the mutant Nef (e.g., mutant SIV Net) down-regulates cell surface expression of endogenous TCR (e.g., TCRα and/or TCRβ). In some embodiments, the mutant Nef protein (e.g. mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the mutant Nef protein (e.g., mutant SIV Net) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. In some embodiments, the mutant Nef protein (e.g., mutant SIV Nef) does not down-regulate cell surface expression of CD4. In some embodiments, the mutant Nef protein (e.g., mutant SIV Nef) down-regulates cell surface expression of CD4. In some embodiments, the mutant Nef protein (e.g., mutant SIV Nef) down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) does not down-regulate cell surface expression of CD28. In some embodiments, the mutant Nef protein (e.g., mutant SIV Nef) down-regulates cell surface expression of CD28. In some embodiments, the mutant Nef protein (e.g., mutant SIV Nef) down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα, and/or TCRβ) no more than about 3% (such as no more than about any of 2% or 1%) differently from that by the wildtvpe Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and does not down-regulates cell surface expression of CD4 and/or CD28. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtvpe Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtvpe Nef.

In some embodiments, the precursor T cell comprises a second nucleic acid encoding a functional exogenous receptor comprising an extracellular ligand binding domain and optionally an intracellular signaling domain. For example, the precursor T cell can be an engineered TCR-T cell (e.g., cTCR-T cell), TAC-T cell, TAC-like-T cell, or CAR-T cell, which is further modified by expressing a Nef protein (e.g., wt Nef, or mutant Nef such as mutant SIV Nef).

In some embodiments, for example when the precursor T cell is not engineered, the method can further comprises a step of introducing into the precursor T cell a second nucleic acid encoding a functional exogenous receptor comprising an extracellular ligand binding domain and optionally an intracellular signaling domain. In some embodiments, the first nucleic acid and the second nucleic acid are on separate vectors. In some embodiments, the first nucleic acid and the second nucleic acid are on the same vector, for example operably linked to the same promoter. In some embodiments, the first nucleic acid is upstream of the second nucleic acid. In some embodiments, the first nucleic acid and the second nucleic acid are connected via a linking sequence, such as a linking sequence comprising any of nucleic acid sequence encoding P2A, T2A, E2A, F2A, BmCPV 2A, BmIFV 2A, (GS)ₙ, (GSCGS)ₙ, (CGGS)ₙ, (GGGGS)ₙ, or nucleic acid sequence of IRES, SV40, CMV, UBC, EF1α, PGK, CAGG, or any combinations thereof, wherein n is an integer of at least one.

In some embodiments, the vector carrying the first and/or second nucleic acids described herein is a viral vector, such as a viral vector selected from the group consisting of an adenoviral vector, an adeno-associated virus vector, a retroviral vector, a lentiviral vector, an episomal vector expression vector, a herpes simplex viral vector, and derivatives thereof. In some embodiments, the vector carrying the first and/or second nucleic acids described herein is a non-viral vector, such as a Piggybac vector or a Sleeping Beauty vector.

In some embodiments according to any of the methods described herein, the modified T cell expressing Nef elicits no or a reduced graft-versus-host disease (GvHD) response in a histoincompatible individual as compared to the GvHD response elicited by a primary T cell isolated from the donor of the precursor T cell.

In some embodiments according to any of the methods described herein, the method further comprises isolating or enriching T cells comprising the first and/or the second nucleic acid. In some embodiments, the method further comprises isolating or enriching TCR-negative T cells from the modified T cell expressing Nef. In some embodiments, the method further comprises formulating the modified T cells expressing Nef with at least one pharmaceutically acceptable carrier.

In some embodiments according to any one of the methods described herein that use a precursor T cell comprising a functional exogenous receptor or comprise a step of introducing an functional exogenous receptor into a precursor T cell, the functional exogenous receptor is a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the cTCR further comprises a hinge domain located between the C-terminus of the extracellular ligand binding domain and the N-terminus of the transmembrane domain. In some embodiments, the hinge domain is derived from CD8α, In some embodiments, the cTCR further comprises a signal peptide located at the N-terminus of the cTCR, such as a signal peptide derived from CD8α.

In some embodiments according to any one of the methods described herein that use a precursor T cell comprising a functional exogenous receptor or comprise a step of introducing an functional exogenous receptor into a precursor T cell, the functional exogenous receptor is a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain.

In some embodiments according to any one of the methods described herein that use a precursor T cell comprising a functional exogenous receptor or comprise a step of introducing an functional exogenous receptor into a precursor T cell, the functional exogenous receptor is a TAC-like chimeric receptor comprising. (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3f); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α.

In some embodiments according to any one of the methods described herein that use a precursor T cell comprising a functional exogenous receptor or comprise a step of introducing an functional exogenous receptor into a precursor T cell, the functional exogenous receptor is a chimeric antigen receptor (CAR), such as a CAR comprises a polypeptide comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) a transmembrane domain; and (c) an intracellular signaling domain. In some embodiments, the antigen-binding fragment is selected from the group of a Camel Ig, Ig NAR, Fab fragments, single chain Fv antibody, and single-domain antibody (sdAb, Nanobody). In some embodiments, the antigen-binding fragment is an sdAb or scFv. In some embodiments, the extracellular ligand binding domain is monovalent. In some embodiments, the extracellular ligand binding domain is multivalent, such as multispecific or multiepitope. In some embodiments, the tumor antigen is selected from the group consisting of Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VECFR2, LewisY, CD24, platelet-derived growth factor receptor-beta (PDGFR-beta), SSEA-4, CD20, Folate receptor alpha, ERBB2 (Her2/neu), MUC1, epidermal growth factor receptor (EGFR), NCAM, Prostase, PAP ELF2M Ephrin B2, IGF-I receptor, CAIX, LMP2, gp100, ber-ab1, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, CPRC5D, CXORF61, CD97. CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MACE A1, ETV6-AML, sperm protein 17, XAGE1, Tie 2, MAD-CT-1, MAD-CT-2, Fos-related antigen 1, p53, p53 mutant, prostein, survivin and telomerase. PCTA-1/Galectin 8, MelanA/MART1, Ras mutant, hTERT, sarcoma translocation breakpoints, ML-IAP, ERG (TMPRSS2 ETS fusion gene), NA17·PAX3. Androgen receptor, Cyclin B1·MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments, the tumor antigen is BCCMA, CD19, or CD20. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152. CD154, and PD-1 In some embodiments, the transmembrane domain is derived from CD8α. In some embodiments, the intracellular signaling domain comprises a primary intracellular signaling domain derived from CD3ζ, CD3γ, CD3ε, CD3δ, FcRγ (FCER1G), FcRβ (Fc Epsilon Rib), CD5, CD22, CD79a, CD79b, CD66d, Fe gamma RIIa, DAP10, and DAP12. In some embodiments, the primary intracellular signaling domain is derived from CD3ζ, CD3γ, or DAP12. In some embodiments, the intracellular signaling domain comprises a co-stimulatory signaling domain derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D. CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the co-stimulatory signaling domain comprises a cytoplasmic domain of CD137 (4-1BB). In some embodiments, the functional exogenous receptor further comprises a hinge domain located between the C-terminus of the extracellular ligand binding domain and the N-terminus of the transmembrane domain. In some embodiments, the hinge domain is derived from CD8α. In some embodiments, the functional exogenous receptor further comprises a signal peptide located at the N-terminus of the polypeptide, such as a signal peptide derived from CD8α.

In some embodiments, there is provided a modified T cell obtained by the methods described herein. In some embodiments, there is provided a pharmaceutical composition comprising the modified T cell, and a pharmaceutically acceptable carrier. In some embodiments, there is provided a method of treating a disease (such as cancer) in an individual (such as human), comprising administering to the individual an effective amount of the pharmaceutical composition.

In another aspect, there is provided a non-naturally occurring Nef protein (also referred to as mutant Nef protein or non-naturally occurring mutant Nef protein), which can comprise one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or one or more mutations at any of amino acid residues listed in Table 11. In another aspect, the non-naturally occurring Nef protein is a mutant SIV Nef protein. In some embodiments, the non-naturally occurring Nef protein comprises an amino acid sequence of any one of SEQ ID NOs: 18-22. In some embodiments, the non-naturally occurring Nef protein is a mutant SIV Nef comprising one of more mutations at amino acid residues at any of: (i) aa 2-4, an 8-10, an 11-13, an 38-40, aa 44-46, an 47-49, aa 50-52, aa 53-55, aa 56-58, aa 59-61, aa 62-64, aa 65-67, an 98-100, aa 107-109, aa 110-112, aa 137-139, aa 152-154, aa 164-166, aa 167-169, an 170-172, aa 173-175, an 176-178, aa 178-179, 179-181aa, aa 182-184, aa 185-187, aa 188-190, aa 191-193, an 194-196, an 203-205, aa 206-208, aa 212-214, aa 215-217, aa 218-220, aa 221-223, aa 8-13, aa 44-67, aa 107-112, aa 164-196, an 203-208, or aa 212-223; (ii) aa 2-4, aa 44-46, aa 56-58, an 59-61, aa 62-64, aa 65-67, an 98-100, an 107-109, an 137-139, aa 152-154, an 164-166, aa 167-169, an 176-178, aa 178-179, an 179-181, aa 185-187, aa 188-190, an 194-196, an 203-205, aa 44-67, an 164-169, an 176-181, an 185-190; (iii) aa 2-4, aa 56-58, aa 59-61, an 62-64, aa 65-67, aa 107-109, aa 137-139, aa 152-154, aa 164-166, aa 167-169, an 170-172, aa 173-175, aa 176-178, 178-179aa, an 179-181, an 182-184, aa 185-187, an 188-190, aa 194-196, aa 203-205, aa 56-67, or an 164-190; or (iv) an 2-4, an 56-58, aa 59-61, aa 62-64, an 65-67, aa 107-109, aa 137-139, an 152-154, aa 164-166, an 167-169, an 176-178, aa 178-179, an 179-181, an 185-187, aa 188-190, an 194-196, an 203-205, aa 56-67, an 164-169, aa 176-181, or an 185-190; wherein the amino acid residue position corresponds to that of wildtype SIV Nef. In some embodiments, the non-naturally occurring Nef (e.g., mutant SIV Nef) down-regulates cell surface expression of endogenous TCR (e.g., TCRα and/or TCRβ). In some embodiments, the non-naturally occurring Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the non-naturally occurring Nef protein (e.g., mutant SIX Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. In some embodiments, the non-naturally occurring Nef protein (e.g., mutant SIV Nef) does not down-regulate cell surface expression of CD4. In some embodiments, the non-naturally occurring Nef protein (e.g., mutant SIV Nef) down-regulates cell surface expression of CD4. In some embodiments, the non-naturally occurring Nef protein (e.g., mutant SIV Net) down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the non-naturally occurring Nef protein (e.g., mutant SIV Nef) does not down-regulate cell surface expression of CD28. In some embodiments, the non-naturally occurring Nef protein (e.g., mutant SIV Nef) down-regulates cell surface expression of CD28. In some embodiments, the non-naturally occurring Nef protein (e.g., mutant SIV Nef) down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the non-naturally occurring Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60% 70%, 80%, 90%, or 95%) more than that by the wildtype Net), and does not down-regulates cell surface expression of CD4 and/or CD28. In some embodiments, the non-naturally occurring Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef.

The non-naturally occurring Nef proteins described herein (e.g., mutant SIV Nef) can be used in any one of the methods described herein.

The present invention further provides kits and articles of manufacture that are useful for the methods described herein.

### BRIEF DESCRIPTION OF THE DRAWINGS

FIGS. 1A-1B demonstrate SIV Nef expression can significantly inhibit T cell activation.

FIG. 1A shows after transducing Jurkat cell line with lentivirus encoding SIV Nef-LNGFR (M071), LNGFR+ cells rate was 66.1%, and magnetic-activated cell sorting (MACS) further enriched LNGFR+ cells to 94.3%. FIG. 1B shows that T cell activation marker CD69 was significantly reduced in LNGFR+ Jurkat cells stimulated with PHA, but not affected in LNGFR+ Jurkat cells stimulated with PMA/ION. “UnT” indicates untransduced Jurkat cells. “TCRα KO” indicates TCRα knock-out Jurkat cell line by CRISPR/Cas method. “Vector” indicates Jurkat cells transduced with empty vector. “M071” represents LNGFR+ Jurkat cell population expressing SIV Nef-P2A-LNGFR and enriched by MACS.

FIG. 2 shows SIV Nef expression affects TCR-mediated signaling pathway by inhibiting cell surface expression of TCR/CD3 complex. “UnT” indicates untransduced Jurkat cells. “TCRα KO” indicates TCRα knock-out Jurkat cell line by CRISPR/Cas method. “Vector” indicates Jurkat cells transduced with empty vector. “M071” represents LNGFR+ Jurkat cell population expressing SIV Nef-P2A-LNGFR and enriched by MACS.

FIG. 3 shows HIV1 Nef and HIV2 Nef expression affects TCR-mediated signaling pathway by inhibiting cell surface expression of TCR/CD3 complex. “UnT” indicates untransduced Jurkat cells. “TCRα KO” indicates TCRα knock-out Jurkat cell line by CRISPR/Cas method. “Vector” indicates Jurkat cells transduced with empty vector. “M071” represents LNGFR+ Jurkat cell population expressing SIV Nef-P2A-LNGFR and enriched by MACS. “HIV1 Nef” represents Jurkat cells expressing HIV1 Nef-T2A-Puro. “HIV2 Nef” represents Jurkat cells expressing HIV2 Nef-T2A-Puro.

FIGS. 4A-4D show cell sorting strategy for SIV Nef-expressing TCR-negative T cells and target cell cytolytic effects. FIG. 4A shows FACS result of BCMA CAR and LNGFR expression on HEK 293T cells co-transfected with SIV Nef-P2A-LNGFR and BCMA CAR lentiviruses after 3 days. FIG. 4B shows TCRαβ positive and negative rates for LNGFR+ T cells co-transfected with SIV Nef-P2A-LNGFR and BCMA CAR-P2A-LNGFR lentiviruses and sorted with MACSelect LNGFR MicroBeads. FIG. 4C shows the TCRαβ, CD3ε, and LNGFR expression ratios in MACS enriched CD3ε negative T cells, which were co-transfected with SIV Nef-P2A-LNGFR and BCMA CAR lentiviruses. FIG. 4D shows specific and non-specific cytolytic effects of CAR+/CD3ε− T cells on RPMI-8226 (BCMA+) and K562 (BCMA−) cell lines. “UnT” indicates untransduced primary T cells. “NC” represents Luc-labeled cells not incubated with primary T cells as negative control. “PC” represents Triton X-100 to lysis all Luc-labeled cells as positive control. “MACS CD3ε neg” represents MACS enriched CD3ε-negative T cell population. “TCRαβ−” represents TCRαp negative T cells after CD3ε sorting. “TCRαβ+” represents TCRαp positive T cells after CD3ε sorting.

FIGS. 5A-5C demonstrate the expression rate of BCMA CAR (CAR pos), TCRαβ (TCRαβ neg) and CD3ε (CD3ε neg) in T cells transfected with SIV Nef+CAR All-in-One lentiviral vector, such as BCMA CAR-P2A-LNGFR-SIV Nef (M072), BCMA CAR-P2A-SIV Nef (M086), BCMA CAR-P2A-(GGGS)₃-SIV Nef (M090), and SIV Nef-P2A-BCMA CAR (M091), SIV Nef-IRES-BCMA CAR (M126), BCMA CAR-IRES-SIV Nef (M159), BCMA CAR-PGK-SIV Nef (M160), and SIV Nef-PGK-BCMA CAR (M161). SIV Nef-P2A-LNGFR (M071) was used as a non-CAR encoding control. “UnT” represents untransduced Jurkat cells. “CAR pos” represents CAR positive T cells. “TCRαβ neg” represents TCRαp negative T cells. “CD3ε neg” represents CD3ε negative T cells.

FIGS. 6A-6D show effects of Nef subtypes and mutants on TCRαβ, CDε, CD28, and CD4 expression on T cells.

FIG. 7 shows TCRαβ negative T cell rates post-MACS enrichment for SIV Nef-IRES-CD20 scFv (Rituximab) CAR (M167) T cells (89.7%), SIV Nef-IRES-CD20 scFv (Leu-16) CAR (M168) T cells (93.3%), SIV Nef-IRES-CD19×CD20 scFv CAR (M169) T cells (92.1%), SIV Nef-IRES-CD19 scFv CAR (M170) T cells (93.6%), SIV Nef-IRES-BCMA BiVHH CAR1 (M171) T cells (93.5%), SIV Nef-IRES-BCMA BiVHH CAR2 (M172) T cells (87.9%), and SIV Nef-IRES-BCMA mono-VHH CAR (M173) T cells (94.0%). Untransduced T cells (UnT) served as control.

FIGS. 8A-8B show CAR-mediated specific tumor cytotoxicity of MACS-sorted TCRαβ negative T cells transduced with various SIV Nef+CAR all-in-one constructs, with MACS-sorted TCRαβ positive T cells transduced with various SIV Nef+CAR all-in-one constructs and un-transduced T cells (UnT) as controls. M167: SIV Nef-IRES-CD20 scFv (Rituximab) CAR T cells. M168: SIV Nef-IRES-CD20 scFv (Leu-16) CAR T cells. M169: SIV Nef-IRES-CD19-CD20 scFv CAR T cells. M170: SIV Nef-IRES-CD19 scFv CAR T cells. M171: SIV Nef-IRES-BCMA BiVHH CAR1 T cells. M172: SIV Nef-IRES-BCMA BiVHH CAR2 T cells. M173: SIV Nef-IRES-BCMA mono-VHH CAR T cells.

FIGS. 9A-9B show TCR-mediated non-specific cytotoxicity of MACS-sorted TCRαβ positive and negative T cells transduced with various SIV Nef+CAR all-in-one constructs. MACS-sorted TCRαβ negative T cells had little or no TCR-mediated non-specific tumor cell killing activity. M167: SIV Nef-IRES-CD20 scFv (Rituximab) CAR T cells. M168: SIV Nef-IRES-CD20 scFv (Leu-16) CAR T cells. M169: SIV Nef-IRES-CD19-CD20 scFv CAR T cells. M170: SIV Nef-IRES-CD19 scFv CAR T cells. M171: SIV Nef-IRES-BCMA BiVHH CAR1 T cells. M172: SIV Nef-IRES-BCMA BiVHH CAR2 T cells. M173: SIV Nef-IRES-BCMA mono-VHH CAR T cells.

FIG. 10A shows TCRαβ negative T cell rate post-MACS enrichment for T cells transduced with BCMA BiVHH CAR1-IRES-SIV Nef M116 transfer plasmid (PLLV-M133 plasmid). FIG. 10B shows CAR-mediated specific tumor cytotoxicity (left panel) and TCR-mediated non-specific cytotoxicity (right panel) of MACS-sorted TCRαβ positive and negative T cells transduced with PLLV-M133 plasmid. Un-transduced T cells (UnT) served as control.

FIG. 11A shows TCRαβ negative T cell rate post-MACS enrichment for T cells transduced with SIV Nef M116-IRES-CD20 chimeric TCR (anti-CD20 scFv (Leu-16)-(GGGGS)₃-CD3ε), referred to as M572. FIG. 11B shows CD20 chimeric TCR-mediated specific tumor cytotoxicity (left panel) and endogenous TCR-mediated non-specific cytotoxicity (right panel) of MACS-sorted TCRαβ positive and negative T cells transduced with PLLV-M572 plasmid. Un-transduced T cells (UnT) served as control.

FIG. 12A shows TCRαβ negative T cell rate post-MACS enrichment for T cells transduced with SIV Nef M116-IRES-CD20 TAC (anti-CD20 scFv (Leu-16)-(GGGGS)₃-huUCHT1.Y177T-GGGGS-CD4 sequence), referred to as PLLV-M574. FIG. 12B shows anti-CD20 TAC-mediated specific tumor cytotoxicity (left panel) and endogenous TCR-mediated non-specific cytotoxicity (right panel) of MACS-sorted TCRαβ positive and negative T cells transduced with M574 plasmid. Un-transduced T cells (UnT) served as control.

FIGS. 13A-13C show regulatory effects of various SIV Nef amino acid residue mutations on the expression of TCRαβ (FIG. 13A), CD4 (FIG. 13B), and CD28 (FIG. 13C), compared to wildtype SIV Nef (M071). Untransduced Jurkat cells (UnT) served as negative control. Jurkat cells transduced with M116 (SIV Nef M16, see Example 6) served as positive control.

### DETAILED DESCRIPTION OF THE PRESENT APPLICATION

The present application provides a method of producing modified T cells (such as TCR-T cells (e.g., cTCR-T cells), TAC-T cells, TAC-like-T cells, or CAR-T cells) that can elicit reduced GvHD response in a histoincompatible individual during treatment, such as cancer immunotherapy. Briefly, a precursor T cell (i.e., the initial T cell to be modified) is modified to express a Nef (Negative Regulatory Factor) protein, which can down-modulate endogenous TCR (hereinafter referred to as “TCR-deficient T cells” or “GvHD-minimized T cells”), such as down-regulating cell surface expression of endogenous TCRα or TCRβ, thereby inhibiting endogenous TCR-mediated signal transduction. These Nef-containing TCR-deficient T cells can then be further engineered to express an exogenous receptor, such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR). The present application also provides an one-step method of producing GvHD-minimized modified T cells (such as TCR-T cells (e.g., cTCR-T cells), TAC-T cells, TAC-like-T cells, or CAR-T cells), either by co-transducing a precursor T cell with a vector encoding Nef and a vector encoding the exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), or by transducing a precursor T cell with an “All-in-One” vector encoding both Nef and exogenous receptor (such as engineered TCR (e.g., traditional engineered TCR, chimeric TCR (cTCR)), TAC. TAC-like chimeric receptor, or CAR (e.g., antibody-based CAR, ligand/receptor-based CAR, or ACTR)). Modified T cells derived from methods described herein can effectively down-regulate cell surface expression of TCR, while preserves the expression and function of the exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). This invention effectively minimizes or eliminates the occurrence of GvHD during allogeneic transplantation, and provides a convenient, effective, and low-cost strategy for universal allogeneic CAR-T, TCR-T (e.g., cTCR-T), TAC-T, or TAC-like-T therapy.

Accordingly, one aspect of the present application provides a method of producing a modified T cell, comprising introducing into a precursor T cell a first nucleic acid encoding a Nef protein (e.g., wt Nef, or mutant Nef such as mutant SIV Nef) and modified T cells obtained by such methods. In another aspect, there are provided modified T cells comprising a first nucleic acid encoding a Nef protein (e.g., wt Nef, or mutant Nef such as mutant SIV Nef), and optionally a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR). In another aspect, there are provided non-naturally occurring Nef proteins (e.g., mutant SIV Nef) useful for making the modified T cells described herein. Also provided are vectors (such as viral vectors) comprising a nucleic acid encoding the Nef protein (e.g., wt Nef, or mutant Nef such as mutant SIV Nef) and optionally a nucleic acid encoding the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR).

### I. Definitions

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein. Antibodies contemplated herein include single-domain antibodies, such as heavy chain only antibodies.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains am referred herein as “V_{H}Hs”. Some V_{H}Hs may also be known as Nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The terms “full-length antibody,” “intact antibody” or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain (such as V_{H}H) and an Fc region. The constant domains may be native sequence constant domains (e.g., human native sequence constant domains) or amino acid sequence variants thereof. In some cases, the intact antibody may have one or more effector functions.

An “antibody fragment” or “antigen-binding fragment” comprises a portion of an intact antibody, preferably the antigen binding and/or the variable region of the intact antibody. Examples of antibody fragments (or antigen-binding fragment) include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′); fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding.

“Functional fragments” of the antibodies described herein comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

As use herein, the term “specifically binds,” “specifically recognizes,” or is “specific for” refers to measurable and reproducible interactions such as binding between a target and an antigen binding protein (such as an antigen-binding domain, a ligand, an engineered TCR, a CAR, or a chimeric receptor), which is determinative of the presence of the target in the presence of a heterogeneous population of molecules including biological molecules. For example, an antigen binding protein that specifically binds a target (which can be an epitope) is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein to the target as measured, e.g., by a radioimmunoassay (RIA). In some embodiments, an antigen binding protein that specifically binds a target has a dissociation constant (Kd) of ≤1 μM, ≤100 nM, ≤10 nM, ≤1 nM, or ≤0.1 nM. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (such as a CAR (e.g., antibody-based CAR, ligand/receptor-based CAR, or ACTR), engineered TCR (e.g., traditional engineered TCR, chimeric TCR (cTCR)), TAC, TAC-like chimeric receptor, or an sdAb, scFv) for a particular epitope of an antigen. Natural antibodies, for example, are monospecific. The term “multispecific” as used herein denotes that an antigen binding protein (such as any of the exogenous receptor described herein or an sdAb) has two or more antigen-binding sites of which at least two bind different antigens. “Bispecific” as used herein denotes that an antigen binding protein (such as any of the exogenous receptor described herein) has two different antigen-binding specificities. The term “monospecific” CAR as used herein denotes an antigen binding protein (such as any of the exogenous receptor described herein or an sdAb, scFv) that has one or more binding sites each of which bind the same antigen.

The term “valent” as used herein denotes the presence of a specified number of binding sites in an antigen binding protein (such as any of the exogenous receptor described herein or an sdAb, scFv). A natural antibody for example or a full length antibody has two binding sites and is bivalent. As such, the terms “trivalent”, “tetravalent”. “pentavalent” and “hexavalent” denote the presence of two binding site, three binding sites, four binding sites, five binding sites, and six binding sites, respectively, in an antigen binding protein (such as any of the exogenous receptor described herein or an sdAb, scFv).

The term “Fc region” herein is used to define a C-terminal region of an immunoglobulin heavy chain, including native-sequence Fc regions and variant Fc regions.

Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Binding affinity” generally refers to the strength of the sum total of non-covalent interactions between a single binding site of a molecule (e.g., an antibody, any of the exogenous receptor described herein such as a CAR) and its binding partner (e.g., an antigen). Unless indicated otherwise, as used herein, “binding affinity” refers to intrinsic binding affinity that reflects a 1:1 interaction between members of a binding pair (e.g., antibody and antigen, or any of the exogenous receptor described herein and an antigen, such as a CAR and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular ligand binding domain specific for one or more antigens (such as tumor antigens), a transmembrane domain, and an intracellular signaling domain of a T cell and/or other receptors. “CAR-T” refers to a T cell that expresses a CAR (e.g., antibody-based CAR, ligand/receptor-based CAR, ACTR). “BCMA CAR” refers to a CAR having an extracellular binding domain specific for BCMA. “Bi-epitope CAR” refers to a CAR having an extracellular binding domain specific for two different epitopes.

An “isolated” nucleic acid molecule (e.g., encoding a Nef protein, engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally. “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present application contemplate any one or more of these aspects of treatment.

As used herein, an “individual” or a “subject” refers to a mammal, including, but not limited to, human, bovine, horse, feline, canine, rodent, or primate. In some embodiments, the individual is a human.

The term “effective amount” used herein refers to an amount of an agent, such as a modified T cell described herein, or a pharmaceutical composition thereof, sufficient to treat a specified disorder, condition or disease such as ameliorate, palliate, lessen, and/or delay one or more of its symptoms (e.g., cancer, infectious disease, GvHD, transplantation rejection, autoimmune disorders, or radiation sickness). In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the agent (e.g., modified T cell) or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. In the case of infectious disease, such as viral infection, the therapeutically effective amount of a modified T cell described herein or composition thereof can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

As used herein. “delaying” the development of a disease means to defer, hinder, slow, retard, stabilize, and/or postpone development of the disease (e.g., cancer, infectious disease, GvHD, transplantation rejection, autoimmune disorders, or radiation sickness). This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

As used herein, the term “autologous” is meant to refer to any material derived from the same individual to whom it is later to be re-introduced into the individual.

“Allogeneic” refers to a graft derived from a different individual of the same species. “Allogeneic T cell” refers to a T cell from a donor having a tissue human leukocyte antigen (HLA) type that matches the recipient. Typically, matching is performed on the basis of variability at three or more loci of the HLA gene, and a perfect match at these loci is preferred. In some instances allogeneic transplant donors may be related (usually a closely HLA matched sibling), syngeneic (a monozygotic “identical” twin of the patient) or unrelated (donor who is not related and found to have very close degree of HLA matching). The HLA genes fall in two categories (Type I and Type II). In general, mismatches of the Type-I genes (i.e., HLA-A, HLA-B, or HLA-C) increase the risk of graft rejection. A mismatch of an HLA Type II gene (i.e., HLA-DR, or HLA-DQB1) increases the risk of graft-versus-host disease (GvHD).

A “patient” as used herein includes any human who is afflicted with a disease (e.g., cancer, viral infection, GvHD). The terms “subject,” “individual,” and “patient” are used interchangeably herein. The term “donor subject” or “donor” refers to herein a subject whose cells are being obtained for further in vitro engineering. The donor subject can be a patient that is to be treated with a population of cells generated by the methods described herein (i.e., an autologous donor), or can be an individual who donates a blood sample (e.g., lymphocyte sample) that, upon generation of the population of cells generated by the methods described herein, will be used to treat a different individual or patient (i.e., an allogeneic donor). Those subjects who receive the cells that were prepared by the present methods can be referred to as “recipient” or “recipient subject.”

The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

The term “TCR-associated signaling molecule” refers to a molecule having a cytoplasmic immunoreceptor tyrosine-based activation motif (ITAM) that is part of the TCR-CD3 complex. TCR-associated signaling molecules include CD3γε, CD3δε, and ζζ (also known as CD3ζ or CD3ζζ).

The term “stimulation”, as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and upregulate or downregulate expression or secretion of a molecule, such as downregulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process. The term “activated T cells” indicates T cells that are currently undergoing cell division, cytokine production, performance of reg. or cytol. Effector functions, and/or has recently undergone the process of “activation.”

The term “down-modulation” of a molecule (e.g., endogenous TCR or CD4) in T cells refers to down-regulate cell surface expression of the molecule, and/or interfering with its signal transduction (e.g., TCR. CD3, CD28-mediated signal transduction), T cell activation, and T cell proliferation. Down modulation of the target receptors via i.e. internalization, stripping, capping or other forms of changing receptors rearrangements on the cell surface may also be encompassed.

The term “functional exogenous receptor” as used herein, refers to an exogenous receptor (such as e.g. CAR (e.g., antibody-based CAR, ligand/receptor-based CAR, ACTR), engineered TCR (e.g., traditional engineered TCR, chimeric TCR (cTCR)), T cell antigen coupler (TAC), or TAC-like chimeric receptor) that retains its biological activity after being introduced into the T cells or Nef-expressing T cell described herein. The biological activity include but are not limited to the ability of the exogenous receptor in specifically binding to a molecule (e.g., cancer antigen, or an antibody for ACTR), properly transducing downstream signals, such as inducing cellular proliferation, cytokine production and/or performance of regulatory or cytolytic effector functions.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a.” “or,” and “the” include plural referents unless the context clearly dictates otherwise.

### II. Modified T Cell Expressing a Nef Protein

The present invention provides modified T cells comprising a Nef and methods of producing such modified T cells. In some embodiments, the T cells further express a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). The present application thus provides a modified T cells co-expressing any one of the Nef protein (e.g., non-naturally occurring Nef protein, such as mutant SIV Nef) and optionally any one of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. The Nef proteins described herein in some embodiments are mutant Nef, such as any of the mutant Nef proteins described herein, e.g., mutant SIV Nef.

In some embodiments, there is provided a modified T cell (e.g., allogeneic T cell) comprising a first nucleic acid encoding a Nef protein (e.g., wt Nef, or mutant Nef such as mutant SIV Nef), wherein the Nef protein upon expression results in down-modulation of the endogenous TCR (e.g., TCRα and/or TCRβ) in the modified T cell. In some embodiments, the down-modulation comprises down-regulating cell surface expression of endogenous TCR. In some embodiments, the cell surface expression of endogenous TCR is down-regulated by at least about any of 50%, 60%, 70%, 80%, 90%, or 95%. In some embodiments, the cell surface expression of endogenous MHC, CD3ε, CD3γ, and/or CD36 is down-regulated by the Nef protein by at least about any of 50%, 60%, 70%, 80%, 90%, or 95%. In some embodiments, the Nef protein does not down-modulate (e.g., down-regulate expression) CD3ζ, or down-modulate CD3ζ by at most about any of 50%, 40%, 30%, 20%, 10%, or 5%. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the modified T cell expressing Nef comprises unmodified endogenous TCR loci. In some embodiments, the modified T cell expressing Nef comprises a modified endogenous TCR locus, such as TCRα or TCRβ. In some embodiments, the endogenous TCR locus is modified by a gene editing system selected from CRISPR-Cas, TALEN, shRNA, and ZFN. In some embodiments, the endogenous TCR locus is modified by a CRISPR-Cas system, comprising a gRNA comprising the nucleic acid sequence of SEQ ID NO: 23.

In some embodiments, the nucleic acid(s) encoding the gene editing system and the first nucleic acid encoding the Nef protein (e.g., wt Nef, or mutant Nef such as mutant SIV Nef) are on the same vector. In some embodiments, the nucleic acid(s) encoding the gene editing system and the first nucleic acid encoding the Nef protein are on different vectors.

In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or one or more mutations at any of amino acid residues listed in Table 11. In some embodiments, the mutation comprises insertion, deletion, point mutation(s), and/or rearrangement. In some embodiments, the mutant Nef comprises an amino acid sequence of any one of SEQ ID NOs: 18-22. In some embodiments, the mutant Nef is a mutant SIV Nef comprising one of more mutations at amino acid residues at any of: (i) an 2-4, an 8-10, an 11-13, an 3840, an 44-46, an 47-49, an 50-52, aa 53-55, an 56-58, aa 59-61, an 62-64, an 65-67, an 98-100, an 107-109, an 110-112, an 137-139, aa 152-154, aa 164-166, aa 167-169, aa 170-172, aa 173-175, aa 176-178, aa 178-179, 179-181aa, aa 182-184, an 185-187, aa 188-190, an 191-193, an 194-196, as 203-205, an 206-208, aa 212-214, aa 215-217, aa 218-220, aa 221-223, an 8-13, aa 44-67, an 107-112, an 164-196, an 203-208, or aa 212-223; (ii) aa 2-4, aa 44-46, aa 56-58, aa 59-61, aa 62-64, aa 65-67, aa 98-100, an 107-109, aa 137-139, aa 152-154, aa 164-166, aa 167-169, aa 176-178, aa 178-179, aa 179-181, an 185-187, aa 188-190, aa 194-196, aa 203-205, aa 44-67, aa 164-169, aa 176-181, aa 185-190; (iii) a 2-4, aa 56-58, aa 59-61, aa 62-64, an 65-67, an 107-109, an 137-139, an 152-154, an 164-166, aa 167-169, aa 170-172, aa 173-175, aa 176-178, 178-179aa, aa 179-181, aa 182-184, aa 185-187, an 188-190, an 194-196, an 203-205, an 56-67, or an 164-190; or (iv) an 2-4, an 56-58, an 59-61, an 62-64, an 65-67, an 107-109, an 137-139, aa 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the mutant Nef reduces down-modulation effect (e.g., downregulation of cell surface expression) on an endogenous CD4 and/or CD28 in the modified T cell compared to a wildtype Nef protein. In some embodiments, the down-regulation of cell surface expression of endogenous CD4 and/or CD28 is reduced by at least about any of 50%, 60%, 70%, 80%, 90%, or 95%. In some embodiments, the modified T cell comprising the first nucleic acid encoding the Nef protein (e.g., wt Nef, or mutant Nef such as mutant SIV Nef) further comprises a second nucleic acid encoding a functional exogenous receptor comprising an extracellular ligand binding domain and optionally an intracellular signaling domain. In some embodiments, the expression of the Nef protein (e.g., wt Nef, or mutant Nef such as mutant SIV Nef) does not down-modulate (e.g., down-regulate cell surface expression) the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%. In some embodiments, the mutant Nef (e.g., mutant SIV Nef) down-regulates cell surface expression of endogenous TCR (e.g., TCRα and/or TCRβ). In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. In some embodiments, the mutant Nef protein (e.g., mutant SIV Nef) does not down-regulate cell surface expression of CD4. In some embodiments, the mutant Nef protein (e.g., mutant SIV Nef) down-regulates cell surface expression of CD4. In some embodiments, the mutant Nef protein (e.g., mutant SIV Nef) down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) does not down-regulate cell surface expression of CD28. In some embodiments, the mutant Nef protein (e.g., mutant SIV Nef) down-regulates cell surface expression of CD28. In some embodiments, the mutant Nef protein (e.g., mutant SIV Nef) down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and does not down-regulates cell surface expression of CD4 and/or CD28. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ), but does not down-modulate (e.g., down-regulate cell surface expression) the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at most about 3% (such as at most about any of 2% or 1%) different from that by the wildtype Nef. In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef.

In some embodiments, there is provided a modified T cell (e.g., allogeneic T cell) comprising a first nucleic acid encoding a Nef protein (e.g., wildtype Nef, or mutant Nef such as mutant SIV Nef), and a second nucleic acid encoding a functional exogenous receptor comprising an extracellular ligand binding domain and optionally an intracellular signaling domain, wherein the Nef protein upon expression results in down-modulation of the endogenous TCR in the modified T cell. In some embodiments, the functional exogenous receptor is an engineered TCR (e.g., traditional engineered TCR, chimeric TCR (cTCR)). In some embodiments, the functional exogenous receptor is T cell antigen coupler (TAC), or TAC-like chimeric receptor. In some embodiments, the functional exogenous receptor is a CAR (e.g., antibody-based CAR, ligand/receptor-based CAR, or ACTR). In some embodiments, the Nef protein (e.g., wt Nef, or mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional chimeric TCR (cTCR) comprising: (a) an extracellular ligand binding domain comprising one or more (such as any one of 1, 2, 3, 4, 5, 6 or more) binding moieties (e.g., sdAbs, scFvs) specifically recognizing one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the one or more binding moieties are antibodies or antigen-binding fragments thereof. In some embodiments, the first, second, and third TCR subunits are the same (e.g., all CD3ε). In some embodiments, the first, second, and third TCR subunits are different. In some embodiments, the cTCR does not comprise the extracellular domain (or a portion thereof) of the TCR subunit (or the extracellular domain of any TCR subunit). In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional chimeric TCR (cTCR) comprising: (a) an extracellular ligand binding domain comprising one or more (such as any one of 1, 2, 3, 4, 5, 6 or more) binding moieties (e.g., sdAbs, scFvs) specifically recognizing one or more epitopes of a tumor antigen (e.g., BCMA, CD19, CD20); (b) an optional linker; and (c) a full length CD3ε (excluding signal peptide); wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, the cTCR further comprises a hinge domain located between the C-terminus of the extracellular ligand binding domain and the N-terminus of the transmembrane domain. In some embodiments, the hinge domain is derived from CD8α. In some embodiments, the cTCR further comprises a signal peptide located at the N-terminus of the cTCR, such as a signal peptide derived from CD8α. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) cTCR. In some embodiments, the functional cTCR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; wherein the first, second, and third TCR co-receptors are all selected from the group consisting of CD4, CD8, and CD28; and wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, the TAC does not comprise the extracellular domain (or a portion thereof) of the TCR co-receptor (or the extracellular domain of any TCR co-receptor). In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC. In some embodiments, the functional TAC is down-modulated (e.g., down-regulated for cell surface expression) by the Nef protein (e.g., wt Nef, or mutant Nef such as mutant SIV Nef) by at most about any of 50%, 40%, 30%, 20%, 100, or 5%.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (c) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3 (excluding signal peptide); wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the TAC-like chimeric receptor does not comprise the extracellular domain (or a portion thereof) of the TCR subunit (or the extracellular domain of any TCR subunit). In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC-like chimeric receptor. In some embodiments, the functional TAC-like chimeric receptor is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) the CAR. In some embodiments, the CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%. In some embodiments, the one or more binding moieties are antibodies or antigen-binding fragments thereof. In some embodiments, the one or more binding moieties are selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., anti-BCMA sdAbs). In some embodiments, the extracellular ligand binding domain comprises two or more sdAbs linked together. In some embodiments, the one or more binding moieties are scFvs (e.g., anti-CD19 scFv, anti-CD20 scFv, or CD19×CD20 scFvs). In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF. In some embodiments, the receptor is derived from an Fc binding domain, such as an extracellular domain of an Fc receptor. In some embodiments, the Fc receptor is a Fcγ receptor (FcγR). In some embodiments, the FcγR is selected from the group consisting of CD16A (FcγRIIIa), CD16B (FcγRIIIb), CD64A, CD64B, CD64C, CD32A, and CD32B. In some embodiments, the antigen is selected from the group consisting of Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha, ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, GPRC5D. CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a. MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8. MelanA/MART1, Ras mutant, hTERT, sarcoma translocation breakpoints, ML-IAP, ERG (TMPRSS2 ETS fusion gene), NA17, PAX3, Androgen receptor. Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1, and any combination thereof. In some embodiments, the antigen is BCMA, CD19, or CD20.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, the down-modulation comprises down-regulating cell surface expression of endogenous TCR. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) the CAR. In some embodiments, the CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) anti-CD19 scFvs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) anti-CD20 scFvs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising an anti-CD19 scFv fused directly or indirectly (e.g., via linker) to an anti-CD20 scFv; (b) a transmembrane domain; and (c) an intracellular signaling domain, wherein the Nef protein upon expression results in down-modulation of the endogenous TCR in the modified T cell. In some embodiments, the down-modulation comprises down-regulating cell surface expression of endogenous TCR. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) the CAR. In some embodiments, the CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the cell surface expression of endogenous TCR is down-regulated by at least about any of 50%, 60%, 70%, 80%, 90%, or 95%. In some embodiments, the cell surface expression of endogenous MHC, CD3ε, CD3γ, and/or CD38 is down-regulated by the Nef protein (e.g., wt Nef, or mutant Nef such as mutant SIV Nef) by at least about any of 50%, 60%, 70%, 80%, 90%, or 95%. In some embodiments, the Nef protein (e.g., wt Nef, or mutant Nef such as mutant SIV Nef) does not down-modulate (e.g., down-regulate expression) CD3ζ, or down-modulate CD3ζ by at most about any of 50%, 40%, 30%, 20%, 10%, or 5%. In some embodiments, the expression of the Nef protein (e.g., wt Nef, or mutant Nef such as mutant SIV Nef) does not down-modulate (e.g., down-regulate cell surface expression) the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein by at most about any of 50%, 40%, 30%, 20%, 10%, or 5%.

In some embodiments, the modified T cell expressing Nef (e.g., wt Nef, or mutant Nef such as mutant SIV Nef) comprises unmodified endogenous TCR loci. In some embodiments, the modified T cell expressing Nef comprises a modified endogenous TCR locus, such as TCRα or TCRβ. In some embodiments, the endogenous TCR locus is modified by a gene editing system selected from CRISPR-Cas, TALEN, and ZFN. In some embodiments, the endogenous TCR locus is modified by a CRISPR-Cas system, comprising a gRNA comprising the nucleic acid sequence of SEQ ID NO: 23. In some embodiments, the nucleic acid(s) encoding the gene editing system and the first nucleic acid encoding the Nef protein (e.g., wt Nef, or mutant Nef such as mutant SIV Nef) are on the same vector. In some embodiments, the nucleic acid(s) encoding the gene editing system and the first nucleic acid encoding the Nef protein are on different vectors.

In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or one or more mutations at any of amino acid residues listed in Table 11. In some embodiments, the one or more mutations comprise insertion, deletion, point mutation(s), and/or rearrangement. In some embodiments, the mutant Nef protein is a mutant SIV Nef protein. In some embodiments, the mutant Nef comprises an amino acid sequence of any one of SEQ ID NOs: 18-22. In some embodiments, the mutant Nef is a mutant SIV Nef comprising one of more mutations at amino acid residues at any of: (i) a 2-4, aa 8-10, aa 11-13, an 38-40, an 44-46, a 47-49, an 50-52, an 53-55, aa 56-58, aa 59-61, aa 62-64, an 65-67, an 98-100, aa 107-109, aa 110-112, an 137-139, aa 152-154, aa 164-166, an 167-169, aa 170-172, an 173-175, an 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190; or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, an 185-187, an 188-190, aa 194-196, an 203-205, aa 56-67, an 164-169, an 176-181, or aa 185-190; wherein the amino acid residue position corresponds to that of wildtype SIV Nef. In some embodiments, the mutant Nef reduces down-modulation effect (e.g., downregulation of cell surface expression) on an endogenous CD4 and/or CD28 in the modified T cell compared to a wildtype Nef protein. In some embodiments, the down-regulation of cell surface expression of endogenous CD4 and/or CD28 is reduced by at least about any of 50%, 60%, 70%, 80%, 90%, or 95%. In some embodiments, the mutant Nef (e.g., mutant SIV Nef) down-regulates cell surface expression of endogenous TCR (e.g., TCRα and/or TCRβ). In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. In some embodiments, the mutant Nef protein (e.g., mutant SIV Nef) does not down-regulate cell surface expression of CD4. In some embodiments, the mutant Nef protein (e.g., mutant SIV Nef) down-regulates cell surface expression of CD4. In some embodiments, the mutant Nef protein (e.g., mutant SIV Nef) down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) does not down-regulate cell surface expression of CD28. In some embodiments, the mutant Nef protein (e.g., mutant SIV Nef) down-regulates cell surface expression of CD28. In some embodiments, the mutant Nef protein (e.g., mutant SIV Nef) down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and does not down-regulates cell surface expression of CD4 and/or CD28. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ), but does not down-modulate (e.g., down-regulate cell surface expression) the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at most about 3% (such as at most about any of 2% or 1%) different from that by the wildtype Nef. In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef.

In some embodiments, the Nef protein is a mutant SIV Nef comprising amino acid mutations (such as amino acid substitutions, e.g., mutating to one or more Ala, such as mutating any of 1, 2, 3, 4, 5, 6, 7, 8, 9, and 10 amino acid residues to Ala) at any of the amino acid mutation sites described in Table 11. In some embodiments, the mutant SIV Nef comprises mutations (e.g., mutating to one or more Ala, such as mutating any of 1, 2, 3, 4, 5, 6, 7, 8, 9, and 10 amino acid residues to Ala) at up to any of 2, 3, 4, 5, 6, 7, 8, 9, and 10 amino acid mutation sites that belong to the same group as described in Table 11. In some embodiments, the mutation (e.g., mutating to one or more Ala, such as mutating any of 1, 2, 3, 4, 5, 6, 7, 8, 9, and 10 amino acid residues to Ala) is within only one amino acid mutation site described in Table 11. In some embodiments, the mutation (e.g., mutating to one or more Ala, such as mutating any of 1, 2, 3, 4, 5, 6, 7, 8, 9, and 10 amino acid residues to Ala) is within two or more amino acid mutation sites that belong to the same group as described in Table 11. In some embodiments, the mutation is within two or more amino acid mutation sites that are consecutive, wherein the two or more amino acid mutation sites belong to the same group as described in Table 11 (e.g., mutations in aa 185-187 and aa 188-190 of Group 3). In some embodiments, the mutation is mutating all amino acid residues (e.g., all mutating to Ala) within the one or more amino acid mutation sites, wherein the amino acid mutation sites belong to the same group as described in Table 11 (e.g., mutating all residues in aa 185-187 and aa 188-190 of Group 3 to Ala). In some embodiments, the mutation is mutating one amino acid residue (e.g., mutating to Ala) from the first amino acid mutation site, and mutating another amino acid residue (e.g., mutating to Ala) from the second amino acid mutation site, wherein the two amino acid mutation sites belong to the same group as described in Table 11. In some embodiments, the mutations are contiguous, i.e., at least 2 mutation sites are close to each other (e.g., mutated residues are at an 8-10 and an 11-13). In some embodiments, the mutations are non-contiguous, i.e., no mutation sites are close to each other (e.g., mutated residues are at an 8-10 and an 44-46).

In some embodiments, the Nef protein is a mutant SIV Nef that down-regulates endogenous TCR (e.g., TCRα and/or TCRβ) cell surface expression. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. For example, in some embodiments, the Nef protein is a mutant SIV Nef comprising one or more (such as any of 1, 2, 3, 4, 5, 6, 7, 8, 9, 10, or up to any of 2, 3, 4, 5, 6, 7, 8, 9, 10, or more) amino acid mutations (such as amino acid substitutions, e.g., mutating to Ala) at amino acid residues at any of: aa 2-4, aa 8-10, aa 11-13 (e.g., aa 8-13), aa 38-40, an 44-46, an 4749, an 50-52, an 53-55, an 56-58, an 59-61, an 62-64, an 65-67 (e.g., an 44-67), an 98-100, an 107-109, an 110-112 (e.g., an 107-112), an 137-139, an 152-154, an 164-166, an 167-169, an 170-172, an 173-175, an 176-178, an 178-179, 179-181aa, an 182-184, an 185-187, an 188-190, an 191-193, an 194-196 (e.g., an 164-196), an 203-205, an 206-208 (e.g., an 203-208), an 212-214, an 215-217, an 218-220, an 221-223 (e.g., an 212-223), wherein the amino acid residue position corresponds to that of wildtype SIV Nef. In some embodiments, the mutations (e.g., mutating to one or more Ala, such as mutating any of 1, 2, 3, 4, 5, 6, 7, 8, 9, and 10 amino acid residues to Ala) are at up to any of 2, 3, 4, 5, 6, 7, 8, 9, and 10 amino acid mutation sites (e.g., mutated residues are at an 8-10 and an 44-46). In some embodiments, the mutation (e.g., mutating to one or more Ala, such as mutating any of 1, 2, 3, 4, 5, 6, 7, 8, 9, and 10 amino acid residues to Ala) is within only one amino acid mutation site (e.g., only within an 8-10). In some embodiments, the mutation (e.g., mutating to one or more Ala, such as mutating any of 1, 2, 3, 4, 5, 6, 7, 8, 9, and 10 amino acid residues to Ala) is within two or more amino acid mutation sites. In some embodiments, the mutations are contiguous, i.e., at least two amino acid mutation sites are next to each other (e.g., mutated residues are at an 8-10 and an 11-13). In some embodiments, the mutations are non-contiguous, i.e., no amino acid mutation sites are close to each other (e.g., mutated residues are at an 8-10 and an 44-46). In some embodiments, the mutation is mutating all amino acid residues (e.g., all mutating to Ala) within the one or more amino acid mutation sites. In some embodiments, the mutation is mutating one amino acid residue (e.g., mutating to Ala) from the first amino acid mutation site, and mutating another amino acid residue (e.g., mutating to Ala) from the second amino acid mutation site.

In some embodiments, the Nef protein is a mutant SIV Nef that down-regulates endogenous TCR (e.g., TCRα and/or TCRβ) and CD4 cell surface expression, wherein the down-regulation of endogenous TCR (e.g., TCRα and/or TCRβ) cell surface expression by the mutant SIV Nef is different from (less or more than) that by wildtype SIV Nef for no more than about 3% (such as no more than about any of 2% or 1%) and wherein the down-regulation of CD4 cell surface expression by the mutant SIV Nef is less than that by wildtype SIV Nef for at least about 3% (such as at least about any of 4%, 5%, 6%, 7%, 8%, 9%, 10%, 20%, 30%, 40%, 50%, 60%, 70%, 80%, 90%, or 95%). In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef, and down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant SIV Nef down-regulates TCRαβ cell surface expression, but does not down-regulates CD4 cell surface expression. For example, in some embodiments, the Nef protein is a mutant SIV Nef comprising one or more (such as any of 1, 2, 3, 4, 5, 6, 7, 8, 9, 10, or up to any of 2, 3, 4, 5, 6, 7, 8, 9, 10, or more) amino acid mutations (such as amino acid substitutions, e.g., mutating to Ala) at amino acid residues at any of: aa 2-4, aa 44-46, aa 56-58, aa 59-61, aa 62-64, aa 65-67 (e.g., aa 44-67), aa 98-100, aa 107-109, aa 137-139, an 152-154, an 164-166, an 167-169 (e.g., an 164-169), an 176-178, aa 178-179, an 179-181 (e.g., an 176-181), an 185-187, an 188-190 (e.g., an 185-190), an 194-196, aa 203-205, wherein the amino acid residue position corresponds to that of wildtype SIV Nef. In some embodiments, the mutations (e.g., mutating to one or more Ala, such as mutating any of 1, 2, 3, 4, 5, 6, 7, 8, 9, and 10 amino acid residues to Ala) are at up to any of 2, 3, 4, 5, 6, 7, 8, 9, and 10 amino acid mutation sites (e.g., mutated residues are at an 2-4 and an 44-46). In some embodiments, the mutation (e.g., mutating to one or more Ala, such as mutating any of 1, 2, 3, 4, 5, 6, 7, 8, 9, and 10 amino acid residues to Ala) is within only one amino acid mutation site (e.g., only within an 2-4). In some embodiments, the mutation (e.g., mutating to one or more Ala, such as mutating any of 1, 2, 3, 4, 5, 6, 7, 8, 9, and 10 amino acid residues to Ala) is within two or more amino acid mutation sites. In some embodiments, the mutations are contiguous, i.e., at least two amino acid mutation sites are next to each other (e.g., mutated residues are at aa 62-64 and aa 65-67). In some embodiments, the mutations are non-contiguous, i.e., no amino acid mutation sites are close to each other (e.g., mutated residues are at aa 2-4 and aa 4446). In some embodiments, the mutation is mutating all amino acid residues (e.g., all mutating to Ala) within the one or more amino acid mutation sites. In some embodiments, the mutation is mutating one amino acid residue (e.g., mutating to Ala) from the first amino acid mutation site, and mutating another amino acid residue (e.g., mutating to Ala) from the second amino acid mutation site.

In some embodiments, the Nef protein is a mutant SIV Nef that down-regulates endogenous TCR (e.g., TCRα and/or TCRβ) and CD28 cell surface expression, wherein the down-regulation of endogenous TCR (e.g., TCRα and/or TCRβ) cell surface expression by the mutant SIV Nef is different from (less or more than) that by wildtype SIV Nef for no more than about 3% (such as no more than about any of 2% or 1%), and wherein the down-regulation of CD28 cell surface expression by the mutant SIV Nef is less than that by wildtype SIV Nef for at least about 3% (such as at least about any of 4%, 5%, 6%, 7%, 8%, 9%, 10%, 20%, 30%, 40%, 50%, 60%, 70%, 80%, 90%, or 95%). In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. and down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant SIV Nef down-regulates TCRαβ cell surface expression, but does not down-regulates CD28 cell surface expression. For example, in some embodiments, the Nef protein is a mutant SIV Nef comprising one or more (such as any of 1, 2, 3, 4, 5, 6, 7, 8, 9, 10, or up to any of 2, 3, 4, 5, 6, 7, 8, 9, 10, or more) amino acid mutations (such as amino acid substitutions, e.g., mutating to Ala) at amino acid residues at any of: an 2-4, aa 56-58, aa 59-61, aa 62-64, aa 65-67 (e.g., aa 56-67), aa 107-109, aa 137-139, an 152-154, aa 164-166, aa 167-169, an 170-172, aa 173-175, an 176-178, 178-179aa, aa 179-181, aa 182-184, an 185-187, an 188-190 (e.g., aa 164-190), aa 194-196, aa 203-205, wherein the amino acid residue position corresponds to that of wildtype SIV Nef. In some embodiments, the mutations (e.g., mutating to one or more Ala, such as mutating any of 1, 2, 3, 4, 5, 6, 7, 8, 9, and 10 amino acid residues to Ala) are at up to any of 2, 3, 4, 5, 6, 7, 8, 9, and 10 amino acid mutation sites (e.g., mutated residues are at aa 2-4 and aa 56-58). In some embodiments, the mutation (e.g., mutating to one or more Ala, such as mutating any of 1, 2, 3, 4, 5, 6, 7, 8, 9, and 10 amino acid residues to Ala) is within only one amino acid mutation site (e.g., only within aa 2-4). In some embodiments, the mutation (e.g., mutating to one or more Ala, such as mutating any of 1, 2, 3, 4, 5, 6, 7, 8, 9, and 10 amino acid residues to Ala) is within two or more amino acid mutation sites. In some embodiments, the mutations are contiguous, i.e., at least two amino acid mutation sites are next to each other (e.g., mutated residues are at aa 62-64 and aa 65-67). In some embodiments, the mutations are non-contiguous, i.e., no amino acid mutation sites are close to each other (e.g., mutated residues are at aa 2-4 and aa 62-64). In some embodiments, the mutation is mutating all amino acid residues (e.g., all mutating to Ala) within the one or more amino acid mutation sites. In some embodiments, the mutation is mutating one amino acid residue (e.g., mutating to Ala) from the first amino acid mutation site, and mutating another amino acid residue (e.g., mutating to Ala) from the second amino acid mutation site.

In some embodiments, the Nef protein is a mutant SIV Nef that down-regulates endogenous TCR (e.g., TCRα and/or TCRβ), CD4, and CD28 cell surface expression. In some embodiments, the Nef protein is a mutant SIV Nef that down-regulates endogenous TCR (e.g., TCRα and/or TCRβ). CD4, and CD28 cell surface expression, wherein the down-regulation of endogenous TCR (e.g., TCRα and/or TCRβ) cell surface expression by the mutant SIV Nef is different from (less or more than) that by wildtype SIV Nef for no more than about 3% (such as no more than about any of 2% or 1%), and wherein the down-regulation of CD4 and CD28 cell surface expression by the mutant SIV Nef is less than that by wildtype SIV Nef for at least about 3% (such as at least about any of 4%, 5%, 6%, 7%, 8%, 9%, 10%, 20%, 30%, 40%, 50%, 60%, 70%, 80%, 90%, or 95%). In some embodiments, the mutant Nef protein (e.g., mutant SIV Nef) down-regulates cell surface expression of endogenous TCR (e.g., TCRα and/or TCRβ) at least about 3% (including equal to 3%, such as at least about any of 3%, 4%, 5%, 6%, 7%, 8%, 9%, 10%, 20%, 30%, 40%, 50%, 60%, 70%, 80%, 90%, or 95%) more than that by the wildtype Nef. and down-regulates cell surface expression of CD4 and CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. For example, in some embodiments, the Nef protein is a mutant SIV Nef comprising one or more (such as any of 1, 2, 3, 4, 5, 6, 7, 8, 9, 10, or up to any of 2, 3, 4, 5, 6, 7, 8, 9, 10, or more) amino acid mutations (such as amino acid substitutions, e.g., mutating to Ala) at amino acid residues at any of: aa 2-4, aa 56-58, aa 59-61, an 62-64, aa 65-67 (e.g., an 56-67), an 107-109, an 137-139, an 152-154, aa 164-166, aa 167-169 (e.g., an 164-169), aa 176-178, aa 178-179, aa 179-181 (e.g., aa 176-181), aa 185-187, aa 188-190 (e.g., aa 185-190), an 194-196, an 203-205, wherein the amino acid residue position corresponds to that of wildtype SIV Nef. In some embodiments, the mutations (e.g., mutating to one or more Ala, such as mutating any of 1, 2, 3, 4, 5, 6, 7, 8, 9, and 10 amino acid residues to Ala) are at up to any of 2, 3, 4, 5, 6, 7, 8, 9, and 10 amino acid mutation sites (e.g., mutated residues are at an 2-4 and an 56-58). In some embodiments, the mutation (e.g., mutating to one or more Ala, such as mutating any of 1, 2, 3, 4, 5, 6, 7, 8, 9, and 10 amino acid residues to Ala) is within only one amino acid mutation site (e.g., only within an 2-4). In some embodiments, the mutation (e.g., mutating to one or more Ala, such as mutating any of 1, 2, 3, 4, 5, 6, 7, 8, 9, and 10 amino acid residues to Ala) is within two or more amino acid mutation sites. In some embodiments, the mutations are contiguous, i.e., at least two amino acid mutation sites are next to each other (e.g., mutated residues are at an 62-64 and an 65-67). In some embodiments, the mutations are non-contiguous, i.e., no amino acid mutation sites are close to each other (e.g., mutated residues are at an 2-4 and an 65-67). In some embodiments, the mutation is mutating all amino acid residues (e.g., all mutating to Ala) within the one or more amino acid mutation sites. In some embodiments, the mutation is mutating one amino acid residue (e.g., mutating to Ala) from the first amino acid mutation site, and mutating another amino acid residue (e.g., mutating to Ala) from the second amino acid mutation site.

In some embodiments, the Nef protein is a mutant SIV Nef that down-regulates TCRαβ cell surface expression more than (such as at least about any of 3%, 4%, 5%, 6%, 7%, 8%, 9%, 10%, 20%, 30%, 40%, 50%, 60%, 70%, 80%, 90%, or 95% more than) a wildtype SIV Nef, but have less (such as at least about any of 3%, 4%, 5%, 6%, 7%, 8%, 9%, 10%, 20%, 30%, 40%, 50%, 60%, 70%, 80%, 90%, or 95% less) down-regulation of CD4 and CD28 cell surface expression compared to a wildtype SIV Nef. For example, in some embodiments, the Nef protein is a mutant SIV Nef comprising one two amino acid mutations (such as amino acid substitutions, e.g., mutating one or both an to Ala) at amino acid residues 178-179aa, wherein the amino acid residue position corresponds to that of wildtype SIV Nef. In some embodiments, the mutant SIV Nef comprises the amino acid sequence of SEQ ID NO: 18.

In some embodiments, the first nucleic acid and the second nucleic acid are on separate vectors. In some embodiments, the first nucleic acid and the second nucleic acid are on the same vector. In some embodiments, the first nucleic acid and the second nucleic acid are operably linked to the same promoter. In some embodiments, the first nucleic acid and the second nucleic acid are operably linked to different promoters. In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α or PGK. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence. In some embodiments, the linking sequence comprises any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one.

In some embodiments, the vector is a viral vector. In some embodiments, the viral vector selected from the group consisting of adenoviral vector, adeno-associated virus vector, retroviral vector, lentiviral vector, herpes simplex viral vector, and derivatives thereof. In some embodiments, the vector is a non-viral vector, such as episomal expression vector, Enhanced Episomal Vector (EEV), PiggyBac Transposase Vector, or Sleeping Beauty (SB) transposon system.

In some embodiments, the modified T cell expressing Nef elicits no or a reduced GvHD response in a histoincompatible individual as compared to the GvHD response elicited by a primary T cell isolated from the donor of the precursor T cell from which the modified T cell is derived.

In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional exogenous receptor comprising an extracellular ligand binding domain and optionally an intracellular signaling domain, wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the functional exogenous receptor is an engineered TCR (e.g., traditional engineered TCR, chimeric TCR (cTCR)). In some embodiments, the functional exogenous receptor is TAC or TAC-like chimeric receptor. In some embodiments, the functional exogenous receptor is a CAR (e.g., anti-antigen CAR, ligand/receptor-based CAR. ACTR). In some embodiments, the functional exogenous receptor is monovalent and monospecific. In some embodiments, the functional exogenous receptor is multivalent and monospecific. In some embodiments, the functional exogenous receptor is multivalent and multispecific. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional chimeric TCR (cTCR) comprising: an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional chimeric TCR (cTCR) comprising: an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); wherein the first, second, and third TCR subunit are all selected from the group consisting of TCRα, TCRβ, TCRγ, TCRδ, CD3ε, CD3γ, and CD3δ; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; wherein the first, second, and third TCR co-receptors are all selected from the group consisting of CD4, CD8, and CD28; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3&, CD3γ, and CD3δ; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, there is provided a modified T cell (e.g., allogeneic T cell) comprising a first nucleic acid encoding a Nef protein (e.g., wt Nef, or mutant Nef such as mutant SIV Nef), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ; wherein the first nucleic acid and the second nucleic acid are on the same vector (e.g., viral vector such as lentiviral vector), and wherein the Nef protein upon expression results in down-modulation of the endogenous TCR in the modified T cell. In some embodiments, the modified T cell expressing Nef comprises unmodified endogenous TCR loci.

In some embodiments, the modified T cell expressing Nef comprises a modified endogenous TCR locus, such as TCRα or TCRβ. In some embodiments, the nucleic acid(s) encoding the gene editing system and the first nucleic acid encoding the Nef protein are on the same vector. In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant SIV Nef comprising one or more mutations at any of amino acid residues listed in Table 11. In some embodiments, the mutant Nef is a mutant SIV Nef comprising one of more mutations at amino acid residues at any of: (i) a 2-4, aa 8-10, aa 11-13, aa 3840, aa 44-46, aa 47-49, aa 50-52, aa 53-55, aa 56-58, aa 59-61, aa 62-64, aa 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, an 188-190, an 191-193, aa 194-196, aa 203-205, aa 206-208, aa 212-214, aa 215-217, aa 218-220, aa 221-223, an 8-13, aa 44-67, an 107-112, an 164-196, an 203-208, or aa 212-223; (ii) an 2-4, aa 44-46, an 56-58, an 59-61, an 62-64, an 65-67, an 98-100, an 107-109, an 137-139, an 152-154, an 164-166, an 167-169, an 176-178, an 178-179, an 179-181, an 185-187, an 188-190, an 194-196, an 203-205, an 44-67, an 164-169, an 176-181, an 185-190: (iii) an 2-4, an 56-58, an 59-61, an 62-64, an 65-67, aa 107-109, aa 137-139, aa 152-154, aa 164-166, aa 167-169, aa 170-172, aa 173-175, aa 176-178, 178-179aa, an 179-181, an 182-184, an 185-187, an 188-190, an 194-196, an 203-205, an 56-67, or an 164-190; or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that ofwildtype SIV Nef. In some embodiments, the first nucleic acid and the second nucleic acid are operably linked to the same promoter. In some embodiments, the first nucleic acid is upstream of the second nucleic acid. In some embodiments, the first nucleic acid is downstream of the second nucleic acid. In some embodiments, the vector is a viral vector. In some embodiments, the Nef protein (e.g., mutant Nef such as mutant SIV Nef) does not down-regulate cell surface expression of CD4 and/or CD28. In some embodiments, the Nef protein (e.g., wildtype Nef, or mutant Nef such as mutant SIV Nef) down-regulates cell surface expression of CD4 and/or CD28. In some embodiments, the Nef protein (e.g., wildtype Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wildtype Nef, or mutant Nef such as mutant SIV Nef) down-regulates cell surface expression of endogenous TCR, but does not down-modulate (e.g., down-regulate cell surface expression) functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof, (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28; wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCR, TCRγ, TCRδ, CD3ε, CD3γ, and CD3δ; and wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a modified T cell (e.g., allogeneic T cell) comprising a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide), wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant SIV Nef comprising one or more mutations at any of amino acid residues listed in Table 11. In some embodiments, the mutant Nef is a mutant SIV Nef comprising one of more mutations at amino acid residues at any of: (i) aa 2-4, aa 8-10, aa 11-13, aa 38-40, an 44-46, aa 47-49, aa 50-52, an 53-55, aa 56-58, an 59-61, aa 62-64, aa 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, an 221-223, an 8-13, an 44-67, aa 107-112, an 164-196, an 203-208, or an 212-223; (ii) an 2-4, an 44-46, an 56-58, an 59-61, an 62-64, an 65-67, an 98-100, an 107-109, an 137-139, an 152-154, an 164-166, aa 167-169, aa 176-178, aa 178-179, aa 179-181, aa 185-187, aa 188-190, aa 194-196, an 203-205, an 44-67, an 164-169, an 176-181, an 185-190; (iii) an 2-4, an 56-58, an 59-61, an 62-64, an 65-67, an 107-109, an 137-139, an 152-154, an 164-166, an 167-169, an 170-172, an 173-175, aa 176-178, 178-179aa, aa 179-181, aa 182-184, aa 185-187, aa 188-190, aa 194-196, an 203-205, an 56-67, or an 164-190; or (iv) an 2-4, an 56-58, an 59-61, aa 62-64, an 65-67, an 107-109, an 137-139, an 152-154, an 164-166, an 167-169, an 176-178, an 178-179, an 179-181, an 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wildtype Nef, or mutant Nef such as mutant SIV Nef) down-regulates cell surface expression of CD4 and/or CD28. In some embodiments, the Nef protein (e.g., wildtype Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC. TAC-like chimeric receptor, or CAR (e.g., antibody-based CAR, ligand/receptor-based CAR, or ACTR)) is down-modulated (e.g., down-regulated for cell surface expression) by the Nef protein (e.g., wildtype Nef, or mutant Nef such as mutant SIV Nef) by at most about any of 50%, 40%, 30%, 20%, 10%, or 5%.

Thus in some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, a first promoter (e.g., PGK), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first promoter (e.g., PGK), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first promoter (e.g., PGK), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional linker; and (c) a full length CD3ε (excluding signal peptide); a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28; a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4, wherein the TCR subunit is selected from the group consisting of TCRα, TCRβ, TCRγ, TCRδ, CD3ε, CD3γ, and CD3δ; a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε) and (g) an optional intracellular signaling domain comprising an intracellular signaling domain of a fourth TCR subunit (e.g., CD3ε), wherein the first, second, third, and fourth TCR subunits are all selected from the group consisting of TCRα, TCRβ, TCRγ, TCRδ, CD3ε, CD3γ, and CD3δ; a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a modified T cell (e.g., allogeneic T cell) comprising a vector (e.g., a viral vector, such as a lentiviral vector) from upstream to downstream: a second promoter (e.g., EF1-α), a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide), wherein the TCR subunit is selected from the group consisting of TCRα, TCRβ, TCRγ, TCRδ, CD3ε, CD3γ, and CD3δ: a first promoter (e.g., PGK); and a first nucleic acid encoding a Nef protein (e.g., wt Nef, or mutant Nef such as mutant SIV Nef); wherein the Nef protein upon expression results in down-modulation of the endogenous TCR in the modified T cell. In some embodiments, the first promoter and the second promoter are the same. In some embodiments, the first promoter and the second promoter are different. In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant SIV Nef comprising one or more mutations at any of amino acid residues listed in Table 11. In some embodiments, the mutant Nef is a mutant SIV Nef comprising one of more mutations at amino acid residues at any of: (i) aa 2-4, aa 8-10, aa 11-13, aa 38-40, aa 44-46, aa 47-49, aa 50-52, aa 53-55, aa 56-58, aa 59-61, aa 62-64, aa 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, an 164-166, an 167-169, an 170-172, an 173-175, an 176-178, 178-179aa, an 179-181, an 182-184, an 185-187, an 188-190, an 194-196, aa 203-205, an 56-67, or an 164-190; or (iv) an 2-4, an 56-58, an 59-61, aa 62-64, aa 65-67, aa 107-109, aa 137-139, aa 152-154, aa 164-166, aa 167-169, aa 176-178, aa 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the first nucleic acid and the second nucleic acid are transcribed under the same promoter. Thus in some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, sequence encoding self-cleaving 2A peptides such as P2A or T2A), an optional second linking sequence (e.g., sequence encoding flexible linker such as (GGGS)₃ linker), and a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., sequence encoding flexible linker such as (GGGS)₃ linker), and a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., sequence encoding flexible linker such as (GGGS)₃ linker), and a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, or nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA. CD19, CD20); (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS); linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3&; wherein the first, second, and third TCR co-receptors are all selected from the group consisting of CD4, CD8, and CD28; and wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a modified T cell (e.g., allogeneic T cell) comprising a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant Nef comprising one or more mutations at any of amino acid residues listed in Table 11. In some embodiments, the mutant Nef is a mutant SIV Nef comprising one of more mutations at amino acid residues at any of: (i) aa 2-4, aa 8-10, an 11-13, an 38-40, aa 44-46, an 47-49, an 50-52, aa 53-55, an 56-58, an 59-61, aa 62-64, aa 65-67, an 98-100, an 107-109, an 110-112, an 137-139, an 152-154, aa 164-166, aa 167-169, aa 170-172, aa 173-175, aa 176-178, aa 178-179, 179-181aa, an 182-184, aa 185-187, aa 188-190, aa 191-193, aa 194-196, aa 203-205, aa 206-208, aa 212-214, an 215-217, an 218-220, an 221-223, an 8-13, an 44-67, an 107-112, an 164-196, an 203-208, or an 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190; or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogencic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence IRES, an optional second linking sequence (e.g., nucleic acid sequence encoding flexible linker such as (GGGS); linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a modified T cell (e.g., allogeneic T cell) comprising a vector (e.g., a viral vector, such as a lentiviral vector) from upstream to downstream: a promoter (e.g., EF1-α), a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3&: a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, there is provided a modified T cell (e.g., allogeneic T cell) comprising a vector (e.g., a viral vector, such as a lentiviral vector) from upstream to downstream: a promoter (e.g., EF1-α); a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); a first linking sequence (e.g., IRES, nucleic acid sequence encoding self-cleaving 2A peptides such as P2A or T2A), an optional second linking sequence (e.g., nucleic acid sequence encoding flexible linker such as (GGGS): linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the cTCR is an anti-CD20 cTCR comprising the amino acid sequence of SEQ ID NO: 64. In some embodiments, there is provided a modified T cell (e.g., allogeneic T cell) comprising a vector (e.g., a viral vector, such as a lentiviral vector) from upstream to downstream: a promoter (e.g., EF1-α); a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof, (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28; a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the first, second, and third TCR co-receptors are the same. In some embodiments, the first, second, and third TCR co-receptors are different. In some embodiments, there is provided a modified T cell (e.g., allogeneic T cell) comprising a vector (e.g., a viral vector, such as a lentiviral vector) from upstream to downstream: a promoter (e.g., EF1-α); a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the TAC is an anti-CD20 TAC comprising the amino acid sequence of SEQ ID NO: 66. In some embodiments, there is provided a modified T cell (e.g., allogeneic T cell) comprising a vector (e.g., a viral vector, such as a lentiviral vector) from upstream to downstream: a promoter (e.g., EF1-α); a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD31, and CD3δ; a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a modified T cell (e.g., allogeneic T cell) comprising a vector (e.g., a viral vector, such as a lentiviral vector) from upstream to downstream: a promoter (e.g., EF1-α); a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ; a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the Nef protein is selected from the group consisting of SIV Nef. HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant SIV Nef comprising one or more mutations at any of amino acid residues listed in Table 11. In some embodiments, the mutant Nef is a mutant SIV Nef comprising one of more mutations at amino acid residues at any of: (i) aa 2-4, aa 8-10, an 11-13, aa 3840, an 44-46, an 47-49, an 50-52, an 53-55, an 56-58, aa 59-61, an 62-64, an 65-67, an 98-100, an 107-109, an 110-112, an 137-139, an 152-154, an 164-166, an 167-169, an 170-172, an 173-175, an 176-178, an 178-179, 179-181aa, an 182-184, an 185-187, an 188-190, an 191-193, aa 194-196, aa 203-205, aa 206-208, aa 212-214, aa 215-217, aa 218-220, aa 221-223, an 8-13, an 44-67, an 107-112, an 164-196, an 203-208, or an 212-223; (ii) an 24, an 44-46, an 56-58, an 59-61, an 62-64, an 65-67, an 98-100, an 107-109, an 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, an 185-190; (iii) an 2-4, an 56-58, an 59-61, aa 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, aa 203-205, an 56-67, or aa 164-190: or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, aa 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α or PGK.

In some embodiments, the linking sequence comprises any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one. In some embodiments, the linking sequence is IRES. In some embodiments, the linking sequence is nucleic acid sequence encoding P2A.

In some embodiments, the vector is a viral vector. In some embodiments, the viral vector selected from the group consisting of adenoviral vector, adeno-associated virus vector, retroviral vector, lentiviral vector, herpes simplex viral vector, and derivatives thereof. In some embodiments, the viral vector is a lentiviral vector. In some embodiments, the vector is a non-viral vector, such as episomal expression vector, Enhanced Episomal Vector (EEV), PiggyBac Transposase Vector, or Sleeping Beauty (SB) transposon system. Further provided are T cells obtained by introducing any of the vectors (e.g., viral vector) described herein. Further provided are T cells obtained by any of the methods described herein.

### Vectors

The present application provides vectors for cloning and expressing any one of Nef protein (e.g., wt Nef, or mutant Nef such as mutant SIV Nef) or functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the Nef protein (e.g., wt Nef, or mutant Nef such as mutant SIV Nef) coding sequence and/or self-inactivating lentiviral vectors carrying exogenous receptor (e.g. such as engineered TCR (e.g., traditional engineered TCR, chimeric TCR (cTCR)). TAC, TAC-like chimeric receptor, or CAR (e.g., antibody-based CAR, ligand/receptor-based CAR, or ACTR)) can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PET), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the Nef-encoding nucleic acid and/or exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR))-encoding nucleic acid to the engineered immune effector cells (e.g., T cell). See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids encoding Nef and/or exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein is introduced to the engineered immune effector cells (e.g., T cell) by a physical method, including, but not limited to electroporation, sonoporation, photoporation, magnetofection, hydroporation.

In some embodiments, the vector (e.g., viral vector such as lentiviral vector) comprises any one of the nucleic acids encoding the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

### Promoters

In some embodiments, the nucleic acid encoding the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC. TAC-like chimeric receptor, or CAR (e.g., antibody-based CAR, ligand/receptor-based CAR, or ACTR)) described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. For example, Michael C. Milone et al. compared the efficiencies of CMV, hEF1α, UbiC and PGK to drive CAR expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein is operably linked to a hEF1α promoter or a PGK promoter.

In some embodiments, the promoter is selected from the group consisting of an EF-1 promoter, a CMV IE gene promoter, an EF-1α promoter, an ubiquitin C promoter, a phosphoglycerate kinase (PGK) promoter, a Rous Sarcoma Virus (RSV) promoter, an Simian Virus 40 (SV40) promoter a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MC1), a beta actin promoter (1-ACT), a simian virus 40 promoter (SV40), and a myeloproliferative sarcoma virus enhancer, negative control region deleted, d1587rev primer-binding site substituted (MND) promoter, an NFAT promoter, a TETON® promoter, and an NFκB promoter.

In some embodiments, the nucleic acid encoding the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein is operably linked to an inducible promoter. Inducible promoters belong to the category of regulated promoters. The inducible promoter can be induced by one or more conditions, such as a physical condition, microenvironment of the engineered immune effector cell (e.g., T cell), or the physiological state of the engineered immune effector cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell. In some embodiments, the inducible promoter can be an NFAT promoter, a TETON® promoter, or an NFκB promoter.

In some embodiments, the vector also contains a selectable marker gene or a reporter gene to select cells expressing the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein from the population of host cells transfected through vectors (e.g., lentiviral vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### Linking Sequence

In some embodiments, the vector comprises more than one nucleic acids encoding the Nef protein (e.g., wt Nef, or mutant Nef such as mutant SIV Nef) and/or the exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, the vector (e.g., viral vector such as a lentiviral vector) comprises a first nucleic acid encoding a Nef protein (e.g., wt Nef, or mutant Nef such as mutant SIV Nef) and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the first nucleic acid is operably linked to the second nucleic acid via a linking sequence. In some embodiments, the linking sequence is an internal ribosome entry site (IRES). IRES is an RNA element that allows for translation initiation in a cap-independent manner. In some embodiments, the linking sequence comprises (e.g., is) nucleic acid sequence encoding a self-cleaving 2A peptide, such as P2A, T2A, E2A, F2A, BmCPV 2A, BmIFV 2A. In some embodiments, the linking sequence is an IRES comprising a nucleic acid sequence of SEQ ID NO: 34. In some embodiments, the linking sequence is a PGK comprising a nucleic acid sequence of SEQ ID NO: 35. In some embodiments, the linking sequence is nucleic acid sequence encoding a P2A peptide comprising an amino acid sequence of SEQ ID NO: 36. In some embodiments, the linking sequence is nucleic acid sequence encoding a T2A peptide comprising an amino acid sequence of SEQ ID NO: 37. In some embodiments, the linking sequence is nucleic acid sequence encoding a peptide linker as described in the below “Peptide linkers” Section under “V. Functional exogenous receptor”, such as a flexible linker. In some embodiments, the flexible linking sequence is selected from the group consisting of nucleic acid sequences encoding (GS)ₙ, (GSGGS)ₙ (GGGS)ₙ, and (GGGGS)ₙ, where n is an integer of at least one). In some embodiments, the linking sequence encodes a selectable marker, such as LNGFR. In some embodiments, the linking sequence comprises one or more types of the linking sequences described herein, such as nucleic acid sequence encoding a self-cleaving 2A peptide (e.g., P2A) followed by a Gly-Ser flexible linker (e.g., (GGGS)₃), or a self-cleaving 2A peptide (e.g., P2A) followed by a selectable marker (e.g., LNGFR).

Thus in some embodiments, there is provided a vector (e.g., viral vector such as lentiviral vector) comprising a first nucleic acid encoding a Nef protein (e.g., wt Nef, or mutant Nef such as mutant SIV Nef). In some embodiments, the Nef protein (e.g., wt Nef, or mutant Nef such as mutant SIV Nef) down-modulates (e.g., down-regulates cell surface expression) endogenous TCR. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4.

In some embodiments, the Nef protein upon expression in a T cell does not down-modulate (e.g., down-regulate expression) CD3ζ, CD4, CD28, and/or the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), or down-modulates CD3ζ, CD4, CD28, and/or the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) by at most about any of 50%, 40%, 30%, 20%, 10%, or 5%. In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises an amino acid sequence of any one of SEQ ID NOs: 18-22. In some embodiments, the mutant Nef is a mutant SIV Nef comprising one or more mutations at any of amino acid residues listed in Table 11. In some embodiments, the mutant Nef is a mutant SIV Nef comprising one of more mutations at amino acid residues at any of: (i) aa 2-4, aa 8-10, aa 11-13, aa 38-40, aa 44-46, aa 47-49, aa 50-52, aa 53-55, an 56-58, an 59-61, an 62-64, an 65-67, aa 98-100, aa 107-109, aa 110-112, aa 137-139, aa 152-154, aa 164-166, aa 167-169, aa 170-172, an 173-175, an 176-178, an 178-179, 179-181aa, an 182-184, an 185-187, an 188-190, an 191-193, aa 194-196, aa 203-205, aa 206-208, aa 212-214, aa 215-217, aa 218-220, aa 221-223, an 8-13, an 44-67, an 107-112, an 164-196, an 203-208, or an 212-223; (ii) an 2-4, an 44-46, an 56-58, aa 59-61, an 62-64, an 65-67, an 98-100, an 107-109, an 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, an 185-190; (iii) an 2-4, an 56-58, an 59-61, aa 62-64, an 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, an 56-67, or aa 164-190; or (iv) a 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef. or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the vector (e.g., viral vector such as lentiviral vector) further comprises a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the first nucleic acid and the second nucleic acid are operably linked to the same promoter. In some embodiments, the first nucleic acid and the second nucleic acid are operably linked to different promoters.

In some embodiments, there is provided a vector (e.g., viral vector such as a lentiviral vector) comprising a first nucleic acid encoding a Nef protein (e.g., wt Nef, or mutant Nef such as mutant SIV Nef) and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the first nucleic acid and the second nucleic acid are operably linked to different promoters (e.g., EF1-α and PGK). In some embodiments, the first nucleic acid is upstream of the second nucleic acid. In some embodiments, the first nucleic acid is downstream of the second nucleic acid.

In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain e.g., In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide). In some embodiments, the cTCR is an anti-CD20 cTCR comprising the amino acid sequence of SEQ ID NO: 64. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ. In some embodiments, the TAC is an anti-CD20 TAC comprising the amino acid sequence of SEQ ID NO: 66. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3γ, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a vector (e.g., a viral vector, such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%. In some embodiments, the Nef protein is a mutant SIV Nef comprising one of more mutations at amino acid residues at any of: (i) aa 2-4, aa 8-10, aa 11-13, as 38-40, aa 44-46, aa 47-49, aa 50-52, as 53-55, as 56-58, as 59-61, as 62-64, as 65-67, as 98-100, aa 107-109, aa 110-112, aa 137-139, aa 152-154, as 164-166, aa 167-169, as 170-172, aa 173-175, aa 176-178, aa 178-179, 179-181aa, aa 182-184, aa 185-187, aa 188-190, as 191-193, as 194-196, as 203-205, as 206-208, as 212-214, aa 215-217, as 218-220, as 221-223, as 8-13, as 44-67, as 107-112, as 164-196, as 203-208, or aa 212-223; (ii) as 2-4, as 44-46, as 56-58, as 59-61, as 62-64, as 65-67, as 98-100, as 107-109, as 137-139, as 152-154, as 164-166, as 167-169, aa 176-178, as 178-179, as 179-181, aa 185-187, as 188-190, as 194-196, as 203-205, as 44-67, as 164-169, as 176-181, as 185-190; (iii) as 2-4, as 56-58, as 59-61, as 62-64, as 65-67, as 107-109, as 137-139, as 152-154, as 164-166, as 167-169, as 170-172, as 173-175, as 176-178, 178-179aa, as 179-181, as 182-184, as 185-187, as 188-190, as 194-196, as 203-205, as 56-67, or aa 164-190; or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein comprises the amino acid sequence of any of SEQ ID NOs: 12-22.

In some embodiments, there is provided a vector (e.g., a viral vector, such as a lentiviral vector) from upstream to downstream: a second promoter (e.g., EF1-α), a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) comprising an extracellular ligand binding domain and optionally an intracellular signaling domain, a first promoter (e.g., PGK), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a second promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first promoter (e.g., PGK), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a second promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first promoter (e.g., PGK), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a second promoter (e.g., EF1-α), a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; a first promoter (e.g., PGK), and a first nucleic acid encoding a Nef protein (e.g., wt Nef, or mutant Nef such as mutant SIV Nef). In some embodiments, the first, second, and third TCR subunits are the same (e.g., all CD3ε). In some embodiments, the first, second, and third TCR subunits are different. In some embodiments, there is provided a vector (e.g., a viral vector, such as a lentiviral vector) from upstream to downstream: a second promoter (e.g., EF1-α), a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); a first promoter (e.g., PGK), and a first nucleic acid encoding a Nef protein (e.g., wt Nef, or mutant Nef such as mutant SIV Nef). In some embodiments, the cTCR is an anti-CD20 cTCR comprising the amino acid sequence of SEQ ID NO: 64. In some embodiments, there is provided a vector (e.g., a viral vector, such as a lentiviral vector) from upstream to downstream: a second promoter (e.g., EF1-α), a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3&; wherein the first, second, and third TCR co-receptors are all selected from the group consisting of CD4, CD8, and CD28; a first promoter (e.g., PGK), and a first nucleic acid encoding a Nef protein (e.g., wt Nef, or mutant Nef such as mutant SIV Nef). In some embodiments, the first, second, and third TCR co-receptors are the same. In some embodiments, the first, second, and third TCR co-receptors are different. In some embodiments, there is provided a vector (e.g., a viral vector, such as a lentiviral vector) from upstream to downstream: a second promoter (e.g., EF1-α), a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ: a first promoter (e.g., PGK), and a first nucleic acid encoding a Nef protein (e.g., wt Nef, or mutant Nef such as mutant SIV Nef). In some embodiments, the TAC is an anti-CD20 TAC comprising the amino acid sequence of SEQ ID NO: 66. In some embodiments, there is provided a vector (e.g., a viral vector, such as a lentiviral vector) from upstream to downstream: a second promoter (e.g., EF1-α), a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; a first promoter (e.g., PGK), and a first nucleic acid encoding a Nef protein (e.g., wt Nef, or mutant Nef such as mutant SIV Nef). In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a vector (e.g., a viral vector, such as a lentiviral vector) from upstream to downstream: a second promoter (e.g., EF1-α), a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ; a first promoter (e.g., PGK), and a first nucleic acid encoding a Nef protein (e.g., wt Nef, or mutant Nef such as mutant SIV Nef). In some embodiments, the Nef protein comprises the amino acid sequence of any of SEQ ID NOs: 12-22. In some embodiments, the Nef protein is a mutant SIV Nef comprising one of more mutations at amino acid residues at any of: (i) an 2-4, an 8-10, aa 11-13, an 38-40, aa 44-46, an 47-49, an 50-52, aa 53-55, an 56-58, an 59-61, an 62-64, aa 65-67, aa 98-100, aa 107-109, aa 110-112, aa 137-139, aa 152-154, aa 164-166, aa 167-169, aa 170-172, an 173-175, an 176-178, an 178-179, 179-181aa, aa 182-184, an 185-187, an 188-190, an 191-193, aa 194-196, aa 203-205, aa 206-208, aa 212-214, aa 215-217, aa 218-220, aa 221-223, an 8-13, an 44-67, an 107-112, an 164-196, an 203-208, or an 212-223; (ii) an 2-4, an 44-46, an 56-58, an 59-61, an 62-64, an 65-67, an 98-100, an 107-109, aa 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, aa 185-190; (iii) an 2-4, an 56-58, an 59-61, aa 62-64, an 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, an 56-67, or an 164-190; or (iv) an 2-4, aa 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef. or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a vector (e.g., viral vector such as a lentiviral vector) comprising a first nucleic acid encoding a Nef protein (e.g., wt Nef, mutant Nef such as mutant SIV Nef) and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the first nucleic acid and the second nucleic acid are operably linked to the same promoter (e.g., EF1-α). In some embodiments, the first nucleic acid is upstream of the second nucleic acid. In some embodiments, the first nucleic acid is downstream of the second nucleic acid. In some embodiments, there is provided a vector (e.g., a viral vector, such as a lentiviral vector) from upstream to downstream: a promoter (e.g., EF1-α), a first nucleic acid encoding a Nef protein (e.g., wt Nef, mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, there is provided a vector (e.g., a viral vector, such as a lentiviral vector) from upstream to downstream: a promoter (e.g., EF1-α), a first nucleic acid encoding a Nef protein (e.g., wt Nef, mutant Nef such as mutant SIV Nef), a first linking sequence IRES, an optional second linking sequence (e.g., nucleic acid sequence encoding flexible linker such as (GGGS); linker), and a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR or ACTR)). In some embodiments, there is provided a vector (e.g., a viral vector, such as a lentiviral vector) from upstream to downstream: a promoter (e.g., EF1-α), a first nucleic acid encoding a Nef protein (e.g., wt Nef, mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC. TAC-like chimeric receptor, or CAR (e.g., antibody-based CAR, ligand/receptor-based CAR or ACTR)). In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., flexible nucleic acid sequence encoding linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide). In some embodiments, the cTCR is an anti-CD20 cTCR comprising the amino acid sequence of SEQ ID NO: 64. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε) (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS); linker), and a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ. In some embodiments, the TAC is an anti-CD20 TAC comprising the amino acid sequence of SEQ ID NO: 66. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ4, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a vector (e.g., a viral vector, such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) a 2-4, aa 8-10, aa 11-13, aa 3840, aa 44-46, aa 47-49, aa 50-52, aa 53-55, aa 56-58, aa 59-61, an 62-64, aa 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, an 8-13, aa 44-67, an 107-112, an 164-196, an 203-208, or aa 212-223; (ii) an 2-4, aa 44-46, an 56-58, an 59-61, an 62-64, an 65-67, an 98-100, an 107-109, an 137-139, an 152-154, an 164-166, an 167-169, aa 176-178, aa 178-179, aa 179-181, aa 185-187, aa 188-190, aa 194-196, aa 203-205, aa 44-67, an 164-169, an 176-181, an 185-190; (iii) an 2-4, an 56-58, an 59-61, an 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, aa 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a vector (e.g., a viral vector, such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α), a second nucleic acid encoding a functional CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef). In some embodiments, there is provided a vector (e.g., a viral vector, such as a lentiviral vector) from upstream to downstream: a promoter (e.g., EF1-α): a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε), wherein the first, second, and third TCR subunit are all selected from the group consisting of TCRα, TCRβ, TCRγ, TCRδ, CD3ε, CD3γ, and CD3δ: a first linking sequence (e.g., IRES, nucleic acid sequence encoding self-cleaving 2A peptides such as P2A or T2A); an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the first, second, and third TCR subunits are the same (e.g., all CD3ε). In some embodiments, the first, second, and third TCR subunits are different. In some embodiments, there is provided a vector (e.g., a viral vector, such as a lentiviral vector) from upstream to downstream: a promoter (e.g., EF1-α); a second nucleic acid encoding a functional chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); a first linking sequence (e.g., IRES, nucleic acid sequence encoding self-cleaving 2A peptides such as P2A or T2A); an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the cTCR is an anti-CD20 cTCR comprising the amino acid sequence of SEQ ID NO: 64. In some embodiments, there is provided a vector (e.g., a viral vector, such as a lentiviral vector) from upstream to downstream: a promoter (e.g., EF1-α); a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4), wherein the TCR subunit is selected from the group consisting of TCRα, TCRβ, TCRγ, TCRδ, CD3ε, CD3γ, and CD3δ, wherein the first, second, and third TCR co-receptors are all selected from the group consisting of CD4, CD8, and CD28; a first linking sequence (e.g., IRES, nucleic acid sequence encoding self-cleaving 2A peptides such as P2A or T2A): an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the first, second, and third TCR co-receptors are the same. In some embodiments, the first, second, and third TCR co-receptors are different. In some embodiments, there is provided a vector (e.g., a viral vector, such as a lentiviral vector) from upstream to downstream: a promoter (e.g., EF1-α); a second nucleic acid encoding a functional T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4, wherein the TCR subunit is selected from the group consisting of TCRα, TCRβ, TCRγ, TCRδ, CD3ε, CD3γ, and CD3δ; a first linking sequence (e.g., 1RES, nucleic acid sequence encoding self-cleaving 2A peptides such as P2A or T2A); an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the TAC is an anti-CD20 TAC comprising the amino acid sequence of SEQ ID NO: 66. In some embodiments, there is provided a vector (e.g., a viral vector, such as a lentiviral vector) from upstream to downstream: a promoter (e.g., EF1-α); a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (c) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; a first linking sequence (e.g., IRES, nucleic acid sequence encoding self-cleaving 2A peptides such as P2A or T2A); an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a vector (e.g., a viral vector, such as a lentiviral vector) from upstream to downstream: a promoter (e.g., EF1-α); a second nucleic acid encoding a functional TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide), wherein the TCR subunit is selected from the group consisting of TCRα, TCRβ, TCRγ, TCRδ, CD3ε, CD3γ, and CD3δ; a first linking sequence (e.g., IRES, nucleic acid sequence encoding self-cleaving 2A peptides such as P2A or T2A); an optional second linking sequence (e.g., nucleic acid sequence encoding flexible linker such as (GGGS)₃ linker); and a first nucleic acid encoding a Nef protein (e.g., wt Nef, or mutant Nef such as mutant SIV Nef). In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, an 8-10, an 11-13, an 38-40, aa 44-46, an 47-49, an 50-52, aa 53-55, aa 56-58, aa 59-61, an 62-64, an 65-67, aa 98-100, an 107-109, an 110-112, an 137-139, aa 152-154, an 164-166, aa 167-169, an 170-172, an 173-175, an 176-178, an 178-179, 179-181aa, aa 182-184, an 185-187, aa 188-190, an 191-193, an 194-196, aa 203-205, an 206-208, aa 212-214, aa 215-217, aa 218-220, aa 221-223, aa 8-13, aa 44-67, aa 107-112, aa 164-196, aa 203-208, or aa 212-223; (ii) an 2-4, an 44-46, aa 56-58, aa 59-61, an 62-64, aa 65-67, aa 98-100, aa 107-109, an 137-139, aa 152-154, an 164-166, an 167-169, an 176-178, an 178-179, an 179-181, an 185-187, aa 188-190, an 194-196, an 203-205, an 44-67, aa 164-169, an 176-181, an 185-190; (iii) an 2-4, an 56-58, an 59-61, an 62-64, an 65-67, an 107-109, an 137-139, aa 152-154, an 164-166, an 167-169, aa 170-172, an 173-175, an 176-178, 178-179aa, an 179-181, an 182-184, an 185-187, an 188-190, an 194-196, an 203-205, an 56-67, or aa 164-190; or (iv) an 2-4, an 56-58, an 59-61, an 62-64, an 65-67, an 107-109, an 137-139, an 152-154, an 164-166, aa 167-169, an 176-178, aa 178-179, aa 179-181, aa 185-187, aa 188-190, aa 194-196, aa 203-205, aa 56-67, aa 164-169, an 176-181, or an 185-190; wherein the amino acid residue position corresponds to that of wildtype SIV Nef. In some embodiments, the Nef protein (e.g., mutant Nef such as mutant SIV Ne) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

### III. Methods of Producing a Modified T Cell

One aspect of the present invention provides methods of producing any one of the modified T cells described above. The method generally involves introducing a second nucleic acid encoding Nef (such as a mutant Nef) and optionally a second nucleic acid encoding a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) into a native or engineered T cell (referred to herein as “precursor T cell”).

In some embodiments, the precursor T cells are derived from the blood, bone marrow, lymph, or lymphoid organs, are cells of the immune system, such as cells of the innate or adaptive immunity. In some aspects, the cells are human cells.

In some embodiments, the precursor T cells are derived from cell lines, e.g., T cell lines. The cells in some embodiments are obtained from a xenogeneic source, for example, from mouse, rat, non-human primate, and pig.

In some embodiments, the precursor T cells are CD4+/CD8-, CD4-/CD8+, CD4+/CD8+, CD4−/CD8−, or combinations thereof. In some embodiments, the T cell is a natural killer T (NKT) cell. In some embodiments, the precursor T cell is an engineered T cell, such as any of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, the precursor T cells produce IL-2, TFN, and/or TNF upon expressing the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein and binding to the target cells, such as BCMA+ tumor cells. In some embodiments, the CD8+ T cells lyse antigen-specific target cells upon expressing the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein and binding to the target cells.

In some embodiments, the T cells are differentiated from a stem cell, such as a hematopoietic stem cell, a pluripotent stem cell, an iPS, or an embryonic stem cell.

In some embodiments, the Nef and/or functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) are introduced to the T cells by transfecting any one of the nucleic acids or any one of the vectors (e.g., non-viral vectors and viral vectors such as lentiviral vectors) described herein. In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is introduced to the T cells by inserting proteins into the cell membrane while passing cells through a microfluidic system, such as CELL SQUEEZE® (see, for example, U.S. Patent Application Publication No. 20140287509).

Methods of introducing vectors (e.g., viral vectors) or isolated nucleic acids into a mammalian cell are known in the art. The vectors described herein can be transferred into a T cell by physical, chemical, or biological methods.

Physical methods for introducing the vector (e.g., viral vectors) into a T cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector (e.g., viral vector) is introduced into the cell by electroporation.

Biological methods for introducing the vector into a T cell include the use of DNA and RNA vectors. Viral vectors have become the most widely used method for inserting genes into mammalian, e.g., human cells.

Chemical means for introducing the vector (e.g., viral vector) into a T cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the Nef proteins (e.g., wt Nef, or mutant Nef such as mutant SIV Nef) and/or functional exogenous receptors (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein may be prepared by a conventional method (e.g., in vitro transcription) and then introduced into the T cell via known methods such as mRNA electroporation. See, e.g., Rabinovich et al., Human Gene Therapy 17:1027-1035.

In some embodiments, the transduced or transfected T cell is propagated ex vivo after introduction of the vector or isolated nucleic acid. In some embodiments, the transduced or transfected T cell is cultured to propagate for at least about any of 1 day, 2 days, 3 days, 4 days, 5 days, 6 days, 7 days, 10 days, 12 days, or 14 days. In some embodiments, the transduced or transfected T cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding any of the Nef proteins (e.g., wt Nef, or mutant Nef such as mutant SIV Nef) and/or functional exogenous receptors (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein in the engineered T cells, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots), Fluorescence-activated cell sorting (FACS), or Magnetic-activated cell sorting (MACS) (also see Example section).

Thus in some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the precursor T cell comprises a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the method further comprises introducing into the precursor T cell a second nucleic acid encoding a functional exogenous receptor comprising an extracellular ligand binding domain and optionally an intracellular signaling domain. In some embodiments, the first nucleic acid and the second nucleic acid are introduced into the T cell sequentially. Thus in some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, Nef-positive and/or endogenous TCR/CD3ε-negative modified T cell is isolated or enriched, then introducing into the enriched modified T cell a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) on another vector, wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, aa 44-46, aa 4749, aa 50-52, aa 53-55, aa 56-58, an 59-61, an 62-64, an 65-67, an 98-100, an 107-109, an 110-112, an 137-139, an 152-154, aa 164-166, an 167-169, an 170-172, aa 173-175, an 176-178, aa 178-179, 179-181aa, an 182-184, aa 185-187, an 188-190, an 191-193, an 194-196, aa 203-205, aa 206-208, an 212-214, aa 215-217, aa 218-220, aa 221-223, aa 8-13, aa 44-67, aa 107-112, aa 164-196, aa 203-208, or an 212-223; (ii) aa 2-4, aa 44-46, an 56-58, aa 59-61, an 62-64, aa 65-67, aa 98-100, aa 107-109, an 137-139, aa 152-154, aa 164-166, an 167-169, an 176-178, an 178-179, an 179-181, aa 185-187, aa 188-190, aa 194-196, aa 203-205, aa 44-67, aa 164-169, aa 176-181, aa 185-190; (iii) an 2-4, an 56-58, an 59-61, an 62-64, an 65-67, an 107-109, an 137-139, an 152-154, an 164-166, an 167-169, an 170-172, aa 173-175, an 176-178, 178-179aa, an 179-181, an 182-184, an 185-187, an 188-190, an 194-196, an 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the first nucleic acid and the second nucleic acid are on the same vector. In some embodiments, the first nucleic acid and the second nucleic acid are operably linked to different promoters. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR or ACTR)), a first promoter (e.g., PGK), a first nucleic acid encoding a Nef protein (e.g., wt Nef, or mutant Nef such as mutant SIV Nef), wherein the Nef protein upon expression results in down-modulation of the endogenous TCR in the modified T cell.

In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence (e.g., IRES, nucleic acid sequence encoding self-cleaving 2A peptides such as P2A or T2A).

Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a functional exogenous receptor comprising an extracellular ligand binding domain and optionally an intracellular signaling domain (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC. TAC-like chimeric receptor, or CAR (e.g., antibody-based CAR, ligand/receptor-based CAR, or ACTR)), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence IRES, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), a first linking sequence encoding P2A, an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, an 8-10, an 11-13, an 38-40, an 44-46, an 47-49, an 50-52, an 53-55, an 56-58, aa 59-61, an 62-64, an 65-67, an 98-100, aa 107-109, an 110-112, an 137-139, an 152-154, an 164-166, aa 167-169, an 170-172, aa 173-175, aa 176-178, aa 178-179, 179-181aa, aa 182-184, aa 185-187, aa 188-190, aa 191-193, an 194-196, an 203-205, an 206-208, an 212-214, an 215-217, an 218-220, an 221-223, an 8-13, aa 44-67, an 107-112, an 164-196, an 203-208, or an 212-223; (ii) an 2-4, an 44-46, aa 56-58, an 59-61, an 62-64, aa 65-67, an 98-100, aa 107-109, an 137-139, an 152-154, an 164-166, an 167-169, an 176-178, an 178-179, an 179-181, an 185-187, an 188-190, an 194-196, aa 203-205, an 44-67, an 164-169, an 176-181, an 185-190; (iii) an 2-4, aa 56-58, aa 59-61, aa 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the Nef protein (e.g., wt Nef, or mutant Nef such as mutant SIV Nef) upon expression down-modulates the endogenous TCR, MHC, CD3ε, CD3γ, and/or CD3δ in the modified T cell, such as down-regulating cell surface expression of endogenous TCR, MHC, CD3ε, CD3γ, and/or CD3δ by at least about any of 50%, 60%, 70%, 80%, 90%, or 95%.

In some embodiments, the modified T cell expressing Nef comprises unmodified endogenous TCR loci. In some embodiments, the modified T cell expressing Nef comprises a modified endogenous TCR locus, such as TCRα or TCRβ. In some embodiments, the endogenous TCR locus is modified by a gene editing system selected from CRISPR-Cas, TALEN, and ZFN.

In some embodiments, the endogenous TCR locus is modified by a CRISPR-Cas system, comprising a gRNA comprising the nucleic acid sequence of SEQ ID NO: 23. In some embodiments, the Nef protein is selected from the group consisting of SIV Nef, HIV1 Nef, HIV2 Nef, and their homologs (such as HIV F2 Nef. HIVC2 Nef, and HIV H2N2 Nef). In some embodiments, the Nef protein is a wildtype Nef. In some embodiments, the Nef protein comprises an amino acid sequence of any one of SEQ ID NOs: 12-17. In some embodiments, the Nef protein is a mutant Nef. In some embodiments, the mutant Nef comprises one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or comprises one or more mutations at any of amino acid residues listed in Table 11. In some embodiments, the mutation comprises insertion, deletion, point mutation(s), and/or rearrangement. In some embodiments, the mutant Nef comprises an amino acid sequence of any one of SEQ ID NOs: 18-22. In some embodiments, the mutant Nef is a mutant SIV Nef comprising one of more mutations at amino acid residues at any of: (i) aa 2-4, aa 8-10, aa 11-13, aa 3840, aa 44-46, aa 47-49, aa 50-52, aa 53-55, aa 56-58, an 59-61, aa 62-64, an 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, an 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, an 98-100, aa 107-109, an 137-139, aa 152-154, aa 164-166, aa 167-169, aa 176-178, aa 178-179, aa 179-181, aa 185-187, an 188-190, an 194-196, an 203-205, an 44-67, an 164-169, an 176-181, an 185-190; (iii) an 2-4, aa 56-58, aa 59-61, aa 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, aa 188-190, an 194-196, an 203-205, an 56-67, an 164-169, an 176-181, or an 185-190; wherein the amino acid residue position corresponds to that of wildtype SIV Nef. In some embodiments, the mutant Nef (e.g., mutant SIV Nef) reduces down-modulation effect (e.g., downregulation of cell surface expression) on an endogenous CD4 and/or CD28 upon expression in the modified T cell compared to a wildtype Nef protein, such as reducing the down-modulation effect by at least about any of 50%, 60%, 70%, 80%, 90%, or 95%. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the Nef protein (e.g., mutant Nef such as mutant SIV Nef) upon expression does not down-modulate (e.g., downregulate expression) CD3ζ, CD4, CD28, and/or the functional exogenous receptor comprising an extracellular ligand binding domain and optionally an intracellular signaling domain (e.g. such as engineered TCR (e.g., traditional engineered TCR chimeric TCR (cTCR)), TAC, TAC-like chimeric receptor, or CAR (e.g., antibody-based CAR, ligand/receptor-based CAR or ACTR)), or down-modulates (e.g., downregulates expression) CD3ζ, CD4, CD28, and/or the functional exogenous receptor comprising an extracellular ligand binding domain and optionally an intracellular signaling domain by at most about any of 50%, 40%, 30%, 20%, 10%, or 5%.

In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α or PGK.

In some embodiments, the linking sequence comprises any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one. In some embodiments, the linking sequence is IRES. In some embodiments, the linking sequence is nucleic acid sequence encoding P2A.

In some embodiments, the vector is a viral vector. In some embodiments, the viral vector selected from the group consisting of adenoviral vector, adeno-associated virus vector, retroviral vector, vaccinia vector, lentiviral vector, herpes simplex viral vector, and derivatives thereof. In some embodiments, the vector is a non-viral vector, such as episomal expression vector, Enhanced Episomal Vector (EEV), PiggyBac Transposase Vector, or Sleeping Beauty (SB) transposon system. In some embodiments, the functional exogenous receptor is an engineered TCR (e.g., traditional engineered TCR, chimeric TCR). In some embodiments, the functional exogenous receptor is TAC, TAC-like chimeric receptor. In some embodiments, the functional exogenous receptor is a non-TCR receptor, such as CAR (e.g., antibody-based CAR, ligand/receptor-based CAR, ACTR).

In some embodiments, the functional exogenous receptor is a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, the one or more binding moieties are antibodies or antigen-binding fragments thereof. In some embodiments, the one or more binding moieties are selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., anti-BCMA sdAbs) or scFvs. In some embodiments, the extracellular ligand binding domain comprises two or more sdAbs linked together. In some embodiments, the extracellular ligand binding domain comprises two or more scFvs linked together. In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF. In some embodiments, the receptor is derived from an Fc binding domain, such as an extracellular domain of an Fe receptor. In some embodiments, the Fc receptor is a Fcγ receptor (FcγR). In some embodiments, the FcγR is selected from the group consisting of CD16A (FcγRIIIa), CD16B (FcγRIIIb), CD64A, CD64B, CD64C, CD32A, and CD32B. In some embodiments, the CAR is monovalent and monospecific. In some embodiments, the CAR is multivalent (e.g., bispecific) and monospecific. In some embodiments, the CAR is multivalent (e.g., bivalent) and multispecific (e.g., bispecific). In some embodiments, the antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof. In some embodiments, the antigen is BCMA, CD19, CD20. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152, CD154, and PD-1. In some embodiments, the transmembrane domain is derived from CD8α. In some embodiments, the intracellular signaling domain comprises a primary intracellular signaling domain of an immune effector cell. In some embodiments, the primary intracellular signaling domain is derived from CD3ζ, CD3γ, CD3ε, CD3δ, FcRγ (FCER1G), FcRβ (Fc Epsilon Rib), CD5, CD22, CD79a, CD79b, CD66d, Fc gamma RIIa, DAP10, and DAP12. In some embodiments, the primary intracellular signaling domain is derived from CD3ζ, DAP12, or CD3γ. In some embodiments, the intracellular signaling domain comprises a co-stimulatory signaling domain. In some embodiments, the co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19. CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the co-stimulatory signaling domain comprises a cytoplasmic domain of CD137. In some embodiments, the CAR described herein further comprises a hinge domain located between the C-terminus of the extracellular ligand binding domain and the N-terminus of the transmembrane domain. In some embodiments, the hinge domain is derived from CD8α. In some embodiments, the CAR further comprises a signal peptide located at the N-terminus of the polypeptide. In some embodiments, the signal peptide is derived from CD8α. In some embodiments, the CAR comprises from N-terminus to C-terminus: a CD8a signal peptide, the extracellular ligand binding domain (e.g., one or more sdAbs specifically recognizing one or more epitopes of BCMA, APRIL/BAFF ligand, or Fc receptor), a CD8a hinge domain, a CD8a transmembrane domain, a co-stimulatory signaling domain derived from CD137, and a primary intracellular signaling domain derived from CD3ζ.

In some embodiments, the functional exogenous receptor is a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the functional exogenous receptor is a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (c) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the functional exogenous receptor is a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε) wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit.

In some embodiments, the modified T cell expressing Nef (e.g., wt Nef, or mutant Nef such as mutant SIV Nef) elicits no or a reduced GvHD response in a histoincompatible individual as compared to the GvHD response elicited by a primary T cell isolated from the donor of the precursor T cell from which the modified T cell is derived. In some embodiments, the method further comprises isolating or enriching T cells comprising the first and/or the second nucleic acid. In some embodiments, the method further comprises isolating or enriching CD3ε-negative T cells from the modified T cell expressing the Nef protein (e.g., wt Nef, or mutant Nef such as mutant SIV Nef). In some embodiments, the method further comprises isolating or enriching endogenous TCRα-negative T cells from the modified T cell expressing the Nef protein (e.g., wt Nef, or mutant Nef such as mutant SIV Nef). In some embodiments, the method further comprises formulating the modified T cells expressing the Nef protein (e.g., wt Nef, or mutant Nef such as mutant SIV Nef) with at least one pharmaceutically acceptable carrier. In some embodiments, the method further comprises administering to an individual an effective amount of the modified T cells expressing the Nef protein (e.g., wt Nef, or mutant Nef such as mutant SIV Nef), or an effective amount of the pharmaceutical formulation comprising the modified T cells expressing the Nef protein and at least one pharmaceutically acceptable carrier. In some embodiments, the individual has cancer. In some embodiments, the individual is a human.

In some embodiments, the functional exogenous receptor is a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. Thus in some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, Nef-positive and/or endogenous TCR/CD3ε-negative modified T cell is isolated or enriched, then introducing into the enriched modified T cell the second nucleic acid encoding the CAR. In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid on another vector encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, the Nef protein comprises an amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, aa 44-46, aa 4749, aa 50-52, aa 53-55, aa 56-58, aa 59-61, aa 62-64, aa 65-67, aa 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, an 203-205, aa 56-67, or an 164-190: or (iv) an 2-4, an 56-58, an 59-61, an 62-64, aa 65-67, an 107-109, aa 137-139, an 152-154, aa 164-166, an 167-169, an 176-178, an 178-179, an 179-181, aa 185-187, an 188-190, an 194-196, aa 203-205, aa 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) CAR. In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, Nef-positive and/or endogenous TCR/CD3ε-negative modified T cell is isolated or enriched, then introducing into the enriched modified T cell the second nucleic acid encoding the cTCR. In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid on another vector encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (c) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the first, second, and third TCR subunits are the same (e.g., all CD3ε). In some embodiments, the first, second, and third TCR subunits are different. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, aa 44-46, aa 47-49, an 50-52, aa 53-55, an 56-58, an 59-61, an 62-64, an 65-67, an 98-100, an 107-109, an 110-112, an 137-139, an 152-154, aa 164-166, aa 167-169, aa 170-172, aa 173-175, aa 176-178, aa 178-179, 179-181aa, an 182-184, an 185-187, aa 188-190, an 191-193, an 194-196, an 203-205, an 206-208, an 212-214, an 215-217, aa 218-220, aa 221-223, an 8-13, aa 44-67, an 107-112, an 164-196, an 203-208, or an 212-223: (ii) an 2-4, an 44-46, an 56-58, an 59-61, an 62-64, an 65-67, an 98-100, an 107-109, an 137-139, aa 152-154, an 164-166, an 167-169, an 176-178, an 178-179, aa 179-181, an 185-187, an 188-190, an 194-196, an 203-205, aa 44-67, aa 164-169, an 176-181, an 185-190; (iii) an 2-4, an 56-58, an 59-61, aa 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, aa 56-67, or an 164-190; or (iv) an 2-4, an 56-58, aa 59-61, an 62-64, an 65-67, an 107-109, an 137-139, aa 152-154, an 164-166, an 167-169, an 176-178, aa 178-179, aa 179-181, aa 185-187, aa 188-190, aa 194-196, aa 203-205, aa 56-67, aa 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) cTCR. In some embodiments, the functional cTCR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, Nef-positive and/or endogenous TCR/CD3ε-negative modified T cell is isolated or enriched, then introducing into the enriched modified T cell the second nucleic acid encoding the TAC. In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid on another vector encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker, (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; wherein the first, second, and third TCR co-receptors are all selected from the group consisting of CD4, CD8, and CD28; and wherein the Nef protein upon expression results in down-modulation of the endogenous TCR in the modified T cell. In some embodiments, the first, second, and third TCR co-receptors are the same (e.g., all CD4). In some embodiments, the first, second, and third TCR co-receptors are different. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, an 11-13, an 38-40, aa 44-46, an 47-49, aa 50-52, an 53-55, an 56-58, an 59-61, an 62-64, aa 65-67, aa 98-100, an 107-109, an 110-112, an 137-139, aa 152-154, aa 164-166, aa 167-169, aa 170-172, aa 173-175, aa 176-178, aa 178-179, 179-181aa, an 182-184, an 185-187, an 188-190, an 191-193, an 194-196, an 203-205, an 206-208, an 212-214, aa 215-217, aa 218-220, aa 221-223, aa 8-13, aa 44-67, aa 107-112, aa 164-196, aa 203-208, or aa 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, an 188-190, an 194-196, an 203-205, an 44-67, an 164-169, an 176-181, an 185-190; (iii) an 2-4, an 56-58, an 59-61, an 62-64, an 65-67, an 107-109, an 137-139, an 152-154, an 164-166, an 167-169, aa 170-172, an 173-175, an 176-178, 178-179aa, an 179-181, an 182-184, an 185-187, an 188-190, an 194-196, an 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, aa 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef. or mutant Nef such as mutant SIV Nef) down-regulates cell surface expression of endogenous TCR, but does not down-modulate (e.g., down-regulate cell surface expression) TAC. In some embodiments, the functional TAC is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ. TCRδ, CD3ε, CD3γ, and CD3δ. In some embodiments, Nef-positive and/or endogenous TCR/CD3ε-negative modified T cell is isolated or enriched, then introducing into the enriched modified T cell the second nucleic acid encoding the TAC-like chimeric receptor. In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid on another vector encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; and wherein the Nef protein upon expression results in down-modulation of the endogenous TCR in the modified T cell. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) an 2-4, an 8-10, an 11-13, an 38-40, aa 44-46, an 47-49, an 50-52, an 53-55, an 56-58, an 59-61, aa 62-64, aa 65-67, aa 98-100, aa 107-109, aa 110-112, aa 137-139, aa 152-154, aa 164-166, aa 167-169, an 170-172, aa 173-175, aa 176-178, aa 178-179, 179-181aa, aa 182-184, aa 185-187, aa 188-190, an 191-193, an 194-196, an 203-205, an 206-208, an 212-214, aa 215-217, an 218-220, an 221-223, an 8-13, an 44-67, an 107-112, an 164-196, an 203-208, or aa 212-223; (ii) an 2-4, an 44-46, an 56-58, an 59-61, an 62-64, an 65-67, aa 98-100, an 107-109, an 137-139, an 152-154, an 164-166, aa 167-169, aa 176-178, aa 178-179, aa 179-181, aa 185-187, aa 188-190, aa 194-196, aa 203-205, aa 44-67, aa 164-169, aa 176-181, aa 185-190: (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190: or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC-like chimeric receptor. In some embodiments, the functional TAC-like chimeric receptor is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the first nucleic acid and the second nucleic acid are on the same vector. In some embodiments, the first nucleic acid and the second nucleic acid are operably linked to different promoters. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD20, CD19); (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19. CD20); (b) a transmembrane domain; and (c) an intracellular signaling domain, a first promoter (e.g., PGK), a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence, e.g., IRES, nucleic acid sequence encoding self-cleaving 2A peptides such as P2A or T2A. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence IRES, an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence IRES, an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) a 2-4, an 8-10, an 11-13, an 38-40, an 44-46, a 47-49, an 50-52, aa 53-55, an 56-58, aa 59-61, aa 62-64, an 65-67, an 98-100, aa 107-109, aa 110-112, an 137-139, aa 152-154, aa 164-166, aa 167-169, aa 170-172, aa 173-175, aa 176-178, aa 178-179, 179-181aa, an 182-184, an 185-187, an 188-190, aa 191-193, an 194-196, an 203-205, an 206-208, aa 212-214, aa 215-217, an 218-220, an 221-223, an 8-13, an 44-67, aa 107-112, an 164-196, an 203-208, or aa 212-223; (ii) aa 2-4, aa 44-46, an 56-58, an 59-61, aa 62-64, aa 65-67, an 98-100, aa 107-109, aa 137-139, aa 152-154, aa 164-166, aa 167-169, aa 176-178, aa 178-179, aa 179-181, an 185-187, aa 188-190, aa 194-196, aa 203-205, aa 44-67, aa 164-169, aa 176-181, aa 185-190: (iii) an 2-4, aa 56-58, an 59-61, an 62-64, an 65-67, an 107-109, an 137-139, an 152-154, an 164-166, an 167-169, aa 170-172, an 173-175, an 176-178, 178-179aa, aa 179-181, an 182-184, an 185-187, an 188-190, aa 194-196, aa 203-205, aa 56-67, or aa 164-190: or (iv) an 2-4, an 56-58, aa 59-61, aa 62-64, aa 65-67, aa 107-109, aa 137-139, aa 152-154, aa 164-166, aa 167-169, aa 176-178, aa 178-179, aa 179-181, an 185-187, an 188-190, aa 194-196, an 203-205, aa 56-67, an 164-169, an 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) CAR. In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the functional exogenous receptor is a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. Thus in some embodiments, there is provided a method of producing a modified T cell (e.g., allogeneic T cell, endogenous TCR-deficient T cell, GvHD-minimized T cell), comprising: introducing into a precursor T cell a first nucleic acid encoding a Nef protein (e.g., wt Nef, or mutant Nef such as mutant SIV Nef), wherein the Nef protein upon expression results in down-modulation of the endogenous TCR in the modified T cell, then introducing into the precursor T cell a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, Nef-positive and/or endogenous TCR/CD3ε-negative modified T cell is isolated or enriched, then introducing into the enriched modified T cell the second nucleic acid encoding the CAR. In some embodiments, the first nucleic acid and the second nucleic acid are introduced into the T cell simultaneously. In some embodiments, the first nucleic acid and the second nucleic acid are on separate vectors. Thus in some embodiments, there is provided a method of producing a modified T cell (e.g., allogeneic T cell, endogenous TCR-deficient T cell, GvHD-minimized T cell), comprising: simultaneously introducing into a precursor T cell a first nucleic acid encoding a Nef protein (e.g., wt Nef, or mutant Nef such as mutant SIV Nef) on one vector, and a second nucleic acid on another vector encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, an 11-13, aa 3840, an 44-46, an 47-49, an 50-52, an 53-55, aa 56-58, an 59-61, an 62-64, an 65-67, an 98-100, an 107-109, an 110-112, aa 137-139, aa 152-154, aa 164-166, aa 167-169, aa 170-172, aa 173-175, aa 176-178, an 178-179, 179-181aa, an 182-184, an 185-187, an 188-190, an 191-193, an 194-196, an 203-205, an 206-208, an 212-214, an 215-217, aa 218-220, an 221-223, aa 8-13, an 44-67, aa 107-112, an 164-196, an 203-208, or aa 212-223: (ii) an 2-4, an 44-46, an 56-58, an 59-61, an 62-64, aa 65-67, aa 98-100, aa 107-109, aa 137-139, aa 152-154, aa 164-166, aa 167-169, aa 176-178, an 178-179, an 179-181, an 185-187, an 188-190, an 194-196, an 203-205, an 44-67, an 164-169, an 176-181, an 185-190: (iii) an 2-4, an 56-58, aa 59-61, aa 62-64, aa 65-67, an 107-109, an 137-139, aa 152-154, aa 164-166, aa 167-169, aa 170-172, aa 173-175, aa 176-178, 178-179aa, aa 179-181, an 182-184, an 185-187, an 188-190, an 194-196, an 203-205, an 56-67, or an 164-190; or (iv) a 2-4, an 56-58, an 59-61, an 62-64, an 65-67, an 107-109, an 137-139, an 152-154, an 164-166, aa 167-169, aa 176-178, aa 178-179, aa 179-181, aa 185-187, aa 188-190, aa 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef. or mutant Nef such as mutant SIV Nef) down-regulates cell surface expression of endogenous TCR, but does not down-modulate (e.g., down-regulate cell surface expression) CAR. In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the first nucleic acid and the second nucleic acid are on the same vector. In some embodiments, the first nucleic acid and the second nucleic acid are operably linked to different promoters. Thus in some embodiments, there is provided a method of producing a modified T cell (e.g., allogeneic T cell, endogenous TCR-deficient T cell. GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, or mutant Nef such as mutant SIV Nef), a second promoter (e.g., PGK), and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain, and (c) an intracellular signaling domain, a first promoter (e.g., PGK), a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, or mutant Nef such as mutant SIV Nef) and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence (e.g., IRES, nucleic acid sequence encoding self-cleaving 2A peptides such as P2A or T2A). Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, or mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, mutant Nef such as mutant SIV Nef), a first linking sequence IRES, an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, mutant Nef such as mutant SIV Nef), a first linking sequence encoding P2A, an optional second linking sequence (e.g., nucleic acid sequence encoding flexible linker such as (GGGS)₃ linker), and a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, or mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence 1RES, an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a CAR comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain, a first linking sequence encoding P2A, an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the extracellular ligand binding domain comprises two or more anti-BCMA sdAbs linked together. In some embodiments, the CAR is monovalent and monospecific. In some embodiments, the CAR is multivalent (e.g., bispecific) and monospecific. In some embodiments, the CAR is multivalent (e.g., bivalent) and multispecific (e.g., bispecific). In some embodiments, the Nef protein comprises the amino acid sequence of any of SEQ ID NOs: 12-22. In some embodiments, the Nef protein is a mutant SIV Nef comprising one of more mutations at amino acid residues at any of (i) aa 2-4, aa 8-10, aa 11-13, aa 38-40, an 44-46, an 47-49, an 50-52, aa 53-55, aa 56-58, aa 59-61, an 62-64, an 65-67, aa 98-100, an 107-109, an 110-112, an 137-139, an 152-154, an 164-166, an 167-169, an 170-172, an 173-175, an 176-178, an 178-179, 179-181aa, an 182-184, an 185-187, an 188-190, an 191-193, an 194-196, an 203-205, an 206-208, an 212-214, an 215-217, an 218-220, an 221-223, an 8-13, an 44-67, an 107-112, an 164-196, an 203-208, or an 212-223; (ii) an 2-4, an 44-46, an 56-58, an 59-61, aa 62-64, an 65-67, an 98-100, an 107-109, an 137-139, an 152-154, an 164-166, an 167-169, aa 176-178, aa 178-179, aa 179-181, aa 185-187, aa 188-190, aa 194-196, aa 203-205, aa 44-67, an 164-169, an 176-181, an 185-190; (iii) an 2-4, an 56-58, an 59-61, an 62-64, an 65-67, an 107-109, aa 137-139, aa 152-154, aa 164-166, aa 167-169, aa 170-172, aa 173-175, aa 176-178, 178-179aa, an 179-181, an 182-184, an 185-187, an 188-190, an 194-196, an 203-205, an 56-67, or an 164-190; or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, aa 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef. or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) CAR. In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, mutant Nef such as mutant SIV Nef), a second promoter (e.g., PGK), and a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof, (d) a transmembrane domain comprising a transmembrane domain of a second TCR subunit (e.g., CD3ε); and (e) an intracellular signaling domain comprising an intracellular signaling domain of a third TCR subunit (e.g., CD3ε), wherein the first, second, and third TCR subunit are all selected from the group consisting of TCRα, TCRβ, TCRγ, TCRδ, CD3ε, CD3γ, and CD3δ, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, a first promoter (e.g., PGK), a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence, e.g., IRES, nucleic acid sequence encoding self-cleaving 2A peptides such as P2A or T2A. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (c) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ, a first linking sequence (e.g., IRES, nucleic acid sequence encoding self-cleaving 2A peptides such as P2A or T2A), an optional second linking sequence (e.g., nucleic acid sequence encoding flexible linker such as (GGGS); linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, an 44-46, an 47-49, an 50-52, an 53-55, an 56-58, an 59-61, an 62-64, an 65-67, an 98-100, aa 107-109, an 110-112, an 137-139, an 152-154, aa 164-166, an 167-169, an 170-172, an 173-175, an 176-178, an 178-179, 179-181aa, an 182-184, aa 185-187, an 188-190, an 191-193, aa 194-196, aa 203-205, aa 206-208, aa 212-214, aa 215-217, aa 218-220, aa 221-223, an 8-13, an 44-67, an 107-112, an 164-196, an 203-208, or an 212-223; (ii) a 2-4, an 44-46, an 56-58, an 59-61, an 62-64, an 65-67, an 98-100, an 107-109, an 137-139, an 152-154, an 164-166, an 167-169, an 176-178, an 178-179, an 179-181, an 185-187, an 188-190, an 194-196, an 203-205, an 44-67, an 164-169, an 176-181, an 185-190: (iii) an 2-4, an 56-58, an 59-61, an 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, an 65-67, an 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190: wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) cTCR. In some embodiments, the functional cTCR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; wherein the first, second, and third TCR co-receptors are all selected from the group consisting of CD4, CD8, and CD28, wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, mutant Nef such as mutant SIV Nef), a second promoter (e.g., PGK), and a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28, a first promoter (e.g., PGK), a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28, wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence, e.g., IRES, nucleic acid sequence encoding self-cleaving 2A peptides such as P2A or T2A. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA. CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28, a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) an 2-4, an 8-10, aa 11-13, aa 38-40, aa 44-46, an 47-49, an 50-52, an 53-55, an 56-58, aa 59-61, an 62-64, an 65-67, aa 98-100, an 107-109, aa 110-112, an 137-139, an 152-154, aa 164-166, aa 167-169, aa 170-172, aa 173-175, aa 176-178, aa 178-179, 179-181aa, an 182-184, an 185-187, an 188-190, aa 191-193, an 194-196, an 203-205, an 206-208, aa 212-214, an 215-217, an 218-220, an 221-223, an 8-13, an 44-67, aa 107-112, an 164-196, an 203-208, or an 212-223; (ii) an 2-4, an 44-46, an 56-58, aa 59-61, an 62-64, an 65-67, an 98-100, an 107-109, aa 137-139, aa 152-154, aa 164-166, aa 167-169, aa 176-178, aa 178-179, aa 179-181, an 185-187, aa 188-190, aa 194-196, aa 203-205, aa 44-67, aa 164-169, aa 176-181, aa 185-190: (iii) an 2-4, an 56-58, an 59-61, an 62-64, an 65-67, an 107-109, an 137-139, an 152-154, an 164-166, aa 167-169, aa 170-172, aa 173-175, aa 176-178, 178-179aa, aa 179-181, aa 182-184, aa 185-187, an 188-190, an 194-196, an 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC. In some embodiments, the functional TAC is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, wherein the first nucleic acid and the second nucleic acid are operably linked to different promoters (e.g., EF1-α and PGK), wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a first promoter (e.g., EF1-α), a first nucleic acid encoding a Nef protein (e.g., wt Nef, mutant Nef such as mutant SIV Nef), a second promoter (e.g., PGK), and a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a second promoter (e.g., EF1-α), a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, a first promoter (e.g., PGK), a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the first nucleic acid and the second nucleic acid are operably linked to the same promoter. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) comprising a first nucleic acid encoding a Nef protein (e.g., wt Nef, mutant Nef such as mutant SIV Nef) and a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, wherein the first nucleic acid and the second nucleic acid are operably linked to the same promoter (e.g., EF1-α), and wherein the Nef protein upon expression results in down-modulation of the endogenous TCR in the modified T cell. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence, e.g., IRES, nucleic acid sequence encoding self-cleaving 2A peptides such as P2A or T2A. Thus in some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a first nucleic acid encoding a Nef protein (e.g., wt Nef, mutant Nef such as mutant SIV Nef), a first linking sequence (e.g., IRES, nucleic acid sequence encoding self-cleaving 2A peptides such as P2A or T2A), an optional second linking sequence (e.g., nucleic acid sequence encoding flexible linker such as (GGGS)₃ linker), and a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε), and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, wherein the Nef protein upon expression results in down-modulation of the endogenous TCR in the modified T cell. In some embodiments, there is provided a method of producing a modified T cell (e.g., allogeneic T cell, endogenous TCR-deficient T cell, GvHD-minimized T cell), comprising introducing into a precursor T cell a vector (e.g., viral vector such as a lentiviral vector) from upstream to downstream: a promoter (e.g., EF1-α), a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ, a first linking sequence (e.g., 1RES, nucleic acid sequence encoding self-cleaving 2A peptides such as P2A or T2A), an optional second linking sequence (e.g., nucleic acid sequence encoding flexible linker such as (GGGS)₃ linker), and a first nucleic acid encoding a Nef protein (e.g., wt Nef, mutant Nef such as mutant SIV Nef), wherein the Nef protein upon expression results in down-modulation of the endogenous TCR in the modified T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, an 11-13, an 38-40, aa 4446, an 47-49, an 50-52, an 53-55, an 56-58, an 59-61, an 62-64, an 65-67, an 98-100, an 107-109, an 110-112, aa 137-139, aa 152-154, aa 164-166, aa 167-169, aa 170-172, aa 173-175, aa 176-178, an 178-179, 179-181aa, an 182-184, an 185-187, an 188-190, an 191-193, an 194-196, an 203-205, aa 206-208, an 212-214, an 215-217, an 218-220, an 221-223, an 8-13, an 44-67, an 107-112, an 164-196, an 203-208, or an 212-223: (ii) an 2-4, an 44-46, an 56-58, an 59-61, an 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190: or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC-like chimeric receptor. In some embodiments, the functional TAC-like chimeric receptor is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the method further comprises formulating the modified T cells expressing the Nef protein (e.g., wt Nef, or mutant Nef such as mutant SIV Nef) with at least one pharmaceutically acceptable carrier. In some embodiments, the method further comprises administering to an individual an effective amount of the modified T cells expressing the Nef protein (e.g., wt Nef, or mutant Nef such as mutant SIV Nef), or an effective amount of the pharmaceutical formulation comprising the modified T cells expressing the Nef protein and at least one pharmaceutically acceptable carrier. In some embodiments, the individual has cancer. In some embodiments, the individual is a human.

### Source of T Cells, Cell Preparation and Culture

Prior to expansion and genetic modification of the T cells (e.g., precursor T cells), a source of T cells is obtained from an individual. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS. PlasmaLyte A, or other saline solution with or without buffer. Alternatively, the undesirable components of the apheresis sample may be removed and the cells directly resuspended in culture media.

In some embodiments, the T cell is provided from an umbilical cord blood bank, a peripheral blood bank, or derived from an induced pluripotent stem cell (iPSC), multipotent and pluripotent stem cell, or a human embryonic stem cell. In some embodiments, the T cells are derived from cell lines. The T cells in some embodiments are obtained from a xenogeneic source, for example, from mouse, rat, non-human primate, and pig. In some embodiments, the T cells are human cells. In some aspects, the T cells are primary cells, such as those isolated directly from a subject and/or isolated from a subject and frozen. In some embodiments, the cells include one or more subsets of T cells, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some cases, the T cell is allogeneic in reference to one or more intended recipients. In some cases, the T cell is suitable for transplantation, such as without inducing GvHD in the recipient.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example. Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids. FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991: Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Whether prior to or after genetic modification of the T cells with the Nef (e.g., wt Nef, or mutant Nef such as mutant SIV Nef) or exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V. DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresis peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a grater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, the methods include assessing expression of one or more markers on the surface of the modified cells or cells to be engineered. In one embodiment, the methods include assessing surface expression of TCR or CD3ε, for example, by affinity-based detection methods such as by flow cytometry. In some aspects, where the method reveals surface expression of the antigen or other marker, the gene encoding the antigen or other marker is disrupted or expression otherwise repressed for example, using the methods described herein.

### Gene-Editing of Endogenous Loci

In some embodiments, the endogenous loci of the T cell such as endogenous TCR loci (e.g., TCRα, TCRβ), is modified by a gene-editing method, prior to or simultaneously with modifying the T cell to express a Nef protein (e.g., wt Nef, or mutant Nef such as mutant SIV Nef) and/or a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, the modification of the endogenous loci is carried out by effecting a disruption in the gene, such as a knock-out, insertion, missense or frameshift mutation, such as a biallelic frameshift mutation, deletion of all or part of the gene, e.g., one or more exon or portion thereof, and/or knock-in. In some embodiments, such locus modification is performed using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

In some embodiments, the modification of endogenous loci (e.g., TCR) is carried out using one or more DNA-binding nucleic acids, such as disruption via an RNA-guided endonuclease (RGEN), or other form of repression by another RNA-guided effector molecule. For example, in some embodiments, the repression is carried out using clustered regularly interspaced short palindromic repeats (CRISPR) and CRISPR-associated (Cas) proteins. See Sander and Joung, Nature Biotechnology. 32 (4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a proto spacer adjacent motif (PAM) sequence, such as typically NGG, or NAG. In this respect, the gRNA is targeted to the desired sequence by modifying the first 20 nucleotides of the guide RNA to correspond to the target DNA sequence. In some embodiments, the gRNA comprises the nucleic acid sequence of SEQ ID NO: 23.

In some embodiments, the CRISPR system induces DSBs at the target site. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In some embodiments, an endogenous locus of a T cell (e.g., endogenous TCR) is modified by CRISPR/Cas system prior to modifying the T cell to express a Nef protein (e.g., wt Nef, or mutant Nef such as mutant SIV Nef) and/or a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, an endogenous loci of a T cell (e.g., endogenous TCR) is modified by CRISPR/Cas system simultaneously with modifying the T cell to express a Nef protein (e.g., wt Nef, or mutant Nef such as mutant SIV Nef) and/or a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the nucleic acid(s) encoding the CRISPR/Cas system and the nucleic acid(s) encoding the Nef protein (e.g., wt Nef, or mutant Nef such as mutant SIV Nef) and/or the functional exogenous receptor (such as engineered TCR (e.g., traditional engineered TCR chimeric TCR (cTCR)), TAC, TAC-like chimeric receptor, or CAR (e.g., antibody-based CAR, ligand/receptor-based CAR or ACTR)) are on the same vector. In some embodiments, the nucleic acid(s) encoding the CRISPR/Cas system and the nucleic acid(s) encoding the Nef protein (e.g., wt Nef, or mutant Nef such as mutant SIV Nef) and/or the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) are on different vectors.

### Isolation and Enrichment of Modified T Cells

In some embodiments, the method described herein further comprise isolating or enriching T cells comprising the first and/or the second nucleic acid. In some embodiments, the method described herein further comprises isolating or enriching CD3ε/γ/δ-negative T cells from the modified T cells expressing the Nef protein (e.g., wt Nef, or mutant Nef such as mutant SIV Nef). In some embodiments, the method described herein further comprises isolating or enriching endogenous TCRα/β-negative T cells from the modified T cell expressing the Nef protein (e.g., wt Nef, or mutant Nef such as mutant SIV Nef). In some embodiments, the method described herein further comprises isolating or enriching CD4+ and/or CD28+ T cells from the modified T cells expressing the Nef protein (e.g., wt Nef, or mutant Nef such as mutant SIV Nef). In some embodiments, the isolation or enrichment of T cells comprises any combinations of the methods described herein.

In some embodiments, the isolation methods include the separation of different cell types based on the absence or presence in the cell of one or more specific molecules, such as surface markers, e.g., surface proteins, intracellular markers, or nucleic acid. In some embodiments, the selection marker is Nef (e.g., wt Nef, or mutant Nef such as mutant SIV Nef), exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), CD4, CD28, CD3ε, CD3γ, CD3δ, CD3ζ, CD69, TCRα, TCRβ, or MHC. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

Also see “Examples” section for isolation and enrichment methods.

### IV. Nef Protein

The methods described herein involve expression of a Nef protein. Also provided are non-naturally occurring mutant Nef proteins (e.g., mutant SIV Nef) which are particularly useful for making the modified T cells described herein.

Wildtype Nef (negative regulatory factor) is a small 27-35 kDa myristoylated protein encoded by primate lentiviruses, including Human Immunodeficiency Viruses (HIV-1 and HIV-2) and Simian Immunodeficiency Virus (SIV). Nef localizes primarily to the cytoplasm but is also partially recruited to the Plasma Membrane (PM). It functions as a virulence factor, which can manipulate the host's cellular machinery and thus allow infection, survival or replication of the pathogen

Nef is highly conserved in all primate lentiviruses. The HIV-2 and SIV Nef proteins are 10-60 amino acids longer than HIV-1 Nef. From N-terminus to C-terminus, a Nef protein comprises the following domains: myristoylation site (involved in CD4 downregulation, MHC I downregulation, and association with signaling molecules, required for inner plasma membrane targeting of Nef and virion incorporation, and thereby for infectivity), N-terminal α-helix (involved in MHC I downregulation and protein kinase recruitment), tyrosine-based AP recruitment (HIV-2/SIV Nef), CD4 binding site (WL residue, involved in CD4 downregulation, characterized for HIV-1 Nef), acidic cluster (involved in MHC I downregulation, interaction with host PACS1 and PACS2), proline-based repeat (involved in MHC I downregulation and SH3 binding), PAK (p21 activated kinase) binding domain (involved in association with signaling molecules and CD4 downregulation), COP I recruitment domain (involved in CD4 downregulation), di-leucine based AP recruitment domain (involved in CD4 downregulation, HIV-1 Nef), and V-ATPase and Raf-1 binding domain (involved in CD4 downregulation and association with signaling molecules).

CD4 is a 55 kDa type I integral cell surface glycoprotein. It is a component of the T cell receptor on MHC class II-restricted cells such as helper/inducer T-lymphocytes and cells of the macrophage/monocyte lineage. It serves as the primary cellular receptor for HIV and SIV.

In some embodiments, the Nef protein is selected from the group consisting of SIV Nef, HIV1 Nef, and HIV2 Nef. In some embodiments, the Nef protein is a wildtype Nef. In some embodiments, the Nef protein comprises an amino acid sequence of any one of SEQ ID NOs: 12-17.

In some embodiments, the Nef protein is obtained or derived from primary HIV-1 subtype C Indian isolates. In some embodiments, the Nef protein is expressed from F2 allele of the Indian isolate encoding the full-length protein (HIV F2-Nef). In some embodiments, the Nef protein is expressed from C2 allele the Indian isolate with in-frame deletions of CD4 binding site, acidic cluster, proline-based repeat, and PAK binding domain (HIV C2-Nef). In some embodiments, the Nef protein is expressed from D2 allele the Indian isolate with in-frame deletions of CD4 binding site (HIV D2-Nef).

In some embodiments, the Nef protein is a mutant Nef, such as Nef proteins comprising one or more of insertion, deletion, point mutation(s), and/or rearrangement. In some embodiments, the present application provide non-naturally occurring mutant Nef proteins, such as non-naturally occurring mutant Nef proteins that do not downregulate an exogenous receptor (such as CAR (e.g., antibody-based CAR, ligand/receptor-based CAR, ACTR), or engineered TCR (e.g., traditional engineered TCR, chimeric TCR, TAC-like chimeric receptor)) when expressed in a T cell. Thus in some embodiments, there is provided a non-naturally occurring mutant Nef protein comprising one or more mutations compared to wildtype Nef, wherein the non-naturally occurring mutant Nef results in no or less downregulate an exogenous receptor compared to a wildtype Nef when expressed in a T cell. The Nef protein may comprise one or more mutations (e.g., non-naturally occurring mutation) in one or more domains or motifs selected from the group consisting of myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, and any combinations thereof.

For example, in some embodiments, the mutant (e.g., non-naturally occurring mutant) Nef comprises one or more mutations in di-leucine based AP recruitment domain. In some embodiments, the mutant (e.g., non-naturally occurring mutant) Nef comprises mutations in di-leucine based AP recruitment domain and PAK binding domain. In some embodiments, the mutant (e.g., non-naturally occurring mutant) Nef comprises mutations in di-leucine based AP recruitment domain, PAK binding domain, COP I recruitment domain, and V-ATPase and Raf-1 binding domain. In some embodiments, the mutant (e.g., non-naturally occurring mutant) Nef comprises one or more mutations in di-leucine based AP recruitment domain, COP I recruitment domain, and V-ATPase and Raf-1 binding domain. In some embodiments, the mutant (e.g., non-naturally occurring mutant) Nef comprises one or more mutations in di-leucine based AP recruitment domain and V-ATPase and Raf-1 binding domain. In some embodiments, the mutant (e.g., non-naturally occurring mutant) Nef comprises a truncation deleting partial or the entire domain.

In some embodiments, the mutant (e.g., non-naturally occurring mutant) Nef protein comprises an amino acid sequence of any one of SEQ ID NOs: 18-22. In some embodiments, the Nef protein comprises one or more mutations (e.g., non-naturally occurring mutation) not in any of the aforementioned domains/motifs. In some embodiments, the mutant (e.g., non-naturally occurring mutant) Nef is a mutant SIV Nef comprising one or more mutations (e.g., mutating at least any of 1, 2, 3, 4, 5, 6, 7, 8, 9, or 10 amino acid residues, such as mutating to Ala) at any of amino acid residues listed in Table 11. In some embodiments, the mutant (e.g., non-naturally occurring mutant) Nef is a mutant SIV Nef comprising one of more mutations (e.g., mutating at least any of 1, 2, 3, 4, 5, 6, 7, 8, 9, or 10 amino acid residues, such as mutating to Ala) at amino acid residues at any of: (i) a 2-4, aa 8-10, aa 11-13, an 38-40, an 44-46, a 47-49, an 50-52, an 53-55, an 56-58, aa 59-61, aa 62-64, an 65-67, an 98-100, aa 107-109, aa 110-112, an 137-139, aa 152-154, aa 164-166, an 167-169, aa 170-172, an 173-175, an 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190; or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, an 185-187, an 188-190, aa 194-196, an 203-205, aa 56-67, an 164-169, an 176-181, or aa 185-190; wherein the amino acid residue position corresponds to that of wildtype SIV Nef.

In some embodiments, the expression of a Nef protein described herein (wildtype or mutant, e.g., non-naturally occurring mutant) in a T cell (e.g., allogeneic T cell) down-modulates endogenous TCR. In some embodiments, endogenous TCR down-modulation comprises down-regulation of cell surface expression of endogenous TCR, CD3ε, CD3δ, and/or CD3γ, and/or interfering with TCR-mediated signal transduction such as T cell activation or T cell proliferation (e.g., by modulating vesicular transport routs that govern the transport of essential TCR proximal machinery such as Lck and LAT to the plasma membrane, and/or by disrupting TCR-induced actin remodeling events essential for the spatio-temporal coordination of TCR proximal signaling machinery). In some embodiments, the cell surface expression of endogenous TCR, CD3ε, CD3δ, and/or CD3γ in a T cell expressing a Nef protein (e.g., wt Nef, or mutant Nef such as mutant SIV Nef) described herein is down-regulated by at least about any of 50%, 60%, 70%, 80%, 90%, or 95% compared to that of a T cell from the same donor source. In some embodiments, the mutant (e.g., non-naturally occurring mutant) Nef that down-modulates (e.g., down-regulates the expression) endogenous TCR is a mutant SIV Nef, which comprises one of more mutations at amino acid residues at any of: (i) an 2-4, an 8-10, an 11-13, an 38-40, an 44-46, an 47-49, an 50-52, an 53-55, an 56-58, an 59-61, an 62-64, an 65-67, an 98-100, an 107-109, an 110-112, aa 137-139, aa 152-154, aa 164-166, aa 167-169, aa 170-172, aa 173-175, aa 176-178, an 178-179, 179-181aa, an 182-184, an 185-187, an 188-190, an 191-193, an 194-196, an 203-205, an 206-208, an 212-214, an 215-217, an 218-220, an 221-223, an 8-13, an 44-67, an 107-112, an 164-196, an 203-208, or an 212-223; (ii) an 2-4, an 44-46, an 56-58, an 59-61, an 62-64, aa 65-67, aa 98-100, aa 107-109, aa 137-139, aa 152-154, aa 164-166, aa 167-169, aa 176-178, aa 178-179, aa 179-181, aa 185-187, aa 188-190, aa 194-196, aa 203-205, aa 44-67, aa 164-169, aa 176-181, aa 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190: or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or as 185-190; wherein the amino acid residue position corresponds to that of wildtype SIV Nef. In some embodiments, the Nef protein comprises the amino acid sequence selected from any of SEQ ID NOs: 12-14 and 18-22. In some embodiments, the mutant Nef (e.g., mutant SIV Nef) down-regulates cell surface expression of endogenous TCR (e.g., TCRα and/or TCRβ). In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef. In some embodiments, the mutant Nef protein (e.g., mutant SIV Nef) down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef. In some embodiments, the mutant Nef protein (e.g., mutant SIV Nef) does not down-regulate cell surface expression of CD4. In some embodiments, the mutant Nef protein (e.g., mutant SIV Nef) down-regulates cell surface expression of CD4. In some embodiments, the mutant Nef protein (e.g., mutant SIV Nef) down-regulates cell surface expression of CD4 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) does not down-regulate cell surface expression of CD28. In some embodiments, the mutant Nef protein (e.g., mutant SIV Nef) down-regulates cell surface expression of CD28. In some embodiments, the mutant Nef protein (e.g., mutant SIV Nef) down-regulates cell surface expression of CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9/c, 10%, 20%, 30%, 40%, 50%, 60%, 70%, 80%, 90%, or 95%) more than that by the wildtype Nef), and does not down-regulates cell surface expression of CD4 and/or CD28. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein (e.g., mutant SIV Nef) down-regulates cell surface expression of endogenous TCR (e.g., TCRα and/or TCRβ), but does not down-modulate (e.g., down-regulate cell surface expression) the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at most about 3% (such as at most about any of 2% or 1%) different from that by the wildtype Nef. In some embodiments, the mutant Nef protein (e.g., mutant SIV Nef) down-regulates cell surface expression of endogenous TCR (e.g., TCRα and/or TCRβ), and down-regulates cell surface expression of the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef.

In some embodiments, the expression of a Nef protein described herein (wildtype or mutant, e.g., non-naturally occurring mutant) in a T cell (e.g., allogeneic T cell) does not alter endogenous CD3ζ expression or CD3ζ-mediated signal transduction, or downregulates endogenous CD3ζ expression and/or down-modulates CD3ζ-mediated signal transduction by at most about any of 50%, 40%, 30%, 20%, 10%, 5%, or less, compared to that of a T cell from the same donor source. In some embodiments, the Nef protein comprises the amino acid sequence selected from any of SEQ ID NOs: 12-14 and 18-22. In some embodiments, the Nef protein is a mutant SIV Nef, which comprises one of more mutations at amino acid residues at any of: (i) aa 2-4, aa 8-10, aa 11-13, aa 38-40, aa 44-46, an 47-49, an 50-52, aa 53-55, an 56-58, an 59-61, an 62-64, aa 65-67, aa 98-100, an 107-109, an 110-112, aa 137-139, an 152-154, an 164-166, an 167-169, an 170-172, an 173-175, an 176-178, an 178-179, 179-181aa, an 182-184, an 185-187, an 188-190, an 191-193, an 194-196, an 203-205, an 206-208, an 212-214, an 215-217, an 218-220, an 221-223, an 8-13, an 44-67, an 107-112, an 164-196, an 203-208, or an 212-223; (ii) an 2-4, an 44-46, an 56-58, an 59-61, an 62-64, an 65-67, an 98-100, an 107-109, an 137-139, an 152-154, an 164-166, an 167-169, an 176-178, an 178-179, an 179-181, an 185-187, an 188-190, an 194-196, an 203-205, an 44-67, an 164-169, an 176-181, an 185-190: (iii) an 2-4, an 56-58, an 59-61, aa 62-64, aa 65-67, aa 107-109, aa 137-139, aa 152-154, aa 164-166, aa 167-169, aa 170-172, an 173-175, an 176-178, 178-179aa, an 179-181, an 182-184, an 185-187, an 188-190, an 194-196, an 203-205, an 56-67, or an 164-190; or (iv) an 2-4, an 56-58, an 59-61, an 62-64, an 65-67, aa 107-109, aa 137-139, aa 152-154, aa 164-166, aa 167-169, aa 176-178, aa 178-179, aa 179-181, an 185-187, an 188-190, an 194-196, an 203-205, an 56-67, an 164-169, an 176-181, or an 185-190; wherein the amino acid residue position corresponds to that of wildtype SIV Nef. The ability of Nef in not affecting or minimally affecting CD3ζ-mediated signal transduction is critical in this invention, because Nef expression is intended for down-modulating endogenous TCR, while eliciting little or no effect on signal transduction of an exogenous receptor (such as CAR (e.g., antibody-based CAR, ligand/receptor-based CAR, ACTR), engineered TCR (e.g., traditional engineered TCR, chimeric TCR. TAC-like chimeric receptor), e.g. or chimeric receptor comprising a ligand binding domain) introduced into the same cell. Nef expression is also desired to elicit little or no effect on expression of an exogenous receptor (such as CAR (e.g., antibody-based CAR, ligand/receptor-based CAR, ACTR), engineered TCR (e.g., traditional engineered TCR, chimeric TCR, TAC-like chimeric receptor), or chimeric receptor comprising a ligand binding domain) introduced into the same cell.

In some embodiments, the expression of a Nef protein described herein (wildtype or mutant, e.g., non-naturally occurring mutant) in a T cell (e.g., allogeneic T cell) does not down-modulate (e.g., down-regulate cell surface expression) exogenous receptor (such as CAR (e.g., antibody-based CAR, ligand/receptor-based CAR, ACTR), engineered TCR (e.g., traditional engineered TCR, chimeric TCR. TAC-like chimeric receptor), or chimeric receptor comprising a ligand binding domain) in the same T cell. In some embodiments, the exogenous receptor (such as CAR (e.g., antibody-based CAR, ligand/receptor-based CAR, ACTR), engineered TCR (e.g., traditional engineered TCR, chimeric TCR, TAC-like chimeric receptor), or chimeric receptor comprising a ligand binding domain) in a modified T cell expressing a Nef protein described herein is down-modulated (e.g., cell surface expression is down-regulated) by at most about any of 50%, 40%, 30%, 20%, 10%, or 5%, compared to when the exogenous receptor is expressed in a T cell from the same donor source without Nef expression. In some embodiments, the cell surface expression and/or the signal transduction of the exogenous receptor (such as CAR (e.g., antibody-based CAR, ligand/receptor-based CAR, ACTR), engineered TCR (e.g., traditional engineered TCR, chimeric TCR, TAC-like chimeric receptor), or chimeric receptor comprising a ligand binding domain) is unaffected, or down-regulated by at most about any of 50%, 40%, 30%, 20%, 10%, or 5%, when the modified T cell expresses a Nef protein described herein. In some embodiments, the Nef protein comprises the amino acid sequence selected from any of SEQ ID NOs: 12-14 and 18-22. In some embodiments, the Nef protein is a mutant SIV Nef, which comprises one of more mutations at amino acid residues at any of: (i) aa 2-4, aa 8-10, aa 11-13, aa 3840, aa 44-46, aa 47-49, aa 50-52, as 53-55, as 56-58, as 59-61, as 62-64, as 65-67, aa 98-100, aa 107-109, aa 110-112, aa 137-139, aa 152-154, aa 164-166, aa 167-169, aa 170-172, as 173-175, as 176-178, as 178-179, 179-181aa, as 182-184, as 185-187, as 188-190, as 191-193, as 194-196, as 203-205, as 206-208, as 212-214, as 215-217, as 218-220, as 221-223, as 8-13, as 44-67, as 107-112, as 164-196, as 203-208, or as 212-223; (ii) as 2-4, as 44-46, as 56-58, as 59-61, as 62-64, as 65-67, as 98-100, as 107-109, as 137-139, as 152-154, as 164-166, as 167-169, as 176-178, as 178-179, as 179-181, as 185-187, as 188-190, as 194-196, as 203-205, as 44-67, as 164-169, as 176-181, as 185-190; (iii) as 2-4, as 56-58, as 59-61, as 62-64, as 65-67, as 107-109, as 137-139, as 152-154, as 164-166, as 167-169, as 170-172, as 173-175, as 176-178, 178-179aa, aa 179-181, aa 182-184, aa 185-187, aa 188-190, aa 194-196, aa 203-205, aa 56-67, or aa 164-190: or (iv) aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190; wherein the amino acid residue position corresponds to that of wildtype SIV Nef.

In some embodiments, the expression of a Nef protein described herein (wildtype or mutant, e.g., non-naturally occurring mutant) in a T cell (e.g., allogeneic T cell) down-modulates endogenous MHC 1, CD4, and/or CD28, such as downregulating cell surface expression of endogenous MHC I, CD4, and/or CD28 (e.g., via endocytosis and degradation). In some embodiments, the cell surface expression of endogenous MHC I, CD4, and/or CD28 in a T cell expressing a Nef protein described herein is down-regulated by at least about any of 50%, 60%, 70%, 80%, 90%, or 95% compared to that of a T cell from the same donor source.

In some embodiments, the expression of a mutant (e.g., non-naturally occurring mutant) Nef protein described herein (e.g., with mutated domains/motifs involved in CD4 or CD28 downregulation) in a T cell (e.g., allogeneic T cell) down-modulates endogenous TCR (and/or MHC I), while having reduced down-modulation effect (at least about 3% (such as at least about any of 4%, 5%, 6%, 7%, 8%, 9%, 10%, 20%, 30%, 40%, 50%, 60%, 70%, 80%, 90%, or 95%) less down-modulation) on endogenous CD4 or CD28 compared to that when a wildtype Nef protein is expressed in a T cell from the same donor source. In some embodiments, the down-modulation effect on endogenous CD4/CD28 comprises down-regulation of cell surface expression of CD4/CD28. In some embodiments, the mutant Nef does not down-modulate (e.g., down-regulate cell surface expression) endogenous CD4. In some embodiments, the mutant Nef does not down-modulate (e.g., down-regulate cell surface expression) endogenous CD28. In some embodiments, the down-regulation of cell surface expression of endogenous CD4 (and/or CD28) is reduced by at least about any of 3%, 4%, 5%, 6%, 7%, 8%, 9%, 10%, 20%, 30%, 40%, 50%, 60%, 70%, 80%, 90%, or 95% when a mutant Nef is expressed in a T cell, compared to that when a wildtype Nef protein is expressed in a T cell from the same donor source. In some embodiments, the expression of a mutant Nef in a T cell down-regulates cell surface expression of endogenous TCR (and/or MHC I) by at least about any of 50%, 60%, 70%, 80%, 90%, 95% compared to that of a T cell from the same donor source, while the down-regulation of cell surface expression of endogenous CD4 (and/or CD28) is reduced by at least about any of 50%, 60%, 70%, 80%, 90%, or 95% compared to that when a wildtype Nef protein is expressed in a T cell from the same donor source. In some embodiments, the mutant Nef protein (e.g., mutant SIV Nef) down-regulates cell surface expression of endogenous TCR (e.g., TCRα and/or TCRβ) no more than about 3% (such as no more than about any of 2% or 1%) differently from that by the wildtype Nef (or down-regulates cell surface expression of endogenous TCR (e.g., TCRα and/or TCRβ) at least about 3% (including equal to 3%; such as at least about any of 3%, 4%, 5%, 6%, 7%, 8%, 9%, 10%, 20%, 30%, 40%, 50%, 60%, 70%, 80%, 90%, or 95%) more than that by the wildtype Nef), and down-regulates cell surface expression of CD4 and/or CD28 at least about 3% (such as at least about any of 4%, 5%, 6%, 7%, 8%, 9%, 10%, 20%, 30%, 40%, 50%, 60%, 70%, 80%, 90%, or 95%) less than that by the wildtype Nef. In some embodiments, the mutant Nef protein comprises an amino acid sequence of any one of SEQ ID NOs: 18-22. In some embodiments, the mutant Nef that has less CD4 and/or CD28 down-regulation effect is a mutant SIV Nef comprising one of more mutations at amino acid residues at any of: (ii) an 2-4, an 44-46, an 56-58, aa 59-61, aa 62-64, an 65-67, an 98-100, an 107-109, an 137-139, an 152-154, an 164-166, an 167-169, an 176-178, an 178-179, an 179-181, an 185-187, aa 188-190, an 194-196, an 203-205, an 44-67, an 164-169, an 176-181, an 185-190: (iii) an 2-4, an 56-58, an 59-61, an 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, an 56-67, or an 164-190; or (iv) an 2-4, aa 56-58, an 59-61, an 62-64, an 65-67, aa 107-109, aa 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef.

In some embodiments, there is provided a non-naturally occurring Nef protein comprising one or more mutations in myristoylation site, N-terminal α-helix, tyrosine-based AP recruitment, CD4 binding site, acidic cluster, proline-based repeat, PAK binding domain. COP I recruitment domain, di-leucine based AP recruitment domain, V-ATPase and Raf-1 binding domain, or any combinations thereof, or comprising one or more mutations not within any of the aforementioned domains/motifs. In some embodiments, there is provided a non-naturally occurring Nef protein comprising one or more mutations at any of: (i) an 2-4, aa 8-10, an 11-13, an 38-40, an 44-46, aa 47-49, an 50-52, an 53-55, an 56-58, an 59-61, an 62-64, an 65-67, an 98-100, aa 107-109, aa 110-112, aa 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, an 218-220, aa 221-223, aa 8-13, aa 44-67, aa 107-112, aa 164-196, aa 203-208, or aa 212-223; (ii) aa 2-4, aa 44-46, aa 56-58, aa 59-61, aa 62-64, aa 65-67, aa 98-100, aa 107-109, an 137-139, aa 152-154, aa 164-166, aa 167-169, aa 176-178, aa 178-179, aa 179-181, aa 185-187, aa 188-190, an 194-196, aa 203-205, an 44-67, an 164-169, aa 176-181, an 185-190; (iii) an 2-4, an 56-58, an 59-61, an 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, aa 188-190, an 194-196, an 203-205, an 56-67, an 164-169, an 176-181, or an 185-190; wherein the amino acid residue position corresponds to that of wildtype SIV Nef. In some embodiments, there is provided a non-naturally occurring Nef protein comprising an amino acid sequence of any one of SEQ ID NOs: 18-22.

Also provided are nucleic acids (e.g., isolated nucleic acid) encoding any of the Nef protein described herein (e.g., wt Nef or mutant Nef, such as non-naturally occurring Nef protein, mutant SIV Nef). Further provided are vectors (e.g., viral vectors such as lentiviral vectors, bacteria expression vectors) comprising a nucleic acid encoding any of the Nef protein described herein (e.g., wt Nef or mutant Nef, such as non-naturally occurring Nef protein, mutant SIV Nef). These vectors can be placed in any of the vectors described herein.

### V. Functional Exogenous Receptor

In some embodiments, the modified T cell expressing a Nef protein described herein (e.g., wt Nef or mutant Nef, such as non-naturally occurring Nef protein, mutant SIV Nef) further expresses a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). The nucleic acid encoding the functional exogenous receptor can be previously present in the precursor T cell or is introduced into the precursor T cell along with (e.g., simultaneously with) the nucleic acid encoding the Nef protein. The functional exogenous receptor can comprise an extracellular ligand binding domain and optionally an intracellular signaling domain. In some embodiments, the functional exogenous receptor is an engineered TCR, such as a traditional engineered TCR (e.g., an engineered TCR specifically recognizing BCMA or BCMA/MHC complex, referred to as “anti-BCMA TCR”) comprising an extracellular ligand binding domain comprising a Vα and a Vβ derived from a wildtype TCR together specifically recognizing an antigen (such as tumor antigen, e.g., BCMA), wherein the Vα, the Vβ, or both, comprise one or more mutations in one or more CDRs relative to the wildtype TCR. T cells expressing traditional engineered TCRs are referred herein as “traditional TCR-T.” In some embodiments, the functional exogenous receptor is a chimeric TCR (cTCR) comprising (a) an extracellular ligand binding domain comprising an antigen-binding fragment (e.g., scFv, sdAb) that specifically recognizes one or more epitopes of a tumor antigen (e.g., BCMA, CD20, CD19); (b) an optional linker; (c) an optional extracellular domain of a first TCR subunit or a portion thereof; (d) a transmembrane domain comprising a transmembrane domain of a second TCR subunit; and (e) an intracellular signaling domain comprising an intracellular signaling domain of a third TCR subunit; wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., CD3ε). In some embodiments, the first, second, and third TCR subunits are different. T cells expressing chimeric TCRs are referred herein as “cTCR-T.” In some embodiments, the functional exogenous receptor is a T cell antigen coupler (TAC) comprising: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an optional extracellular domain derived from a first TCR co-preceptor (such as CD4, CD28, or CD8, e.g., CD8α); (f) a transmembrane comprising a transmembrane of a second TCR co-receptor (such as CD4, CD28, or CD8, e.g., CD8α); and (g) an optional intracellular signaling domain comprising intracellular signaling domain of a third TCR co-receptor (such as CD4, CD28, or CD8, e.g., CD8α). In some embodiments, the first, second, and third TCR co-receptors are the same (e.g., all CD4). In some embodiments, the first, second, and third TCR co-receptors are different. For example, in some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) a full length TCR co-receptor (e.g., CD4, CD8 (e.g., CD8α), or CD28). T cells expressing TACs are referred herein as “TAC-T.” In some embodiments, the functional exogenous receptor is a T cell antigen coupler (TAC)-like chimeric receptor comprising: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a first TCR subunit (e.g., CD3ε); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the first, second, third, and fourth TCR subunits are the same (e.g., CD3ε). In some embodiments, the second, third, and fourth TCR subunits are the same (e.g., CD3ε). In some embodiments, the first, second, third, and fourth TCR subunits are different (e.g., CD3ε). In some embodiments, the second, third, and fourth TCR subunits are the same (e.g., CD3ε) but different from the first TCR subunit (e.g., TCRα). In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a first TCR subunit (e.g., CD3ε); (d) an optional second linker; and (e) a full length second TCR subunit (e.g., CD3ε); wherein the first and second TCR subunits are both selected from the group consisting of TCRα, TCRβ, TCRγ, TCRδ, CD3ε, CD3γ, and CD3δ. In some embodiments, the first and second TCR subunits are the same (e.g., both CD3ε). In some embodiments, the first (e.g., TCRα) and second (e.g., CD3ε) TCR subunits are different. T cells expressing TAC-like chimeric receptors are referred herein as “TAC-like-T.” In some embodiments, the functional exogenous receptor is a non-TCR receptor. In some embodiments, the non-TCR receptor is a chimeric antigen receptor (CAR) comprising: (a) an extracellular ligand binding domain comprising one or more (such as any one of 1, 2, 3, 4, 5, 6 or more) binding moieties (e.g., receptor domain or antibody-based binding domain such as sdAb, scFv) specifically recognizing an antigen (e.g., any of the antigens described herein, such as BCMA, CD20, CD19); (b) a transmembrane domain; and (c) an intracellular signaling domain. In some embodiments, the extracellular ligand binding domain of the CAR comprises one or more (such as any one of 1, 2, 3, 4, 5, 6 or more) binding moieties comprising an antigen-binding fragments (hereinafter referred to as “anti-antigen CAR”, or “antibody-based CAR”, e.g. “anti-BCMA CAR”), such as sdAbs (e.g., anti-BCMA sdAbs) or scFvs (e.g., anti-CD20 scFv, anti-CD19 scFv). In some embodiments, the extracellular ligand binding domain of the CAR comprises one or more binding moieties comprising at least one domain derived from a ligand or the extracellular domain of a receptor (hereinafter also referred to as “ligand/receptor-based CAR”), wherein the ligand or receptor is a cell surface antigen. In some embodiments, the ligand is derived from APRIL or BAFF (ligands of BCMA). T cells expressing CARs are referred herein as “CAR-T.” CARs comprising an extracellular ligand binding domain comprising one or more binding moieties comprising APRIL or BAFF are hereinafter referred to as “BCMA-ligand CAR.” In some embodiments, the receptor is derived from an Fc binding domain, such as an extracellular domain of an Fc receptor (e.g., FcγR). CARs comprising an extracellular ligand binding domain comprising one or more binding moieties comprising an Fc binding domain (e.g., FcγR) is hereinafter also referred to as “antibody-coupled T cell receptor (ACTR)”. T cells expressing ACTRs are referred herein as “ACTR-T.” In some embodiments, when an Fc-containing protein is administered to or co-expressed in an ACTR-T cell, the Fc-containing protein confers binding specificity of the ACTR-expressing T cell to an antigen described herein. In some embodiments, the Fc-containing protein is an Fc-containing antibody (e.g., full-length antibody such as anti-BCMA full-length antibody) or an Fc-fusion protein, such as antigen-binding fragment-Fc fusion protein (e.g., anti-BCMA sdAb-Fc fusion protein, or anti-BCMA HCAb), Fc-receptor/ligand fusion protein (e.g., APRIL-Fc fusion protein), Fc-fusion protein comprising a variable region of a TCR fused to an Fc region of an immunoglobulin G (IgG) (“TCR-Fc fusion protein”, such as anti-BCMA TCR-Fc fusion protein). The ACTR/Fc-containing protein system is hereinafter referred to as “anti-antigen ACTR”, such as “anti-BCMA ACTR”.

Also provided are nucleic acids (e.g., isolated nucleic acid) encoding any of the functional exogenous receptor described herein (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). Further provided are vectors (e.g., viral vectors such as lentiviral vectors) comprising a nucleic acid encoding any of the functional exogenous receptor described herein (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)).

### Antigens

The extracellular ligand binding domain of the functional exogenous receptor described herein (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) can specifically recognize any antigen on a target cell. In some embodiments, the antigen is a cell surface molecule. In some embodiments, the antigen acts as a cell surface marker on target cells associated with a special disease state. In some embodiments, the antigen is a tumor antigen. In some embodiments, the extracellular ligand binding domain specifically recognizes a single tumor antigen. In some embodiments, the extracellular ligand binding domain specifically recognizes one or more epitopes of a single tumor antigen. In some embodiments, the extracellular ligand binding domain specifically recognizes two or more tumor antigens. In some embodiments, the tumor antigen is associated with a B cell malignancy, such as B-cell lymphoma or multiple myeloma (MM). Tumors express a number of proteins that can serve as a target antigen for an immune response, particularly T cell mediated immune responses. The antigens specifically recognized by the extracellular ligand binding domain may be antigens on a single diseased cell or antigens that are expressed on different cells that each contribute to the disease. The antigens specifically recognized by the extracellular ligand binding domain may be directly or indirectly involved in the diseases.

Tumor antigens are proteins that are produced by tumor cells that can elicit an immune response, particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA. HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19. CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: differentiation antigens such as MART-1/MelanA (MART-I), gp 100 (Pmel 17), tyrosinase. TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16. TA-90\Mac-2 binding protein\cyclophilin C-associated protein, TAAL6, TAG72, TLP, and TPS.

In some embodiments, the tumor antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof. In some embodiments, the antigen is expressed on a B cell. In some embodiments, the antigen is BCMA, CD19, or CD20.

In some embodiments, the antigen is a pathogen antigen, such as a fungal, viral, or bacterial antigen. In some embodiments, the fungal antigen is from Aspergillus or Candida. In some embodiments, the viral antigen is from Herpes simplex virus (HSV), respiratory syncytial virus (RSV), metapneumovirus (hMPV), rhinovirus, parainfluenza (PIV), Epstein-Barr virus (EBV), Cytomegalovirus (CMV), JC virus (John Cunningham virus), BK virus, HIV, Zika virus, human coronavirus, norovirus, encephalitis virus, or Ebola.

In some embodiments, the cell surface antigen is a ligand or receptor. In some embodiments, the extracellular ligand binding domain comprises one or more binding moieties comprising at least one domain derived from a ligand or the extracellular domain of a receptor, wherein the ligand or receptor is a cell surface antigen described herein. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A, CD64B, CD64C, CD32A, and CD32B.

### Chimeric Antigen Receptors (CARs)

In some embodiments, the modified T cell expressing a Nef protein described herein (e.g., wt Nef or mutant Nef, such as non-naturally occurring Nef protein, mutant SIV Nef) further expresses a CAR comprising: (a) an extracellular ligand binding domain comprising one or more (such as any one of 1, 2, 3, 4, 5, 6 or more) binding moieties specifically recognizing an antigen (such as any of the antigens described herein, e.g., BCMA, CD19, CD20); (b) a transmembrane domain, and (c) an intracellular signaling domain. In some embodiments, the one or more binding moieties are antibodies or antigen-binding fragments thereof. In some embodiments, the one or more binding moieties are derived from four-chain antibodies. In some embodiments, the one or more binding moieties are derived from camelid antibodies. In some embodiments, the one or more binding moieties are derived from human antibodies. In some embodiments, the one or more binding moieties are selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., anti-BCMA sdAbs). In some embodiments, the extracellular ligand binding domain comprises two or more sdAbs linked together. In some embodiments, the one or more binding moieties are scFvs (e.g., anti-CD19 scFv, anti-CD20 scFv, or CD19×CD20 bispecific scFvs). In some embodiments, the one or more binding moieties are non-antibody binding proteins, such as polypeptide ligands or engineered proteins that bind to an antigen. In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A. CD64B, CD64C, CD32A, and CD32B. In some embodiments, the CAR is monovalent and monospecific. In some embodiments, the CAR is multivalent (e.g., bispecific) and monospecific. In some embodiments, the CAR is multivalent (e.g., bivalent) and multispecific (e.g., bispecific). In some embodiments, the antigen is selected from the group consisting of CD19, CD20, CD22, CD30, CD33, CD3δ, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof. In some embodiments, the antigen is BCMA, CD19, or CD20. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152, CD154, and PD-1. In some embodiments, the transmembrane domain is derived from CD8α. In some embodiments, the intracellular signaling domain comprises a primary intracellular signaling domain of an immune effector cell. In some embodiments, the primary intracellular signaling domain is derived from CD3ζ, CD3γ, CD3ε, CD3δ, FcRγ (FCER1G), FcRβ (Fc Epsilon RIb), CD5, CD22, CD79a, CD79b, CD66d, Fc gamma RIIa, DAP10, and DAP12. In some embodiments, the primary intracellular signaling domain is derived from DAP12, CD3ζ, or CD3γ. In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the co-stimulatory signaling domain comprises a cytoplasmic domain of CD137. In some embodiments, the CAR described herein further comprises a hinge domain located between the C-terminus of the extracellular ligand binding domain and the N-terminus of the transmembrane domain. In some embodiments, the hinge domain is derived from CD8α. In some embodiments, the CAR further comprises a signal peptide located at the N-terminus of the polypeptide. In some embodiments, the signal peptide is derived from CD8α. In some embodiments, the CAR comprises a polypeptide comprising from N-terminus to C-terminus: a CD8a signal peptide, the extracellular ligand binding domain (e.g., one or more sdAbs specifically recognizing one or more epitopes of BCMA, APRIL/BAFF ligand, or Fc receptor), a CD8a hinge domain, a CD8a transmembrane domain, a co-stimulatory signaling domain derived from CD137, and a primary intracellular signaling domain derived from CD3ζ.

In some embodiments, the CAR of the present application is an “anti-BCMA CAR”. In some embodiments, the CAR comprises a polypeptide comprising from N-terminus to C-terminus: a CD8a signal peptide, an extracellular ligand binding domain comprising an anti-BCMA sdAb, a CD8a hinge domain, a CD8a transmembrane domain, a co-stimulatory signaling domain derived from CD137, and a primary intracellular signaling domain derived from CD3ζ. In some embodiments, the CAR comprises a polypeptide comprising from N-terminus to C-terminus: a CD8α signal peptide, an extracellular ligand binding domain comprising a first anti-BCMA sdAb and a second anti-BCMA sdAb, a CD8α hinge domain, a CD8α transmembrane domain, a co-stimulatory signaling domain derived from CD137, and a primary intracellular signaling domain derived from CD3ζ. In some embodiments, the first anti-BCMA sdAb and the second anti-BCMA sdAb are the same. In some embodiments, the first anti-BCMA sdAb and the second anti-BCMA sdAb are different. In some embodiments, the first anti-BCMA sdAb and the second anti-BCMA sdAb specifically bind to the same BCMA epitope. In some embodiments, the first anti-BCMA sdAb and the second anti-BCMA sdAb specifically bind to different BCMA epitopes. In some embodiments, the anti-BCMA CAR comprises an amino acid sequence selected from SEQ ID NOs: 59-61.

In some embodiments, the CAR of the present application is an anti-CD19 CAR. In some embodiments, the extracellular ligand binding domain of the anti-CD19 CAR comprises an anti-CD19 scFv. In some embodiments, the anti-CD19 CAR comprises the amino acid sequence of SEQ ID NO: 58.

In some embodiments, the CAR of the present application is an anti-CD20 CAR. In some embodiments, the extracellular ligand binding domain of the anti-CD20 CAR comprises an anti-CD20 scFv. In some embodiments, the anti-CD20 scFv is derived from anti-CD20 antibodies such as rituximab (e.g., Rituxan®, MabThera®) or Leu-16. In some embodiments, the anti-CD20 CAR comprises the amino acid sequence of SEQ ID NO: 55 or 56.

In some embodiments, the CAR of the present application is an anti-CD19/anti-CD20 bispecific CAR (also referred herein as CD19×CD20 CAR). In some embodiments, the extracellular ligand binding domain of the CD19×CD20 CAR comprises an anti-CD20 scFv and/or an anti-CD19 scFv. In some embodiments, the CD19×CD20 CAR comprises the amino acid sequence of SEQ ID NO: 57.

In some embodiments, the CAR of the present application is a “BCMA-ligand CAR”. In some embodiments, the CAR comprises a polypeptide comprising from N-terminus to C-terminus: a CD8α signal peptide, an extracellular ligand binding domain comprising one or more binding moieties comprising at least one domain derived from APRIL or BAFF, a CD8α hinge domain, a CD8α transmembrane domain, a co-stimulatory signaling domain derived from CD137, and a primary intracellular signaling domain derived from CD3ζ. In some embodiments, the extracellular ligand binding domain comprises an APRIL domain. In some embodiments, the extracellular ligand binding domain comprises a BAFF domain. In some embodiments, the extracellular ligand binding domain comprises an APRIL domain and a BAFF domain.

In some embodiments, the CAR of the present application is an antibody coupled TCR (ACTR). Engineered T cells bearing the ACTR can bind to an Fc-containing protein (such as a monoclonal antibody, e.g., anti-BCMA antibody) which then acts as a bridge to the tumor cells. In some embodiments, the CAR comprises a polypeptide comprising from N-terminus to C-terminus: a CD8α signal peptide, an extracellular ligand binding domain comprising one or more binding moieties comprising an Fc binding domain (such as Fc receptor, e.g., FcγR), a CD8α hinge domain, a CD8α transmembrane domain, a co-stimulatory signaling domain derived from CD137, and a primary intracellular signaling domain derived from CD3ζ. In some embodiments, the FcγR is selected from the group consisting of CD16A (FcγRIIIa), CD16B (FcγRIIIIb), CD64A, CD64B, CD64C. CD32A, and CD32B.

Any CAR known in the art or developed by the inventors, including the CARs described in PCT/CN2017/096938 and PCT/CN2016/094408 (the contents of which are incorporated herein by reference in their entirety), may be used to construct the CARs described herein. Exemplary structures of CARs are shown in FIGS. 15A-15D of PCT/CN2017/096938.

Multivalent and/or Multispecific CAR

In some embodiments, the CAR described herein is a multivalent CAR comprising: (a) an extracellular ligand binding domain comprising two or more (such as any one of 2, 3, 4, 5, 6 or more) binding moieties specifically recognizing an antigen (e.g., any of the antigens described herein); (b) a transmembrane domain; and (c) an intracellular signaling domain. In some embodiments, one or more of the binding moieties are antigen binding fragments. In some embodiments, one or more of the binding moieties comprise single-domain antibodies (e.g., anti-BCMA sdAbs, BCMA VHHs). In some embodiments, one or more of the binding moieties are derived from camelid antibodies. In some embodiments, one or more of the binding moieties are derived from a four-chain antibody. In some embodiments, one or more of the binding moieties are scFvs (e.g., anti-CD20 scFv, anti-CD19 scFv). In some embodiments, one or more of the binding moieties are derived from human antibodies. In some embodiments, one or more of the binding moieties are polypeptide ligands or other non-antibody polypeptides that specifically bind to the antigen. In some embodiments, the multivalent CAR is monospecific, i.e., the multivalent CAR targets a single antigen, and comprises two or more binding sites for the single antigen. In some embodiments, the multivalent CAR is multispecific, i.e., the multivalent CAR targets more than one antigen, and the multivalent CAR comprises two or more binding sites for at least one antigen. The binding moieties specific for the same antigen may bind to the same epitope of the antigen (i.e., “mono-epitope CAR”) or bind to different epitopes (i.e., “multi-epitope CAR” such as bi-epitope CAR or tri-epitope CAR) of the antigen. The binding sites specific for the same antigen may comprise the same or different sdAbs. In some embodiments, the antigen is selected from the group consisting of CD19, CD20, CD22, CD30, CD33, CD3δ, BCMA, CS1, CD138, CD123/IL3Rα, c-Met, gp100, MUC1, IGF-1 receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof. In some embodiments, the antigen is BCMA. CD19, or CD20.

In some embodiments, the CAR described herein is a multivalent (such as bivalent, trivalent, or of higher number of valencies) CAR comprising: (a) an extracellular ligand binding domain comprising a plurality (such as at least about any one of 2, 3, 4, 5, 6, or more) of binding moieties (e.g., sdAb, scFv) specifically binding to an antigen (such as a tumor antigen, e.g., BCMA, CD19, CD20); (b) a transmembrane domain; and (c) an intracellular signaling domain. In some embodiments, the CAR described herein is a multivalent (such as bivalent, trivalent, or of higher number of valencies) CAR comprising: (a) an extracellular ligand binding domain comprising a plurality (such as at least about any one of 2, 3, 4, 5, 6, or more) of sdAbs specifically binding to an antigen (such as a tumor antigen, e.g., BCMA, CD19, CD20); (b) a transmembrane domain; and (c) an intracellular signaling domain. In some embodiments, the CAR described herein is a multivalent (such as bivalent, trivalent, or of higher number of valencies) CAR comprising: (a) an extracellular ligand binding domain comprising a first binding moiety (e.g., sdAb, scFv) specifically binding to a first epitope of an antigen (such as a tumor antigen, e.g., BCMA, CD19, CD20), and a second binding moiety (e.g., sdAb, scFv) specifically binding to a second epitope of the antigen (such as a tumor antigen, e.g., BCMA, CD19, CD20); (b) a transmembrane domain; and (c) an intracellular signaling domain. In some embodiments, the first epitope and the second epitope are different. In some embodiments, the first epitope and the second epitope are the same. In some embodiments, the first binding moiety is an sdAb and the second binding moiety is derived from a human antibody (e.g., an scFv). In some embodiments, the first and second binding moieties are both sdAbs or scFvs. In some embodiments, the first binding moiety is an sdAb and the second binding moiety is a polypeptide ligand or receptor (e.g., APRIL, BAFF, Fc receptor). In some embodiments, the multivalent CAR specifically binds to two different epitopes on an antigen. In some embodiments, the multivalent CAR specifically binds to three or more different epitopes on an antigen. In some embodiments, the CAR described herein is a bivalent CAR comprising: (a) an extracellular ligand binding domain comprising a first sdAb specifically binding to a first epitope of an antigen (such as a tumor antigen, e.g., BCMA), and a second sdAb specifically binding to a second epitope of the antigen (such as a tumor antigen, e.g., BCMA); (b) a transmembrane domain; and (c) an intracellular signaling domain. In some embodiments, the CAR described herein is a bivalent CAR comprising: (a) an extracellular ligand binding domain comprising a first scFv specifically binding to a first epitope of an antigen (such as a tumor antigen, e.g., BCMA, CD19, CD20), and a second scFv specifically binding to a second epitope of the antigen (such as a tumor antigen, e.g., BCMA, CD19, CD20); (b) a transmembrane domain; and (c) an intracellular signaling domain (also referred herein as CD19×CD20 CAR). In some embodiments, the first epitope and the second epitope are different. In some embodiments, the first epitope and the second epitope are the same. In some embodiments, the CAR described herein is a bivalent and bispecific CAR comprising: (a) an extracellular ligand binding domain comprising a first scFv specifically binding to CD19 and a second scFv specifically binding to CD20; (b) a transmembrane domain; and (c) an intracellular signaling domain. e.g.In some embodiments, the antigen is selected from the group consisting of CD19, CD20, CD22, CD30, CD33, CD3δ, BCMA, CS1, CD138, CD123/IL3Rα, c-Met, gp100, MUC1, IGF-I receptor. EpCAM, EGFR/EGFRvIII, HER2, IGF1R, mesothelin, PSMA, WT1. ROR1, CEA, GD-2, NY-ESO-1, MAGE A3, GPC3, Glycolipid F77, PD-L1, PD-L2, and any combination thereof. In some embodiments, the antigen is BCMA, CD19, or CD20.

In some embodiments, the CAR described herein is a bivalent CAR comprising: (a) an extracellular ligand binding domain comprising a first sdAb specifically binding to a first epitope of BCMA (“anti-BCMA sdAb1” or “anti-BCMA VHH1”), and a second sdAb specifically binding to a second epitope of BCMA (“anti-BCMA sdAb2” or “anti-BCMA VHH2”); (b) a transmembrane domain; and (c) an intracellular signaling domain. In some embodiments, anti-BCMA sdAb1 and anti-BCMA sdAb2 are the same. In some embodiments, anti-BCMA sdAb1 and anti-BCMA sdAb2 are different.

### Extracellular Ligand Binding Domain

The extracellular ligand binding domain of the functional exogenous receptor (e.g., chimeric TCR, TAC, TAC-like chimeric receptor, CAR (e.g., antibody-based CAR, ligand/receptor-based CAR, or ACTR)) described herein comprises one or more (such as any one of 1, 2, 3, 4, 5, 6 or more) binding moieties, such as sdAbs. In some embodiments, the one or more binding moieties are antibodies or antigen-binding fragments thereof. In some embodiments, the one or more binding moieties are derived from four-chain antibodies. In some embodiments, the one or more binding moieties are derived from camelid antibodies. In some embodiments, the one or more binding moieties are derived from human antibodies. In some embodiments, the one or more binding moieties are selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., anti-BCMA sdAbs). In some embodiments, the one or more binding moieties are scFvs (e.g., anti-CD19 scFv, anti-CD20 scFv, or CD19-CD20 scFvs). In some embodiments, the one or more binding moieties are non-antibody binding proteins, such as polypeptide ligands or engineered proteins that bind to an antigen. In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A, CD64B, CD64C, CD32A, and CD32B. The binding moieties can be fused to each other directly via peptide bonds, or via peptide linkers.

Single-Domain Antibodies (sdAbs)

In some embodiments, the functional exogenous receptor (e.g., chimeric TCR, TAC, TAC-like chimeric receptor, CAR (e.g., antibody-based CAR, ligand/receptor-based CAR, or ACTR)) comprises an extracellular ligand binding domain comprising one or more sdAbs. The sdAbs may be of the same of different origins, and of the same or different sizes. Exemplary sdAbs include, but are not limited to, heavy chain variable domains from heavy-chain only antibodies (e.g., V_{H}H or V_{NAR}), binding molecules naturally devoid of light chains, single domains (such as V_{H} or V_{L}) derived from conventional 4-chain antibodies, humanized heavy-chain only antibodies, human sdAbs produced by transgenic mice or rats expressing human heavy chain segments, and engineered domains and single domain scaffolds other than those derived from antibodies. Any sdAbs known in the art or developed by the inventors, including the sdAbs described in PCT/CN2017/096938 and PCT/CN2016/094408 (the contents of which are incorporated herein by reference in their entirety), may be used to construct the functional exogenous receptor (e.g., chimeric TCR, TAC, TAC-like chimeric receptor, CAR (e.g., antibody-based CAR, ligand/receptor-based CAR, or ACTR)) described herein. Exemplary structures of CARs are shown in FIGS. 15A-15D of PCT/CN2017/096938. The sdAbs may be derived from any species including, but not limited to mouse, rat, human, camel, llama, lamprey, fish, shark, goat, rabbit, and bovine. Single-domain antibodies contemplated herein also include naturally occurring sdAb molecules from species other than Camelidae and sharks.

In some embodiments, the sdAb is derived from a naturally occurring single-domain antigen binding molecule known as heavy chain antibody devoid of light chains (also referred herein as “heavy chain only antibodies”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a V_{H}H to distinguish it from the conventional V_{H} of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such V_{H}Hs are within the scope of the present application.

V_{H}H molecules from Camelids are about 10 times smaller than IgG molecules. They are single polypeptides and can be very stable, resisting extreme pH and temperature conditions. Moreover, they can be resistant to the action of proteases which is not the case for conventional 4-chain antibodies. Furthermore, in vitro expression of V_{H}H s produces high yield, properly folded functional V_{H}Hs. In addition, antibodies generated in Camelids can recognize epitopes other than those recognized by antibodies generated in vitro through the use of antibody libraries or via immunization of mammals other than Camelids (see, for example, WO9749805). As such, multispecific or multivalent CARs comprising one or more V_{H}H domains may interact more efficiently with targets than multispecific or multivalent CARs comprising antigen binding fragments derived from conventional 4-chain antibodies. Since V_{H}Hs are known to bind into ‘unusual’ epitopes such as cavities or grooves, the affinity of CARs comprising such V_{H}Hs may be more suitable for therapeutic treatment than conventional multispecific polypeptides.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region ofNAR (“IgNARs”) are described in WO 03/014161 and Streltsov (2005) Protein Sci. 14:2901-2909.

In some embodiments, the sdAb is recombinant. CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) V_{H} domain from a conventional 4-chain antibody by one or more of the amino acid residues that occur at the corresponding position(s) in a V_{H}H domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678, Davies and Riechmann FEBS Letters 339: 285-290, 1994; Davies and Riechmann Protein Engineering 9 (6): 531-537, 1996; Riechmann J. Mol. Biol. 259: 957-969, 1996; and Riechmann and Muyldermans J. Immunol. Meth. 231: 25-38, 1999).

In some embodiments, the sdAb is a human sdAb produced by transgenic mice or rats expressing human heavy chain segments. See, e.g., US20090307787A1, U.S. Pat. No. 8,754,287, US20150289489A1. US20100122358A1, and WO2004049794. In some embodiments, the sdAb is affinity matured.

in some embodiments, naturally occurring V_{H}H domains against a particular antigen or target, can be obtained from (naïve or immune) libraries of Camelid V_{H}H sequences. Such methods may or may not involve screening such a library using said antigen or target, or at least one part, fragment, antigenic determinant or epitope thereof using one or more screening techniques known per se. Such libraries and techniques are for example described in WO 99/37681. WO 01/90190, WO 03/025020 and WO 03/035694. Alternatively, improved synthetic or semi-synthetic libraries derived from (naïve or immune) V_{H}H libraries may be used, such as V_{H}H libraries obtained from (naïve or immune) V_{H}H libraries by techniques such as random mutagenesis and/or CDR shuffling, as for example described in WO 00/43507.

In some embodiments, the sdAbs are generated from conventional four-chain antibodies. See, for example, EP 0 368 684, Ward et al. (Nature 1989 Oct. 12; 341 (6242): 544-6), Holt et al., Trends Biotechnol., 2003, 21(11):484-490; WO 06/030220; and WO 06/003388.

### Peptide Linkers

The various binding moieties (such as sdAbs, ligand/receptor domains) in the multispecific or multivalent functional exogenous receptors (e.g., chimeric TCR, TAC, TAC-like chimeric receptor, CAR (e.g., antibody-based CAR, ligand/receptor-based CAR, or ACTR)) described herein may be fused to each other via peptide linkers. In some embodiments, the binding moieties (such as sdAbs, ligand/receptor domains) are directly fused to each other without any peptide linkers. The peptide linkers connecting different binding moieties (such as sdAbs, ligand/receptor domains) may be the same or different. Different domains of the functional exogenous receptors (e.g., chimeric TCR, TAC, TAC-like chimeric receptor, CAR (e.g., antibody-based CAR, ligand/receptor-based CAR, or ACTR)) may also be fused to each other via peptide linkers.

Each peptide linker in a functional exogenous receptor (e.g., chimeric TCR, TAC, TAC-like chimeric receptor, CAR (e.g., antibody-based CAR, ligand/receptor-based CAR, or ACTR)) may have the same or different length and/or sequence depending on the structural and/or functional features of the sdAbs and/or the various domains (e.g., ligand/receptor domains). Each peptide linker may be selected and optimized independently. The length, the degree of flexibility and/or other properties of the peptide linker(s) used in the functional exogenous receptors (e.g., chimeric TCR, TAC, TAC-like chimeric receptor, CAR (e.g., antibody-based CAR, ligand/receptor-based CAR, or ACTR)) may have some influence on properties, including but not limited to the affinity, specificity or avidity for one or more particular antigens or epitopes. For example, longer peptide linkers may be selected to ensure that two adjacent domains do not sterically interfere with one another. For example, in a multivalent or multispecific functional exogenous receptors (e.g., chimeric TCR, TAC, TAC-like chimeric receptor, CAR (e.g., antibody-based CAR, ligand/receptor-based CAR, or ACTR)) described herein that comprise sdAbs directed against a multimeric antigen, the length and flexibility of the peptide linkers are preferably such that it allows each sdAb in the multivalent functional exogenous receptor (e.g., chimeric TCR, TAC, TAC-like chimeric receptor, CAR (e.g., antibody-based CAR, ligand/receptor-based CAR, or ACTR)) to bind to the antigenic determinant on each of the subunits of the multimer. In some embodiments, a short peptide linker may be disposed between the transmembrane domain and the intracellular signaling domain of a functional exogenous receptor (e.g., chimeric TCR, TAC, TAC-like chimeric receptor, CAR (e.g., antibody-based CAR, ligand/receptor-based CAR, or ACTR)). In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. &ee, for example, WO1996/34103. In some embodiments, the peptide linker is a flexible linker. Exemplary flexible linkers include glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. In some embodiments, the peptide linker comprises the amino acid sequence GGGGS (SEQ ID NO: 40), (GGGGS)₂ (SEQ ID NO: 41), (GGGS)₃ (SEQ ID NO: 42), (GGGS)₄ (SEQ ID NO: 43), GGGGSGGGGSGGGGGGSGSGGGGS (SEQ ID NO: 44), GGGGSGGGGSGGGGGGSGSGGGGSGGGGSGGGGS (SEQ ID NO: 45), (GGGGS)₃ (SEQ ID NO: 46), or (GGGGS)₄ (SEQ ID NO: 47).

In some embodiments, the various peptide linkers and their properties described herein also apply to the peptides encoded by the linking sequence employed between the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) and the Nef protein described herein (e.g., wt Nef or mutant Nef, such as non-naturally occurring mutant Nef, mutant SIV Nef). For example, a peptide linker comprises flexible residues (such as glycine and serine) may be added in between the functional exogenous receptor (e.g., chimeric TCR, TAC, TAC-like chimeric receptor, CAR (e.g., antibody-based CAR, ligand/receptor-based CAR, or ACTR)) and the Nef protein (e.g., wt Nef, mutant Nef) when nucleic acids encoding them are on the same vector, to provide enough space for proper folding of both the functional exogenous receptor and the Nef protein, and/or to facilitate cleaving the linking sequence in between (e.g., P2A, T2A). For example, the (GGGS)₃ linker used for the BCMA CAR-P2A-(GGGS)₃-SIV Nef construct described herein.

### Transmembrane Domain

The functional exogenous receptors (e.g., chimeric TCR, TAC, TAC-like chimeric receptor, CAR (e.g., antibody-based CAR, ligand/receptor-based CAR, or ACTR)) of the present application comprise a transmembrane domain that can be directly or indirectly fused to the extracellular ligand binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment. e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28. CD3 epsilon, CD45, CD4, CD5, CD8, CD9, CD16, CD22, CD33, CD3γ, CD64, CD80, CD86, CD134, CD137, CD154, KIRDS2, OX40, CD2, CD27, LFA-1 (CDIIa, CD18), ICOS (CD278), 4-1BB (CD137), GITR, CD40, BAFFR, HVEM (LIGHTR). SLAMF7, NKp80 (KLRF1), CD160, CD19, IL-2R beta. IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CDIIa, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162). LTBR, PAG/Cbp, NKp44, NKp30, NKp46, NKG2D, and/or NKG2C. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152, CD154, and PD-1. In some embodiments, the transmembrane domain is derived from CD8α. In some embodiments, the transmembrane domain is derived from CD28.

Transmembrane domains for use in the functional exogenous receptors (e.g., chimeric TCR, TAC. TAC-like chimeric receptor, CAR (e.g., antibody-based CAR, ligand/receptor-based CAR, or ACTR)) described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the functional exogenous receptors (e.g., chimeric TCR. TAC, TAC-like chimeric receptor, CAR (e.g., antibody-based CAR, ligand/receptor-based CAR, or ACTR)) comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

### Intracellular Signaling Domain

The functional exogenous receptors (e.g., chimeric TCR, TAC, TAC-like chimeric receptor, CAR (e.g., antibody-based CAR, ligand/receptor-based CAR, or ACTR)) of the present application comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “*TAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix (6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, CD3γ, CD3ε, CD3δ, FcRγ (FCER1G), FcRβ (Fc Epsilon Rib), CD5, CD22, CD79a, CD79b. CD66d, Fc gamma RIIa, DAP10, and DAP12. In some embodiments, ITAM-containing primary cytoplasmic signaling sequence is derived from CD3γ, DAP12, or CD3ζ.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain is a cytoplasmic signaling domain of wildtype CD3ζ.

### Co-Stimulatory Signaling Domain

Many immune effector cells (e.g., T cells) require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains, for example, two copies of the co-stimulatory signaling domain of CD28 or CD137 (4-1BB). In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD30 and one or more co-stimulatory signaling domains (e.g., 4-1BB). In some embodiments, the one or more co-stimulatory signaling domains and the primary intracellular signaling domain (such as cytoplasmic signaling domain of CD30 are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ. Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or cosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL 1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1. Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR. TIM-4, TSLP, TSLP R, lymphocyte function associated antigen-1 (LFA-1), and NKG2C.

In some embodiments, the one or more co-stimulatory signaling domain is derived from a co-stimulatory molecule selected from the group consisting of CARD11, CD2 (LFA-2), CDT CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19. CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof.

In some embodiments, the one or more co-stimulatory signaling domain is derived from a co-stimulatory molecule selected from the group consisting of CD27, CD28, 4-1BB, OX40, CD30, CD40, CD3, LFA-1, CD2, CD7, LIGHT, NKG2C, B7-H3 and ligands that specially bind to CD83.

In some embodiments, the intracellular signaling domain in the CAR of the present application comprises a co-stimulatory signaling domain derived from 4-1BB (CD137). In some embodiments, the intracellular signaling domain comprises a cytoplasmic signaling domain of CD3ζ and a co-stimulatory signaling domain of 4-1BB.

In some embodiments, the intracellular signaling domain in the CAR of the present application comprises a co-stimulatory signaling domain derived from CD28. In some embodiments, the intracellular signaling domain comprises a cytoplasmic signaling domain of CD3ζ and a co-stimulatory signaling domain of CD28.

In some embodiments, the intracellular signaling domain in the CAR of the present application comprises a co-stimulatory signaling domain of CD28 and a co-stimulatory signaling domain of CD137. In some embodiments, the intracellular signaling domain comprises a cytoplasmic signaling domain of CD3ζ a co-stimulatory signaling domain of CD28, and a co-stimulatory signaling domain of CD137. In some embodiments, the intracellular signaling domain comprises a polypeptide comprising from the N-terminus to the C-terminus: a co-stimulatory signaling domain of CD28, a co-stimulatory signaling domain of CD137, and a cytoplasmic signaling domain of CD3ζ.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wildtype counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Hinge

The functional exogenous receptor (e.g., chimeric TCR, TAC, TAC-like chimeric receptor, CAR (e.g., antibody-based CAR, ligand/receptor-based CAR, or ACTR)) of the present application may comprise a hinge domain that is located between the C-terminus of the extracellular ligand binding domain and the N-terminus of the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the functional exogenous receptors (e.g., chimeric TCR, TAC, TAC-like chimeric receptor, CAR (e.g., antibody-based CAR, ligand/receptor-based CAR, or ACTR)) described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (G×S)ₙ linker, wherein x and n, independently can be an integer between 3 and 12, including 3, 4, 5, 6, 7, 8, 9, 10, 11, 12, or more.

### Signal Peptide

The functional exogenous receptors (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) of the present application may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the functional exogenous receptors (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein will be evident to one of skill in the art. In some embodiments, the signal peptide is derived from a molecule selected from the group consisting of CD8α, GM-CSF receptor a, and IgG1 heavy chain. In some embodiments, the signal peptide is derived from CD8α.

ACTR is a chimeric protein that combines the Fc receptor (CD16) with the signal transduction domains (4-1BB/CD3ζ). Engineered T cells bearing the ACTR can bind to a monoclonal antibody which then acts as a bridge to the tumor cells.

In some embodiments the functional exogenous receptor is a chimeric receptor comprising (a) an extracellular ligand binding domain that comprises at least one domain derived from a ligand or the extracellular domain of a receptor, wherein the ligand or receptor is a cell surface antigen (e.g., NKG2D, BCMA, IL-3, IL-13); (b) a transmembrane domain; and (c) an intracellular signaling domain.

In some embodiments, the extracellular ligand binding domain comprises at least one domain derived from a ligand of BCMA, e.g., APRIL or BAFF. In some embodiments, the extracellular ligand binding domain comprises an antigen-binding fragment (e.g., sdAb) that specifically recognizes one or more epitopes of BCMA.

### T Cell Antigen Couplers (TACs)

In some embodiments, the functional exogenous receptor of the present application is a T cell antigen coupler (TAC). In some embodiments, the TAC comprises: a) an extracellular ligand binding domain comprising an antigen-binding fragment (e.g., scFv, sdAb) that specifically recognizes one or more epitopes of a tumor antigen (e.g., BCMA, CD19, CD20); b) an optional first linker; c) an extracellular TCR binding domain (e.g., scFv, sdAb) that specifically recognizes the extracellular domain of a TCR subunit (e.g., CD3ε); d) an optional second linker; e) a transmembrane domain comprising a transmembrane domain of a first TCR co-receptor (such as CD4, CD28, or CD8, e.g., CD8α); and f) an intracellular signaling domain comprising an intracellular signaling domain of a second TCR co-receptor (such as CD4, CD28, or CD8, e.g., CD8α). In some embodiments, the first and second TCR co-receptors are both selected from CD4, CD28, and CD8 (e.g. CD8α). In some embodiments, the first and second TCR co-receptors are the same. In some embodiments, the first and second TCR co-receptors are different. e.g., the first TCR co-receptor is CD4 and the second TCR co-receptor is CD8 (e.g., CD8), or the second TCR co-receptor is CD4 and the first TCR co-receptor is CD8 (e.g., CD8α). In some embodiments, the TAC comprises: a) an extracellular ligand binding domain comprising an antigen-binding fragment (e.g., scFv, sdAb) that specifically recognizes one or more epitopes of a tumor antigen (e.g., BCMA, CD19, CD20); b) an optional first linker; c) an extracellular TCR binding domain that specifically recognizes the extracellular domain of a TCR subunit (e.g., CD3ε); d) an optional second linker; and e) a transmembrane domain comprising a transmembrane domain of a TCR co-receptor (such as CD4, CD28, or CD8, e.g., CD8α). In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an optional extracellular domain derived from a first TCR co-preceptor (such as CD4, CD28, or CD8, e.g., CD8α) or a portion thereof; (f) a transmembrane domain comprising a transmembrane domain of a second TCR co-receptor (such as CD4, CD28, or CD8, e.g., CD8α); and (g) an optional intracellular signaling domain comprising a intracellular signaling domain of a third TCR co-receptor (such as CD4, CD28, or CD8, e.g., CD8α). In some embodiments, the first, second, and third TCR co-receptors are all selected from CD4, CD28, and CD8 (e.g. CD8α). In some embodiments, the first, second, and third TCR co-receptors are the same (e.g., are all CD4). In some embodiments, the first, second, and third TCR co-receptors are different. In some embodiments, the intracellular signaling domain of the TAC comprises an intracellular signaling domain of a TCR co-receptor, such as CD4, CD28, or CD8 (e.g., CD8α). In some embodiments, the transmembrane domain of the TAC comprises a transmembrane domain of a TCR co-receptor, such as CD4, CD28, or CD8 (e.g., CD8α). In some embodiments, the TAC does not comprise an extracellular domain (or a portion thereof) of the TCR co-receptor (such as CD4. CD28, or CD8 (e.g., CD8α)). In some embodiments, the TAC does not comprise an extracellular domain or a portion thereof of any TCR co-receptor. In some embodiments, the TAC further comprises a hinge domain located between the C-terminus of the extracellular TCR binding domain (e.g., scFv or sdAb) and the N-terminus of the transmembrane domain (e.g., when there is no extracellular domain of a TCR co-preceptor, and the extracellular TCR binding domain is at C-terminus of the extracellular ligand binding domain). In some embodiments, the TAC further comprises a hinge domain located between the C-terminus of the extracellular ligand binding domain and the N-terminus of the transmembrane domain (e.g., when there is no extracellular domain of a TCR co-preceptor, and the extracellular TCR binding domain is at N-terminus of the extracellular ligand binding domain). Any of the hinge domain and linkers described in the above “Hinge” and “Peptide linkers” subsections can be used herein in TAC. In some embodiments, the TAC does not comprise an intracellular co-stimulatory domain. In some embodiments, the extracellular target binding domain is N-terminal to the extracellular TCR binding domain. In some embodiments, the extracellular ligand binding domain is C-terminal to the extracellular TCR binding domain. In some embodiments, the extracellular ligand binding domain is N-terminal to the extracellular TCR binding domain. In some embodiments, the TCR subunit is selected from the group consisting of TCRα, TCRβ, TCRγ, TCRδ, CD3ε, CD3γ, and CD3δ. In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is monomeric, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, the TAC further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain. In some embodiments, extracellular ligand binding domain comprising an antigen-binding fragment which is an sdAb that specifically binds BCMA (i.e., anti-BCMA sdAb), such as any of the anti-BCMA sdAbs disclosed in PCT/CN2016/094408 and PCT/CN2017/096938, the content of which are incorporated herein by reference in their entirety.

Thus in some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker: (c) an extracellular TCR binding domain that specifically recognizes the extracellular domain of a TCR subunit (e.g., CD3ε); (d) an optional second linker; (e) an optional extracellular domain (full or partial domain) derived from CD4; (f) a transmembrane derived from CD4; and (g) an optional intracellular signaling domain derived from CD4. In some embodiments, the TAC is an anti-CD20 TAC comprising the amino acid sequence of SEQ ID NO: 66. In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an optional extracellular domain (full or partial domain) derived from CD8 (e.g., CD8α); (f) a transmembrane derived from CD8 (e.g., CD8α); and (g) an optional intracellular signaling domain derived from CD8 (e.g., CD8α). In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an optional extracellular domain (full or partial domain) derived from CD28; (f) a transmembrane derived from CD28; and (g) an optional intracellular signaling domain derived from CD28. In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) full length CD4 (excluding signal peptide). In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) full length CD8 (e.g., CD8α; excluding signal peptide). In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) full length CD28 (excluding signal peptide). In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is monomeric. i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, the TAC further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain.

In some embodiments, the TAC comprises the structure (from N-terminus to C-terminus): anti-CD20 scFv-(GGGGS)₃-anti-CD3 scFv-(GGGGS)-CD4 sequence. In some embodiments, the anti-CD20 scFv is derived from Leu-16 antibody. In some embodiments, the anti-CD3 scFv is derived from UCHT1 (e.g., huUCHT1), F6A, L2K, or OKT3. In some embodiments, the CD4 sequence comprises partial extracellular domain, full transmembrane domain, and full intracellular domain of CD4, such as aa 375-458 of a full length CD4 (aa 1 counts starting from signal peptide of CD4). In some embodiments, the TAC comprises amino acid sequence of SEQ ID NO: 66.

### T Cell Antigen Coupler (TAC)-Like Chimeric Receptors

In some embodiments, the functional exogenous receptor of the present application is a T cell antigen coupler (TAC)-like chimeric receptor. In some embodiments, the TAC-like chimeric receptor comprises: a) an extracellular ligand binding domain comprising an antigen-binding fragment (e.g., scFv, sdAb) that specifically recognizes one or more epitopes of a tumor antigen (e.g., BCMA, CD19, CD20); b) an optional first linker; c) an extracellular TCR binding domain (e.g., scFv, sdAb) that specifically recognizes the extracellular domain of a first TCR subunit (e.g., CD3ε) d) an optional second linker; e) a transmembrane domain comprising a transmembrane domain of a second TCR subunit (e.g., CD3ε); and f) an intracellular domain comprising an intracellular domain of a third TCR subunit (e.g., CD3ε); wherein the first, second, and third TCR subunits are all selected from the group consisting of CD3ε, CD3γ, CD3δ, TCRα, TCRβ, TCRγ, and TCRδ. In some embodiments, the second and third TCR subunits are the same, e.g., both are CD3s. In some embodiments, the first, second, and third TCR subunits are the same, e.g., all are CD3s. In some embodiments, the first TCR subunit and the second and third TCR subunits are different, e.g., the first TCR subunit is TCRα and the second and third TCR subunits are CD3ε. In some embodiments, the first, second, and third TCR subunits are all different. In some embodiments, the TAC-like chimeric receptor comprises: a) an extracellular ligand binding domain comprising an antigen-binding fragment (e.g., scFv, sdAb) that specifically recognizes one or more epitopes of a tumor antigen (e.g., BCMA, CD19, CD20); b) an optional first linker; c) an extracellular TCR binding domain (e.g., scFv, sdAb) that specifically recognizes the extracellular domain of a first TCR subunit (e.g., CD3ε); d) an optional second linker; and e) a transmembrane domain comprising a transmembrane domain of a second TCR subunit (e.g., CD3ε); wherein the first and second TCR subunits are both selected from the group consisting of CD3ε, CD3γ, CD3δ, TCRα, TCRβ, TCRγ, and TCRδ. In some embodiments, the first and second TCR subunits are the same, e.g., both are CD3&. In some embodiments, the first and second TCR subunits are different, e.g., the first TCR subunit is TCRα and the second TCR subunit is CD3s. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a first TCR subunit (e.g., CD3ε); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same (e.g., all CD3ε). In some embodiments, the first TCR subunit and the second, third, and fourth TCR subunits are different, e.g., the first TCR subunit is TCRα and the second, third, and fourth TCR subunits are CD3ε. In some embodiments, the first, second, third, and fourth TCR subunits are all different. In some embodiments, the intracellular signaling domain of the TAC-like chimeric receptor comprises an intracellular signaling domain of a TCR subunit, wherein the TCR subunit is selected from the group consisting of CD3ε, CD3γ, CD3δ, TCRα, TCRβ, TCRγ, and TCRδ. In some embodiments, the transmembrane domain of the TAC-like chimeric receptor comprises a transmembrane domain of a TCR subunit, wherein the TCR subunit is selected from the group consisting of CD3ε, CD3γ, CD3δ, TCRα, TCRβ, TCRγ, and TCRδ. In some embodiments, the TAC-like chimeric receptor does not comprise an extracellular domain of the TCR subunit or a portion thereof. In some embodiments, the TAC-like chimeric receptor does not comprise an extracellular domain of any TCR subunit. In some embodiments, the TAC-like chimeric receptor further comprises a hinge domain located between the C-terminus of the extracellular TCR binding domain and the N-terminus of the transmembrane domain (e.g., when there is no extracellular domain of a TCR subunit or a portion thereof, and the extracellular TCR binding domain is at C-terminus of the extracellular ligand binding domain). In some embodiments, the TAC-like chimeric receptor further comprises a hinge domain located between the C-terminus of the extracellular ligand binding domain and the N-terminus of the transmembrane domain (e.g., when there is no extracellular domain of a TCR subunit or a portion thereof, and the extracellular TCR binding domain is at N-terminus of the extracellular ligand binding domain). Any of the hinge domain and linkers described in the above “Hinge” and “Peptide linkers” subsections can be used herein in TAC-like chimeric receptor. In some embodiments, the TAC-like chimeric receptor does not comprise an intracellular signaling domain. In some embodiments, the TAC-like chimeric receptor does not comprise an intracellular co-stimulatory domain. In some embodiments, the extracellular ligand binding domain is N-terminal to the extracellular TCR binding domain. In some embodiments, the extracellular ligand binding domain is C-terminal to the extracellular TCR binding domain. In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is monomeric, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, the TAC-like chimeric receptor further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain. In some embodiments, extracellular ligand binding domain comprising an antigen-binding fragment which is an sdAb that specifically binds BCMA (i.e., anti-BCMA sdAb), such as any of the anti-BCMA sdAbs disclosed in PCT/CN2016/094408 and PCT/CN2017/096938, the content of which are incorporated herein by reference in their entirety.

Thus in some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from CD3ε; (f) a transmembrane derived from CD3ε; and (g) an optional intracellular signaling domain derived from CD3ε. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from CD3γ; (f) a transmembrane derived from CD3γ; and (g) an optional intracellular signaling domain derived from CD3γ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from CD3δ; (f) a transmembrane derived from CD3δ; and (g) an optional intracellular signaling domain derived from CD3δ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from TCRα; (f) a transmembrane derived from TCRα; and (g) an optional intracellular signaling domain derived from TCRα. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from TCRβ; (f) a transmembrane derived from TCRβ; and (g) an optional intracellular signaling domain derived from TCRβ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; (e) an optional extracellular domain derived from TCRγ; (f) a transmembrane derived from TCRγ; and (g) an optional intracellular signaling domain derived from TCRγ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker, (e) an optional extracellular domain derived from TCRδ; (f) a transmembrane derived from TCRδ; and (g) an optional intracellular signaling domain derived from TCRδ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) a full length CD3 (excluding signal peptide). In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker, (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length CD3γ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length CD3δ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length TCRα. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker: (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length TCRβ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length TCRγ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., any of TCRα, TCRβ, TCRγ, TCRδ, CD3ε, CD3γ, CD3δ); (d) an optional second linker; and (e) full length TCRδ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a first TCR subunit (e.g., CD3ε); (d) an optional second linker; (e) an optional hinge; (f) a transmembrane derived from a second TCR subunit (e.g., CD3ε); and (g) an intracellular signaling domain derived from a second TCR subunit (e.g., CD3ε); wherein the first and second TCR subunits are both selected from the group consisting of TCRα, TCRβ, TCRγ, TCRδ, CD3ε, CD3γ, and CD3δ. In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19. CD20). In some embodiments, the extracellular ligand binding domain is monomeric, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, the TAC-like chimeric receptor further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain.

In some embodiments, the TAC-like chimeric receptor comprises: a) an extracellular ligand binding domain comprising an antigen-binding fragment (e.g., scFv, sdAb) that specifically recognizes one or more epitopes of a tumor antigen (e.g., BCMA, CD20, CD19); b) an optional first linker; c) an extracellular TCR binding domain that specifically recognizes the extracellular domain of a TCR subunit (e.g., CD3ε); d) an optional second linker; e) a transmembrane domain comprising a transmembrane domain of a first TCR subunit; and f) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRγ and/or the second TCR subunit is TCRγ. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the TAC-like chimeric receptor does not comprise an extracellular domain of the first and/or the second TCR subunits. In some embodiments, the TAC-like chimeric receptor does not comprise an extracellular domain of any TCR subunits. In some embodiments, the TAC-like chimeric receptor polypeptide does not comprise an intracellular co-stimulatory domain. In some embodiments, the extracellular ligand binding domain is N-terminal to the extracellular TCR binding domain. In some embodiments, the extracellular ligand binding domain is C-terminal to the extracellular TCR binding domain. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) a full length CD3ε (excluding signal peptide); wherein the TCR subunit is selected from the group consisting of TCRα, TCRβ, TCRγ, TCRδ, CD3, CD3γ, and CD3δ. In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is monomeric, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, the TAC-like chimeric receptor further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain.

### Engineered TCRs

In some embodiments, the modified T cell expressing a Nef protein described herein (e.g., wt Nef or mutant Nef, such as non-naturally occurring Nef protein such as mutant SIV Nef) further expresses an engineered TCR (e.g., an engineered TCR specifically recognizing BCMA or BCMA/MHC complex) comprising an extracellular ligand binding domain comprising a Vα and a Vβ derived from a wild type TCR together specifically recognizing an antigen (such as any of the antigens described herein, e.g., tumor antigen, BCMA), wherein the Vα, the Vβ, or both, comprise one or more mutations in one or more CDRs relative to the wild type TCR (hereinafter also referred to as “traditional engineered TCR”). In some embodiments, the mutation leads to amino acid substitutions, such as conservative amino acid substitutions. In some embodiments, the engineered TCR binds to the same cognate peptide-MHC bound by the wild type TCR. In some embodiments, the engineered TCR binds to the same cognate peptide-MHC with higher affinity compared to that bound by the wild type TCR. In some embodiments, the engineered TCR binds to the same cognate peptide-MHC with lower affinity compared to that bound by the wild type TCR. In some embodiments, the engineered TCR binds to a non-cognate peptide-MHC not bound by the wild type TCR. In some embodiments, the engineered TCR is a single chain TCR (scTCR). In some embodiments, the engineered TCR is a dimeric TCR (dTCR). In some embodiments, the wild type TCR binds HLA-A2. In some embodiments, the engineered TCR further comprises an intracellular signaling domain, such as a primary intracellular signaling domain derived from CD3ζ.

In some embodiments, the modified T cell expressing a Nef protein described herein (e.g., wt Nef, or mutant Nef such as non-naturally occurring Nef protein, mutant SIV Nef) further expresses an engineered TCR comprising an extracellular ligand binding domain comprising a Vα and a Vβ derived from a wild type TCR together specifically recognizing BCMA or BCMA-MHC complex, wherein the Vα, the VO, or both, comprise one or more mutations in one or more CDRs relative to the wild type TCR. In some embodiments, the engineered anti-BCMA TCR has higher binding affinity to BCMA than the wildtype anti-BCMA TCR. In some embodiments, the engineered TCR further comprises an intracellular signaling domain, such as a primary intracellular signaling domain derived from CD3ζ.

In some embodiments, the engineered TCR of the present application is a chimeric TCR (cTCR). In some embodiments, cTCR comprises an extracellular ligand binding domain comprising an antigen-binding fragment (such as antibody-based antigen binding domain, e.g., scFv or sdAb) that specifically recognizes one or more epitopes of a tumor antigen (e.g., BCMA, CD19, CD20), fused (directly or indirectly) to the full length or a portion of a TCR subunit, wherein the TCR subunit is selected from the group consisting of TCRα, TCRβ, TCRγ, TCRδ, CD3γ, CD3ε, and CD3δ. The fusion polypeptide can be incorporated into a functional TCR complex along with other endogenous TCR subunits and confer antigen specificity to the TCR complex. In some embodiments, the cTCR extracellular ligand binding domain is fused to the full length or a portion of the CD3ε subunit. The intracellular signaling domain of the cTCR can be derived from the intracellular signaling domain of a TCR subunit, such as intracellular signaling domain of CD3ε. The transmembrane domain of cTCR can be derived from a TCR subunit. In some embodiments, the cTCR intracellular signaling domain and the cTCR transmembrane domain are derived from the same TCR subunit, e.g., both from CD3s. In some embodiments, the cTCR extracellular ligand binding domain and the TCR subunit (full or a portion thereof) can be fused via a linker (such as a GS linker). In some embodiments, the cTCR further comprises an extracellular domain of a TCR subunit or a portion thereof, which can be the same or different from the TCR subunit from which the cTCR intracellular signaling domain and/or cTCR transmembrane domain are derived from. Thus in some embodiments, the cTCR comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional linker; (c) an optional extracellular domain of a first TCR subunit or a portion thereof; (d) a transmembrane domain comprising a transmembrane domain of a second TCR subunit; and (e) an intracellular signaling domain comprising an intracellular signaling domain of a third TCR subunit; wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., CD3ε). In some embodiments, the first, second, and third TCR subunits are different. In some embodiments, the cTCR further comprises a hinge domain located between the C-terminus of the extracellular ligand binding domain and the N-terminus of the transmembrane domain (e.g., when there is no extracellular domain of a TCR subunit or a portion thereof). Any of the hinge domain and linkers described in the above “Hinge” and “Peptide linkers” subsections can be used here in cTCR. In some embodiments, the extracellular ligand binding domain is monovalent and monospecific, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is monomeric, i.e., comprising a single antigen-binding fragment (e.g., scFv, sdAb) that specifically recognizes an epitope of a tumor antigen (e.g., BCMA, CD19, CD20). In some embodiments, the extracellular ligand binding domain is multivalent and monospecific, i.e., comprising two or more antigen-binding fragments (e.g., scFv, sdAb) that specifically recognize the same epitope of a tumor antigen (e.g., BCMA, CD19, CD20). In some embodiments, the extracellular ligand binding domain is multivalent and multispecific, i.e., comprising two or more antigen-binding fragments (e.g., scFv, sdAb) that specifically recognize two or more epitopes of the same tumor antigen or different tumor antigens (e.g., BCMA, CD19, CD20). In some embodiments, extracellular ligand binding domain comprising an antigen-binding fragment which is an sdAb that specifically binds BCMA (i.e., anti-BCMA sdAb), such as any of the anti-BCMA sdAbs disclosed in PCT/CN2016/094408 and PCT/CN2017/096938, the content of which are incorporated herein by reference in their entirety.

Thus, for example, in some embodiments, the modified T cell expressing a Nef protein described herein (e.g., wt Nef, or mutant Nef such as non-naturally occurring Nef protein, mutant SIV Nef) further expresses an anti-CD20 chimeric TCR comprising: a) an extracellular ligand binding domain comprising an antigen-binding fragment (e.g., scFv, sdAb) specifically recognizing CD20; b) an optional linker (such as a GS liner, e.g., (GGGGS)₃); c) an optional extracellular domain of a first TCR subunit or a portion thereof (e.g., CD3ε); d) a transmembrane domain comprising a transmembrane domain of a second TCR subunit (e.g., CD3ε), and e) an intracellular signaling domain comprising an intracellular signaling domain of a third TCR subunit (e.g., CD3ε), wherein the first, second, and third TCR subunits are all selected from the group consisting of TCRα, TCRβ, TCRγ, TCRδ, CD3ε, CD3γ, and CD3δ. In some embodiments, the first, second, and third TCR subunits are the same. In some embodiments, the first, second, and third TCR subunits are different. In some embodiments, the anti-CD20 cTCR comprises: a) anti-CD20 scFv: b) a linker (such as a GS liner, e.g., (GGGGS)₃); and c) a full length CD3ε (excluding signal peptide). In some embodiments, the cTCR is an anti-CD20 cTCR comprising the amino acid sequence of SEQ ID NO: 64. In some embodiments, the modified T cell expressing a Nef protein described herein (e.g., wt Nef, or mutant Nef such as non-naturally occurring Nef protein, mutant SIV Nef) further expresses an anti-BCMA chimeric TCR comprising: a) an extracellular ligand binding domain comprising an antigen-binding fragment (e.g., scFv, sdAb) specifically recognizing BCMA; b) an optional linker (such as a GS liner, e.g., (GGGGS)₃); c) an optional extracellular domain of a first TCR subunit or a portion thereof (e.g., CD3ε); d) a transmembrane domain comprising a transmembrane domain of a second TCR subunit (e.g., CD3ε), and e) an intracellular signaling domain comprising an intracellular signaling domain of a third TCR subunit (e.g., CD3ε), wherein the first, second, and third TCR subunits are all selected from the group consisting of TCRα, TCRβ, TCRγ, TCRδ, CD3ε, CD3γ, and CD3&. In some embodiments, the first, second, and third TCR subunits are the same. In some embodiments, the first, second, and third TCR subunits are different. In some embodiments, the anti-BCMA cTCR comprises: a) anti-BCMA sdAb; b) a linker (such as a GS liner, e.g., (GGGGS)₃); and c) full length CD3ε (excluding signal peptide). In some embodiments, the cTCR transmembrane domain, the cTCR intracellular signaling domain, and the optional extracellular domain of a TCR subunit or a portion thereof are derived from the same TCR subunit. In some embodiments, the cTCR transmembrane domain, the cTCR intracellular signaling domain, and the optional extracellular domain of a TCR subunit or a portion thereof are derived from CD3s. In some embodiments, the cTCR comprises the extracellular ligand binding domain fused to the N-terminus of a full length CD3ε (excluding signal peptide). In some embodiments, the anti-CD20 cTCR has the structure of anti-CD20 scFv-(GGGGS)₃-CD3ε, such as SEQ ID NO: 64. In some embodiments, the anti-BCMA cTCR has the structure of anti-BCMA sdAb-(GGGGS)₃-CD3ε.

### VI. Pharmaceutical Compositions

Further provided by the present application are pharmaceutical compositions comprising any one of the modified T cells (e.g., allogeneic T cells, endogenous TCR-deficient T cell, GvHD-minimized T cell) expressing a Nef protein (e.g., wt Nef, or mutant Nef such as mutant SIV Nef) and/or a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein, and a pharmaceutically acceptable carrier. Pharmaceutical compositions can be prepared by mixing a chimeric antibody immune effector cell engager having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine. Vitamin E, sodium metabisulfite; preservatives, isotonicifiers, stabilizers, metal complexes (e.g. Zn-protein complexes); chelating agents such as EDTA and/or non-ionic surfactants.

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, omithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/mL to about 1.0 mg/mL, preferably about 0.07 mg/mL to about 0.2 mg/mL.

Suitable non-ionic surfactants include polysorbates (20, 40, 60, 65, 80, etc.), polyoxamers (184, 188, etc.), PLURONIC@ polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions described herein may also contain more than one active compound or agent as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

### VII. Methods of Treatment

The present application further provides methods of treating a disease (such as cancer, infectious disease, GvHD, transplantation rejection, autoimmune disorders, or radiation sickness) in an individual comprising administering to the individual an effective amount of any one of the pharmaceutical compositions or the modified T cells (e.g., allogeneic T cell, endogenous TCR-deficient T cell, GvHD-minimized T cell) expressing a Nef protein (e.g., wt Nef, or mutant Nef such as mutant SIV Nef) and/or a functional exogenous receptor (such as CAR (e.g., antibody-based CAR, ligand/receptor-based CAR, ACTR), or engineered TCR (e.g., traditional engineered TCR, chimeric TCR. TAC-like chimeric receptor)) described herein.

The methods described herein are suitable for treating various cancers, including both solid cancer and liquid cancer. The methods are applicable to cancers of all stages, including early stage, advanced stage and metastatic cancer. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In some embodiments, the methods described herein are suitable for treating a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the methods described herein are suitable for treating a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm. Burkitt's lymphoma, diffuse large B cell lymphoma, follicular lymphoma, hairy cell leukemia, small cell- or a large cell-follicular lymphoma, malignant lymphoproliferative conditions. MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm. Waldenstrom macroglobulinemia, or pre-leukemia.

In some embodiments, the cancer is multiple myeloma. In some embodiments, the cancer is stage I, stage II or stage III, and/or stage A or stage B multiple myeloma based on the Durie-Salmon staging system. In some embodiments, the cancer is stage I, stage 11 or stage III multiple myeloma based on the International staging system published by the International Myeloma Working Group (IMWG). In some embodiments, the cancer is monoclonal gammopathy of undetermined significance (MGUS). In some embodiments, the cancer is asymptomatic (smoldering/indolent) myeloma. In some embodiments, the cancer is symptomatic or active myeloma. In some embodiments, the cancer is refractory multiple myeloma. In some embodiments, the cancer is metastatic multiple myeloma. In some embodiments, the individual did not respond to a previous treatment for multiple myeloma. In some embodiments, the individual has progressive disease after a previous treatment of multiple myeloma. In some embodiments, the individual has previously received at least about any one of 2, 3, 4, or more treatment for multiple myeloma. In some embodiments, the cancer is relapsed multiple myeloma.

In some embodiments, the individual has active multiple myeloma. In some embodiments, the individual has clonal bone marrow plasma cells of at least 10%. In some embodiments, the individual has a biopsy-proven bony or extramedullary plasmacytoma. In some embodiments, the individual has evidence of end organ damage that can be attributed to the underlying plasma cell proliferative disorder. In some embodiments, the individual has hypercalcemia, e.g., serum calcium >0.25 mmol/L (>1 mg/dL) higher than the upper limit of normal or >2.75 mmol/L (>11 mg/dL). In some embodiments, the individual has renal insufficiency, e.g., creatinine clearance <40 mL per minute or serum creatinine >177 mol/L (>2 mg/dL). In some embodiments, the individual has anemia, e.g., hemoglobin value of >20 g/L below the lowest limit of normal, or a hemoglobin value <100 g/L. In some embodiments, the individual has one or more bone lesions, e.g., one or more osteolytic lesion on skeletal radiography. CT, or PET/CT. In some embodiments, the individual has one or more of the following biomarkers of malignancy (MDEs); (1) 60% or greater clonal plasma cells on bone marrow examination; (2) serum involved/uninvolved free light chain ratio of 100 or greater, provided the absolute level of the involved light chain is at least 100 mg/L; and (3) more than one focal lesion on MRI that is at least 5 mm or greater in size.

In some embodiments, the methods described herein are suitable for treating an autoimmune disease. Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjögren's syndrome, multiple sclerosis (MS), Hashimoto's thyroiditis, Graves' disease, idiopathic thrombocytopenic purpura, and rheumatoid arthritis (RA).

In some embodiments, the methods described herein are suitable for treating an inflammatory diseases, including autoimmune diseases are also a class of diseases associated with B-cell disorders. Examples of autoimmune diseases include, but are not limited to, acute idiopathic thrombocytopenic purpura, chronic idiopathic thrombocytopenic purpura, dermatomyositis. Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum. Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans. Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris. Wegener's granulomatosis, membranous nephropathy, amyotrophic lateral sclerosis, tabes dorsalis, giant cell arteritis/polymyalgia, pemiciousanemia, rapidly progressive glomerulonephritis, psoriasis, and fibrosing alveolitis. The most common treatments are corticosteroids and cytotoxic drugs, which can be very toxic. These drugs also suppress the entire immune system, can result in serious infection, and have adverse effects on the bone marrow, liver, and kidneys. Other therapeutics that has been used to treat Class III autoimmune diseases to date have been directed against T cells and macrophages. There is a need for more effective methods of treating autoimmune diseases, particularly Class III autoimmune diseases.

Administration of the pharmaceutical compositions may be carried out in any convenient manner, including by injection, ingestion, transfusion, implantation or transplantation. The compositions may be administered to a patient transarterially, subcutaneously, intradermally, intratumorally, intranodally, intramedullary, intramuscularly, intravenously, or intraperitoneally. In some embodiments, the pharmaceutical composition is administered systemically. In some embodiments, the pharmaceutical composition is administered to an individual by infusion, such as intravenous infusion. Infusion techniques for immunotherapy are known in the art (see, e.g., Rosenberg el al., New Eng. J. of Med. 319: 1676 (1988)). In some embodiments, the pharmaceutical composition is administered to an individual by intradermal or subcutaneous injection. In some embodiments, the compositions are administered by intravenous injection. In some embodiments, the compositions are injected directly into a tumor, or a lymph node. In some embodiments, the pharmaceutical composition is administered locally to a site of tumor, such as directly into tumor cells, or to a tissue having tumor cells.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti. J. and Chappell, W. “The Use of Interspecies Scaling in Toxicokinetics.” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

In some embodiments, wherein the pharmaceutical composition comprises any one of the modified T cells expressing Nef (e.g., wt Nef, or mutant Nef such as mutant SIV Nef) and/or exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein, the pharmaceutical composition is administered at a dosage of at least about any of 10⁴, 10⁵, 10⁶, 10⁷, 10⁸, or 10⁹ cells/kg of body weight of the individual. In some embodiments, the pharmaceutical composition is administered at a dosage of any of about 10⁴ to about 10⁵, about 10⁵ to about 10⁶, about 10⁶ to about 10⁷, about 10⁷ to about 10⁸, about 10⁹ to about 10⁹, about 10⁴ to about 10⁹, about 10⁴ to about 10⁶, about 10⁶ to about 10⁸, or about 10⁵ to about 10⁷ cells/kg of body weight of the individual. In some embodiments, the pharmaceutical composition is administered at a dose of at least about any 1×10⁵, 2×10⁵, 3-10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷ cells/kg or more. In some embodiments, the pharmaceutical composition is administered at a dose of about 3-10⁵ to about 7×10⁶ cells/kg, or about 3×10⁶ cells/kg.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

Moreover, dosages may be administered by one or more separate administrations, or by continuous infusion. In some embodiments, the pharmaceutical composition is administered in split doses, such as about any one of 2, 3, 4, 5, or more doses. In some embodiments, the split doses are administered over about a week. In some embodiments, the dose is equally split. In some embodiments, the split doses are about 20%, about 30%, about 40%, or about 50% of the total dose. In some embodiments, the interval between consecutive split doses is about 1 day, 2 days, 3 days or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a CAR comprising a polypeptide comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain; and (2) a pharmaceutically acceptable carrier. In some embodiments, the CAR comprises a polypeptide comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, the CAR is monospecific. In some embodiments, the CAR is multivalent. In some embodiments, the CAR is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, an 44-46, aa 47-49, aa 50-52, aa 53-55, aa 56-58, an 59-61, aa 62-64, an 65-67, an 98-100, an 107-109, aa 110-112, an 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or an 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, an 188-190, an 194-196, aa 203-205, aa 44-67, an 164-169, an 176-181, an 185-190; (iii) an 2-4, an 56-58, an 59-61, an 62-64, an 65-67, an 107-109, an 137-139, an 152-154, an 164-166, aa 167-169, aa 170-172, aa 173-175, aa 176-178, 178-179aa, aa 179-181, aa 182-184, an 185-187, an 188-190, an 194-196, aa 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) CAR In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; and (2) a pharmaceutically acceptable carrier. In some embodiments, the first, second, and third TCR subunits are the same (e.g., all CD3ε). In some embodiments, the first, second, and third TCR subunits are different. In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide); and (2) a pharmaceutically acceptable carrier. In some embodiments, the cTCR is monospecific. In some embodiments, the cTCR is multivalent. In some embodiments, the cTCR is multispecific. In some embodiments, the cTCR is an anti-CD20 cTCR comprising the amino acid sequence of SEQ ID NO: 64. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) a 2-4, an 8-10, an 11-13, an 38-40, an 44-46, a 47-49, an 50-52, an 53-55, an 56-58, aa 59-61, aa 62-64, an 65-67, an 98-100, an 107-109, aa 110-112, an 137-139, aa 152-154, an 164-166, an 167-169, an 170-172, an 173-175, an 176-178, an 178-179, 179-181aa, aa 182-184, aa 185-187, aa 188-190, aa 191-193, aa 194-196, aa 203-205, aa 206-208, aa 212-214, aa 215-217, aa 218-220, aa 221-223, an 8-13, aa 44-67, aa 107-112, an 164-196, an 203-208, or an 212-223; (ii) an 2-4, an 44-46, an 56-58, an 59-61, an 62-64, an 65-67, an 98-100, an 107-109, an 137-139, an 152-154, an 164-166, an 167-169, an 176-178, an 178-179, an 179-181, an 185-187, an 188-190, an 194-196, an 203-205, an 44-67, an 164-169, an 176-181, an 185-190; (iii) a 2-4, an 56-58, an 59-61, an 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, aa 194-196, aa 203-205, aa 56-67, or aa 164-190: or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) cTCR. In some embodiments, the functional cTCR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε) (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3&; wherein the first, second, and third TCR co-receptors are all selected from the group consisting of CD4, CD8, and CD28; and (2) a pharmaceutically acceptable carrier. In some embodiments, the first, second, and third TCR co-receptors are the same. In some embodiments, the first, second, and third TCR co-receptors are different. In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ; and (2) a pharmaceutically acceptable carrier. In some embodiments, the TAC is an anti-CD20 TAC comprising the amino acid sequence of SEQ ID NO: 66. In some embodiments, the TAC is monospecific. In some embodiments, the TAC is multivalent. In some embodiments, the TAC is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) an 2-4, an 8-10, aa 11-13, an 38-40, aa 44-46, an 47-49, an 50-52, aa 53-55, an 56-58, an 59-61, an 62-64, aa 65-67, aa 98-100, aa 107-109, aa 110-112, aa 137-139, aa 152-154, aa 164-166, aa 167-169, aa 170-172, an 173-175, an 176-178, an 178-179, 179-181aa, aa 182-184, an 185-187, an 188-190, an 191-193, aa 194-196, aa 203-205, aa 206-208, aa 212-214, aa 215-217, aa 218-220, aa 221-223, an 8-13, an 44-67, an 107-112, an 164-196, an 203-208, or an 212-223; (ii) an 2-4, an 44-46, an 56-58, an 59-61, an 62-64, an 65-67, an 98-100, an 107-109, aa 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, aa 185-190; (iii) an 2-4, an 56-58, an 59-61, aa 62-64, an 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, an 56-67, or an 164-190; or (iv) an 2-4, aa 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef. or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC. In some embodiments, the functional TAC is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef, or mutant Nef such as mutant SIV Nef) and a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof, (f) a transmembrane domain comprising a transmembrane domain of a third TCR subunit (e.g., CD3ε), and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; and (2) a pharmaceutically acceptable carrier. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, there is provided a method of treating an individual having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a Nef protein (e.g., wt Nef. or mutant Nef such as mutant SIV Nef) and a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ; and (2) a pharmaceutically acceptable carrier. In some embodiments, the TAC is monospecific. In some embodiments, the TAC is multivalent. In some embodiments, the TAC is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, aa 44-46, aa 47-49, aa 50-52, aa 53-55, aa 56-58, aa 59-61, aa 62-64, an 65-67, aa 98-100, aa 107-109, an 110-112, an 137-139, aa 152-154, aa 164-166, an 167-169, aa 170-172, an 173-175, an 176-178, an 178-179, 179-181aa, an 182-184, aa 185-187, aa 188-190, aa 191-193, aa 194-196, aa 203-205, aa 206-208, aa 212-214, an 215-217, an 218-220, aa 221-223, aa 8-13, aa 44-67, an 107-112, an 164-196, an 203-208, or aa 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, aa 188-190, aa 194-196, aa 203-205, aa 44-67, aa 164-169, aa 176-181, aa 185-190; (iii) a 2-4, an 56-58, an 59-61, an 62-64, an 65-67, an 107-109, an 137-139, an 152-154, an 164-166, an 167-169, an 170-172, an 173-175, an 176-178, 178-179aa, an 179-181, an 182-184, an 185-187, an 188-190, an 194-196, an 203-205, an 56-67, or an 164-190: or (iv) an 2-4, an 56-58, an 59-61, an 62-64, aa 65-67, an 107-109, an 137-139, an 152-154, an 164-166, an 167-169, an 176-178, an 178-179, an 179-181, an 185-187, an 188-190, an 194-196, aa 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC-like chimeric receptor. In some embodiments, the functional TAC-like chimeric receptor is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the Nef protein (e.g., wt Nef, or mutant Nef such as mutant SIV Nef) down-modulates endogenous TCR, MHC, CD3ε, CD3γ, and/or CD3S in the modified T cell, such as down-regulating cell surface expression of endogenous TCR, MHC, CD3s. CD3γ, and/or CD3δ by at least about any of 50%, 60%, 70%, 80%, 90%, or 95%. In some embodiments, the Nef protein (e.g., mutant Nef such as mutant SIV Nef) does not down-modulate (e.g., down-regulate expression) CD3ζ, CD4, CD28, and/or the exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)), or down-modulates CD3γ, CD4, CD28, and/or the exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR) by at most about any of 50%, 40%, 30%, 20%, 10%, or 5%.

In some embodiments, the Nef protein is selected from the group consisting of SIV Nef, HIV1 Nef, HIV2 Nef, and their homologs. In some embodiments, the Nef protein is a wildtype Nef. In some embodiments, the Nef protein is a mutant Nef. In some embodiments, the mutant Nef comprises one or more mutations in myristoylation site, N-terminal α-helix, tyrosine-based AP recruitment, CD4 binding site, acidic cluster, proline-based repeat, PAK binding domain, COP 1 recruitment domain, di-leucine based AP recruitment domain, V-ATPase and Raf-1 binding domain, or any combinations thereof. In some embodiments, the mutation comprises insertion, deletion, point mutation(s), and/or rearrangement. In some embodiments, the Nef protein comprises an amino acid sequence of any one of SEQ ID NOs: 12-22. In some embodiments, the Nef protein is a mutant SIV Nef that comprises one or more mutations (e.g., mutating at least 1, 2, 3, 4, 5, 6, 7, 8, 9, 10, or more amino acid residues, such as mutating to Ala) at any of amino acid residues listed in Table 11. In some embodiments, the Nef protein is a mutant SIV Nef comprising one of more mutations at amino acid residues at any of: (i) aa 2-4, aa 8-10, aa 11-13, a 38-40, an 44-46, aa 4749, an 50-52, an 53-55, aa 56-58, aa 59-61, an 62-64, an 65-67, an 98-100, an 107-109, an 110-112, an 137-139, an 152-154, an 164-166, an 167-169, an 170-172, aa 173-175, aa 176-178, aa 178-179, 179-181aa, aa 182-184, aa 185-187, aa 188-190, aa 191-193, an 194-196, aa 203-205, an 206-208, an 212-214, aa 215-217, an 218-220, an 221-223, an 8-13, an 44-67, aa 107-112, an 164-196, an 203-208, or an 212-223: (ii) a 2-4, an 44-46, an 56-58, aa 59-61, aa 62-64, aa 65-67, aa 98-100, an 107-109, an 137-139, aa 152-154, an 164-166, aa 167-169, aa 176-178, aa 178-179, aa 179-181, aa 185-187, aa 188-190, aa 194-196, aa 203-205, an 44-67, aa 164-169, an 176-181, aa 185-190; (iii) an 2-4, an 56-58, an 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190; or (iv) a 2-4, an 56-58, aa 59-61, an 62-64, an 65-67, an 107-109, aa 137-139, aa 152-154, aa 164-166, aa 167-169, aa 176-178, aa 178-179, aa 179-181, aa 185-187, an 188-190, an 194-196, aa 203-205, an 56-67, an 164-169, an 176-181, or an 185-190: wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the disease is cancer. In some embodiments, the cancer is multiple myeloma, such as relapsed or refractory multiple myeloma. In some embodiments, the treatment effect comprises causing an objective clinical response in the individual. In some embodiments, Stringent Clinical Response (sCR) is obtained in the individual. In some embodiments, the treatment effect comprises causing disease remission (partial or complete) in the individual. In some the clinical remission is obtained after no more than about any one of 6 months, 5 months, 4 months, 3 months, 2 months, 1 months or less after the individual receives the pharmaceutical composition. In some embodiments, the treatment effect comprises preventing relapse or disease progression of the cancer in the individual. In some embodiments, the relapse or disease progression is prevented for at least about 6 months, 1 year, 2 years, 3 years, 4 years, 5 years or more. In some embodiments, the treatment effect comprises prolonging survival (such as disease free survival) in the individual. In some embodiments, the treatment effect comprises improving quality of life in an individual. In some embodiments, the treatment effect comprises inhibiting growth or reducing the size of a solid or lymphatic tumor.

In some embodiments, the size of the solid or lymphatic tumor is reduced for at least about 10% (including for example at least about any of 20%, 30%, 40%, 60%, 70%, 80%, 90%, or 100%). In some embodiments, a method of inhibiting growth or reducing the size of a solid or lymphatic tumor in an individual is provided. In some embodiments, the treatment effect comprises inhibiting tumor metastasis in the individual. In some embodiments, at least about 10% (including for example at least about any of 20%, 30%, 40%, 60%, 70%, 80%, 90%, or 100%) metastasis is inhibited. In some embodiments, a method of inhibiting metastasis to lymph node is provided. In some embodiments, a method of inhibiting metastasis to the lung is provided. In some embodiments, a method of inhibiting metastasis to the liver is provided. Metastasis can be assessed by any known methods in the art, such as by blood tests, bone scans, x-ray scans, CT scans, PET scans, and biopsy.

The invention is also directed to methods of reducing or ameliorating, or preventing or treating, diseases and disorders using the modified T cells (e.g., allogeneic T cell) expressing Nef (or Nef+ functional exogenous receptor) described herein, isolated populations thereof, or pharmaceutical compositions comprising the same. In some embodiments, the modified T cells (e.g., allogeneic T cell) expressing Nef (or Nef+ functional exogenous receptor) described herein, isolated populations thereof, or pharmaceutical compositions comprising the same are used to reduce or ameliorate, or prevent or treat, cancer, infection, one or more autoimmune disorders, radiation sickness, or to prevent or treat graft versus host disease (GvHD) or transplantation rejection in a subject undergoing transplant surgery.

The modified T cells (e.g., allogeneic T cell) expressing Nef (or Nef+ functional exogenous receptor), isolated populations thereof, or pharmaceutical compositions comprising the same are useful in altering autoimmune or transplant rejection because these T cells can be grown in TGF-β during development and will differentiate to become induced T regulatory cells. In one embodiment, the functional exogenous receptor (e.g. such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is used to give these induced T regulatory cells the functional specificity that is required for them to perform their inhibitory function at the tissue site of disease. Thus, a large number of antigen-specific regulatory T cells are grown for use in patients. The expression of FoxP3, which is essential for T regulatory cell differentiation, can be analyzed by flow cytometry, and functional inhibition of T cell proliferation by these T regulatory cells can be analyzed by examining decreases in T cell proliferation after anti-CD3 stimulation upon co-culture.

Another embodiment of the invention is directed to the use of modified T cells (e.g., allogeneic T cell) expressing Nef (or Nef+ functional exogenous receptor), isolated populations thereof, or pharmaceutical compositions comprising the same for the prevention or treatment of radiation sickness. One challenge after radiation treatment or exposure (e.g. dirty bomb exposure, radiation leak) or other condition that ablates bone marrow cells (certain drug therapies) is to reconstitute the hematopoietic system. In patients undergoing a bone marrow transplant, the absolute lymphocyte count on day 15 post-transplant is correlated with successful outcome. Those patients with a high lymphocyte count reconstitute well, so it is important to have a good lymphocyte reconstitution. The reason for this effect is unclear, but it may be due to lymphocyte protection from infection and/or production of growth factors that favors hematopoietic reconstitution.

In some embodiments, the present invention also provides a method of increasing persistence and/or engraftment of donor T cells in an individual, comprising 1) providing an allogeneic T cell; and 2) introducing into the allogeneic T cell a first nucleic acid encoding a Nef protein (e.g., wt Nef, or mutant Nef such as mutant SIV Nef), wherein the Nef protein upon expression results in down-modulation of the endogenous TCR of the allogeneic T cell. In some embodiments, the allogeneic T cell is an allogeneic CAR-T cell, engineered TCR-T cell (e.g., cTCR-T cell). TAC-T cell. TAC-like-T cell. In some embodiments, the method further comprises introducing into the allogeneic T cell a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the second nucleic acid encodes a CAR. In some embodiments, the CAR comprises a polypeptide comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain. In some embodiments, the CAR comprises a polypeptide comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, the CAR comprises a polypeptide comprising: (a) an extracellular ligand binding domain comprising one or more (such as any one of 1, 2, 3, 4, 5, 6 or more) anti-CD19 scFvs; (b) a transmembrane domain; and (c) an intracellular signaling domain. In some embodiments, the CAR comprises a polypeptide comprising: (a) an extracellular ligand binding domain comprising one or more (such as any one of 1, 2, 3, 4, 5, 6 or more) anti-CD20 scFvs; (b) a transmembrane domain; and (c) an intracellular signaling domain. In some embodiments, the CAR comprises a polypeptide comprising: (a) an extracellular ligand binding domain comprising one anti-CD20 scFv and one anti-CD19 scFv fused directly or indirectly (e.g., via a linker) together; (b) a transmembrane domain; and (c) an intracellular signaling domain. In some embodiments, the second nucleic acid encodes a traditional engineered TCR. In some embodiments, the second nucleic acid encodes an ACTR. In some embodiments, the second nucleic acid encodes a cTCR. In some embodiments, the cTCR comprises (a) an extracellular ligand binding domain comprising an antigen-binding fragment (e.g., scFv, sdAb) that specifically recognizes one or more epitopes of a tumor antigen (e.g., BCMA, CD20, CD19); (b) an optional linker; (c) an optional extracellular domain of a first TCR subunit or a portion thereof; (d) a transmembrane domain comprising a transmembrane domain of a second TCR subunit; and (e) an intracellular signaling domain comprising an intracellular signaling domain of a third TCR subunit; wherein the first, second, and third TCR subunit are all selected from the group consisting of TCRα, TCRβ, TCRγ, TCRδ, CD3ε, CD3γ, and CD3δ. In some embodiments, the cTCR comprises (a) an extracellular ligand binding domain comprising an antigen-binding fragment (e.g., scFv, sdAb) that specifically recognizes one or more epitopes of a tumor antigen (e.g., BCMA, CD20, CD19); (b) an optional linker; and (e) a full length CD3ε (excluding signal peptide). In some embodiments, the cTCR is an anti-CD20 cTCR comprising the amino acid sequence of SEQ ID NO: 64. In some embodiments, the second nucleic acid encodes a TAC. In some embodiments, the TAC comprises (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an optional extracellular domain derived from a first TCR co-preceptor (such as CD4, CD28, or CD8. e.g., CD8α); (f) a transmembrane comprising a transmembrane of a second TCR co-receptor (such as CD4, CD28, or CD8, e.g., CD8α); and (g) an optional intracellular signaling domain comprising intracellular signaling domain of a third TCR co-receptor (such as CD4, CD28, or CD8, e.g., CD8α). In some embodiments, the TAC comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ. In some embodiments, the TAC is an anti-CD20 TAC comprising the amino acid sequence of SEQ ID NO: 66. In some embodiments, the second nucleic acid encodes a TAC-like chimeric receptor. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a first TCR subunit (e.g., CD3ε); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the TAC-like chimeric receptor comprises: (a) an extracellular ligand binding domain comprising an antigen-binding fragment (e.g., scFv, sdAb) that specifically recognizes one or more epitopes of a tumor antigen (e.g., BCMA, CD20, CD19); (b) an optional first linker; (c) an extracellular TCR binding domain that specifically recognizes the extracellular domain of a TCR subunit (e.g., CD3ε); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ. In some embodiments, the functional exogenous receptor (such as CAR (e.g., antibody-based CAR, ligand/receptor based CAR, ACTR), engineered TCR (e.g., traditional engineered TCR, cTCR), TAC. TAC-like chimeric receptor) is monovalent and monospecific. In some embodiments, the functional exogenous receptor (such as CAR (e.g., antibody-based CAR, ligand/receptor based CAR. ACTR), engineered TCR (e.g., traditional engineered TCR, cTCR), TAC, TAC-like chimeric receptor) is multivalent and monospecific. In some embodiments, the functional exogenous receptor (such as CAR (e.g., antibody-based CAR, ligand/receptor based CAR, ACTR), engineered TCR (e.g., traditional engineered TCR, cTCR), TAC, TAC-like chimeric receptor) is multispecific (and multivalent). In some embodiments, the first nucleic acid and the second nucleic acid are on separate vectors. In some embodiments, the first nucleic acid and the second nucleic acid are on the same vector. Thus in some embodiments, the present invention provides a method of increasing persistence and/or engraftment of donor T cells in an individual, comprising 1) providing an allogeneic T cell; and 2) introducing into the allogeneic T cell a vector (e.g., viral vector, lentiviral vector) comprising a first nucleic acid encoding a Nef protein (e.g., wt Nef, or mutant Nef such as mutant SIV Nef), and a second nucleic acid encoding a e.g.e.g. functional exogenous receptor described herein (such as CAR (e.g., antibody-based CAR, ligand/receptor based CAR, ACTR), engineered TCR (e.g., traditional engineered TCR, cTCR), TAC, TAC-like chimeric receptor); wherein the Nef protein upon expression results in down-modulation of the endogenous TCR of the allogeneic T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, aa 11-13, aa 38-40, aa 44-46, aa 47-49, aa 50-52, aa 53-55, aa 56-58, aa 59-61, an 62-64, aa 65-67, an 98-100, an 107-109, an 110-112, an 137-139, an 152-154, aa 164-166, aa 167-169, aa 170-172, aa 173-175, aa 176-178, aa 178-179, 179-181aa, an 182-184, aa 185-187, aa 188-190, aa 191-193, aa 194-196, aa 203-205, aa 206-208, aa 212-214, an 215-217, an 218-220, an 221-223, aa 8-13, an 44-67, an 107-112, aa 164-196, an 203-208, or aa 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, an 188-190, an 194-196, an 203-205, an 44-67, an 164-169, an 176-181, an 185-190; (iii) an 2-4, an 56-58, an 59-61, an 62-64, an 65-67, an 107-109, an 137-139, an 152-154, an 164-166, aa 167-169, aa 170-172, aa 173-175, aa 176-178, 178-179aa, aa 179-181, aa 182-184, aa 185-187, an 188-190, an 194-196, an 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR. CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the first nucleic acid and the second nucleic acid are operably linked to the same promoter. In some embodiments, the first nucleic acid and the second nucleic acid are operably linked to different promoters. In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α or PGK. In some embodiments, the first nucleic acid is upstream of the second nucleic acid. In some embodiments, the first nucleic acid is downstream of the second nucleic acid. In some embodiments, the first nucleic acid and the second nucleic acid are connected via a linking sequence. In some embodiments, the linking sequence is any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one. In some embodiments, the linking sequence is IRES or nucleic acid encoding P2A. In some embodiments, the vector is a viral vector.

In some embodiments, the viral vector selected from the group consisting of adenoviral vector, adeno-associated virus vector, retroviral vector, vaccinia vector, lentiviral vector, herpes simplex viral vector, and derivatives thereof. In some embodiments, the vector is a non-viral vector, such as episomal expression vector, Enhanced Episomal Vector (EEV), PiggyBac Transposase Vector, or Sleeping Beauty (SB) transposon system.

In some embodiments, the present invention also provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a first nucleic acid encoding a Nef protein (e.g., wt Nef, or mutant Nef such as mutant SIV Nef), wherein the Nef protein upon expression results in down-modulation of the endogenous TCR of the allogeneic T cell. In some embodiments, the allogeneic T cell is an allogeneic CAR-T cell, TCR-T cell (e.g., cTCR-T cell), TAC-T cell, or TAC-like-T cell. In some embodiments, the method further comprises introducing into the allogeneic T cell a second nucleic acid encoding a functional exogenous receptor comprising an extracellular ligand binding domain and optionally an intracellular signaling domain (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the second nucleic acid encodes a CAR. In some embodiments, the CAR comprises a polypeptide comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain: and (c) an intracellular signaling domain. In some embodiments, the CAR comprises a polypeptide comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, the second nucleic acid encodes a cTCR comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the second nucleic acid encodes a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19. CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the second nucleic acid encodes a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ. In some embodiments, the TAC is an anti-CD20 TAC comprising the amino acid sequence of SEQ ID NO: 66. In some embodiments, the second nucleic acid encodes a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the second nucleic acid encodes a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRδ, TCRγ, TCRδ, CD3ε, CD3γ, and CD3δ. In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is monospecific. In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is multivalent. In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is multispecific. In some embodiments, the first nucleic acid and the second nucleic acid are on separate vectors. In some embodiments, the first nucleic acid and the second nucleic acid are on the same vector. Thus in some embodiments, the present invention also provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a vector comprising a first nucleic acid encoding a Nef protein (e.g., wt Nef, or mutant Nef such as mutant SIV Nef), and a second nucleic acid encoding a CAR comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD19, CD20); (b) a transmembrane domain; and (c) an intracellular signaling domain; wherein the Nef protein upon expression results in down-modulation of the endogenous TCR of the allogeneic T cell. In some embodiments, there is provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a vector comprising a first nucleic acid encoding a Nef protein (e.g., wt Nef, or mutant Nef such as mutant SIV Nef), and a second nucleic acid encoding a chimeric TCR (cTCR) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ; wherein the Nef protein upon expression results in down-modulation of the endogenous TCR of the allogeneic T cell. In some embodiments, there is provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a vector comprising a first nucleic acid encoding a Nef protein (e.g., wt Nef, or mutant Nef such as mutant SIV Nef), and a second nucleic acid encoding a T cell antigen coupler (TAC) comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε) (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3&; and wherein the first, second, and third TCR co-receptors are all selected from the group consisting of CD4, CD8, and CD28; wherein the Nef protein upon expression results in down-modulation of the endogenous TCR of the allogeneic T cell. In some embodiments, there is provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a vector comprising a first nucleic acid encoding a Nef protein (e.g., wt Nef, or mutant Nef such as mutant SIV Nef), and a second nucleic acid encoding a TAC-like chimeric receptor comprising: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3&); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ; wherein the Nef protein upon expression results in down-modulation of the endogenous TCR of the allogeneic T cell. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) a 2-4, an 8-10, an 11-13, aa 3840, aa 44-46, an 47-49, an 50-52, aa 53-55, an 56-58, an 59-61, aa 62-64, an 65-67, an 98-100, aa 107-109, an 110-112, an 137-139, an 152-154, aa 164-166, aa 167-169, aa 170-172, aa 173-175, aa 176-178, aa 178-179, 179-181aa, an 182-184, an 185-187, an 188-190, an 191-193, an 194-196, an 203-205, an 206-208, an 212-214, an 215-217, an 218-220, an 221-223, an 8-13, an 44-67, an 107-112, an 164-196, an 203-208, or an 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, an 188-190, an 194-196, an 203-205, an 44-67, an 164-169, an 176-181, an 185-190; (iii) an 2-4, an 56-58, an 59-61, an 62-64, an 65-67, an 107-109, an 137-139, an 152-154, an 164-166, aa 167-169, aa 170-172, aa 173-175, aa 176-178, 178-179aa, aa 179-181, aa 182-184, aa 185-187, an 188-190, an 194-196, an 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)). In some embodiments, the functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the present invention also provides a method of reducing GvHD or transplantation rejection of an allogeneic CAR-T cell, comprising introducing into the allogeneic CAR-T cell a nucleic acid encoding a Nef protein (e.g., wt Nef, or mutant Nef such as mutant SIV Nef), wherein the Nef protein upon expression results in down-modulation of the endogenous TCR of the allogeneic CAR-T cell. In some embodiments, the CAR comprises a polypeptide comprising: (a) an extracellular ligand binding domain comprising one or more (such as any one of 1, 2, 3, 4, 5, 6 or more) binding moieties (e.g., sdAbs, scFvs) specifically recognizing an antigen (e.g., BCMA, CD20, CD19); (b) a transmembrane domain; and (c) an intracellular signaling domain. In some embodiments, the CAR comprises a polypeptide comprising: (a) an extracellular ligand binding domain comprising one or more (such as any one of 1, 2, 3, 4, 5, 6 or more) anti-BCMA sdAbs; (b) a transmembrane domain; and (c) an intracellular signaling domain. In some embodiments, the CAR is monospecific. In some embodiments, the CAR is multivalent. In some embodiments, the CAR is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, an 8-10, an 11-13, aa 3840, an 44-46, an 4749, an 50-52, an 53-55, aa 56-58, an 59-61, an 62-64, an 65-67, an 98-100, an 107-109, an 110-112, aa 137-139, an 152-154, an 164-166, an 167-169, an 170-172, an 173-175, aa 176-178, an 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or an 212-223; (ii) an 2-4, aa 44-46, aa 56-58, an 59-61, an 62-64, aa 65-67, an 98-100, aa 107-109, an 137-139, aa 152-154, an 164-166, an 167-169, an 176-178, an 178-179, aa 179-181, an 185-187, aa 188-190, an 194-196, an 203-205, an 44-67, aa 164-169, an 176-181, an 185-190; (iii) aa 2-4, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, aa 56-67, or aa 164-190; or (iv) aa 2-4, aa 56-58, aa 59-61, aa 62-64, aa 65-67, aa 107-109, an 137-139, aa 152-154, aa 164-166, aa 167-169, an 176-178, aa 178-179, aa 179-181, an 185-187, aa 188-190, an 194-196, aa 203-205, aa 56-67, aa 164-169, an 176-181, or aa 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) CAR. In some embodiments, the functional CAR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the present invention also provides a method of reducing GvHD or transplantation rejection of an allogeneic cTCR-T cell, comprising introducing into the allogeneic cTCR-T cell a nucleic acid encoding a Nef protein (e.g., wt Nef, or mutant Nef such as mutant SIV Nef), wherein the Nef protein upon expression results in down-modulation of the endogenous TCR of the allogeneic cTCR-T cell. In some embodiments, the cTCR comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; (c) an optional extracellular domain of a first TCR subunit (e.g., CD3ε) or a portion thereof; (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the cTCR comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional linker; and (c) a full length CD3ε (excluding signal peptide). In some embodiments, the cTCR is monospecific. In some embodiments, the cTCR is multivalent. In some embodiments, the cTCR is multispecific. In some embodiments, the cTCR is an anti-CD20 cTCR comprising the amino acid sequence of SEQ ID NO: 64. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, aa 8-10, an 11-13, aa 38-40, an 44-46, an 47-49, an 50-52, an 53-55, an 56-58, an 59-61, an 62-64, an 65-67, an 98-100, aa 107-109, aa 110-112, aa 137-139, aa 152-154, aa 164-166, aa 167-169, aa 170-172, aa 173-175, an 176-178, an 178-179, 179-181aa, an 182-184, an 185-187, an 188-190, aa 191-193, an 194-196, an 203-205, an 206-208, an 212-214, an 215-217, an 218-220, an 221-223, an 8-13, an 44-67, an 107-112, an 164-196, an 203-208, or aa 212-223; (ii) an 2-4, an 44-46, an 56-58, an 59-61, an 62-64, an 65-67, an 98-100, an 107-109, an 137-139, an 152-154, an 164-166, an 167-169, an 176-178, an 178-179, an 179-181, an 185-187, an 188-190, aa 194-196, an 203-205, an 44-67, an 164-169, an 176-181, an 185-190: (iii) an 2-4, aa 56-58, aa 59-61, an 62-64, an 65-67, an 107-109, an 137-139, an 152-154, aa 164-166, an 167-169, aa 170-172, an 173-175, aa 176-178, 178-179aa, aa 179-181, aa 182-184, aa 185-187, aa 188-190, aa 194-196, aa 203-205, aa 56-67, or an 164-190; or (iv) a 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, aa 188-190, an 194-196, an 203-205, aa 56-67, an 164-169, aa 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) cTCR In some embodiments, the functional cTCR is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the present invention also provides a method of reducing GvHD or transplantation rejection of an allogeneic TAC-T cell, comprising introducing into the allogeneic TAC-T cell a nucleic acid encoding a Nef protein (e.g., wt Nef, or mutant Nef such as mutant SIV Nef), wherein the Nef protein upon expression results in down-modulation of the endogenous TCR of the allogeneic TAC-T cell. In some embodiments, the TAC comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., CD3ε); (d) an optional second linker; (e) an extracellular domain of CD4 or a portion thereof; (f) a transmembrane domain of CD4; and (g) an intracellular signaling domain of CD4; wherein the TCR subunit is selected from the group consisting of TCRα, TCRβ, TCRγ, TCRδ, CD3ε, CD3γ, and CD3δ. In some embodiments, the TAC is an anti-CD20 TAC comprising the amino acid sequence of SEQ ID NO: 66. In some embodiments, the TAC is monospecific. In some embodiments, the TAC is multivalent. In some embodiments, the TAC is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) aa 2-4, an 8-10, an 11-13, an 38-40, a 44-46, a 47-49, aa 50-52, an 53-55, an 56-58, an 59-61, an 62-64, an 65-67, an 98-100, an 107-109, an 110-112, an 137-139, aa 152-154, aa 164-166, aa 167-169, aa 170-172, aa 173-175, aa 176-178, aa 178-179, 179-181aa, aa 182-184, aa 185-187, aa 188-190, aa 191-193, aa 194-196, aa 203-205, aa 206-208, aa 212-214, aa 215-217, aa 218-220, aa 221-223, aa 8-13, aa 44-67, aa 107-112, aa 164-196, aa 203-208, or an 212-223; (ii) an 2-4, an 44-46, an 56-58, an 59-61, an 62-64, an 65-67, an 98-100, an 107-109, aa 137-139, aa 152-154, aa 164-166, aa 167-169, aa 176-178, aa 178-179, aa 179-181, an 185-187, an 188-190, an 194-196, aa 203-205, aa 44-67, an 164-169, an 176-181, an 185-190; (iii) an 2-4, an 56-58, an 59-61, an 62-64, an 65-67, an 107-109, an 137-139, an 152-154, an 164-166, aa 167-169, aa 170-172, aa 173-175, aa 176-178, 178-179aa, aa 179-181, aa 182-184, an 185-187, an 188-190, an 194-196, aa 203-205, an 56-67, or an 164-190; or (iv) an 2-4, an 56-58, an 59-61, an 62-64, an 65-67, an 107-109, an 137-139, an 152-154, an 164-166, an 167-169, an 176-178, an 178-179, an 179-181, an 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC. In some embodiments, the functional TAC is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

In some embodiments, the present invention also provides a method of reducing GvHD or transplantation rejection of an allogeneic TAC-like-T cell, comprising introducing into the allogeneic TAC-like-T cell a nucleic acid encoding a Nef protein (e.g., wt Nef, or mutant Nef such as mutant SIV Nef), wherein the Nef protein upon expression results in down-modulation of the endogenous TCR of the allogeneic TAC-like-T cell. In some embodiments, the TAC-like chimeric receptor comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the TAC-like chimeric receptor comprises: (a) an extracellular ligand binding domain comprising an antigen-binding fragment (e.g., sdAb, scFv) that specifically recognizes one or more epitopes of a tumor antigen (e.g., BCMA, CD19, CD20); (b) an optional first linker; (c) an extracellular TCR binding domain that specifically recognizes the extracellular domain of a TCR subunit (e.g., TCRα); (d) an optional second linker; and (e) a full length CD3ε (excluding signal peptide); wherein the TCR subunit is selected from the group consisting of TCRα, TCRβ, TCRγ, TCRδ, CD3ε, CD3γ, and CD3δ. In some embodiments, the TAC-like chimeric receptor is monospecific. In some embodiments, the TAC-like chimeric receptor is multivalent. In some embodiments, the TAC-like chimeric receptor is multispecific. In some embodiments, the Nef protein comprises the amino acid sequence of any of SEQ ID NOs: 12-22. In some embodiments, the Nef protein is a mutant SIV Nef comprising one of more mutations at amino acid residues at any of: (i) an 2-4, an 8-10, an 11-13, an 38-40, an 44-46, an 47-49, an 50-52, aa 53-55, an 56-58, an 59-61, an 62-64, an 65-67, aa 98-100, aa 107-109, aa 110-112, aa 137-139, aa 152-154, aa 164-166, aa 167-169, aa 170-172, an 173-175, an 176-178, an 178-179, 179-181aa, an 182-184, an 185-187, an 188-190, an 191-193, an 194-196, an 203-205, an 206-208, an 212-214, aa 215-217, an 218-220, an 221-223, an 8-13, an 44-67, an 107-112, an 164-196, an 203-208, or an 212-223; (ii) an 2-4, an 44-46, an 56-58, an 59-61, aa 62-64, an 65-67, an 98-100, an 107-109, an 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, an 185-190; (iii) an 2-4, an 56-58, an 59-61, an 62-64, an 65-67, aa 107-109, aa 137-139, aa 152-154, aa 164-166, aa 167-169, aa 170-172, aa 173-175, aa 176-178, 178-179aa, an 179-181, an 182-184, an 185-187, an 188-190, an 194-196, an 203-205, an 56-67, or an 164-190; or (iv) a 2-4, an 56-58, an 59-61, an 62-64, an 65-67, an 107-109, an 137-139, aa 152-154, aa 164-166, aa 167-169, aa 176-178, aa 178-179, aa 179-181, aa 185-187, an 188-190, an 194-196, an 203-205, an 56-67, an 164-169, an 176-181, or an 185-190; wherein the amino acid residue position corresponds to that of wildtype SIV Nef. In some embodiments, the Nef protein (e.g., mutant Nef such as mutant SIV Nef) does not down-regulate cell surface expression of CD4 and/or CD28. In some embodiments, the Nef protein (e.g., wt Nef, or mutant Nef such as mutant SIV Nef) down-regulates cell surface expression of CD4 and/or CD28. In some embodiments, the Nef protein (e.g., wt Nef, or mutant Nef such as mutant SIV Nef) down-regulates cell surface expression of TCR, CD4, and CD28. In some embodiments, the Nef protein (e.g., mutant Nef such as mutant SIV Nef) down-regulates cell surface expression of TCR, but does not down-regulates cell surface expression of CD4 and/or CD28. In some embodiments, the Nef protein (e.g., mutant Nef such as mutant SIV Nef) down-regulates cell surface expression of TCR and CD4, but does not down-regulates cell surface expression of CD28. In some embodiments, the Nef protein (e.g., mutant Nef such as mutant SIV Nef) down-regulates cell surface expression of TCR and CD28, but does not down-regulates cell surface expression of CD4. In some embodiments, the Nef protein (e.g., wt Nef, or mutant Nef such as mutant SIV Nef) down-regulates cell surface expression of endogenous TCR, but does not down-modulate (e.g., down-regulate cell surface expression) TAC-like chimeric receptor. In some embodiments, the functional TAC-like chimeric receptor is down-modulated (e.g., down-regulated for cell surface expression) by the Nef protein (e.g., wt Nef, or mutant Nef such as mutant SIV Nef) by at most about any of 50%, 40%, 30%, 20%, 10%, or 5%.

### VIII. Kits and Articles of Manufacture

Further provided are kits, unit dosages, and articles of manufacture comprising any one of the modified T cells (e.g., allogeneic T cell, endogenous TCR-deficient T cell, GvHD-minimized T cell) expressing a Nef protein (e.g., wt Nef, or mutant Nef such as mutant SIV Nef) and/or a functional exogenous receptor (such as engineered TCR (e.g., traditional engineered TCR, chimeric TCR (cTCR)), TAC, TAC-like chimeric receptor, or CAR (e.g., antibody-based CAR, ligand/receptor-based CAR, or ACTR)) described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer, autoimmune disease, or infectious disease) as described herein, or reducing/preventing GvHD or transplantation rejection when treating a disease or disorder, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. The kits or article of manufacture may include multiple unit doses of the pharmaceutical composition and instructions for use, packaged in quantities sufficient for storage and use in pharmacies, for example, hospital pharmacies and compounding pharmacies.

### Exemplary Embodiments

Embodiment 1. A method of producing a modified T cell, comprising: introducing into a precursor T cell a first nucleic acid encoding a Nef protein, wherein the Nef protein upon expression results in down-modulation of the endogenous T cell receptor (TCR) in the modified T cell.
Embodiment 2. The method of embodiment 1, wherein the down-modulation comprises down-regulating cell surface expression of endogenous TCR.
Embodiment 3. The method of embodiment 2, wherein the cell surface expression of endogenous TCR is down-regulated by at least about 50%.
Embodiment 4. The method of embodiment 2 or 3, wherein the cell surface expression of endogenous TCR is down-regulated by at least about 60%.
Embodiment 5. The method of any one of embodiments 2-4, wherein the cell surface expression of endogenous TCR is down-regulated by at least about 70%.
Embodiment 6. The method of any one of embodiments 2-5, wherein the cell surface expression of endogenous TCR is down-regulated by at least about 80%.
Embodiment 7. The method of any one of embodiments 2-6, wherein the cell surface expression of endogenous TCR is down-regulated by at least about 90%.
Embodiment 8. The method of any one of embodiments 2-7, wherein the cell surface expression of endogenous TCR is down-regulated by at least about 95%.
Embodiment 9. The method of any one of embodiments 1-8, wherein the modified T cell comprises unmodified endogenous TCR loci.
Embodiment 10. The method of any one of embodiments 1-8, wherein the modified T cell comprises a modified endogenous TCR locus.
Embodiment 11. The method of embodiment 10, wherein the modified T cell comprises a modified endogenous TCRα locus.
Embodiment 12. The method of embodiment 10 or 11, wherein the endogenous TCR locus is modified by a CRISPR-Cas (Clustered Regularly Interspaced Short Palindromic Repeats (CRISPR)-CRISPR associated (Cas)) system.
Embodiment 13. The method of embodiment 12, wherein the CRISPR-Cas system comprises a guide RNA (gRNA) comprising the nucleic acid sequence of SEQ ID NO: 23.
Embodiment 14. The method of any one of embodiments 1-13, wherein the Nef protein is selected from the group consisting of SIV Nef, HIV1 Nef, and HIV2 Nef.
Embodiment 15. The method of any one of embodiments 1-14, wherein the Nef protein is a wildtype Nef.
Embodiment 16. The method of embodiment 15, wherein the wildtype Nef comprises an amino acid sequence of any one of SEQ ID NOs: 12-17.
Embodiment 17. The method of any one of embodiments 1-14, wherein the Nef protein is a mutant Nef.
Embodiment 18. The method of embodiment 17, wherein the mutant Nef comprises one or more mutations in myristoylation site, N-terminal α-helix, tyrosine-based AP recruitment, CD4 binding site, acidic cluster, proline-based repeat, PAK binding domain. COP I recruitment domain, di-leucine based AP recruitment domain, V-ATPase and Raf-1 binding domain, or any combinations thereof, or one or more mutations at any of amino acid residues listed in Table 11.
Embodiment 19. The method of embodiment 17 or 18, wherein the mutation comprises insertion, deletion, point mutation(s), and/or rearrangement.
Embodiment 20. The method of any one of embodiments 17-19, wherein the mutant Nef comprises:

(i) an amino acid sequence of any one of SEQ ID NOs: 18-22;

(ii) one of more mutations at amino acid residues at any of: aa 2-4, aa 8-10, aa 11-13, aa 38-40, aa 44-46, an 47-49, aa 50-52, aa 53-55, aa 56-58, aa 59-61, aa 62-64, an 65-67, an 98-100, an 107-109, aa 110-112, an 137-139, an 152-154, an 164-166, an 167-169, an 170-172, an 173-175, aa 176-178, aa 178-179, 179-181aa, aa 182-184, aa 185-187, aa 188-190, aa 191-193, aa 194-196, aa 203-205, an 206-208, an 212-214, an 215-217, an 218-220, an 221-223, an 8-13, aa 44-67, an 107-112, an 164-196, an 203-208, or an 212-223, wherein the amino acid residue position corresponds to that of wildtype SIV Nef;

(iii) one of more mutations at amino acid residues at any of: an 2-4, an 44-46, an 56-58, an 59-61, aa 62-64, aa 65-67, an 98-100, an 107-109, an 137-139, an 152-154, an 164-166, an 167-169, an 176-178, an 178-179, an 179-181, an 185-187, an 188-190, an 194-196, an 203-205, an 44-67, an 164-169, an 176-181, an 185-190, wherein the amino acid residue position corresponds to that of wildtype SIV Nef;

(iv) one of more mutations at amino acid residues at any of: an 2-4, an 56-58, an 59-61, an 62-64, an 65-67, an 107-109, an 137-139, an 152-154, an 164-166, an 167-169, an 170-172, an 173-175, aa 176-178, 178-179aa, aa 179-181, aa 182-184, aa 185-187, aa 188-190, aa 194-196, an 203-205, an 56-67, or an 164-190, wherein the amino acid residue position corresponds to that of wildtype SIV Nef; or

(v) one of more mutations at amino acid residues at any of: an 2-4, an 56-58, an 59-61, an 62-64, aa 65-67, aa 107-109, aa 137-139, aa 152-154, aa 164-166, aa 167-169, aa 176-178, aa 178-179, an 179-181, an 185-187, an 188-190, an 194-196, an 203-205, an 56-67, an 164-169, an 176-181, or an 185-190, wherein the amino acid residue position corresponds to that of wildtype SIV Nef.

Embodiment 21. The method of any one of embodiments 1-20, wherein the precursor T cell comprises a second nucleic acid encoding a functional exogenous receptor comprising an extracellular ligand binding domain and optionally an intracellular signaling domain.
Embodiment 22. The method of any one of embodiments 1-20, further comprising introducing into the precursor T cell a second nucleic acid encoding a functional exogenous receptor comprising an extracellular ligand binding domain and optionally an intracellular signaling domain.
Embodiment 23. The method of embodiment 22, wherein the first nucleic acid and the second nucleic acid are introduced into the T cell sequentially.
Embodiment 24. The method of embodiment 22, wherein the first nucleic acid and the second nucleic acid are introduced into the T cell simultaneously.
Embodiment 25. The method of embodiment 24, wherein the first nucleic acid and the second nucleic acid are on separate vectors.
Embodiment 26. The method of embodiment 24, wherein the first nucleic acid and the second nucleic acid are on the same vector.
Embodiment 27. The method of embodiment 26, wherein the first nucleic acid and the second nucleic acid are operably linked to the same promoter.
Embodiment 28. The method of embodiment 27, wherein the first nucleic acid is upstream of the second nucleic acid.
Embodiment 29. The method of embodiment 27, wherein the first nucleic acid is downstream of the second nucleic acid.
Embodiment 30. The method of any one of embodiments 26-29, wherein the first nucleic acid and the second nucleic acid are connected via a linking sequence.
Embodiment 31. The method of embodiment 30, wherein the linking sequence is any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one.
Embodiment 32. The method of any one of embodiments 25-31, wherein the vector is a viral vector or a non-viral vector.
Embodiment 33. The method of any one of embodiments 1-32, wherein the modified T cell elicits no or a reduced graft-versus-host disease (GvHD) response in a histoincompatible individual as compared to the GvHD response elicited by a primary T cell isolated from the donor of the precursor T cell.
Embodiment 34. The method of any one of embodiments 1-33, further comprising isolating or enriching T cells comprising the first and/or the second nucleic acid.
Embodiment 35. The method of any one of embodiments 1-34, further comprising isolating or enriching CD3ε-negative T cells from the modified T cell expressing the Nef protein.
Embodiment 36. The method of any one of embodiments 1-35, further comprising isolating or enriching endogenous TCRα-negative T cells from the modified T cell expressing the Nef protein.
Embodiment 37. The method of any one of embodiments 1-36, further comprising formulating the modified T cells expressing the Nef protein with at least one pharmaceutically acceptable carrier.
Embodiment 38. The method of any one of embodiments 1-37, further comprising administering to an individual an effective amount of the modified T cells expressing the Nef protein.
Embodiment 39. The method of embodiment 38, wherein the individual has cancer.
Embodiment 40. The method of embodiment 38 or 39, wherein the individual is a human.
Embodiment 41. The method of any one of embodiments 21-40, wherein the functional exogenous receptor is an engineered TCR.
Embodiment 42. The method of embodiment 41, wherein the engineered TCR is a chimeric TCR (cTCR) comprising:

(a) an extracellular ligand binding domain comprising an antigen-binding fragment that specifically recognizes one or more epitopes of a tumor antigen;

(b) an optional linker;

(c) an optional extracellular domain of a first TCR subunit or a portion thereof:

(d) a transmembrane domain comprising a transmembrane domain of second TCR subunit; and

(e) an intracellular signaling domain comprising an intracellular signaling domain of a third TCR subunit;

wherein the first, second, and third TCR subunit are all selected from the group consisting of TCRα, TCRβ, TCRγ, TCRδ, CD3ε, CD3γ, and CD3δ.

Embodiment 43. The method of embodiment 42, wherein the first, second, and third TCR subunits are the same.
Embodiment 44. The method of embodiment 42, wherein the first, second, and third TCR subunits are different.
Embodiment 45. The method of any one of embodiments 21-40, wherein the functional exogenous receptor is a T cell antigen coupler (TAC) comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a TCR subunit;

(d) an optional second linker;

(e) an optional extracellular domain of a first TCR co-receptor or a portion thereof;

(f) a transmembrane domain comprising a transmembrane domain of a second TCR co-receptor; and

(g) an optional intracellular signaling domain comprising an intracellular signaling domain of a third TCR co-receptor;

wherein the TCR subunit is selected from the group consisting of TCRα, TCRβ, TCRγ, TCRδ, CD3ε, CD3γ, and CD3δ; and

wherein the first, second, and third TCR co-receptors are selected from the group consisting of CD4, CD8, and CD28.

Embodiment 46. The method of embodiment 45, wherein the first, second, and third TCR co-receptors are the same.
Embodiment 47. The method of embodiment 45, wherein the first, second, and third TCR co-receptors are different.
Embodiment 48. The method of any one of embodiments 21-40, wherein the functional exogenous receptor is a T cell antigen coupler (TAC)-like chimeric receptor comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a first TCR subunit;

(d) an optional second linker;

(e) an optional extracellular domain of a second TCR subunit, or a portion thereof;

(f) a transmembrane domain comprising a transmembrane domain of a third TCR subunit; and

(g) an optional intracellular signaling domain comprising an intracellular signaling domain of a fourth TCR subunit;

wherein the first, second, third, and fourth TCR subunits are all selected from the group consisting of TCRα, TCRβ, TCRγ, TCRδ, CD3ε, CD3γ, and CD3δ.

Embodiment 49. The method of embodiment 48, wherein at least the second, third, and fourth TCR subunits are the same.
Embodiment 50. The method of embodiment 48, wherein the first, second, third, and fourth TCR subunits are different.
Embodiment 51. The method of any one of embodiments 21-40, wherein the functional exogenous receptor is a non-TCR receptor.
Embodiment 52. The method of embodiment 51, wherein the non-TCR receptor is a chimeric antigen receptor (CAR).
Embodiment 53. The method of embodiment 52, wherein the CAR comprises a polypeptide comprising:

(a) an extracellular ligand binding domain comprising an antigen-binding fragment that specifically recognizes one or more epitopes of a tumor antigen;

(b) a transmembrane domain; and

(c) an intracellular signaling domain.

Embodiment 54. The method of embodiment 52, wherein the CAR is an antibody-coupled TCR (ACTR) comprising:

(a) an extracellular ligand binding domain comprising an antigen-binding fragment which is an Fc receptor;

(b) a transmembrane domain; and

(c) an intracellular signaling domain.

Embodiment 55. The method of any one of embodiments 42-53, wherein the antigen-binding fragment is selected from the group consisting of a Camel Ig, Ig NAR, Fab fragments, Fab′ fragments, F(ab)′2 fragments, F(ab)′3 fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody).
Embodiment 56. The method of embodiment 55, wherein the antigen-binding fragment is an sdAb or scFv.
Embodiment 57. The method of any one of embodiments 42-56, wherein the extracellular ligand binding domain is monovalent.
Embodiment 58. The method of any one of embodiments 42-57, wherein the extracellular ligand binding domain is multivalent.
Embodiment 59. The method of embodiment 58, wherein the extracellular ligand binding domain is multispecific.
Embodiment 60. The method of any one of embodiments 42-53, 55, 56, 58, and 59, wherein the extracellular ligand binding domain comprises a first sdAb and a second sdAb.
Embodiment 61. The method of any one of embodiments 42-53, 55, 56, 58, and 59, wherein the extracellular ligand binding domain comprises a first scFv and a second scFv.
Embodiment 62. The method of any one of embodiments 42-53 and 55-61, wherein the tumor antigen is selected from the group consisting of CD19, CD20, CD22, CD30, CD33, CD3δ, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof.
Embodiment 63. The method of embodiment 62, wherein the tumor antigen is BCMA, CD19, or CD20.
Embodiment 64. The method of embodiment 63, wherein the extracellular ligand binding domain comprises one or more sdAbs or scFvs specifically recognizing one or more epitopes of BCMA, CD19 or CD20.
Embodiment 65. The method of any one of embodiments 53-64, wherein the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152, CD154, and PD-1.
Embodiment 66. The method of embodiment 65, wherein the transmembrane domain is derived from CD8α.
Embodiment 67. The method of any one of embodiments 42-66, wherein the intracellular signaling domain comprises a primary intracellular signaling domain of an immune effector cell.
Embodiment 68. The method of embodiment 67, wherein the primary intracellular signaling domain is derived from CD3ζ CD3γ, CD3ε, CD3δ, FcR7 (FCER1G), FcR8 (Fc Epsilon Rib), CD5, CD22, CD79a, CD79b, CD66d, Fc gamma RIIa, DAP10, and DAP12.
Embodiment 69. The method of embodiment 68, wherein the primary intracellular signaling domain is derived from CD3ζ, CD3γ, or DAP12.
Embodiment 70. The method of any one of embodiments 53-69, wherein the intracellular signaling domain comprises a co-stimulatory signaling domain.
Embodiment 71. The method of embodiment 70, wherein the co-stimulatory signaling domain is derived from a co-stimulatory molecule selected from the group consisting of CARD11, CD2 (LFA-2), CDT CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR. LAT, GADS, SLP-76, PAG/Cbp, NKp44, NKp30, NKp46, NKG2D, CD83, CD150 (SLAMF1). CD152 (CTLA-4), CD223 (LAG3), CD273 (PD-L2), CD274 (PD-L1), DAP10, TRIM, ZAP70, a ligand that specifically binds with CD83, and any combination thereof.
Embodiment 72. The method of embodiment 71, wherein the co-stimulatory signaling domain comprises a cytoplasmic domain of CD137 (4-1BB).
Embodiment 73. The method of any one of embodiments 42-72, further comprising a hinge domain located between the C-terminus of the extracellular ligand binding domain and the N-terminus of the transmembrane domain.
Embodiment 74. The method of embodiment 73, wherein the hinge domain is derived from CD8α.
Embodiment 75. The method of any one of embodiments 42-74, further comprising a signal peptide located at the N-terminus of the functional exogenous receptor.
Embodiment 76. The method of embodiment 75, wherein the signal peptide is derived from CD8α.
Embodiment 77. The method of any one of embodiments 53 and 55-76, wherein the CAR comprises a polypeptide comprising from N-terminus to C-terminus: a signal peptide derived from CD8α, one or more sdAbs specifically recognizing one or more epitopes of BCMA, a hinge domain derived from CD8α, a transmembrane domain derived from CD8α, a co-stimulatory signaling domain derived from CD137 (4-1BB), and a primary intracellular signaling domain derived from CD3ζ.
Embodiment 78. A modified T cell obtained by the method of any one of embodiments 1-77.
Embodiment 79. A modified T cell comprising a first nucleic acid encoding a Nef protein, wherein the Nef protein upon expression results in down-modulation of the endogenous TCR in the modified T cell.
Embodiment 80. The modified T cell of embodiment 79, wherein the down-modulation comprises down-regulating cell surface expression of endogenous TCR.
Embodiment 81. The modified T cell of embodiment 80, wherein the cell surface expression of endogenous TCR is down-regulated by at least about 50%.
Embodiment 82. The modified T cell of embodiment 80 or 81, wherein the cell surface expression of endogenous TCR is down-regulated by at least about 60%.
Embodiment 83. The modified T cell of any one of embodiments 80-82, wherein the cell surface expression of endogenous TCR is down-regulated by at least about 70%.
Embodiment 84. The modified T cell of any one of embodiments 80-83, wherein the cell surface expression of endogenous TCR is down-regulated by at least about 80%.
Embodiment 85. The modified T cell of any one of embodiments 80-84, wherein the cell surface expression of endogenous TCR is down-regulated by at least about 90%.
Embodiment 86. The modified T cell of any one of embodiments 80-85, wherein the cell surface expression of endogenous TCR is down-regulated by at least about 95%.
Embodiment 87. The modified T cell of any one of embodiments 79-86, wherein the modified T cell comprises unmodified endogenous TCR loci.
Embodiment 88. The modified T cell of any one of embodiments 79-86, wherein the modified T cell comprises a modified endogenous TCR locus.
Embodiment 89. The modified T cell of embodiment 88, wherein the modified T cell comprises a modified endogenous TCRα locus.
Embodiment 90. The modified T cell of embodiment 88 or 89, wherein the endogenous TCR locus is modified by a CRISPR-Cas system.
Embodiment 91. The modified T cell of embodiment 90, wherein the CRISPR-Cas system comprises a gRNA comprising the nucleic acid sequence of SEQ ID NO: 23.
Embodiment 92. The modified T cell of any one of embodiments 79-91, wherein the Nef protein is selected from the group consisting of SIV Nef, HIV1 Nef, and HIV2 Nef.
Embodiment 93. The modified T cell of any one of embodiments 79-92, wherein the Nef protein is a wildtype Nef.
Embodiment 94. The modified T cell of embodiment 93, wherein the wildtype Nef comprises an amino acid sequence of any one of SEQ ID NOs: 12-17.
Embodiment 95. The modified T cell of any one of embodiments 79-92, wherein the Nef protein is a mutant Nef.
Embodiment 96. The modified T cell of embodiment 95, wherein the mutant Nef comprises one or more mutations in myristoylation site, N-terminal α-helix, tyrosine-based AP recruitment, CD4 binding site, acidic cluster, proline-based repeat, PAK binding domain, COP 1 recruitment domain, di-leucine based AP recruitment domain, V-ATPase and Raf-1 binding domain, or any combinations thereof, or one or more mutations at any of amino acid residues listed in Table 11.
Embodiment 97. The modified T cell of embodiment 95 or 96, wherein the mutation comprises insertion, deletion, point mutation(s), and/or rearrangement.
Embodiment 98. The modified T cell of any one of embodiments 95-97, wherein the mutant Nef comprises:

(i) an amino acid sequence of any one of SEQ ID NOs: 18-22;

(ii) one of more mutations at amino acid residues at any of: aa 2-4, aa 8-10, as 11-13, as 38-40, as 44-46, as 47-49, aa 50-52, as 53-55, as 56-58, aa 59-61, as 62-64, as 65-67, as 98-100, as 107-109, as 110-112, as 137-139, as 152-154, aa 164-166, as 167-169, as 170-172, as 173-175, as 176-178, aa 178-179, 179-181aa, as 182-184, aa 185-187, as 188-190, as 191-193, as 194-196, aa 203-205, as 206-208, as 212-214, as 215-217, as 218-220, as 221-223, as 8-13, as 44-67, aa 107-112, as 164-196, aa 203-208, or aa 212-223, wherein the amino acid residue position corresponds to that of wildtype SIV Nef;

(iii) one of more mutations at amino acid residues at any of: as 2-4, as 44-46, as 56-58, as 59-61, as 62-64, as 65-67, as 98-100, as 107-109, as 137-139, as 152-154, as 164-166, as 167-169, aa 176-178, aa 178-179, aa 179-181, aa 185-187, aa 188-190, aa 194-196, aa 203-205, aa 44-67, aa 164-169, aa 176-181, aa 185-190, wherein the amino acid residue position corresponds to that of wildtype SIV Nef;

(iv) one of more mutations at amino acid residues at any of: aa 24, aa 56-58, aa 59-61, aa 62-64, aa 65-67, aa 107-109, aa 137-139, aa 152-154, aa 164-166, aa 167-169, aa 170-172, aa 173-175, aa 176-178, 178-179aa, aa 179-181, aa 182-184, aa 185-187, aa 188-190, aa 194-196, aa 203-205, an 56-67, or an 164-190, wherein the amino acid residue position corresponds to that of wildtype SIV Nef; or

(v) one of more mutations at amino acid residues at any of: an 2-4, an 56-58, an 59-61, an 62-64, aa 65-67, aa 107-109, aa 137-139, aa 152-154, aa 164-166, aa 167-169, aa 176-178, aa 178-179, an 179-181, an 185-187, an 188-190, an 194-196, an 203-205, an 56-67, an 164-169, an 176-181, or an 185-190, wherein the amino acid residue position corresponds to that of wildtype SIV Nef.

Embodiment 99. The modified T cell of any one of embodiments 79-98, further comprising a second nucleic acid encoding a functional exogenous receptor comprising an extracellular ligand binding domain and optionally an intracellular signaling domain.
Embodiment 100. The modified T cell of embodiment 99, wherein the first nucleic acid and the second nucleic acid are on separate vectors.
Embodiment 101. The modified T cell of embodiment 99, wherein the first nucleic acid and the second nucleic acid are on the same vector.
Embodiment 102. The modified T cell of embodiment 101, wherein the first nucleic acid and the second nucleic acid are operably linked to the same promoter.
Embodiment 103. The modified T cell of embodiment 102, wherein the first nucleic acid is upstream of the second nucleic acid.
Embodiment 104. The modified T cell of embodiment 102, wherein the first nucleic acid is downstream of the second nucleic acid.
Embodiment 105. The modified T cell of any one of embodiments 101-104, wherein the first nucleic acid and the second nucleic acid are connected via a linking sequence.
Embodiment 106. The modified T cell of embodiment 105, wherein the linking sequence is any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α, PGK, CAGG, or any combinations thereof, wherein n is an integer of at least one.
Embodiment 107. The modified T cell of any one of embodiments 100-106, wherein the vector is a viral vector.
Embodiment 108. The modified T cell of embodiment 107, wherein the viral vector is selected from the group consisting of adenoviral vector, adeno-associated virus vector, retroviral vector, and lentiviral vector.
Embodiment 109. The modified T cell of embodiment 108, wherein the viral vector is a lentiviral vector.
Embodiment 110. The modified T cell of any one of embodiments 79-109, wherein the modified T cell elicits no or a reduced GvHD response in a histoincompatible individual as compared to the GvHD response elicited by a primary T cell isolated from the donor of the precursor T cell from which the modified T cell is derived.
Embodiment 111. The modified T cell of any one of embodiments 99-110, wherein the functional exogenous receptor is an engineered TCR.
Embodiment 112. The method of embodiment 111, wherein the engineered TCR is a chimeric TCR (cTCR) comprising:

(a) an extracellular ligand binding domain comprising an antigen-binding fragment that specifically recognizes one or more epitopes of a tumor antigen;

(b) an optional linker;

(c) an optional extracellular domain of a first TCR subunit or a portion thereof;

(d) a transmembrane domain comprising a transmembrane domain of second TCR subunit; and

(e) an intracellular signaling domain comprising an intracellular signaling domain of a third TCR subunit;

wherein the first, second, and third TCR subunit are all selected from the group consisting of TCRα, TCRβ, TCRγ, TCRδ, CD3ε, CD3-, and CD3δ.

Embodiment 113. The method of embodiment 12, wherein the first, second, and third TCR subunits are the same.
Embodiment 114. The method of embodiment 112, wherein the first, second, and third TCR subunits are different.
Embodiment 115. The method of any one of embodiments 99-110, wherein the functional exogenous receptor is a T cell antigen coupler (TAC) comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a TCR subunit;

(d) an optional second linker:

(e) an optional extracellular domain of a first TCR co-receptor or a portion thereof;

(f) a transmembrane domain comprising a transmembrane domain of a second TCR co-receptor; and

(g) an optional intracellular signaling domain comprising an intracellular signaling domain of a third TCR co-receptor:

wherein the TCR subunit is selected from the group consisting of TCRα, TCRβ, TCRγ, TCRδ, CD3ε, CD3γ, and CD3δ; and

wherein the first, second, and third TCR co-receptors are selected from the group consisting of CD4, CD8, and CD28.

Embodiment 116. The method of embodiment 115, wherein the first, second, and third TCR co-receptors are the same.
Embodiment 117. The method of embodiment 115, wherein the first, second, and third TCR co-receptors are different.
Embodiment 118. The method of any one of embodiments 99-110, wherein the functional exogenous receptor is a T cell antigen coupler (TAC)-like chimeric receptor comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a first TCR subunit:

(d) an optional second linker:

(e) an optional extracellular domain of a second TCR subunit, or a portion thereof;

(f) a transmembrane domain comprising a transmembrane domain of a third TCR subunit; and

(g) an optional intracellular signaling domain comprising an intracellular signaling domain of a fourth TCR subunit;

wherein the first, second, third, and fourth TCR subunits are all selected from the group consisting of TCRα, TCRβ, TCRγ, TCRδ, CD3ε, CD3t, and CD3δ.

Embodiment 119. The method of embodiment 118, wherein at least the second, third, and fourth TCR subunits are the same.
Embodiment 120. The method of embodiment 118, wherein the first, second, third, and fourth TCR subunits are different.
Embodiment 121. The modified T cell of any one of embodiments 99-110, wherein the functional exogenous receptor is a non-TCR receptor.
Embodiment 122. The modified T cell of embodiment 121, wherein the non-TCR receptor is a CAR.
Embodiment 123. The modified T cell of embodiment 122, wherein the CAR comprises a polypeptide comprising:

(a) an extracellular ligand binding domain comprising an antigen-binding fragment that specifically recognizes one or more epitopes of a tumor antigen;

(b) a transmembrane domain; and

(c) an intracellular signaling domain.

Embodiment 124. The method of embodiment 122, wherein the CAR is an antibody-coupled TCR (ACTR) comprising:

(a) an extracellular ligand binding domain comprising an antigen-binding fragment which is an Fc receptor;

(b) a transmembrane domain; and

(c) an intracellular signaling domain.

Embodiment 125. The modified T cell of any one of embodiments 112-124, wherein the antigen-binding fragment is selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody).
Embodiment 126. The modified T cell of embodiment 125, wherein the antigen-binding fragment is an sdAb or scFv.
Embodiment 127. The modified T cell of any one of embodiments 112-126, wherein the extracellular ligand binding domain is monovalent.
Embodiment 128. The modified T cell of any one of embodiments 112-126, wherein the extracellular ligand binding domain is multivalent.
Embodiment 129. The modified T cell of embodiment 128, wherein the extracellular ligand binding domain is multispecific.
Embodiment 130. The modified T cell of embodiment 128 or 129, wherein the extracellular ligand binding domain comprises a first sdAb and a second sdAb.
Embodiment 131. The modified T cell of embodiment 128 or 129, wherein the extracellular ligand binding domain comprises a first scFv and a second scFv.
Embodiment 132. The modified T cell of any one of embodiments 112-123 and 125-131, wherein the tumor antigen is selected from the group consisting of CD19, CD20, CD22, CD30, CD33, CD3δ, BCMA, CS1, CD138, CD123/IL3Rα, c-Met, gp100, MUCd, IGF-I receptor, EpCAM, EGFR/EGFRvIII. HER2, IGF1R, mesothelin, PSMA, WT1, ROR1, CEA, GD-2, NY-ESO-1, MAGE A3, GPC3, Glycolipid F77, PD-L1, PD-L2, and any combination thereof.
Embodiment 133. The modified T cell of embodiment 132, wherein the tumor antigen is BCMA, CD19, or CD20.
Embodiment 134. The modified T cell of embodiment 133, wherein the extracellular ligand binding domain comprises one or more sdAbs specifically recognizing one or more epitopes of BCMA.
Embodiment 135. The modified T cell of any one of embodiments 123-134, wherein the transmembrane domain is derived from a molecule selected from the group consisting of α, β, or (chain of the T-cell receptor, CD3ζ, CD3ε, CD4, CD5, CD8α, CD9, CD16, CD22, CD27, CD28, CD33, CD3γ, CD45, CD64, CD80, CD86, CD134, CD137 (4-1BB), CD152, CD154, and PD-1.
Embodiment 136. The modified T cell of embodiment 135, wherein the transmembrane domain is derived from CD8α.
Embodiment 137. The modified T cell of any one of embodiments 112-136, wherein the intracellular signaling domain comprises a primary intracellular signaling domain of an immune effector cell.
Embodiment 138. The modified T cell of embodiment 137, wherein the primary intracellular signaling domain is derived from CD3ζ, CD3γ, CD3ε, CD3δ, FcRγ (FCER1G), FcRn (Fc Epsilon Rib), CD5, CD22, CD79a, CD79b. CD66d, Fc gamma RIIa, DAP10, and DAP12.
Embodiment 139. The modified T cell of embodiment 138, wherein the primary intracellular signaling domain is derived from CD3γ, CD3γ, or DAP12.
Embodiment 140. The modified T cell of any one of embodiments 123-139, wherein the intracellular signaling domain comprises a co-stimulatory signaling domain.
Embodiment 141. The modified T cell of embodiment 140, wherein the co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT). CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR. BAFFR. NKp80 (KLRF1), CD160, CD19, CD4, IPO-3, CD353 (BLAME, SLAMF8), LTBR, LAT. GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10. TRIM, ZAP70, a ligand that specifically binds with CD83, and any combination thereof.
Embodiment 142. The modified T cell of embodiment 141, wherein the co-stimulatory signaling domain comprises a cytoplasmic domain of CD137 (4-1BB).
Embodiment 143. The modified T cell of any one of embodiments 112-142, further comprising a hinge domain located between the C-terminus of the extracellular ligand binding domain and the N-terminus of the transmembrane domain.
Embodiment 144. The modified T cell of embodiment 143, wherein the hinge domain is derived from CD8α.
Embodiment 144. The modified T cell of any one of embodiments 112-144, further comprising a signal peptide located at the N-terminus of the functional exogenous receptor.
Embodiment 145. The modified T cell of embodiment 144, wherein the signal peptide is derived from CD8α.
Embodiment 146. The modified T cell of any one of embodiments 123 and 125-145, wherein the CAR comprises a polypeptide comprising from N-terminus to C-terminus: a signal peptide derived from CD8α, one or more sdAbs specifically recognizing one or more epitopes of BCMA, a hinge domain derived from CD8α, a transmembrane domain derived from CD8α, a co-stimulatory signaling domain derived from CD137 (4-1BB), and a primary intracellular signaling domain derived from CD3ζ.
Embodiment 147. A pharmaceutical composition comprising the modified T cell of any one of embodiments 78-147, and a pharmaceutically acceptable carrier.
Embodiment 148. A method of treating a disease in an individual, comprising administering to the individual an effective amount of the pharmaceutical composition of embodiment 147.
Embodiment 149. The method of embodiment 148, wherein the disease is cancer.
Embodiment 150. The method of embodiment 148 or 149, wherein the individual is histoincompatible with the donor of the precursor T cell from which the modified T cell is derived.
Embodiment 151. The method of any one of embodiments 148-150, wherein the individual is a human.
Embodiment 152. An non-naturally occurring Nef protein, comprising one or more mutations in myristoylation site, N-terminal α-helix, tyrosine-based AP recruitment, CD4 binding site, acidic cluster, proline-based repeat, PAK binding domain, COP 1 recruitment domain, di-leucine based AP recruitment domain, V-ATPase and Raf-1 binding domain, or any combinations thereof, or one or more mutations at any of amino acid residues listed in Table 11.
Embodiment 153. The non-naturally occurring Nef protein of embodiment 152, wherein the mutation is selected from insertion, deletion, point mutation(s), and/or rearrangement.
Embodiment 154. The non-naturally occurring Nef protein of embodiment 152 or 153, wherein the non-naturally occurring Nef protein upon expression in a T cell has reduced down-modulation effect on an endogenous CD4 and/or CD28 in the T cell compared to a wildtype Nef protein.
Embodiment 155. The non-naturally occurring Nef protein of embodiment 154, wherein the down-modulation comprises down-regulating cell surface expression of the endogenous CD4 and/or CD28.
Embodiment 156. The non-naturally occurring Nef protein of embodiment 155, wherein the down-regulation of cell surface expression of endogenous CD4 and/or CD28 is reduced by at least about 50%.
Embodiment 157. The non-naturally occurring Nef protein of embodiment 155 or 156, wherein the down-regulation of cell surface expression of endogenous CD4 and/or CD28 is reduced by at least about 60%.
Embodiment 158. The non-naturally occurring Nef protein of any one of embodiments 155-157, wherein the down-regulation of cell surface expression of endogenous CD4 and/or CD28 is reduced by at least about 70%.
Embodiment 159. The non-naturally occurring Nef protein of any one of embodiments 155-158, wherein the down-regulation of cell surface expression of endogenous CD4 and/or CD28 is reduced by at least about 80%.
Embodiment 160. The non-naturally occurring Nef protein of any one of embodiments 155-159, wherein the down-regulation of cell surface expression of endogenous CD4 and/or CD28 is reduced by at least about 90%.
Embodiment 161. The non-naturally occurring Nef protein of any one of embodiments 155-160, wherein the down-regulation of cell surface expression of endogenous CD4 and/or CD28 is reduced by at least about 95%.
Embodiment 162. The non-naturally occurring Nef protein of any one of embodiments 152-161, wherein the isolated Nef protein upon expression in a T cell results in down-modulation of the endogenous TCR in the T cell.
Embodiment 163. The non-naturally occurring Nef protein of embodiment 162, wherein the down-modulation comprises down-regulating cell surface expression of the endogenous TCR.
Embodiment 164. The non-naturally occurring Nef protein of embodiment 163, wherein the cell surface expression of endogenous TCR is down-regulated by at least about 50%.
Embodiment 165. The non-naturally occurring Nef protein of embodiment 163 or 164, wherein the cell surface expression of endogenous TCR is down-regulated by at least about 60%.
Embodiment 166. The non-naturally occurring Nef protein of any one of embodiments 163-165, wherein the cell surface expression of endogenous TCR is down-regulated by at least about 70%.
Embodiment 167. The non-naturally occurring Nef protein of any one of embodiments 163-166, wherein the cell surface expression of endogenous TCR is down-regulated by at least about 80%.
Embodiment 168. The non-naturally occurring Nef protein of any one of embodiments 163-167, wherein the cell surface expression of endogenous TCR is down-regulated by at least about 90%.
Embodiment 169. The non-naturally occurring Nef protein of any one of embodiments 163-168, wherein the cell surface expression of endogenous TCR is down-regulated by at least about 95%.
Embodiment 170. The non-naturally occurring Nef protein of any one of embodiments 152-169, wherein the Nef protein is selected from the group consisting of SIV Nef, HIV1 Nef, and HIV2 Nef.
Embodiment 171. The non-naturally occurring Nef protein of any one of embodiments 152-170, comprising:

(i) an amino acid sequence of any one of SEQ ID NOs: 18-22;

(ii) one of more mutations at amino acid residues at any of: aa 2-4, aa 8-10, as 11-13, as 38-40, as 44-46, as 47-49, as 50-52, as 53-55, as 56-58, as 59-61, as 62-64, as 65-67, as 98-100, as 107-109, as 110-112, as 137-139, as 152-154, as 164-166, as 167-169, as 170-172, as 173-175, as 176-178, as 178-179, 179-181aa, as 182-184, as 185-187, as 188-190, as 191-193, as 194-196, as 203-205, as 206-208, as 212-214, as 215-217, as 218-220, as 221-223, as 8-13, as 44-67, as 107-112, as 164-196, as 203-208, or as 212-223, wherein the amino acid residue position corresponds to that of wildtype SIV Nef;

(iii) one of more mutations at amino acid residues at any of: as 2-4, as 44-46, as 56-58, as 59-61, as 62-64, as 65-67, as 98-100, as 107-109, as 137-139, as 152-154, as 164-166, as 167-169, as 176-178, as 178-179, as 179-181, as 185-187, as 188-190, as 194-196, as 203-205, as 44-67, as 164-169, as 176-181, as 185-190, wherein the amino acid residue position corresponds to that of wildtype SIV Nef;

(iv) one of more mutations at amino acid residues at any of, as 2-4, as 56-58, as 59-61, as 62-64, as 65-67, as 107-109, as 137-139, as 152-154, as 164-166, as 167-169, as 170-172, as 173-175, as 176-178, 178-179aa, as 179-181, as 182-184, as 185-187, as 188-190, as 194-196, aa 203-205, aa 56-67, or aa 164-190, wherein the amino acid residue position corresponds to that of wildtype SIV Nef; or

(v) one of more mutations at amino acid residues at any of: aa 2-4, aa 56-58, aa 59-61, aa 62-64, aa 65-67, aa 107-109, aa 137-139, aa 152-154, aa 164-166, aa 167-169, aa 176-178, aa 178-179, aa 179-181, aa 185-187, aa 188-190, aa 194-196, aa 203-205, aa 56-67, aa 164-169, aa 176-181, or aa 185-190, wherein the amino acid residue position corresponds to that of wildtype SIV Nef.

Embodiment 172. A viral vector comprising a first nucleic acid encoding a Nef protein.
Embodiment 173. The viral vector of embodiment 172, wherein the Nef protein is selected from the group consisting of SIV Nef, HIV1 Nef, and HIV2 Nef.
Embodiment 174. The viral vector of embodiment 172 or 173, wherein the Nef protein is a wildtype Nef.
Embodiment 175. The viral vector of embodiment 174, wherein the wildtype Nef comprises an amino acid sequence of any one of SEQ ID NOs: 12-17.
Embodiment 176. The viral vector of embodiment 172 or 173, wherein the Nef protein is a mutant Nef.
Embodiment 177. The viral vector of embodiment 176, wherein the mutant Nef comprises one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or one or more mutation at any of amino acid residues listed in Table 11.
Embodiment 178. The viral vector of embodiment 176 or 177, wherein the mutation comprises insertion, deletion, point mutation(s), and/or rearrangement.
Embodiment 179. The viral vector of any one of embodiments 176-178, wherein the mutant Nef comprises:

(i) an amino acid sequence of any one of SEQ ID NOs: 18-22;

(ii) one of more mutations at amino acid residues at any of: aa 2-4, aa 8-10, aa 11-13, aa 38-40, aa 44-46, aa 47-49, aa 50-52, aa 53-55, an 56-58, aa 59-61, an 62-64, an 65-67, an 98-100, aa 107-109, an 110-112, aa 137-139, an 152-154, an 164-166, an 167-169, an 170-172, an 173-175, an 176-178, aa 178-179, 179-181aa, an 182-184, an 185-187, aa 188-190, an 191-193, aa 194-196, aa 203-205, an 206-208, an 212-214, an 215-217, an 218-220, an 221-223, an 8-13, an 44-67, aa 107-112, aa 164-196, aa 203-208, or aa 212-223, wherein the amino acid residue position corresponds to that of wildtype SIV Nef;

(iii) one of more mutations at amino acid residues at any of: aa 2-4, aa 44-46, aa 56-58, aa 59-61, aa 62-64, aa 65-67, aa 98-100, aa 107-109, aa 137-139, an 152-154, an 164-166, an 167-169, aa 176-178, aa 178-179, aa 179-181, aa 185-187, aa 188-190, aa 194-196, aa 203-205, an 44-67, an 164-169, an 176-181, an 185-190, wherein the amino acid residue position corresponds to that of wildtype SIV Nef;

(iv) one of more mutations at amino acid residues at any of: an 2-4, an 56-58, an 59-61, an 62-64, aa 65-67, aa 107-109, aa 137-139, aa 152-154, aa 164-166, aa 167-169, aa 170-172, aa 173-175, aa 176-178, 178-179a, aa 179-181, aa 182-184, aa 185-187, aa 188-190, aa 194-196, an 203-205, an 56-67, or an 164-190, wherein the amino acid residue position corresponds to that of wildtype SIV Nef; or

(v) one of more mutations at amino acid residues at any of: an 2-4, an 56-58, an 59-61, an 62-64, aa 65-67, aa 107-109, aa 137-139, aa 152-154, aa 164-166, aa 167-169, aa 176-178, aa 178-179, an 179-181, an 185-187, an 188-190, an 194-196, an 203-205, an 56-67, an 164-169, an 176-181, or an 185-190, wherein the amino acid residue position corresponds to that of wildtype SIV Nef.

Embodiment 180. The viral vector of any one of embodiments 172-179, further comprising a second nucleic acid encoding a functional exogenous receptor comprising an extracellular ligand binding domain and optionally an intracellular signaling domain.
Embodiment 181. The viral vector of embodiment 180, wherein the first nucleic acid and the second nucleic acid are operably linked to the same promoter.
Embodiment 182. The viral vector of embodiment 181, wherein the promoter is selected from the group consisting of an EF-1 promoter, a CMV IE gene promoter, an EF-1α promoter, an ubiquitin C promoter, and a phosphoglycerate kinase (PGK) promoter.
Embodiment 183. The viral vector of embodiment 181 or 182, wherein the first nucleic acid is upstream of the second nucleic acid.
Embodiment 184. The viral vector of embodiment 181 or 182, wherein the first nucleic acid is downstream of the second nucleic acid.
Embodiment 185. The viral vector of any one of embodiments 180-184, wherein the first nucleic acid and the second nucleic acid are connected via a linking sequence.
Embodiment 186. The viral vector of embodiment 185, wherein the linking sequence is any of nucleic acid sequence encoding P2A, T2A, E2A, F2A, BmCPV 2A, BmIFV 2A, (GS)ₙ, (GSGGS)ₙ, (GGGS)ₙ, (GGGGS)ₙ, or nucleic acid sequence of IRES, SV40, CMV, UBC, EF1α.
PGK, CAGG, or any combinations thereof, wherein n is an integer of at least one.
Embodiment 187. The viral vector of any one of embodiments 172-186, wherein the viral vector is selected from the group consisting of adenoviral vector, adeno-associated virus vector, retroviral vector, and lentiviral vector.
Embodiment 188. The viral vector of embodiment 187, wherein the viral vector is a lentiviral vector.
Embodiment 189. The viral vector of any one of embodiments 180-188, wherein the functional exogenous receptor is an engineered TCR.
Embodiment 190. The method of embodiment 189, wherein the engineered TCR is a chimeric TCR (cTCR) comprising:

(a) an extracellular ligand binding domain comprising an antigen-binding fragment that specifically recognizes one or more epitopes of a tumor antigen;

(b) an optional linker;

(c) an optional extracellular domain of a first TCR subunit or a portion thereof:

(d) a transmembrane domain comprising a transmembrane domain of second TCR subunit; and

(e) an intracellular signaling domain comprising an intracellular signaling domain of a third TCR subunit;

wherein the first, second, and third TCR subunit are all selected from the group consisting of TCRα, TCRβ, TCRγ, TCRδ, CD3ε, CD3γ, and CD3δ.

Embodiment 191. The method of embodiment 190, wherein the first, second, and third TCR subunits are the same.
Embodiment 192. The method of embodiment 190, wherein the first, second, and third TCR subunits are different.
Embodiment 193. The method of any one of embodiments 180-188, wherein the functional exogenous receptor is a T cell antigen coupler (TAC) comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a TCR subunit;

(d) an optional second linker;

(e) an optional extracellular domain of a first TCR co-receptor or a portion thereof;

(f) a transmembrane domain comprising a transmembrane domain of a second TCR co-receptor; and

(g) an optional intracellular signaling domain comprising an intracellular signaling domain of a third TCR co-receptor;

wherein the TCR subunit is selected from the group consisting of TCRα, TCRβ, TCRγ, TCRδ, CD3ε, CD3γ, and CD3δ; and

wherein the first, second, and third TCR co-receptors are selected from the group consisting of CD4, CD8, and CD28.

Embodiment 194. The method of embodiment 193, wherein the first, second, and third TCR co-receptors are the same.
Embodiment 195. The method of embodiment 193, wherein the first, second, and third TCR co-receptors are different.
Embodiment 196. The method of any one of embodiments 180-188, wherein the functional exogenous receptor is a T cell antigen coupler (TAC)-like chimeric receptor comprising:

(a) an extracellular ligand binding domain comprising an antigen-binding fragment that specifically recognizes one or more epitopes of a tumor antigen;

(b) an optional first linker;

(c) an extracellular TCR binding domain that specifically recognizes the extracellular domain of a first TCR subunit;

(d) an optional second linker;

(e) an optional extracellular domain of a second TCR subunit, or a portion thereof;

(f) a transmembrane domain comprising a transmembrane domain of a third TCR subunit; and

(g) an optional intracellular signaling domain comprising an intracellular signaling domain of a fourth TCR subunit;

wherein the first, second, third, and fourth TCR subunits are all selected from the group consisting of TCRα, TCRβ, TCRγ, TCRδ, CD3ε, CD3γ, and CD3δ.

Embodiment 197. The method of embodiment 196, wherein at least the second, third, and fourth TCR subunits are the same.
Embodiment 198. The method of embodiment 196, wherein the first, second, third, and fourth TCR subunits are different.
Embodiment 199. The viral vector of any one of embodiments 180-188, wherein the functional exogenous receptor is a non-TCR receptor.
Embodiment 200. The viral vector of embodiment 199, wherein the non-TCR receptor is a CAR.
Embodiment 201. The viral vector of embodiment 200, wherein the CAR comprises a polypeptide comprising:

(a) an extracellular ligand binding domain comprising an antigen-binding fragment that specifically recognizes one or more epitopes of a tumor antigen;

(b) a transmembrane domain; and

(c) an intracellular signaling domain.

Embodiment 202. The method of embodiment 200, wherein the CAR is an antibody-coupled TCR (ACTR) comprising:

(a) an extracellular ligand binding domain comprising an antigen-binding fragment which is an Fc receptor:

(b) a transmembrane domain; and

(c) an intracellular signaling domain.

Embodiment 203. The viral vector of any one of embodiments 190-201, wherein the antigen-binding fragment is selected from the group consisting of a Camel Ig, Ig NAR, Fab fragments, Fab′ fragments, F(ab)′₂ fragments, F(ab)′₃ fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody).
Embodiment 204. The viral vector of embodiment 203, wherein the antigen-binding fragment is an sdAb or scFv.
Embodiment 205. The viral vector of any one of embodiments 190-204, wherein the extracellular ligand binding domain is monovalent.
Embodiment 206. The viral vector of any one of embodiments 190-204, wherein the extracellular ligand binding domain is multivalent.
Embodiment 207. The viral vector of embodiment 206, wherein the extracellular ligand binding domain is multispecific.
Embodiment 208. The viral vector of embodiment 206 or 207, wherein the extracellular ligand binding domain comprises a first sdAb and a second sdAb.
Embodiment 209. The viral vector of embodiment 206 or 207, wherein the extracellular ligand binding domain comprises a first scFv and a second scFv.
Embodiment 210. The viral vector of any one of embodiments 190-201 and 203-209, wherein the tumor antigen is selected from the group consisting of CD19, CD20, CD22, CD30, CD33, CD3δ, BCMA, CS1. CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof.
Embodiment 211. The viral vector of embodiment 210, wherein the tumor antigen is BCMA, CD19, or CD20.
Embodiment 212. The viral vector of embodiment 211, wherein the extracellular ligand binding domain comprises one or more sdAbs specifically recognizing one or more epitopes of BCMA.
Embodiment 213. The viral vector of embodiment 211, wherein the extracellular ligand binding domain comprises one or more scFvs specifically recognizing one or more epitopes of CD19 or CD20.
Embodiment 214. The viral vector of any one of embodiments 201-213, wherein the transmembrane domain is derived from a molecule selected from the group consisting of α, β, or (chain of the T-cell receptor, CD3ζ, CD3ε, CD4, CD5, CD8α, CD9, CD16, CD22, CD27, CD28. CD33, CD3γ, CD45, CD64, CD80, CD86, CD134, CD137 (4-1BB), CD152, CD154, and PD-1.
194. The viral vector of embodiment 193, wherein the transmembrane domain is derived from CD8α.
Embodiment 215. The viral vector of any one of embodiments 190-214, wherein the intracellular signaling domain comprises a primary intracellular signaling domain of an immune effector cell.
Embodiment 216. The viral vector of embodiment 215, wherein the primary intracellular signaling domain is derived from CD3, CD3γ, CD3ε, CD3δ, FcRγ (FCER1G), FcRβ (Fc Epsilon Rib), CD5, CD22, CD79a. CD79b, CD66d, Fe gamma RIIa, DAP10, and DAP12.
Embodiment 217. The viral vector of embodiment 216, wherein the primary intracellular signaling domain is derived from CD3ζ, CD3γ, or DAP12.
Embodiment 218. The viral vector of any one of embodiments 201-217, wherein the intracellular signaling domain comprises a co-stimulatory signaling domain.
Embodiment 219. The viral vector of embodiment 218, wherein the co-stimulatory signaling domain is derived from a co-stimulatory molecule selected from the group consisting of CARD11. CD2 (LFA-2), CD7, CD27, CD28, CD30, CD40, CD54 (ICAM-1), CD134 (OX40), CD137 (4-1BB), CD162 (SELPLG). CD258 (LIGHT), CD270 (HVEM, LIGHTR). CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, CD353 (BLAME, SLAMF8), LTBR. LAT. GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof.
Embodiment 220. The viral vector of embodiment 219, wherein the co-stimulatory signaling domain comprises a cytoplasmic domain of CD137 (4-1BB).
Embodiment 221. The viral vector of any one of embodiments 190-220, further comprising a hinge domain located between the C-terminus of the extracellular ligand binding domain and the N-terminus of the transmembrane domain.
Embodiment 222. The viral vector of embodiment 221, wherein the hinge domain is derived from CD8α.
Embodiment 223. The viral vector of any one of embodiments 190-222, further comprising a signal peptide located at the N-terminus of the functional exogenous receptor.
Embodiment 224. The viral vector of embodiment 23, wherein the signal peptide is derived from CD8α.
Embodiment 225. The viral vector of any one of embodiments 201 and 203-224, wherein the CAR comprises a polypeptide comprising from N-terminus to C-terminus: a signal peptide derived from CD8α, one or more sdAbs specifically recognizing one or more epitopes of BCMA, a hinge domain derived from CD8α, a transmembrane domain derived from CD8α, a co-stimulatory signaling domain derived from CD137 (4-1BB), and a primary intracellular signaling domain derived from CD3ζ.
Embodiment 226. The viral vector of any one of embodiments 201 and 203-225, comprising from upstream to downstream: the first nucleic acid encoding the Nef protein, a third nucleic acid encoding P2A, IRES, or PGK, an optional fourth nucleic acid encoding (GGGS)₃ linker, and the second nucleic acid encoding the CAR comprising an extracellular ligand binding domain comprising one or more sdAbs specifically recognizing one or more epitopes of BCMA.
Embodiment 227. The viral vector of any one of embodiments 201 and 203-225, comprising from upstream to downstream: the second nucleic acid encoding the CAR comprising an extracellular ligand binding domain comprising one or more sdAbs specifically recognizing one or more epitopes of BCMA, a third nucleic acid encoding P2A, IRES, or PGK, an optional fourth nucleic acid encoding (GGGS)₃ linker, and the first nucleic acid encoding the Nef protein.
Embodiment 228. A modified T cell obtained by introducing the viral vector of any one of embodiments 172-227 into a precursor T cell.
Embodiment 229. The modified T cell of embodiment 228, wherein the modified T cell elicits no or a reduced GvHD response in a histoincompatible individual as compared to the GvHD response elicited by a primary T cell isolated from the donor of the precursor T cell from which the modified T cell is derived.
Embodiment 230. A pharmaceutical composition comprising the modified T cell of embodiment 228 or 229, and a pharmaceutically acceptable carrier.
Embodiment 231. A method of treating a disease in an individual, comprising administering to the individual an effective amount of the pharmaceutical composition of embodiment 230.
Embodiment 232. The method of embodiment 231, wherein the disease is cancer.
Embodiment 233. The method of embodiment 231 or 232, wherein the individual is histoincompatible with the donor of the precursor T cell from which the modified T cell is derived.
Embodiment 234. The method of any one of embodiments 231-233, wherein the individual is a human.

### EXAMPLES

The examples and exemplary embodiments below are intended to be purely exemplary of the invention and should therefore not be considered to limit the invention in any way. The following examples and detailed description are offered by way of illustration and not by way of limitation.

### Example 1. SIV Nef Inhibits TCR-Mediated Signal Transduction

This example describes the design and preparation of exemplary T cells expressing SIV Nef, and the effects of SIV Nef on TCR-mediated signal transduction.

1. Construction of SIV Nef-P2A-LNGFR transfer plasmid and Jurkat cell line expressing SIV Nef-P2A-LNGFR

pLVX-Puro is an HIV-1-based, lentiviral expression vector. To construct pLVX-hEF1α vector, pLVX-Puro (Clontech) vector was enzymatically digested using C/al and EcoRI to remove the constitutively active human cytomegalovirus immediate early promoter (P_{CMV IE}) located just upstream of the multiple cloning site (MCS), then human EF1a promoter (GenBank: J04617.1) was cloned into the digested vector. Next, a fusion gene encoding SIV Nef, P2A, and LNGFR (low-affinity nerve growth factor receptor) was constructed sequentially. The SIV Nef-P2A-LNGFR fusion gene (SEQ ID NO: 24) was then cloned into the pLVX-hEF1α plasmid, resulting in recombinant SIV Nef-P2A-LNGFR transfer plasmid (hereinafter referred to as “PLLV-M071”, abbreviated as “M071”). M071 recombinant transfer plasmids were purified, mixed proportionally with packaging plasmids psPAX2 and envelope plasmids pMD2.G, then co-transduced into HEK 293T cells. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, which were stored at −80° C.

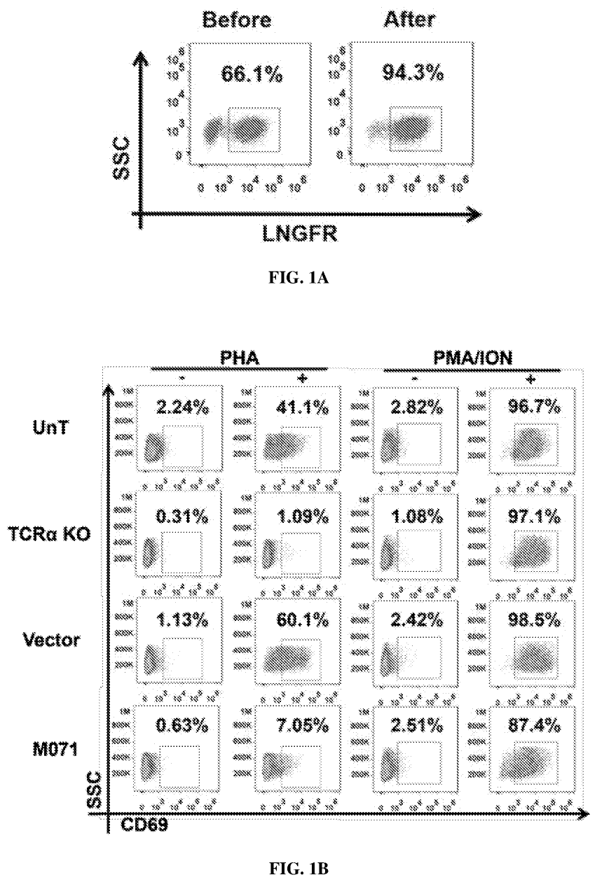

Jurkat cells (Clone E6-1, ATCC® TIB-152™) were cultured in 90% RPMI 1640 medium (Life Technologies, #22400-089), 10% Fetal Bovine Serum (FBS, Life Technologies, #10099-141), and 1% Penicillin-Streptomycin (Life Technologies, #15140-122). Lentiviruses carrying SIV nef-P2A-LNGFR fusion gene were added into the supernatant of Jurkat cell culture for transduction. LNGFR was used as selection marker for SIV Nef+ cells. 60 hrs post transduction, 5×10⁵ Jurkat cells were subject to flow cytometry analysis using the Life Attune N×T FACS (FACS) as below, LNGFR+ cells percentage was 66.1%. Another part of the 1×10⁷ cells were resuspended with DPBS then supplemented with 20 μL MACSelect LNGFR MicroBeads (Miltenyi Biotec, #130-091-330), and incubated on ice for 15 min for magnetic labeling. After incubation, PBE buffer (sodium phosphate/EDTA) was added to adjust the volume to 500 μL. The cell suspension was then subject a magnetic separation and enrichment according to the MACS kit protocols (Miltenyi Biotec kit, #130-091-330), resulting in 94.3% LNGFR+ Jurkat cell line expressing SIV Nef-P2A-LNGFR (FIG. 1A).

### FACS (Fluorescence-Activated Cell Sorter)

Briefly, cell suspension was centrifuged at room temperature 1000 rpm/min, and the supernatant was discarded. Cells were resuspended with DPBS, then antibody was added and incubated at 4° C. for 30 min. In this Example, the antibody used was 1 μL PerCP/Cy5.5 anti-human CD271 NGFR antibody (BioLegend®, #345111). After incubation, the cell suspension was centrifuged at room temperature 1000 rpm/min, the supernatant was discarded, then cells were resuspended with 1 mL DPBS. The centrifugation and resuspension with DPBS step was repeated once. Then cells were resuspended with 0.4 mL DPBS for FACS.

### MACS (Magnetic-Activated Cell Sorting)

Briefly, cell suspension was centrifuged at room temperature 1000 rpm/min, the supernatant was discarded. Cells were resuspended with DPBS then supplemented with 20 μL MACSelect LNGFR MicroBeads (Miltenyi Biotec, #130-091-330), and incubated on ice for 15 min for magnetic labeling. After incubation, PBE buffer (sodium phosphate/EDTA) was added to adjust the volume to 500 μL. The cell suspension was then subject to magnetic separation and enrichment according to the MACS kit protocols.

### 2. Construction of TCRα Knock-Out (KO) Construct and TCRα-Deficient Jurkat Cell Line

gRNA sequence targeting human TRAC (T Cell Receptor Alpha Constant; GenBank: NC_018925.2) was designed for CRISPR/Cas9 technology (SEQ ID NO: 23), and sub-cloned into lentiCRISPR v2 vector (Addgene Plasmid, #52961, contains a puromycin selectable marker) to construct TCRα KO recombinant plasmid. TCRα KO recombinant plasmids were purified, mixed proportionally with packaging plasmids psPAX2 and envelope plasmids pMD2.G, then co-transduced into HEK 293T cells. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, which were stored at −80° C.

Jurkat cells (Clone E6-1, ATCC® TIB-152™) were cultured as above. Lentiviruses carrying TCRα KO sequences were added into the supernatant of Jurkat cell culture for transduction. 72 hrs post transduction, puromycin of a final concentration of 1 μg/mL was added. Culture medium was changed every three days and supplemented with puromycin of the same concentration to further screen for single cell clones.

### 3. SIV Nef Inhibits TCR/CD3-Mediated Signal Transduction

To test if SIV Nef over-expression affects signal transduction via TCR/CD3, LNGFR+ Jurkat cells, untransduced Jurkat cells (UnT), TCRα KO Jurkat cells, and Jurkat cells transduced with empty vector were isolated by MACS as described above, then induced with phytohaemagglutinin (PHA, 2 μg/mL) for T cell activation. PHA binds to sugars on glycosylated surface proteins, including TCRs, and thereby crosslinks them. This triggers calcium-dependent signaling pathways leading to NFAT (nuclear factor of activated T cells) activation. 3 days after PHA-stimulation, 5×10⁵ cells from each Jurkat cell types were tested for CD69+ rate using FACS. FACS was performed as above, 1 μL PE anti-human CD69 Antibody (BioLegend®, #310906) was used.

As shown in FIG. 1B (left 2 columns of panels), after PHA stimulation, CD69 positive rate was 41.1% for untransduced Jurkat cells, 1.09% for TCRα KO Jurkat cells, 60.1% for Jurkat cells expressing empty vector, and 7.05% for M071 (SIV Nef-P2A-LNGFR) LNGFR+ enriched Jurkat cells. These results demonstrated that TCR-mediated T cell activation was significantly inhibited (P<0.05) upon SIV Nef over-expression.

4. SIV Nef does not Inhibit Signal Transduction Downstream of TCR/CD3 Complex

To test if SIV Nef over-expression affects signal transduction downstream of TCR/CD3, Jurkat cell lines were induced with mixture of PMA (Phorbol 12-myristate 13-acetate, 10 ng/mL) and ION (Ionomycin, 250 ng/mL), then the expression of T-cell activation marker CD69 was tested with FACS (see method above). PMA is a specific activator of Protein Kinase C (PKC) and hence of NF-κB. ION is a membrane permeable calcium ionophore facilitating the transfer of Ca²⁺ into and out of cells, and can be used to increase intracellular calcium levels. The combination of PMA and ION bypasses the signal transduction apparatus and activates transcription factors NF-κB and NFAT, leading to T-cell activation. As demonstrated in FIG. 1B (see right two columns of panels), upon PMA/ION stimulation, CD69+ rates of untransduced Jurkat cells (UnT), TCRα KO Jurkat cells, Jurkat cells expressing empty vector, and M071 LNGFR+ enriched Jurkat cells were 96.7%, 97.1%, 98.5%, and 87.4%, respectively. These results demonstrated that downstream intranuclear signal transduction was not significantly affected upon SIV Nef over-expression.

To summarize, the above results demonstrated that Nef significantly inhibits TCR-mediated upstream T-cell activation, but does not affect intranuclear signal transduction downstream of TCR.

### Example 2. SIV Nef Down-Regulates TCR/CD3 Complex Expression on T Cell Surface

5×10⁵ untransduced Jurkat cells (UnT), TCRα KO Jurkat cells, Jurkat cells expressing empty vector, and M071 LNGFR+ enriched Jurkat cells as described in Example 1 were obtained and subject to FACS for CD3ε and TCRαβ positive rate examination. FACS was performed as in Example 1. 1 μL PE/Cy7 anti-human CD3 Antibody (BioLegend®, #300316) or 1 μL PE/Cy5 anti-human TCR α/β Antibody (BioLegend®, #306710) was used for FACS.

As a separate experiment, 1×10⁶ untransduced Jurkat cells (UnT), TCRα KO Jurkat cells, Jurkat cells expressing empty vector, and M071 LNGFR+ enriched Jurkat cells as described in Example 1 were collected, centrifuged at mom temperature 1000 rpm/min, the supernatant was discarded. Cells were resuspended with DPBS, 100 μL Immunol Staining Fix Solution (Beyotime, #P0098) was added for fixation for 20 min at room temperature. The cell suspension was then centrifuged at room temperature 1500 rpm/min, supernatant was discarded. Cells were resuspended with 200 μL DPBS+Triton X-100 (0.1%), at room temperature for 20 min. Then 1 μL FITC conjugated CD3ζ/CD247 Antibody (Life Technologies, #A15754) was added and the cell suspension was incubated for 15 min at room temperature. After incubation, the cell suspension was centrifuged at room temperature 1500 rpm/min, the supernatant was discarded, then cells were resuspended with 1 mL DPBS. The centrifugation and resuspension with DPBS step was repeated once. Then cells were resuspended with 0.4 mL DPBS for FACS for CD3ζ positive rate examination.

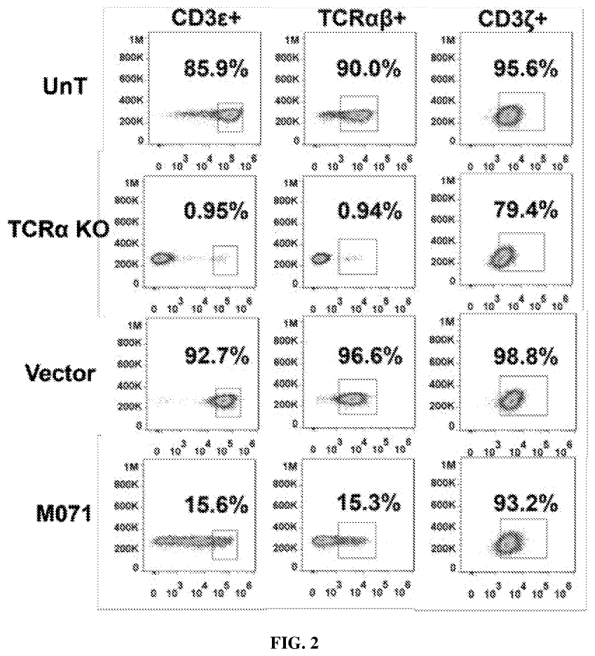

As shown in FIG. 2, Jurkat cells with high CD3ε expression, and cells expressing TCRαβ were significantly reduced upon SIV Nef expression. Compare CD3ε+ rate of 15.6% and TCRα3+ rate of 15.3% for M071 LNGFR+ enriched Jurkat cells with those of untransduced (UnT) Jurkat cells (85.9% and 90.0%, respectively). CD3ζ+ rate was 93.2% for M071 LNGFR+ enriched Jurkat cells, which did not significantly differ from that of untransduced Jurkat cells (95.6%). As a control, empty vector expression does not affect Jurkat cell expression of CD3ε, TCRαβ, and CD3ζ, as compared to that in untransduced (UnT) Jurkat cells.

These results demonstrated that SIV Nef over-expression significantly down-regulates T cell surface expression of TCR/CD3 complex (while CD3ζ expression is unaffected), which in turn affects TCR-mediated T cell activation.

### Example 3. SIV Nef Homologs HIV1 Nef and HIV2 Nef Inhibit TCR/CD3-Mediated Signal Transduction

### 1. Construction of HIV1 Nef/HIV2 Nef-T2A-Puro Transfer Plasmids and Cell Lines

Based on UniProt database analysis, HIV1 Nef and HIV2 Nef were recovered as SIV Nef homologs. Fusion genes HIV1 nef-T2A-Puro (SEQ ID NO: 25) and HIV2 nef-T2A-Puro (SEQ ID NO: 26) were constructed, and cloned into pLVX-hEF1α expression vector (constructed as in Example 1) to form recombinant transfer plasmids HIV1 Nef-T2A-Puro (hereinafter referred to as “HIV1” transfer plasmid) and HIV2 Nef-T2A-Puro (hereinafter referred to as “HIV2” transfer plasmid), respectively. HIV1 transfer plasmids were purified, mixed proportionally with packaging plasmids psPAX2 and envelope plasmids pMD2.G, then co-transduced into HEK 293T cells. HIV2 transfer plasmids were purified and similarly transduced into HEK 293T cells with psPAX2 and pMD2.G plasmids. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, which were stored at −80° C.

Jurkat cells (Clone E6-1, ATCC® TIB-152™) were cultured as in Example 1. Lentiviruses carrying HIV1 Nef-T2A-Puro or HIV2 Nef-T2A-Puro fusion sequence were added into the supernatant of Jurkat cell culture for transduction. Puromycin was used as selectable marker for HIV1/HIV2 Nef+ cells. 72 hrs post transduction, Puromycin of a final concentration of 1 μg/mL was added. Culture medium was changed every three days and supplemented with puromycin of the same concentration to further screen for single cell clones.

### 2. HIV1 Nef/HIV2 Nef Down-Regulates TCR/CD3 Complex Expression on T Cell Surface

5×10⁵ HIV1 Nef+ Jurkat cells, HIV2 Nef+ Jurkat cells, untransduced Jurkat cells (UnT), TCRα KO Jurkat cells, Jurkat cells expressing empty vector, M071 LNGFR+ enriched Jurkat cells as described in Example 1 were subject to FACS for CD3ε and TCRαβ positive rate examination. FACS was performed as in Example 1, 1 μL PE/Cy7 anti-human CD3 Antibody (BioLegend®, #300316) or 1 μL PE/Cy5 anti-human TCR α/β Antibody (BioLegend®, #306710) was used for FACS. As a separate experiment, 1×10⁶ HIV1 Nef+ Jurkat cells, HIV2 Nef+ Jurkat cells, untransduced (UnT) Jurkat cells, TCRα KO Jurkat cells, Jurkat cells expressing empty vector, and M071 LNGFR+ enriched Jurkat cells as described in Example 1 were collected, fixed, and subject to FACS for CD3ζ positive rate examination as in Example 2. 1 μL FITC conjugated CD3ζ/CD247 Antibody (Life Technologies, #A15754) was used for FACS.

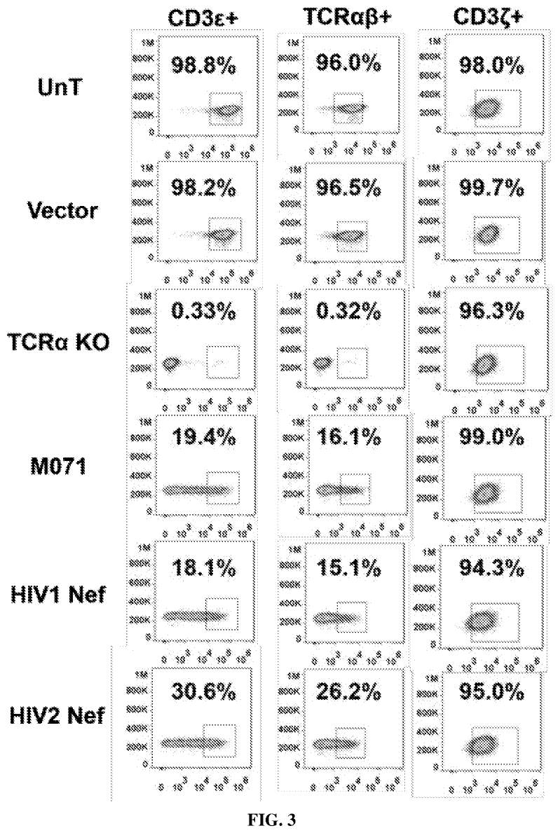

As shown in FIG. 3, TCRαβ+ rate was 16.1%, 15.1%, and 26.2% for M071 LNGFR+ enriched Jurkat cells, HIV1 Nef+ Jurkat cells, and HIV2 Nef+ Jurkat cells, respectively, which was significantly lower than that of Jurkat cells expressing empty vector (96.5%; P<0.05). CD3ε+ rate was 19.4%, 18.1%, and 30.6% for M071 LNGFR+ enriched Jurkat cells, HIV1 Nef+ Jurkat cells, and HIV2 Nef+ Jurkat cells, respectively, which was significantly lower than that of Jurkat cells expressing empty vector (98.2%; P<0.05). TCRα KO Jurkat cells served as positive control, which showed significantly decreased TCRα+ and CD3ε+ rates. On the other hand, CD3ζ+ rate was 99.0%, 94.3%, and 95.0% for M071 LNGFR+ enriched Jurkat cells, HIV1 Nef+ Jurkat cells, and HIV2 Nef+ Jurkat cells, respectively, which did not significantly differ from that untransduced (UnT) Jurkat cells or Jurkat cells expressing empty vector (98.0% and 99.7%, respectively; P>0.05).

These results demonstrate that over-expression of SIV Nef homologs HIV1 Nef and HIV2 Nef can effectively down-regulate cell surface expression of TCR/CD3 (while CD3ζ expression is unaffected), which in turn inhibit TCR/CD3-mediated T cell activation. The inhibition efficiency of HIV1 Nef and HIV2 Nef is comparable to that of SIV Nef.

### Example 4. SIV Nef Inhibits TCR-Mediated Cell Lysis of Target Cells

### 1. Construction of Anti-BCMA CAR and Anti-BCMA CAR-LNGFR Transfer Plasmids

Anti-BCMA CAR (hereinafter referred to as “BCMA CAR”) was constructed as described in WO2017025038 and WO2018028647 Examples. The entire content of WO2018028647, including the sequences of all BCMA CAR constructs described therein, are specifically incorporated herein by reference.

Briefly, a nucleic acid sequence encoding a CAR backbone polypeptide comprising from the N-terminus to the C-terminus: a CD8α hinge domain (“CD8α Hinge”), a CD8 transmembrane domain (“CD8 TM”), a CD28 cytoplasmic domain (“CD28 cyto”), and/or a 4-1BB (CD137) cytoplasmic domain (“4-4BB cyto”), and a CD3ζ cytoplasmic domain (“CD3ζ”) was chemically synthesized and cloned into a pre-modified lentiviral vector downstream and operably linked to a constitutive hEF1α promoter (pLVX-hEF1α constructed as in Example 1).

To construct BCMA CAR-P2A-LNGFR transfer plasmid carrying the fusion sequence BCMA CAR-P2A-LNGFR, BCMA CAR-P2A-LNGFR gene was directly constructed by Genscript.

BCMA CAR-P2A-LNGFR transfer plasmids were purified, mixed proportionally with packaging plasmids psPAX2 and envelope plasmids pMD2.G, then co-transduced into HEK 293T cells. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, which were stored at −80° C.

### 2. Test of SIV Nef and Anti-BCMA CAR Co-Expression

BCMA CAR and SIV Nef-P2A-LNGFR plasmids were co-transfected into HEK 293T cells using the regular polyethylenimine (PEI) transduction protocol. 3 days post-transfection, 5×10⁵ cells were examined for BCMA CAR+ and LNGFR+ expression with FACS. FACS was performed as described in Example 1. 1 μL FITC-Labeled Human BCMA/TNFRSF17 Protein. Fc Tag (ACROBiosystems, #BCA-HF254), and 1 μL PerCP/Cy5.5 anti-human CD271 NGFR Antibody (BioLegend®, #345111) were mixed and used for FACS.

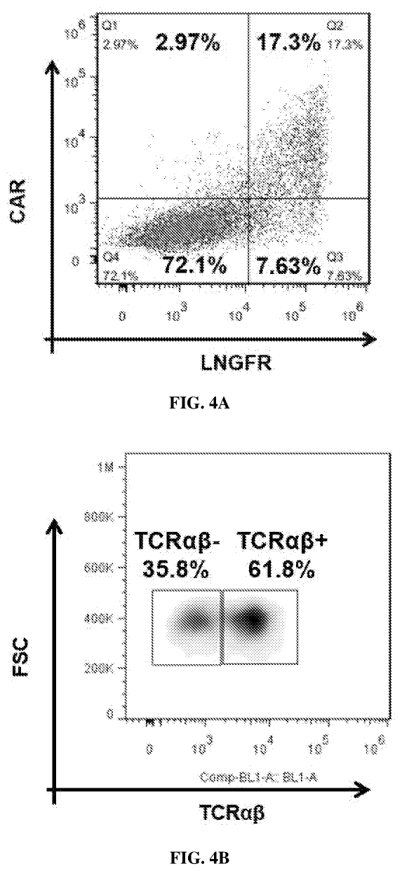

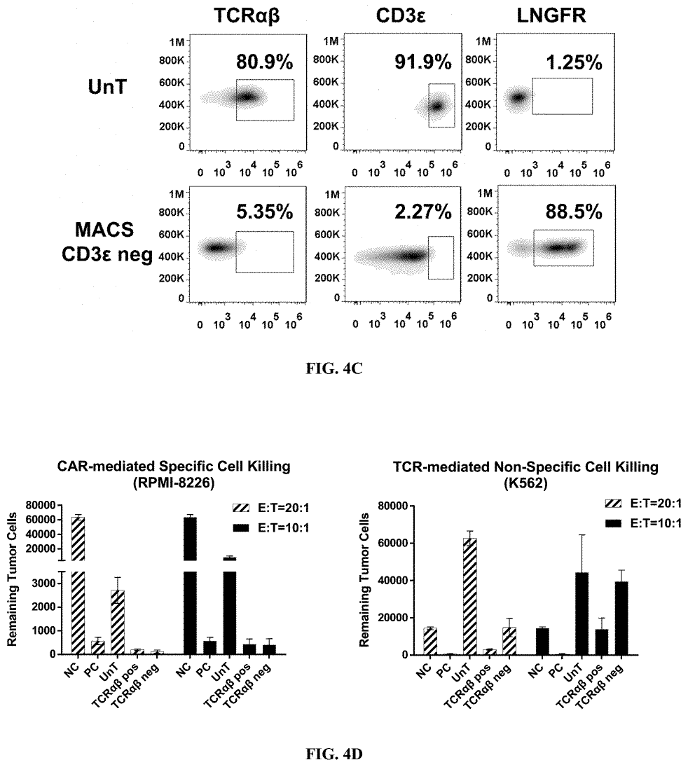

As shown in FIG. 4A, BCMA CAR and SIV Nef-P2A-LNGFR plasmids co-transfected into HEK 293T cells produced 17.3% BCMA CAR+/LNGFR+ double positive cells. This indicated that BCMA CAR could still effectively express on cell surface upon over-expression of SIV Nef.

### 3. SIV Nef Significantly Inhibits TCR Cell Surface Expression in Primary T Lymphocytes

50 mL peripheral blood was extracted from volunteers. Peripheral blood mononuclear cells (PBMCs) were isolated via density gradient centrifugation. Pan T Cell Isolation Kit (Miltenyi Biotec, #130-096-535) was used to magnetically label PBMCs and isolate and purify T lymphocytes. Human T cell activation/expansion kit (Miltenyi Biotec, #130-091-441) were used for the activation and expansion of purified T lymphocytes. Activated T lymphocytes were collected and resuspended in RPMI 1640 medium (Thermo Fisher SCIENTIFIC, #22400-089). 3 days after activation, 5×10⁶ activated T lymphocytes were co-transduced with lentiviruses encoding SIV Nef-P2A-LNGFR and lentiviruses encoding BCMA CAR-P2A-LNGFR. T cell suspension was added into 6-well plate, and incubated overnight in 37° C., 5% CO₂ incubator. 5 days post transduction, 1×10⁷ T cells were harvested and separated using MACS (as described in Example 1, with MACSelect LNGFR MicroBeads (Miltenyi Biotec, #130-091-330)) to obtain LNGFR+ T cells. Sorted LNGFR+ T cells were allowed to expand and enrich for 2 days, then 5×10⁵ LNGFR+ T cells were tested by FACS for TCRαβ positive rate. FACS was performed as in Example 1, 1 μL PE/Cy5 anti-human TCR α/si Antibody (BioLegend®, #306710) was used.

As shown in FIG. 4B, co-transducing T cells with lentiviruses encoding BCMA CAR-P2A-LNGFR and lentiviruses encoding SIV Nef-P2A-LNGFR led to 35.8% TCRαβ− cells in the LNGFR+ T cell population. This indicated that BCMA CAR-P2A-LNGFR and SIV Nef-P2A-LNGFR co-transduction could effectively produce TCRαβ negative CAR-T cells.

### 4. Sort and Enrich TCR/CD3-Depleted T Lymphocytes

Lentiviruses encoding BCMA CAR-P2A-LNGFR and lentiviruses encoding SIV Nef-P2A-LNGFR were used to co-transduce 5×10⁷ primary T lymphocytes. 5 days post transduction, Pan T Cell Isolation Kit (Miltenyi Biotec, #130-096-535) was used to magnetically label cells, and separate and purify CD3 negative T lymphocytes according to the kit protocols. Sorted CD3ε negative T cells were allowed to expand and enrich for 2 days, then 5×10⁵ cells (for each FACS experiment) were collected and examined by FACS for TCRαβ, CD3ε, and LNGFR positive rates within the CD3ε negative T cell population. FACS was performed as in Example 1, 1 μL PE/Cy5 anti-human TCR α/β Antibody (BioLegend®, #306710) was used for TCRαβ+ test, 1 μL PE/Cy7 anti-human CD3 Antibody (BioLegend®, #300316) was used for CD3ε+ test, and 1 μL PerCP/Cy5.5 anti-human CD271 NGFR Antibody (BioLegend®, #345111) was used for LNGFR+ test.

As shown in FIG. 4C, of the MACS-sorted CD3ε negative T cell population co-transduced with lentiviruses encoding BCMA CAR-P2A-LNGFR and lentiviruses encoding SIV Nef-P2A-LNGFR, TCRαβ+ rate was 5.35%, CD3ε+ rate was 2.27%, and LNGFR+ rate was 88.5% (see “MACS CD3 neg panels”); while for MACS sorted untransduced (UnT) T cells, TCRαβ+ rate was 80.9%, CD3ε+ rate was 91.9%, and LNGFR+ rate was only 1.25%. This indicated that CD3ε negative cell sorting by MACS could further isolate and enrich TCR negative primary T lymphocytes.

### 5. TCR-Mediated Cytolytic Activity Against Target Cells Significantly Reduced in TCR/CD3-Depleted T Lymphocytes

Different groups of sorted CD3ε negative T cells co-transduced with lentiviruses encoding BCMA CAR-P2A-LNGFR and lentiviruses encoding SIV Nef-P2A-LNGFR obtained from the above steps were mixed under 20: 1 or 10: 1 effector to target (E:T) cell ratios with multiple myeloma (MM) cell line RPMI-8226 (BCMA+, with Luciferase (Luc) marker) or chronic myelogenous leukemia (CML) cell line K562 (BCMA−, with Luc marker), and incubated in Corning@ 384-well solid white plate for 12 hrs. ONE-Glo™ Luciferase Assay System (Promega, #E6120) was used to measure luciferase production. 25 μL ONE-Glo™ Reagent was added to each well of the 384-well plate, incubated, then placed onto Spark™ 10M multimode microplate reader (TECAN) for Luciferase measurements, in order to calculate cytolytic effects of different T lymphocytes on target cells.

Specific and non-specific cytolytic effects of CAR+/CD3ε− T cells on RPMI-8226 and K562 cell lines were further studied. As shown in FIG. 4D, enriched CD3ε negative T cells expressing BCMA CAR-P2A-LNGFR and SIV Nef-P2A-LNGFR, regardless of TCR expression level (CD3ε+/TCRαβ+. “TCR pos”, or CD3ε−/TCRαβ−, “TCR neg”), effectively mediated anti-BCMA CAR-specific tumor cell killing on RPMI-8226 cells (BCMA+) with a lysing rate of above 90%, which was significantly higher than that in untransduced T-cells (“UnT”; P<0.05). On the other hand, enriched CD3ε negative T cells expressing BCMA CAR-P2A-LNGFR and SIV Nef-P2A-LNGFR induced TCR-mediated non-specific tumor cell killing in K562 cell line (BCMA−), as cells expressing TCRαβ (CD3ε+/TCRαβ+) had more cell lysis than cells not expressing TCRαβ (CD3ε−/TCRαβ+), and that such cytolytic effect was comparable to that in un-transduced T-cells (UnT; P>0.05). Luc-labeled cells not incubated with primary T cells served as negative control (NC). 0.25% Triton X-100 chemical lysis group served as positive controls (PC). Higher E:T rate resulted in more cell-killing, on both RPMI-8226 and K562 cell lines, likely due to lentivirus-mediated tumor cell killing. These results indicated that the expression of SIV Nef effectively inhibited TCRαβ-mediated T-cell activation, resulting in reduced TCR-mediated cytolytic effects on target cells.

### Example 5. Obtaining TCR/CD3-Depleted CAR-T Cells in One Step

### 1. Construction of SIV Nef+CAR All-In-One Vector and Jurkat Cell Line

Fusion gene sequences BCMA CAR-P2A-LNGFR-SIV Nef, BCMA CAR-P2A-SIV Nef, BCMA CAR-P2A-(GGGS)₃-SIV Nef, SIV Nef-P2A-BCMA CAR, SIV Nef-IRES-CAR, CAR-IRES-SIV Nef, CAR-PGK-SIV Nef and SIV Nef-PGK-CAR were chemically synthesized, and cloned into pLVX-hEF1α vector (see Example 1) for the construction of recombinant transfer plasmids BCMA CAR-P2A-LNGFR-SIV Nef (“PLLV-M072” or “M072”), BCMA CAR-P2A-SIV Nef (“PLLV-M086” or “M086”), BCMA CAR-P2A-(GGGS)₃-SIV Nef (“PLLV-M090” or “M090”), SIV Nef-P2A-BCMA CAR (“PLLV-M091” or “M091”), SIV Nef-IRES-BCMA CAR (“PLLV-M126” or “M126”), BCMA CAR-IRES-SIV Nef (“PLLV-M159” or “M159”), BCMA CAR-PGK-SIV Nef (“PLLV-M160” or “M160”), and SIV Nef-PGK-BCMA CAR (“PLLV-M161” or “M161”). The transfer plasmids were purified, mixed proportionally with packaging plasmids psPAX2 and envelope plasmids pMD2.G, then co-transduced into HEK 293T cells. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, which were stored at −80° C.

### 2. Obtaining TCR/CD3-Depleted CAR-T Cells in One Step

Lentiviruses carrying BCMA CAR-P2A-LNGFR-SIV Nef, BCMA CAR-P2A-SIV Nef, BCMA CAR-P2A-(GGGS)₃-SIV Nef, SIV Nef-P2A-BCMA CAR, SIV Nef-IRES-BCMA CAR. BCMA CAR-IRES-SIV Nef, BCMA CAR-PGK-SIV Nef and SIV Nef-PGK-BCMA CAR sequences, respectively, were added into cultured Jurkat cell suspension for transduction. Lentivirus SIV Nef-P2A-LNGFR (M071) was used as a non-CAR encoding control. Untransduced Jurkat cells (“UnT”) were used as negative control. 72 hrs post-transduction, suspension containing 5×10⁵ cells were collected and prepared for FACS as described in Example 1 to examine positive rates of CD3ε, TCRαβ, and BCMA CAR. 1 μL PE/Cy7 anti-human CD3 Antibody (BioLegend®, #300316), 1 μL PE/Cy5 anti-human TCR α/β Antibody (BioLegend®, #306710), or 1 μL FITC-Labeled Human BCMA/TNFRSF17 Protein, Fc Tag (ACROBiosystems, #BCA-HF254) was used for FACS.

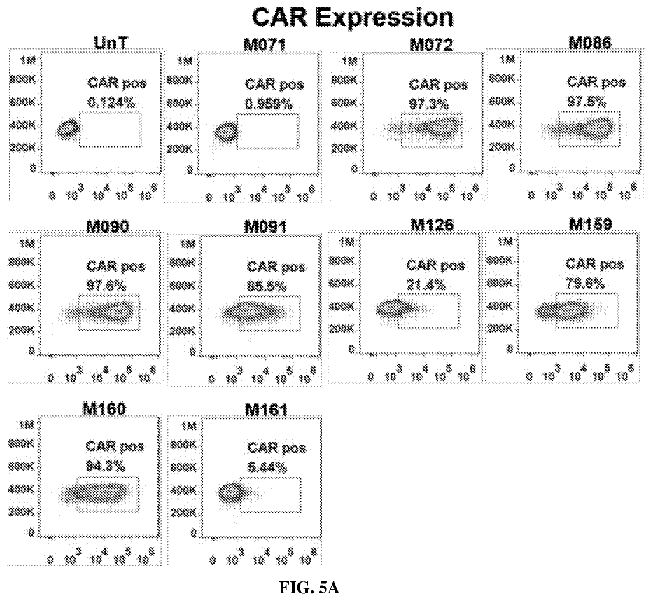

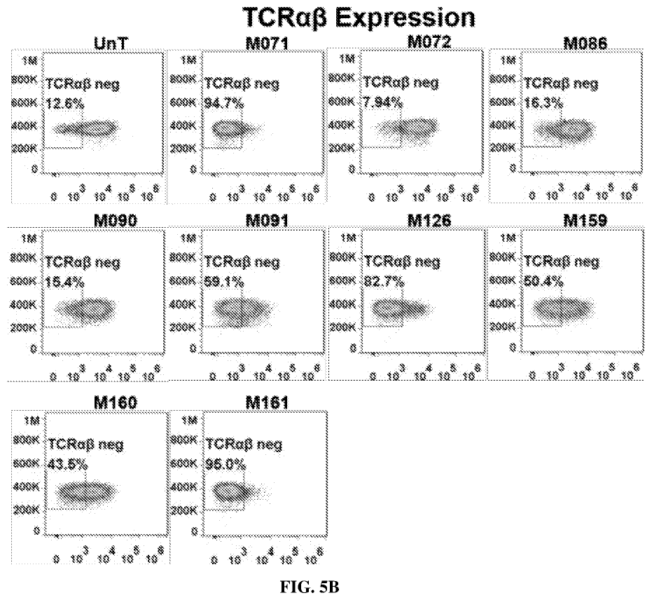

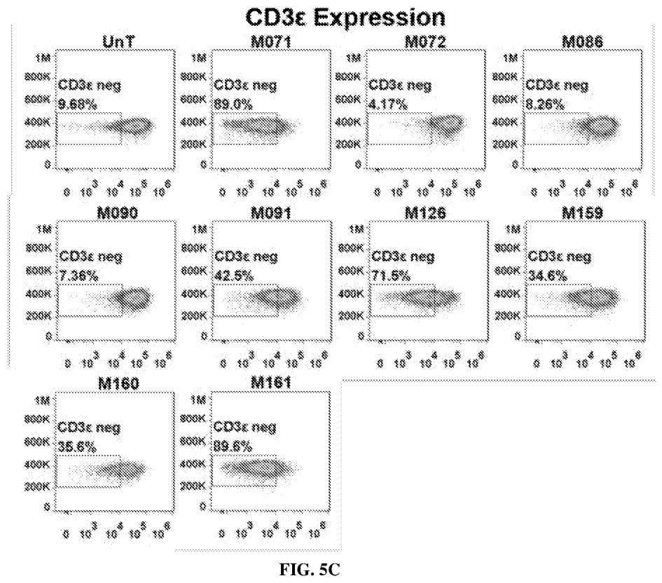

As can be seen from FIGS. 5A-5C, TCRαβ− rates of SIV Nef-P2A-CAR (M091), SIV Nef-IRES-CAR (M126), CAR-IRES-SIV Nef (M159), CAR-PGK-SIV Nef (M160), SIV Nef-PGK-CAR (M161) were 59.1%, 82.7%, 50.4%, 43.5%, 95.0%, respectively, that TCRαβ significantly downregulated compare with the UnT group (12.6%). Meanwhile, CAR-P2A-LNGFR-SIV Nef (M072), CAR-P2A-SIV Nef (M086), CAR-P2A-(GGGS)₃-SIV Nef (M090) TCRαβ− rates were 7.94%, 16.3%, 15.4%, respectively, which without significant difference compare with untransduced (UnT) group (12.6%). These above results indicated that CAR constructed at C′terminus of SIV Nef via a self-cleaving peptide P2A (M091) could down-regulate cell surface expression of TCR/CD3, while preserving sufficient CAR expression. But meanwhile, it is possible that the N-terminal spatial structure of SIV Nef protein is critical in down-regulating cell surface expression of TCR/CD3 complex, and the M072, M086 and M090 vector residual cleaved P2A amino acid at N′ terminus of Nef protein significantly affected its function. Table 5 below summarizes the effects of SIV Nef CAR All-in-One plasmids on the expression of CD3ε, TCRαβ, and BCMA CAR.

TABLE 5
All-in-One plasmids and effects on the expression of TCR/CD3 complex and CAR

| | | | TCR/CD3 Complex Expression | | |
|---|---|---|---|---|---|
| | | CAR | Down- | TCRαβ | CD3ε |
| Group | Structure | Expression % | regulation | neg % | neg % |
| UnT | Positive Control | 0.124 | N/A | 12.6 | 9.68 |
| M071 | SIV Nef-P2A-LNGFR | 0.959 | Yes | 94.7 | 89.0 |
| M072 | CAR-P2A-LNGFR -SIV Nef | 97.3 | No | 7.94 | 4.17 |
| M086 | CAR-P2A-SIV Nef | 97.5 | No | 16.3 | 8.26 |
| M090 | CAR-P2A-(GGGS)₃-SIV Nef | 97.6 | No | 15.4 | 7.36 |
| M091 | SIV Nef-P2A-CAR | 85.5 | Yes | 59.1 | 42.5 |
| M126 | SIV Nef-IRES-CAR | 21.4 | Yes | 82.7 | 71.5 |
| M159 | CAR-IRES-SIV Nef | 79.6 | Yes | 50.4 | 34.6 |
| M160 | CAR-PGK-SIV Nef | 94.3 | Yes | 43.5 | 35.6 |
| M161 | SIV Nef-PGK-CAR | 5.44 | Yes | 95.0 | 89.6 |

### Example 6. Nef Mutants and Subtypes with Reduced Negative Effect on T Cell CD4 and CD28 Expression

Certain amino acids on Nef protein can bind to CD4 and CD28, and then down-regulate CD4, CD28 expression on T cells (see Table 6). In order to reduce the negative effect of Nef on CD4 and CD28 expression and function, we further designed and constructed subtype Nef (HIV F2-Nef. HIV C2-Nef, HIV HV2NZ-Nef) and mutant Nef (referred to as “mutNef”).

TABLE 6
Functions of Nef amino acids
(Derived from V. Piguet and D. Trono, “A Structure-function Analysis of the Nef Protein of
Primate Lentiviruses”, 1999, [Rev Med Virol] 448-459)

| Amino acid | Domain | Function |
|---|---|---|
| G | myristoylation site | CD4 downregulation + MHC I |
| | | downmodulation |
| | | association with signaling molecules |
| RGKP | N-terminal α-helix (MHC I | MHC I downmodulation + |
| | downregulation + | association with signaling molecules |
| | protein kinase recruitment) | |
| DDEEE | acidic cluster (MHC-I | MHC I downmodulation |
| | downregulation) | |
| PVQPRVPLRQMTY | proline-based repeat (MHC-1 | MHC I downmodidation + |
| | downregulation + SH3 binding) | association with signaling molecules |
| RR | PAK binding | CD4 downregulation + association with |
| | | signaling molecules |
| AR | COP I recruitment | CD4 downregulation |
| LM | di-leucine based AP recruitment | CD4 downregulation |
| | (HIV-1 Nef) | |
| ED | V-ATPase and Raf-1 binding | CD4 downregulation + association with |
| | | signaling molecules |

### 1. Construction of Subtype or Mutant Nef Plasmids and Various Nef-Expressing Cell Lines

We designed SIV Nef sequence with mutated amino acids crucial for CD4 and CD28 binding. We also designed some other SIV Nef mutants and HIV Nef homologs. Subtype Nef-P2A-LNGFR or mutNef-P2A-LNGFR fusion sequences were chemically synthesized, then cloned into pLVX-hEF1α plasmid as described in Example 1, resulting in subtype Nef-P2A-LNGFR and mutNef-P2A-LNGFR transfer plasmids (see Table 7 for subtype or mutated Nef structure and “SEQUENCE LISTING” section for mutant sequences). M016 (scrambled sequence) served as negative control. M071 (wildtype SIV Nef-P2A-LNGFR) as constructed in Example 1 was used as positive control.

TABLE 7
Nef mutants and subtypes’ structure

| Vector | Protein | Type | Nef Information |
|---|---|---|---|
| M016 | Negative Control | — | Negative control, scrambled sequences |
| M071 | SIV Nef | — | Positive control, wildtype SIV Nef Protein |
| M116 | SIV Nef Mutant 1 or | Mutation | Mutate SIV Nef “di-leucine based AP recruitment domain” |
| | SIV Nef M116 | | |
| M117 | SIV Nef Mutant 2 | Mutation | Mutate SIV Nef “di-leucine based AP recruitment domain” |
| | | Mutation | Mutate SIV Nef “PAK binding domain” |
| M118 | SIV Nef Mutant 3 | Mutation | Mutate SIV Nef “PAK binding domain” |
| | | | Mutate SIV Nef “COP I recruitment domain” |
| | | | Mutate SIV Nef “di-leucine based AP recruitment domain” |
| | | | Mutate SIV Nef “V-ATPase and Raf-1 binding domain” |
| M119 | HIV F2-Nef | Subtype | HIV F2-Nef Protein |
| M120 | HIV C2-Nef | Subtype | HIV C2-Nef Protein |
| M121 | HIV HV2NZ-Nef | Subtype | HIV HV2NZ-Nef Protein |
| M142 | SIV Nef Mutant 4 | Mutation | Mutate SIV Nef “COP I recruitment domain” |
| | | | Mutate SIV Nef “di-leucine based AP recruitment domain” |
| | | | Mutate SIV Nef “V-ATPase and Raf-1 binding domain” |
| M143 | SIV Nef Mutant 5 | Mutation | Mutate SIV Nef “di-leucine based AP recruitment domain” |
| | | | Mutate SIV Nef “V-ATPase and Raf-1 binding domain” |

*M119, M120, and M121 mutNef sequences were derived from S. R. Das and S. Jameel, 2005 (Biology of the HIV Nef protein, Indian J Med Res., 121(4):315-332). Other mutants were self-designed.

Transfer plasmids from Table 7 were purified, mixed proportionally with packaging plasmids psPAX2 and envelope plasmids pMD2.G, then co-transduced into HEK 293T cells. 60 hrs post transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, then stored at −80° C.

Jurkat cells (Clone E6-1, ATCC® TIB-152™) were cultured as in Example 1. Lentiviruses carrying fusion sequence as in Table 6 were added into the supernatant of Jurkat cell culture for transduction. Puromycin was used as selectable marker for Nef+ cells. 72 hrs post transduction, puromycin of a final concentration of 1 μg/mL was added. Culture medium was changed every three days and supplemented with puromycin of the same concentration to further screen for single cell clones.

### 2. Testing the Effects of Subtype or Mutant Nef on CD4 and CD28 Expression on T Cells

72 hrs post-transduction, suspension containing 5×10⁵ Jurkat cells were collected and prepared for FACS as described in Example 1 to examine positive rates of CD3ε, TCRαβ, CD4 and CD28. 1 μL PE/Cy7 anti-human CD3 Antibody (BioLegend®, #300316), 1 μL PE/Cy5 anti-human TCR α/β Antibody (BioLegend®, #306710), 1 μL PE-CD4 or APC anti-human CD28 Antibody was used for FACS.

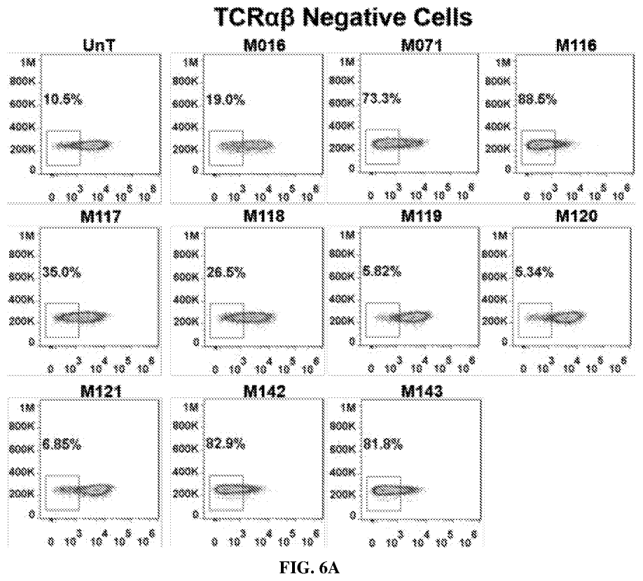

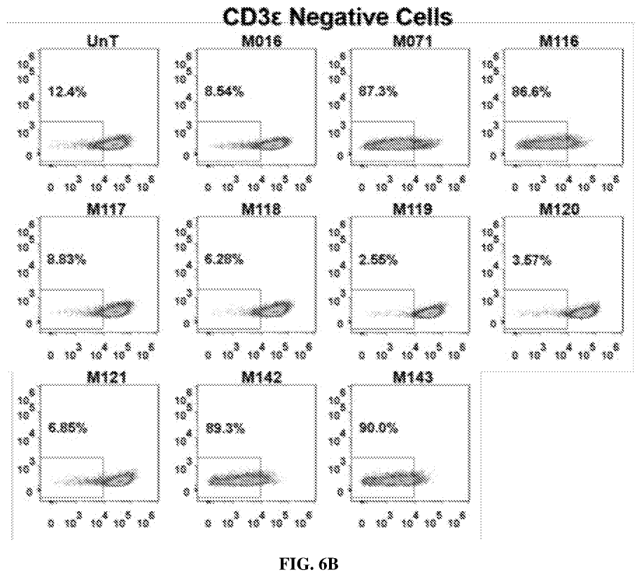

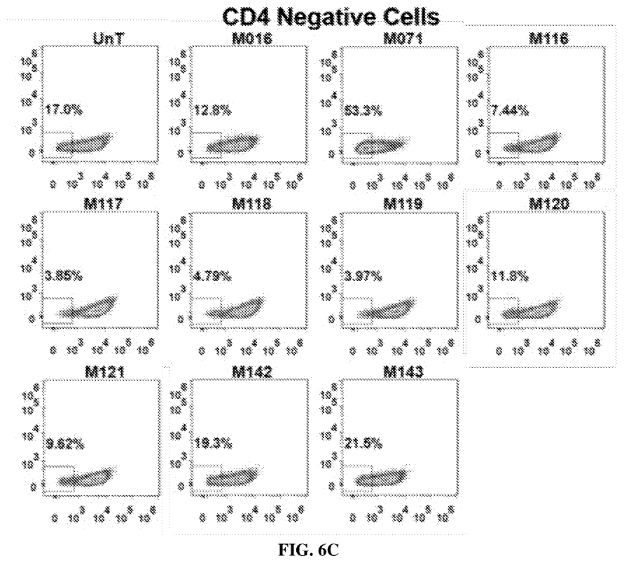

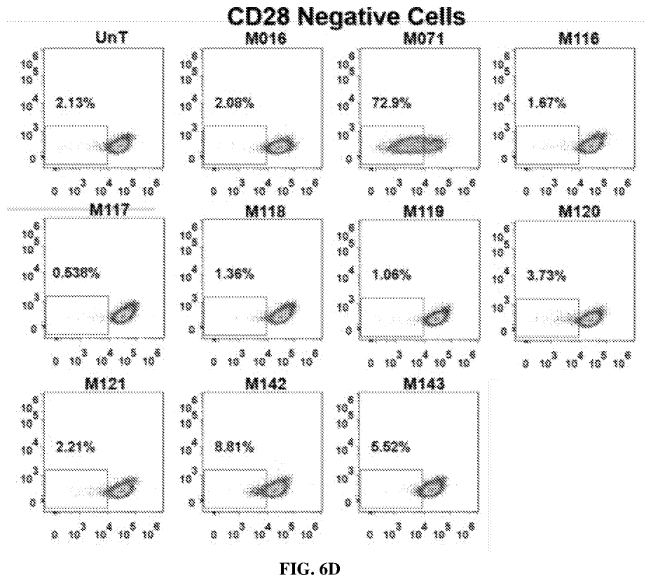

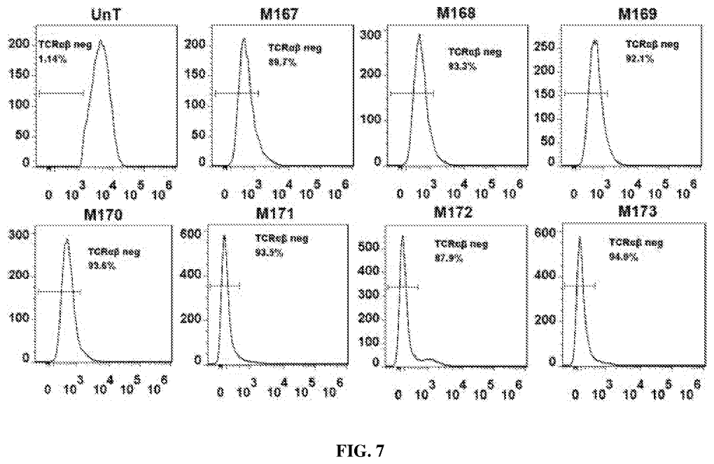

As can be seen from FIGS. 6A-6D, Jurkat cells transduced with M116 (SIV Nef Mutant 1 or SIV Nef M116) exhibited 88.5% TCRαβ-negative rate, 86.6% CD3ε-negative rate, which is not significantly different from those of cells transduced with M071 (wt SIV Nef; P>0.05), exhibiting 73.3% TCRαβ-negative rate, 87.3% CD3ε-negative rate. On the other hand, Jurkat cells transduced with M116 (SIV Nef Mutant 1 or SIV Nef M116) exhibited only 7.44% CD4-negative rate and 1.67% CD28-negative rate, which was significantly lower than 53.3% CD4-negative rate and 72.9% CD28-negative rate in Jurkat cells transduced with M071 (wt SIV Nef; P<0.05). This result indicated that compared to widetype SIV Nef protein, SIV Nef Mutant 1 in M116 plasmid could effectively down-regulate TCR/CD3 complex expression on T cell surface, while having very little down-regulation effect on CD4 and CD28 expression. M117 (SIV Nef Mutant 2), M118 (SIV Nef Mutant 3). M142 (SIV Nef Mutant 4) and M143 (SIV Nef Mutant 5) had similar effect effectively down-regulating TCR/CD3 complex expression while preserving CD4 and CD28 expression on T cell surface (i.e., having much less CD4 down-regulation effect and/or CD28 down-regulation effect). HIV subtype Nef proteins (Ml 19, M120, and M121) also had very little down-regulation effect on CD4 expression, but their down-regulation effect on TCR/CD3 complex on T cell surface was not as good as SIV Nef mutants (M116, M117, M118, M142 and M143). Table 8 summarizes the effects of different Nef subtypes and mutants on TCRαβ, CD3ε, CD4 and CD28 expression on T cells.

TABLE 8
Effects of Nef subtypes or mutants on TCRαβ, CD3ε, CD4 and CD28 expression on T cells

| Markers | UnT | M016 | M071 | M116 | M117 | M118 | M119 | M120 | M121 | M142 | M143 |
|---|---|---|---|---|---|---|---|---|---|---|---|
| TCRαβ neg | 10.5% | 19.0% | 73.3% | 88.5% | 35.0% | 26.5% | 5.82% | 5.34% | 6.85% | 82.9% | 81.8% |
| CD3ε neg | 12.4% | 8.54% | 87.3% | 86.6% | 8.83% | 6.28% | 2.55% | 3.57% | 6.85% | 89.3% | 90.0% |
| CD4 neg | 17.0% | 12.8% | 53.3% | 7.44% | 3.85% | 4.79% | 3.97% | 11.8% | 9.62% | 19.3% | 21.5% |
| CD28 neg | 2.13% | 2.08% | 72.9% | 1.67% | 0.54% | 1.36% | 1.06% | 3.73% | 2.21% | 8.81% | 5.52% |

### Example 7. Use of SIV Nef in CAR-T Cell Immunotherapy

### 1. Construction of SIV Nef CAR All-In-One Vector

Fusion gene sequences SIV Nef-IRES-CD20 scFv (Rituximab) CAR (SEQ ID NO: 48), SIV Nef-IRES-CD20 scFv (Leu-16) CAR (SEQ ID NO: 49), SIV Nef-IRES-CD19-CD20 scFv CAR (SEQ ID NO: 50), SIV Nef-IRES-CD19 scFv CAR (SEQ ID NO: 51). SIV Nef-IRES-BCMA BiVHH CAR1 (SEQ ID NO: 52). SIV Nef-IRES-BCMA BiVHH CAR2 (SEQ ID NO: 53), SIV Nef-IRES-BCMA mono-VHH CAR (SEQ ID NO: 54) were chemically synthesized, then cloned into pLVX-hEF1α expression vector (see Example 1) for the construction of recombinant transfer plasmids PLLV-M167, PLLV-M168, PLLV-M169, PLLV-M170, PLLV-M171, PLLV-M172, and PLLV-M173, respectively (see Table 9).

Transfer plasmids were purified and similarly transduced into HEK 293T cells with psPAX2 and pMD2.G plasmids. 60 hours post-transduction, viral supernatant was collected and centrifuged at 4° C., 3000 rpm for 5 min. The supernatant was filtered using 0.45 μm filter, and further concentrated using 500 KD hollow fiber membrane tangential flow filtration to obtain concentrated lentiviruses, then stored at −80° C.

TABLE 9
Exemplary SIV Nef + CAR All-in-One vectors

| Vector | Fusion | | Nucleic acid | CAR amino |
|---|---|---|---|---|
| name | gene | Fusion gene structure | sequence | add sequence |
| PLLV-M167 | M167 | SIV Nef-IRES-CD20 scFv (Rituximab) CAR | SEQ ID NO:48 | SEQ ID NO:55 |
| PLLV-M168 | M168 | SIV Nef-IRES-CD20 scFv (Leu-16) CAR | SEQ ID NO:49 | SEQ ID NO:56 |
| PLLV-M169 | M169 | SIV Nef-IRES-CD19 × xCD20 scFv CAR | SEQ ID NO:50 | SEQ ID NO:57 |
| PLLV-M170 | M170 | SIV Nef-IRES-CD19 scFv CAR | SEQ ID NO:51 | SEQ ID NO:58 |
| PLLV-M171 | M171 | SIV Nef-IRES-BCMA BiVHH CAR1 | SEQ ID NO:52 | SEQ ID NO:59 |
| PLLV-M172 | M172 | SIV Nef-IRES-BCMA BiVHH CAR2 | SEQ ID NO:53 | SEQ ID NO:60 |
| PLLV-M173 | M173 | SIV Nef-IRES-BCMA mono-VHH CAR | SEQ ID NO:54 | SEQ ID NO:61 |

### 2. Obtaining TCR Negative CAR-T Cells in One Step

T cells were obtained from thawed PBMC using Pan T cell Isolation Kit (Miltenyi Biotec, #130-096-535). Isolated T cells were seeded in 10 cm cell culture dishes, then supplemented with MicroBeads (Miltenyi Biotec, #130-111-160) according to the manufacturer's instructions, and incubated in 37° C., 5% CO₂ incubator for 72 h.

Lentiviruses carrying SIV Nef-IRES-CD19 scFv CAR, SIV Nef-IRES-CD20 scFv CAR, SIV Nef-IRES-CD19×CD20 scFv CAR, SIV Nef-IRES-BCMA BiVHH CARL, SIV Nef-IRES-BCMA BiVHH CAR2, or SIV Nef-IRES-BCMA mono-VHH CAR sequences were added into cultured primary T cell suspension for transduction, respectively. After transduction, TCR negative CAR-T cells were isolated and enriched using TCRαβ cell isolation kit (Miltenyi Biotec, #130-092-614) with MACS.

One day after MACS, suspension containing enriched 5×10⁵ TCRαβ negative cells were collected and prepared for FACS to examine the expression of TCRαβ.

As can be seen from FIG. 7, TCRα negative T cell rates post-MACS enrichment were pretty high for T cells transduced with SIV Nef+CAR all-in-one constructs, while un-transduced T cells (UnT) post MACS only produced 1.14% of TCRαβ negative rate. See, post-MACS TCRαβ negative rate for SIV Nef-IRES-CD20 scFv (Rituximab) CAR (M167) T cells (89.7%), SIV Nef-IRES-CD20 scFv (Leu-16) CAR (M168) T cells (93.3%), SIV Nef-IRES-CD19×CD20 scFv CAR (M169) T cells (92.1%), SIV Nef-IRES-CD19 scFv CAR (M170) T cells (93.6%), SIV Nef-IRES-BCMA BiVHH CAR1 (M171) T cells (93.5%), SIV Nef-IRES-BCMA BiVHH CAR2 (M172) T cells (87.9%), and SIV Nef-IRES-BCMA mono-VHH CAR (M173) T cells (94.0%).

MACS-sorted TCRαβ negative T cells, MACS-sorted TCRαβ positive T cells, and un-transduced T cells (UnT) from the above steps were then mixed under different effector to target (E:T) cell ratios with target cells or tumor cells respectively, and incubated in Corning® 384-well solid white plate for 12 h. K562-CD20 is CD20 transduced myelogenous leukemia cell line. Raji is B-cell lymphoma cell line (CD19+, CD20+, BCMA−). K562-CD19 is CD19 transduced myelogenous leukemia cell line. RPMI-8226 is multiple myeloma cell line expressing BCMA. One-Glo™ Luciferase Assay System (TAKARA, #B6120) was used to measure luciferase activity. 25 μL One-Glo™ Reagent was added to each well of the 384-well plate. After incubation, fluorescence was measured using Spark™ 10M multimode microplate reader (TECAN), in order to calculate cytotoxicity rates of different T lymphocytes on target cells. The scenario was MACS-sorted TCRα positive T cells would show TCR-mediated non-specific killing activity as TCRs were not depleted by SIV Nef, while MACS-sorted TCRαβ negative T cells were nearly depleted of TCRs thus would show mainly CAR-mediated specific killing activity.

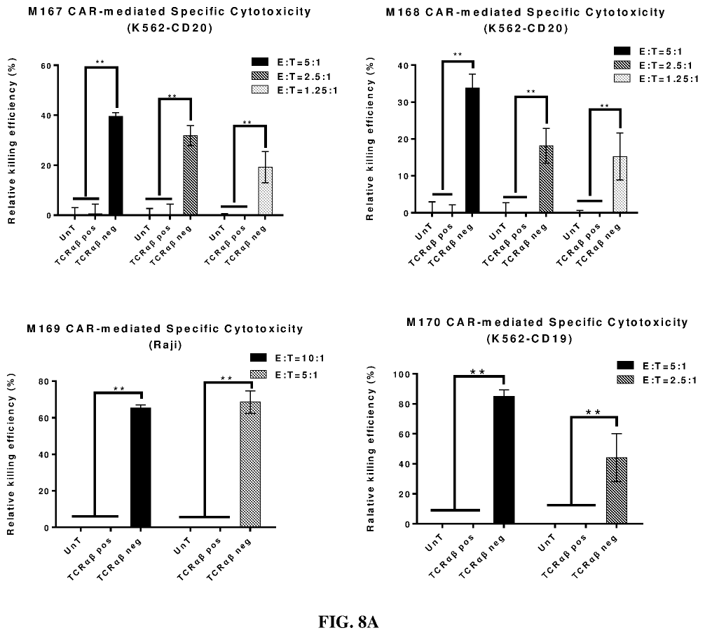

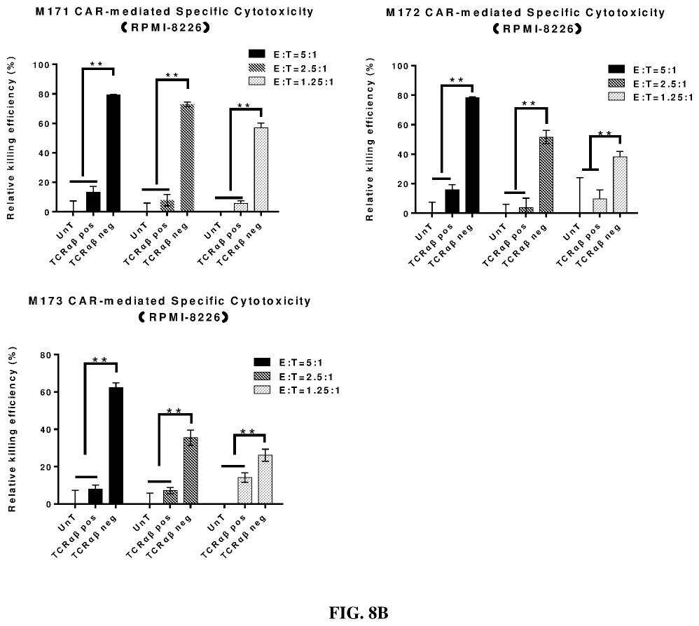

FIGS. 8A-8B demonstrate CAR-mediated specific tumor cytotoxicity of MACS-sorted TCRαβ negative T cells transduced with various SIV Nef+CAR all-in-one constructs, with MACS-sorted TCRαβ positive T cells transduced with various SIV Nef+CAR all-in-one constructs and un-transduced T cells (UnT) as controls. As can be seen from FIGS. 8A-8B, MACS-sorted TCRαβ negative T cells showed significantly higher tumor cell killing activity compared to MACS-sorted TCRαβ positive T cells and un-transduced T cells, and tumor cell killing activity is positively correlated with E:T ratio (the higher the E:T, the better the CAR-mediated killing efficacy).

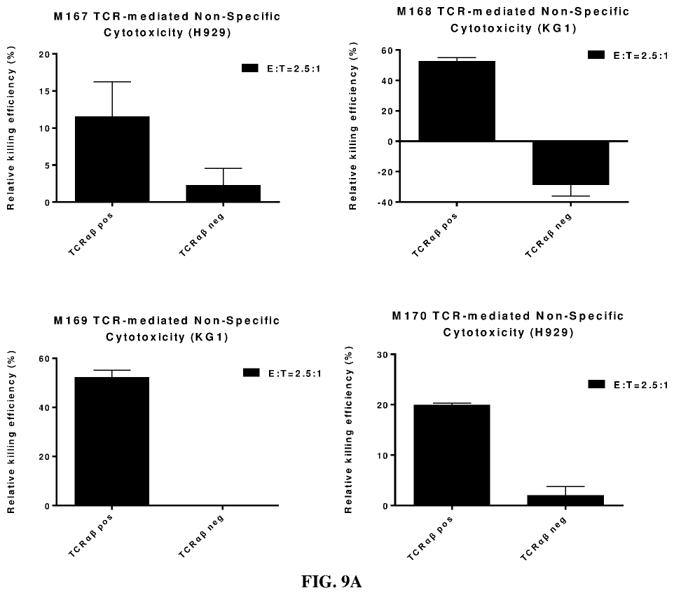

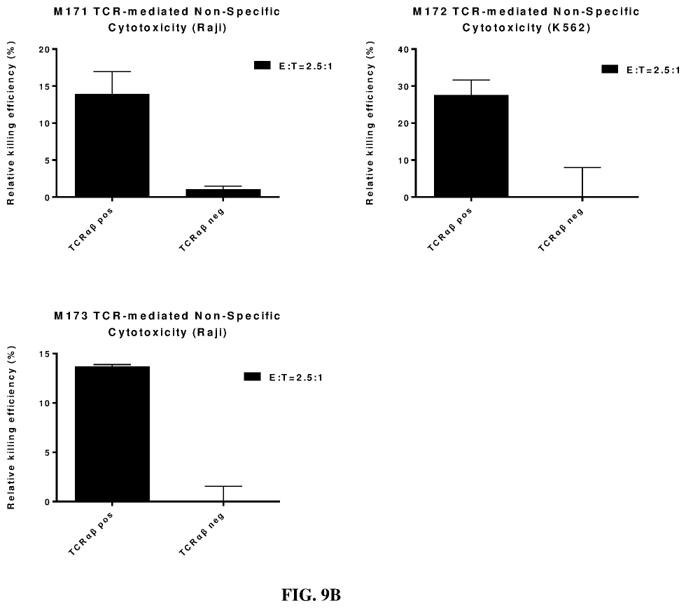

FIGS. 9A-9B demonstrate TCR-mediated non-specific killing efficiency of MACS-sorted TCRαβ positive and negative T cells transduced with various SIV Nef+CAR all-in-one constructs. H929 is human multiple myeloma cell line (CD19−, CD20−). KG1 is human acute myeloid leukemia cell line (CD19-). Raji is Burkitt lymphoma cell line (CD19+, CD20+, BCMA−). K562 is myelogenous leukemia cell line (CD20-, CD19-, BCMA−). As can be seen from FIGS. 9A-9B, MACS-sorted TCRαβ negative T cells (expressing CAR and little or no TCR) had little or no killing activity on target cells, as expected, because corresponding CAR-antigen (e.g., CD19, CD20, BCMA) was not expressed on the corresponding tested target cells; while MACS-sorted TCRαβ positive T cells (expressing only TCR) caused much higher TCR-mediated non-specific tumor cell killing.

These results indicate that SIV Nef+CAR all-in-one vectors are effective and easy to generate TCRαβ negative CAR-T cells, which can effectively cause CAR-mediated specific tumor cell killing (P<0.05) and without TCR-mediated non-specific cytotoxicity. Thus, SIV Nef+CAR all-in-one vectors as exemplified herein can effectively reduce TCRαβ expression and function on primary T cells, while maintaining CAR expression and CAR-mediated specific cytotoxicity on target cells.

### Example 8. Use of SIV Nef Mutant in CAR-T Cell Immunotherapy

SIV Nef M116 sequence (see SIV Nef Mutant 1 in Example 6) was used in this experiment for constructing SIV Nef+CAR all-in-one vector. Fusion gene sequence BCMA BiVHH CAR1-IRES-SIV Nef M116 (SEQ ID NO: 62) was chemically synthesized, and cloned into the PLVX-hEF1α expression plasmid (see Example 1), resulting in recombinant BCMA BiVHH CAR1-IRES-SIV Nef M116 transfer plasmid (hereinafter referred to as “PLLV-M133”). PLLV-M133 recombinant transfer plasmid was purified, mixed proportionally with packaging plasmid psPAX2 and envelope plasmid pMD2.G, then co-transduced into HEK 293T cells. 60 hours post-transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, then stored at −80° C.

Primary T cells were obtained as described in Example 7, then transduced with lentiviruses carrying PLLV-M133. After transduction, TCR negative CAR-T cells were isolated and enriched using TCRαβ cell isolation kit (Miltenyi Biotec, #130-092-614) with MACS. One day after MACS, suspension containing enriched 5×10⁵ TCRαβ negative cells were collected and prepared for FACS to examine the expression of TCRαβ.

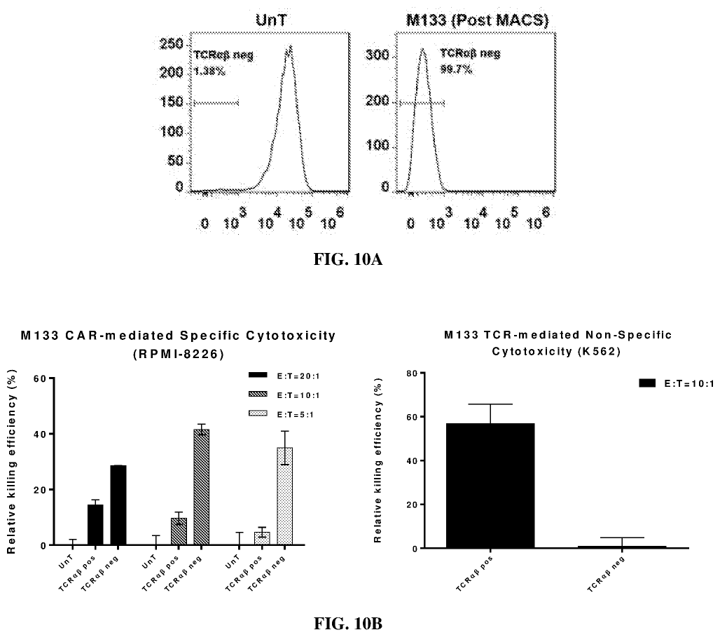

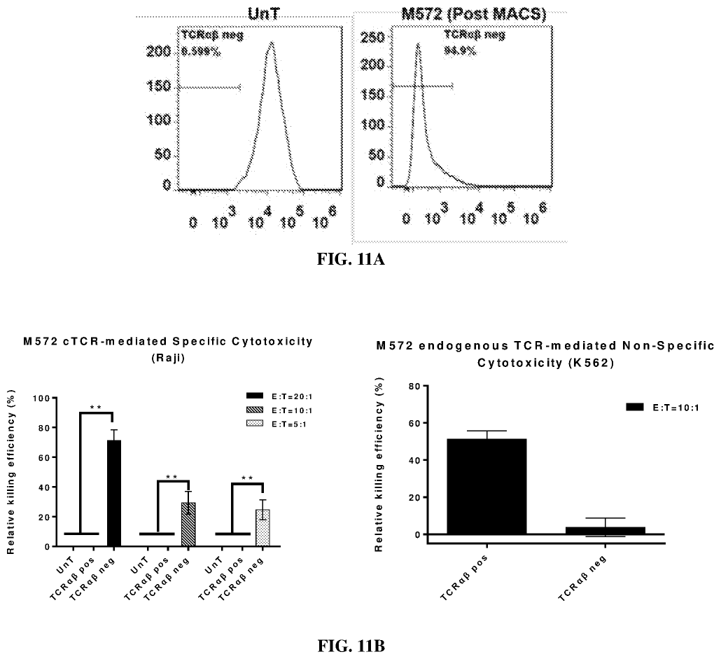

As shown in FIG. 10A, TCRαβ negative M133 CAR-T cell ratio post-MACS enrichment was 99.7%. Un-transduced T cells served as control, which only showed 1.38% TCRαβ negative rate.

Cytotoxicity assay was conducted as described in Example 7. K562 is myelogenous leukemia cell line (BCMA−). RPMI-8226 is multiple myeloma cell line expressing BCMA. As can be seen from FIG. 10B left panel, MACS-sorted TCRαβ negative M133 CAR-T cells led to significantly higher cytotoxicity (34.99±6.20%) on RPMI-8226 cells (BCMA+) compared to MACS-sorted TCRαβ positive M133 T cells and un-transduced T cells, reflecting CAR-T mediated specific tumor cell killing. MACS-sorted TCRαβ4 negative M133 T cells had almost no TCR-mediated non-specific cell killing (0.90±3.45%) on K562 cells (BCMA−), MACS-sorted TCRαβ positive T cells caused much higher TCR-mediated non-specific cell killing (FIG. 10B right panel).

These results indicate that SIV Nef+CAR all-in-one vector carrying mutant SIV Nef sequence is also effective and easy to generate TCRαβ negative CAR-T cells, and the organizations of SIV Nef-IRES-CAR and CAR-IRES-SIV Nef both work effectively (compare Examples 7 and 8). Both sequence organizations can effectively reduce TCRαβ expression and function on primary T cells, while maintaining CAR expression and CAR-mediated specific cytotoxicity on target cells.

### Example 9. Use of SIV Nef Mutant in Chimeric TCR-T (cTCR-T) Cell Immunotherapy

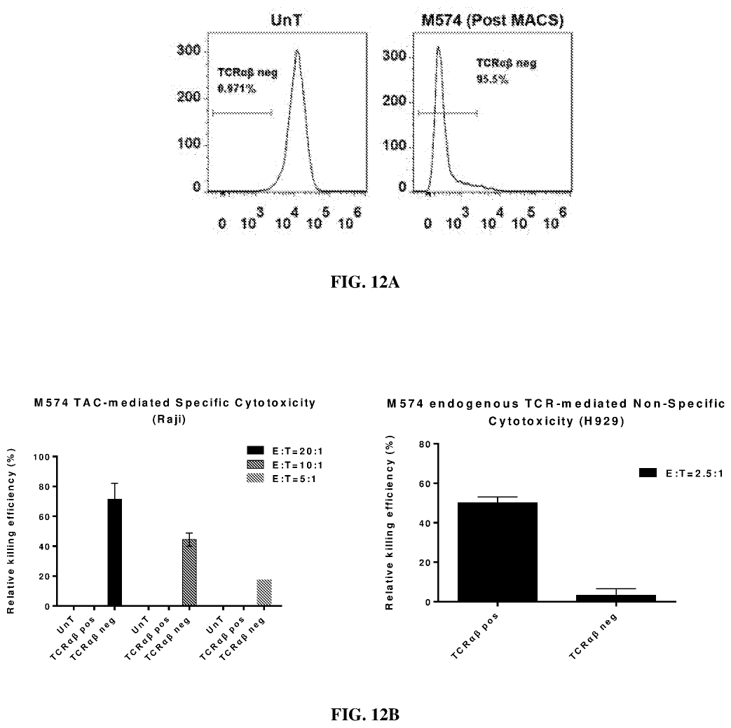

SIV Nef M116 sequence (see M116, SIV NEF Mutant 1 in Example 6) and anti-CD20 chimeric TCR (cTCR) sequence were used in this experiment for constructing SIV-Nef+ chimeric TCR all-in-one vector. Anti-CD20 cTCR has the structure of anti-CD20 scFv (Leu-16)-(GGGGS)₃-CD3ε (full length except signal peptide), with the amino acid sequence of SEQ ID NO:64. By incorporating the anti-CD20 cTCR fusion polypeptide into the native TCR complex, the modified TCR complex can recognize tumor cells expressing CD20 without the need for HLA, and engage the complete TCR machinery to drive complete T cell functions required for potent, modulated and durable tumor killing. Fusion gene sequence SIV Nef M116-IRES-CD20 cTCR (SEQ ID NO: 63) was chemically synthesized, and cloned into the PLVX-hEF1α expression plasmid (see Example 1), resulting in recombinant SIV Nef M116-IRES-CD20 cTCR transfer plasmid (hereinafter referred to as “PLLV-M572”). PLLV-M572 recombinant transfer plasmids were purified, mixed proportionally with packaging plasmid psPAX2 and envelope plasmid pMD2.G, then co-transduced into HEK 293T cell. 60 hours post-transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, then stored at −80° C. TCRαβ negative CD20-cTCR positive T cells were prepared by transducing primary T cells with lentiviruses carrying PLLV-M572 and then MACS enrichment, as described in Example 7. The expression of TCRαβ was examined according to similar methods as described above. As shown in FIG. 11A, TCRαβ negative T-cell rate post-MACS enrichment was 94.9% for T cells transduced with PLLV-M572, while untransduced T cells only had 0.599% TCRαβ negative rate.

Cytotoxicity assay was conducted as described in Example 7. K562 is myelogenous leukemia cell line (CD20−. CD19−, BCMA−). Raji is Burkitt lymphoma cell line (CD19+, CD20+, BCMA−). As can be seen from FIG. 11B left panel, MACS-sorted TCRαβ negative CD20 cTCR-T cells led to significantly higher cytotoxicity (49.21±22.96%) on Raji cells (CD20+) compared to MACS-sorted TCRαβ positive M572 T cells and untransduced T cells, reflecting CD20 cTCR mediated specific tumor cell killing. Further, the killing efficacy of MACS-sorted TCRαβ negative CD20 cTCR-T cells was higher with higher E:T ratio. MACS-sorted TCRαβ negative CD20 cTCR-T cells had little endogenous TCR mediated non-specific cell killing (3.76±4.31%) on K562 cells (CD20-), while MACS-sorted TCRαβ positive M572 T cells caused much higher endogenous TCR-mediated non-specific cell killing (FIG. 11B right panel).

These surprising results indicate that SIV Nef+cTCR all-in-one vector could effectively down-regulate endogenous TCRαβ expression and function on primary T cells, without affecting the function of TCR complex integrated with exogenous cTCR. Further, TCRαβ negative cTCR-T cells effectively mediated cTCR specific cytotoxicity on tumor cells (P<0.05), with little or no endogenous TCR-mediated non-specific cytotoxicity.

### Example 10. Use of SIV Nef Mutant in TAC-T Cell Immunotherapy

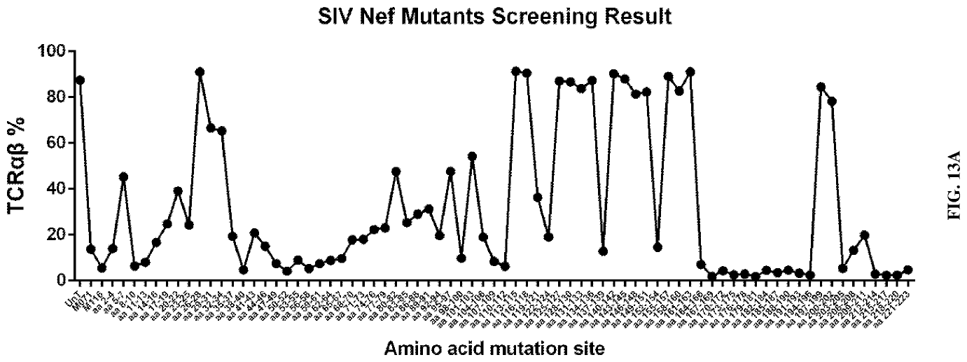

SIV Nef M116 (see M116, SIV NEF Mutant 1 in Example 6) was used in this experiment for constructing SIV Nef+CD20 TAC all-in-one vector. Anti-CD20 TAC comprises the structure of anti-CD20 scFv (Leu-16)-(GGGGS)₃-huUCHT1.Y177T-GGGGS-CD4 (partial extracellular domain+transmembrane domain+intracellular domain), with the amino acid sequence of SEQ ID NO: 66. huUCHT1 targets CD3s. Fusion gene sequence SIV Nef M116-IRES-CD20 TAC (SEQ ID NO: 65) was chemically synthesized, and cloned into the PLVX-hEF1α expression plasmid (see Example 1), resulting in recombinant SIV Nef-IRES-CD20 TAC transfer plasmid (hereinafter referred to as “PLLV-M574”). PLLV-M574 recombinant transfer plasmid was purified, mixed proportionally with packaging plasmid psPAX2 and envelope plasmid pMD2.G, then co-transduced into HEK 293T cells. 60 hours post-transduction, viral supernatant was collected, and centrifuged at 4° C., 3000 rpm for 5 min. The supernatant was filtered using 0.45 μm filter, then further concentrated using 500 KD hollow fiber membrane tangential flow filtration to obtain concentrated lentiviruses, then stored at −80° C. TCRαβ negative TAC-T cells were prepared by transducing primary T cells with lentiviruses carrying PLLV-M574 and then MACS enrichment, as described in Example 7. The expression of TCRαβ was examined according to similar methods as described above. As shown in FIG. 12A, TCRαβ negative rate of T cells transduced with PLLV-M574 post-MACS enrichment was 95.5%, while untransduced T cells (UnT) only had 0.971% TCRαβ negative rate.

Cytotoxicity assay was conducted as described in Example 7. Raji is Burkitt lymphoma cell line (CD20+). H929 is human multiple myeloma cell line (CD20-). As can be seen from FIG. 12B left panel, MACS-sorted TCRαβ negative CD20 TAC-T cells caused significantly higher cytotoxicity (54.58±20.03%) on Raji cells (CD20+) compared to MACS-sorted TCRαβ positive M574 T cells and untransduced T cells, reflecting anti-CD20 TAC mediated specific tumor cell killing. Further, the killing efficacy of MACS-sorted TCRαβ negative CD20 TAC-T cells was higher with higher E:T ratio. MACS-sorted TCRαβ negative CD20 TAC-T cells had little endogenous TCR mediated non-specific cell killing (3.33±2.80%) on H929 cells (CD20-), while MACS-sorted TCRα P positive M574 T cells caused much higher endogenous TCR-mediated non-specific cell killing (FIG. 12B right panel).

These surprising results indicate that SIV Nef+TAC all-in-one vector can effectively down-regulate endogenous TCRαβ expression and function on primary T cells, without affecting the expression and function of TAC. Further, TCRαβ negative TAC-T cells effectively mediate TAC specific cytotoxicity on tumor cells (P<0.05), with little or no endogenous TCR-mediated non-specific cytotoxicity.

### Example 11. Test of SIV Nef Domain Elements for TCRαβ, CD4 and CD28 Regulation

Full length SIV Nef has 223 amino acids. Certain amino acids on Nef protein can bind to CD4 and CD28, and then down-regulate CD4. CD28 expression on T cells (see Example 6, Table 6). In order to test the effects of various Nef domain elements on TCRαβ, CD4 and CD28 expression and function, 74 mutants were designed by mutating every three consecutive amino acids to Alanine-Alanine-Alanine (AAA) across the full-length sequence except for the first Methionine. These mutant nucleic acid sequences were chemically synthesized, and cloned into the PLVX-hEF1α expression plasmid (see Example 1), resulting in 74 recombinant SIV Nef mutant transfer plasmids. Lentiviruses carrying each recombinant transfer plasmid were prepared as described above, see, e.g., Example 7. Jurkat cells were infected by 74 lentiviruses, respectively, and positive cell clones were selected using 1 μg/mL puromycin for 2 week. The expression of TCRαβ, CD4 and CD28 on Jurkat cells were examined by FACS as described above, see, e.g., Example 1. Untransduced Jurkat cells served as negative control. Jurkat cells transduced with M071 (wildtype SIV Nef) or M116 (SIV Nef M116, see Example 6) served as positive controls. At least 3% regulation was considered as the cutoff for further evaluation of the effect of SIV Nef mutants on the regulation of TCRαβ, CD4, and CD28 expression. For example, if the down-regulation level of TCRαβ by mutant SIV Nef was between 0% (including 0%) and less than 3% different from that down-regulated by wildtype SIV Nef, or if the mutant SIV Nef down-regulates TCRαβ more than (or equal to) 3% compared to that by wildtype SIV Nef, the mutant SIV Nef was considered to have “similar (or more) TCRαβ3 down-regulation compared to wildtype SIV Nef.” If the mutant SIV Nef down-regulates CD4 (and/or CD28) less than 3% compared to that by wildtype SIV Nef, such mutant SIV Nef was considered to have “less CD4 down-regulation compared to wildtype SIV Nef” (and/or “less CD28 down-regulation compared to wildtype SIV Nef”).

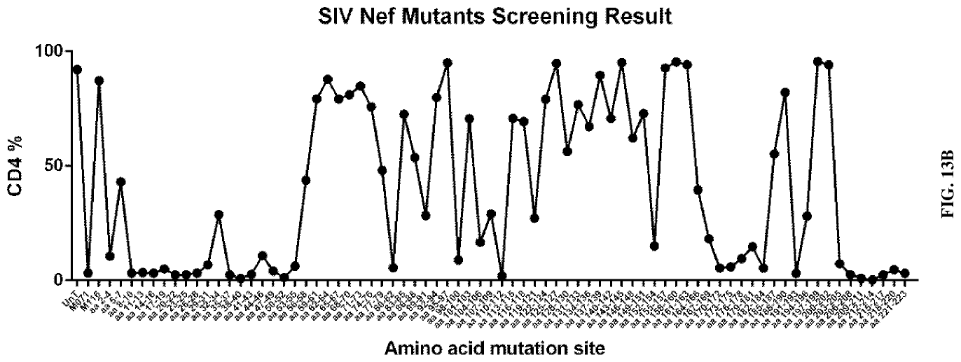

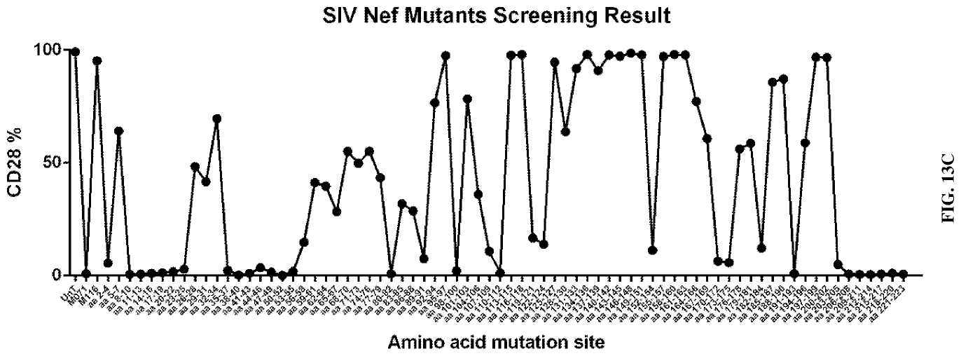

As shown in FIGS. 13A-13C, 74 SIV Nef mutants were screened side-by-side for their abilities in regulating TCRαβ, CD4 and CD28 expression, compared to wildtype SIV Nef (M071). Mutation positions and their corresponding functions are listed in Table 10. This screening experiment resulted in various sets of SIV Nef mutants with distinct regulatory functions. 34 SIV Nef mutants maintained the down-regulation effect on TCRαβ expression compared to wildtype SIV Nef (M071). Of these, 18 mutants further showed less down-regulation of CD4 compared to wildtype SIV Nef, 19 mutants further showed less down-regulation of CD28 compared to wildtype SIV Nef, and 16 mutants were found to not only maintain the TCRαβ3 downregulation effect of wildtvpe SIV Nef (M071), but also have less down-regulation effect on CD4 and CD28 compared to wildtype SIV Nef (M071). See Table 11 for detailed summary.

TABLE 10
Regulatory effects of SIV Nef mutants compared to wildtype SIV Nef (M071)

| Construct/ | Similar (or more) TCRαβ | Less CD4 | Less CD28 |
|---|---|---|---|
| amino acid | downregulation | downregulation | downregulation |
| mutation | compared to wildtype | compared to wildtype | compared to wildtype |
| site | SIV Nef | SIV Nef | SIV Nef |
| M116 (aa | + | + | + |
| 178-179) | | | |
| aa 2-4 | + | + | + |
| aa 5-7 | − | + | + |
| aa 8-10 | + | − | − |
| aa 11-13 | + | − | − |
| aa 14-16 | − | − | − |
| aa 17-19 | − | − | − |
| aa 20-22 | − | − | − |
| aa 23-25 | − | − | − |
| aa 26-28 | − | − | + |
| aa 29-31 | − | + | + |
| aa 32-34 | − | + | + |
| aa 35-37 | − | − | − |
| aa 38-40 | + | − | − |
| aa 41-43 | − | − | − |
| aa 44-46 | + | + | − |
| aa 47-49 | + | − | − |
| aa 50-52 | + | − | − |
| aa 53-55 | + | − | − |
| aa 56-58 | + | + | + |
| aa 59-61 | + | + | + |
| aa 62-64 | + | + | + |
| aa 65-67 | + | + | + |
| aa 68-70 | − | + | + |
| aa 71-73 | − | + | + |
| aa 74-76 | − | + | + |
| aa 77-79 | − | + | + |
| aa 80-82 | − | − | − |
| aa 83-85 | − | + | + |
| aa 86-88 | − | + | + |
| aa 89-91 | − | + | + |
| aa 92-94 | − | + | + |
| aa 95-97 | − | + | + |
| aa 98-100 | + | + | − |
| aa 101-103 | − | + | + |
| aa 104-106 | − | + | + |
| aa 107-109 | + | + | + |
| aa 110-112 | + | − | − |
| aa 113-115 | − | + | + |
| aa 116-118 | − | + | + |
| aa 119-121 | − | + | + |
| aa 122-124 | − | + | + |
| aa 125-127 | − | + | + |
| aa 128-130 | − | + | + |
| aa 131-133 | − | + | + |
| aa 134-136 | − | + | + |
| aa 137-139 | + | + | + |
| aa 140-142 | − | + | + |
| aa 143-145 | − | + | + |
| aa 146-148 | − | + | + |
| aa 149-151 | − | + | + |
| aa 152-154 | + | + | + |
| aa 155-157 | − | + | + |
| aa 158-160 | − | + | + |
| aa 161-163 | − | + | + |
| aa 164-166 | + | + | + |
| aa 167-169 | + | + | + |
| aa 170-172 | + | − | + |
| aa 173-175 | + | − | + |
| aa 176-178 | + | + | + |
| aa 179-181 | + | + | + |
| aa 182-184 | + | − | + |
| aa 185-187 | + | + | + |
| aa 188-190 | + | + | + |
| aa 191-193 | + | − | − |
| aa 194-196 | + | + | + |
| aa 197-199 | − | + | + |
| aa 200-202 | − | + | + |
| aa 203-205 | + | + | + |
| aa 206-208 | + | − | − |
| aa 209-211 | − | − | − |
| aa 212-214 | + | − | − |
| aa 215-217 | + | − | − |
| aa 218-220 | + | − | − |
| aa 221-223 | + | − | − |

TABLE 11
Summary of the effects of SIV Nef amino acid mutation
sites on TCRαβ, CD4, and CDS expression

| | Effects (compared to | |
|---|---|---|
| Group | wildtype SIV Nef) | Amino add (aa) mutation sites |
| 1 | Similar (or more) | aa 2-4, aa 8-10, aa 11-13 (e.g., aa 8-13), aa 38-40, aa 44-46, aa 47-49, |
| | TCRαβ down- | aa 50-52, aa 53-55, aa 56-58, aa 59-61, aa 62-64, aa 65-67 (e.g., aa 44- |
| | regulation | 67), aa 98-100, aa 107-109, aa 110-112 (e.g., aa 107-112), aa 137-139, |
| | | aa 152-154, aa 164-166, aa 167-169, aa 170-172, aa 173-175, aa 176- |
| | | 178, aa 178-179, 179-181aa, aa 182-184, aa 185-187, aa 188-190, aa |
| | | 191-193, aa 194-196 (e.g., aa 164-196), aa 203-205, aa 206-208 (e.g., |
| | | aa 203-208), aa 212-214, aa 215-217, aa 218-22.0, aa 221-22.3 (e.g., aa |
| | | 2.12.-22.3) |
| 2 | Similar (or more) | aa 2-4, aa 44-46, aa 56-58, aa 59-61, aa 62-64, aa 65-67 (e.g., aa 44- |
| | TCRαβ down- | 67), aa 98-100, aa 107-109, aa 137-139, aa 152-154, aa 164-166, aa |
| | regulation, less CD4 | 167-169 (e.g., aa 164-169), aa 176-178, aa 178-179, aa 179-181 (e.g., |
| | down-regulation | aa 176-181), aa 185-187, aa 188-190 (e.g., aa 185-190), aa 194-196, aa |
| | | 203-205 |
| 3 | Similar (or more) | aa 2-4, aa 56-58, aa 59-61, aa 62-64, aa 65-67 (e.g., aa 56-67), aa 107- |
| | TCRαβ down- | 109, aa 137-139, aa 152-154, aa 164-166, aa 167-169, aa 170-172, aa |
| | regulation, less CD28 | 173-175, aa 176-178, 178-179aa, aa 179-181, aa 182-184, aa 185-187, |
| | down-regulation | aa 188-190 (e.g., aa 164-190), aa 194-196, aa 203-205 |
| 4 | Similar (or more) | aa 2-4, aa 56-58, aa 59-61, aa 62-64, aa 65-67 (e.g., aa 56-67), aa 107- |
| | TCRαβ down- | 109, aa 137-139, aa 152-154, aa 164-166, aa 167-169 (e.g., aa 164- |
| | regulation, less CD4 | 169), aa 176-178, aa 178-179, aa 179-181 (e.g., aa 176-181), aa 185- |
| | down-regulation, less | 187, aa 188-190 (e.g., aa 185-190), aa 194-196, aa 2.03-205 |
| | CD28 down- | |
| | regulation | |

SEQUENCE LISTING

SEQ ID NO: 1 (wildtype SIV Nef nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAAGAAGAGAAAAG
ATCCTGAATTTGTATGCCTTGAACGAGTGGGGAATAATAGATGATTGGCAAGCTTACTCACCAG
GCCCCGGGGATAAGGTACCCGAGAGTCTTTGGCTTCTGCTTTAAGCTAGTCCCAGTGGACCTGC
ATGAGGAGGCACGCAACTGTGAGAGACACTGTCTGATGCATCCAGCACAGATGGGGGAAGAT
CCTGATGGAATAGATCATGGAGAAGTCTTGGTCTGGAAGTTTGACCCGAAGTTGGCGGTGGA
GTACCGCCCGGACATGTTTAAGGACATGCACGAACATGCAAAGCGC
SEQ ID NO: 2 (HIV1 Nef nucleic acid sequence)
ATGGGTGGCAAGTGGTCAAAAAGTAGTGTGATTGGATGGCCTACTGTAAGGGAAAGAATGAG
ACGAGCTGAGCCAGCAGCAGATAGGGTGGGAGCAGCATCTCGAGACCTCCAAAAACATGGA
GCAATCACAAGTAGCAATACAGCAGCTACCAATGCTGCTTGTGCCTGGCTAGAAGCACAAGA
GGAGGAGGAGGTGGGTTTTCCAGTCACACCTCAGGTACCTTTAAGACCAATGACTTACAAGG
CAGCTGTAGATCTTAGCCACTTTTTAAAAGAAAAGGGGGGACTGGAAGGGCTAATTCACTCC
CAAAGAAGACAAGATATCCTTGATCTGTGGATCTACCACACACAAGGCTACTTCCCTGATTgG
CAGAACTACACACCAGGGCCAGGGGTCAGATATCCACTGACCTTTGGATGGTGCTACAAGCT
AGTACCAGTTGAGCCAGATAAGATAGAAGAGGCCAATAAAGGAGAGAACACCAGCTTGTTAC
ACCCTGTGAGCCTGCATGGGATGGATGACCCGGAGAGAGAAGTGTTAGAGTGGAGGTTTGAC
AGCCGCCTAGCATTTCATCACGTGGCCCGAGAGCTGCATCCGGAGTACTTCAAGAACTGC
SEQ ID NO: 3 (HIV2 Nef nucleic acid sequence)
ATGGGTGCGAGTGGATCCAAGAAGCTTTCCAAGCATTCGCGAGGACTACGAGAGAGACTCTT
GCGGGCGCGTGGGGATGGTTATGGGAAGCAGCGCGACGCATCGGGAGGGGAATACTCGCAGT
TCCAAGAAGAATCAGGCAGGGAGCAGAACTCGCCCTCCTGTGAGGGACAGCAGTATCAGCA
GGGAGAGTACATGAACAGCCCATGGAGAAACCCAGCAACAGAAAGACAGAAAGATTTGTAT
AGGCAGCAAAATATGGATGATGTAGATTCTGATGATGATGACCTAATAGGAGTTCCTGTTACAC
CAAGAGTACCACGGAGAGAAATGACCTATAAATTGGCAATAGATATGTCACATTTTATAAAAG
AAAAAGGGGGACTGCAAGGGATGTTTTACAGTAGGAGGAGACATAGAATCCTAGACATATAC
CTAGAAAAAGAGGAAGGGATAATACCAGATTGGCAGAATTATACTCATGGGCCAGGAGTAAG
GTACCCAATGTACTTCGGGTGGCTGTGGAAGCTAGTATCAGTAGAACTCTCACAAGAGGCAG
AGGAAGATGAGGCCAACTGCTTAGTACACCCAGCACAAACAAGCAGACATGATGATGAGCAT
GGGGAGACATTAGTGTGGCAGTTTGACTCCATGCTGGCCTATAACTACAAGGCCTTCACTCTG
TACCCAGAAGAGTTTGGGCACAAGTCAGGATTGCCAGAGAAAGAATGGAAGGCAAAACTGA
AAGCAAGAGGGATACCATATAGTGAA
SEQ ID NO: 4 (HIV F2-Nef nucleic acid sequence)
ATGGGTGGCAAGTGGTCAAAATGCAGCATAGTTGGATGGCCTGATATAAGAGAGAGAATGAG
ACGAACTGAGCCAGCAGCAGAGCCAGCAGCAGAAGGAGTAGGAGCAGCGTCTCAAGACTTA
GATAAACATGGAGCACTTACAAGTAGCAACACAAACACCACTAATGCTGATTGTGCTTGGCCG
GAAGCGCAAGAGGATGAAGGAGAAGTAGGCTTTGCCAGTCAGACCTCAGAGTCCTTTAAGAC
CAATGACTTATAAGGGAGCATTTGATCTCGGCTTCTTTTTAAAAGAAGGGGGACTGGAAGGGT
TAATTTACTCTAAGAAAAGGCAAGAGATCCTTGATTTGTGGGTCTATCATACACAAGGCTACTT
CCCTGATTGGCAAAACTACACACCGGGACCAGGGGTCAGATACCCACTGACTTTTGGGTGGT
GCTTCAAGCTGGTACCAGTTGACCCAAAGGCAGTAGAAGAGGCCAACGAAGGAGAAGACAA
CTGTCTGCTACACCCAGTGTGCCAGCATGGAATGGAGGATGAACACAGAGAAGTATTAATGTG
GAAGTTTGACAGTCAACTAGCACGCAGACACATGGCCCGAGAGCTACATCCGGAGTTCTACA
AAGACTGC
SEQ ID NO: 5 (HIV C2-Nef nucleic acid sequence)
ATGGGTGGCAAGTGGTCAAAATGCAGCATAGGTGGATGGCCTCAGATAAGAGAGAGAATGAG
ACGAACTGAGCCAGCAGTAGAGCCAGCAGCAGAGCCAGCAGCAGAAGGAGTAGGAGCAGC
GTGGCTACTTCCTGATTGGCAAAACTACACACCGGGACCAGGAGTCAGATACCCACTGACTTT
TGGGTGGTGCTTCAAGCTGGTACCAGTAGACCCAGGGGCAGTAGAAGAGGCCAACGAAGGA
GAAAACAACTGTTTGCTACACCCGGTGTGCCAGCATGGAATGGAGGATGAGCAAAGAGAAGT
ATTAGTGTGCAAGTTTGACAGTCTACTAGCACGCAGACACATGGCCCGCGAGCTACATCCGGA
GTTCTACAAAGACTGC
SEQ ID NO: 6 (HIV HV2NZ-Nef nucleic acid sequence)
ATGGGTGCGAGTGGATCCAAGAAGCGTTCCAAGCCCTTGCAAGGACTACAAGAGAGACTCTT
GCAGGCGCGGGGAGAGACTTGTGGAGGGCGCTGCAACGAATCGGGAGGGGGATACTTGCAG
TCCCACGAAGGATCAGGCAGGGAGCAGAACTCGCCCTCCTGTGAGGGACAGCGATATCAGCA
GGGAGATTTTGTAAATACCCCATGGAGAACCCCAGCAGCAGAAAGGGAGAAAGAATTGTACA
AACAGCAAAATATGGATGATGTAGATCTAGATGATGATGACCAAGTAGGATTCCCTGTCACAC
CAAGAGTACCATTAAGACCAATGACATTCAAATTGGCAGTAGATATGTCTCATTTTATAAAAGA
AAAAGGGGGACTGGAAGGGCTGTTTTATAGTCAGAGAAGACATAGAATCTTAGACTTATACTT
AGACAAGGCTTTTACTCTGTACCCAGAGGAATTTGGGCATAATTCAGGACTGCCAGAGAAAG
AGTGGAAGGCGAGACTGAAAGCAAGGGGAATACCATTTAGT
SEQ ID NO: 7 (SIV Nef Mutant 1/SIV Nef M116 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAAGAAGAGAAAAG
ATCCTGAATTTGTATGCCTTGAACGAGTGGGGAATAATAGATGATTGGCAAGCTTACTCACCAG
GCCCGGGGATAAGGTACCCGAGAGTCTTTGGCTTCTGCTTTAAGCTAGTCCCAGTGGACCTGC
ATGAGGAGGCACGCAACTGTGAGAGACACTGTgctgcaCATCCAGCACAGATGGGGGAAGATCC
TGATGGAATAGATCATGGAGAAGTCTTGGTCTGGAAGTTTGACCCGAAGTTGGCGGTGGAGT
ACCGCCCGGACATGTTTAAGGACATGCACGAACATGCAAAGCGC
SEQ ID NO: 8 (SIV Nef Mutant 2 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAgctgcaGAAAAGAT
CCTGAATTTGTATGCCTTGAACGAGTGGGGAATAATAGATGATTGGCAAGCTTACTCACCAGG
CCCGGGGATAAGGTACCCGAGAGTCTTTGGCTTCTGCTTTAAGCTAGTCCCAGTGGACCTGCA
TGAGGAGGCACGCAACTGTGAGAGACACTGTgctgcaCATCCAGCACAGATGGGGGAAGATCCT
GATGGAATAGATCATGGAGAAGTCTTGGTCTGGAAGTTTGACCCGAAGTTGGCGGTGGAGTA
CCGCCCGGACATGTTTAAGGACATGCACGAACATGCAAAGCGC
SEQ ID NO: 9 (SIV Nef Mutant 3 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAgctgcaGAAAAGAT
CCTGAATTTGTATGCCTTGAACGAGTGGGGAATAATAGATGATTGGCAAGCTTACTCACCAGG
CCCGGGGATAAGGTACCCGAGAGTCTTTGGCTTCTGCTTTAAGCTAGTCCCAGTGGACCTGCA
TGAGGAGcatcacAACTGTGAGAGACACTGTgctgcaCATCCAGCACAGATGGGGgcggcaCCTGATG
GAATAGATCATGGAGAAGTCTTGGTCTGGAAGTTTGACCCGAAGTTGGCGGTGGAGTACCGC
CCGGACATGTTTAAGGACATGCACGAACATGCAAAGCGC
SEQ ID NO: 10 (SIV Nef Mutant 4 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAAGAAGAGAAAAG
ATCCTGAATTTGTATGCCTTGAACGAGTGGGGAATAATAGATGATTGGCAAGCTTACTCACCAG
GCCCGGGGATAAGGTACCCGAGAGTCTTTGGCTTCTGCTTTAAGCTAGTCCCAGTGGACCTGC
ATGAGGAGcatcacAACTGTGAGAGACACTGTgctgcaCATCCAGCACAGATGGGGgcggcaCCTGAT
GGAATAGATCATGGAGAAGTCTTGGTCTGGAAGTTTGACCCGAAGTTGGCGGTGGAGTACCG
CCCGGACATGTTTAAGGACATGCACGAACATGCAAAGCGC
SEQ ID NO: 11 (SIV Nef Mutant 5 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAATGACTCCAG
ACGGCCGCCGCCTGCAAGAAGGAGACACCTTTGATGAGTGGGATGATGATGAAGAAGAAGTA
GGCTTCCCTGTGCAACCTCGAGTCCCCTTAAGACAGATGACCTATAAATTAGCAGTGGACTTT
TCCCACTTTTTAAAATCAAAGGGGGGACTGGATGGGATATATTACTCTGAAAGAAGAGAAAAG
ATCCTGAATTTGTATGCCTTGAACGAGTGGGGAATAATAGATGATTGGCAAGCTTACTCACCAG
GCCCGGGGATAAGGTACCCGAGAGTCTTTGGCTTCTGCTTTAAGCTAGTCCCAGTGGACCTGC
ATGAGGAGGCACGCAACTGTGAGAGACACTGTgctgcaCATCCAGCACAGATGGGGgcggcaCCTG
ATGGAATAGATCATGGAGAAGTCTTGGTCTGGAAGTTTGACCCCGAAGTTGGCGGTGGAGTAC
CGCCCGGACATGTTTAAGGACATGCACGAACATGCAAAGCGC
SEQ ID NO: 12 (wildtype SIV Nef amino acid sequence)
MGSSNSKRQQQGLLKLWRGLRGKPGADWVLLSDPLIGQSSTVQEEECGKALKKSWGKGKMTPD
GRRLQEGDTFDEWDDDEEEVGFPVQPRVPLRQMTYKLAVDFSHFLKSKGGLDGIYYSERREKIL
NLYALNEWGIIDDWQAYSPGPGIRYPRVFGFCFKLVPVDLHEEARNCERHCLMHPAQMGEDPDGI
DHGEVLVWKFDPKLAVEYRPDMFKDMHEHAKR
SEQ ID NO: 13 (HIV1 Nef amino acid sequence)
MGGKWSKSSVIGWPTVRERMRRAEPAADRVGAASRDLEKHGAITSSNTAATNAACAWLEAQE
EEEVGFPVTPQVPLRPMTYKAAVDLSHFLKEKGGLEGLIHSQRRQDILDLWIYHTQGYFPDWQN
YTPGPGVRYPLTFGWCYKLVPVEPDKIEEANKGENTSLLHPVSLHGMDDPEREVLEWRFDSRLA
FHHVARELHPEYFKNC
SEQ ID NO: 14 (HIV2 Nef amino acid sequence)
MGASGSKKLSKHSRGLRERLLRARGDGYGKQRDASGGEYSQFQEESGREQNSPSCEGQQYQQG
EYMNSPQRNPATERQKDLYRQQNMDDVDSDDDDLIGVPVTPRVPRREMTYKLAIDMSHFIKEK
GGLQGMFYSRRRHRILDIYLEKEEGIIPDWQNYTHGPGVRYPMYFGWLWKLVSVELSQEAEEDE
ANCLVHPAQTSRHDDEHGETLVWQFDSMLAYNYKAFTTLYPEEFGHKSGLPEKEWKAKLKARGI
PYSE
SEQ ID NO: 15 (HIV F2-Nef amino acid sequence)
MGGKWSKCSIVGWPDIRERMRRTEPAAEPAAEGVGAASQDLDKHGALTSSNTNTTNADCAWPE
AQEDEGEVGFAVRPQSPLRPMTYKGAFDLGFFLKEGGLEGIYSKKRQEILDLWVYHTQGYFPD
WQNYTPGPGVRYPLTFGWCFKLVPVDPKAVEEANEGEDNCLLHPVCQHGMEDEHREVLMWKF
DSQLARRHMARELHPEFYKDC
SEQ ID NO: 16 (HIV C2-Nef amino acid sequence)
MGGKWSKCSIGGWPQIRERMRRTEPAVEPAAEPAAEGVGAAWLLPDWQNYTPGPGVRYPLTFG
WCFKLVPVDPGAVEEANEGENNCLLHPVCQHGMEDEQREVLVCKFDSLLARRHMARELHPEFY
KDC
SEQ ID NO: 17 (HIV HV2NZ-Nef amino acid sequence)
MGASGSKKRSKPLQGLQERLLQARGETCGGRCNESGGGYLQSHEGSGREQNSPSCEGQRYQQG
DFVNTPWRTPAAEREKELYKQQNMDDVDLDDDDQVGFPVTPRVPLRPMTFKLAVDMSHFIKEK
GGLEGLFYSQRRHRILDLYLDKAFTLYPEEFGHNSGLPEKEWKARLKARGIPFS
SEQ ID NO: 18 (SIV Nef Mutant 1/SIV Nef M116 amino acid sequence)
MGSSNSKRQQQGLLKLWRGLRGKPGADWVLLSDPLIGQSSTVQEECGKALKKSWGKGKMTPD
GRRLQEGDTFDEWDDDEEEVGFPVQPRVPLRQMTYKLAVDFSHFLKSKGGLDGIYYSERREKIL
NLYALNEWGIIDDWQAYSPGPIRYPRVFGFCFKLVPVDLHEEARNCERHCAAHPAQMGEDPDGI
DHGEVLVWKFDPKLAVEYRPDMFKDMHEHAKR
SEQ ID NO: 19 (SIV Nef Mutant 2 amino acid sequence)
MGSSNSKRQQQGLLKLWRGLRGKPGADWVLLSDPLIGQSSTVQEECGKALKKSWGKGKMTPD
GRRLQEGDTFDEWDDDEEEVGFPVQPRVPLRQMTYKLAVDFSHFLKSKGGLDGIYYSEAAEKIL
NLYALNEWGIIDDWQAYSPGPIRYPRVFGFCFKLVPVDLHEEARNCERHCAAHPAQMGEDPDGI
DHGEVLVWKFDPKLAVEYRPDMFKDMHEHAKR
SEQ ID NO: 20 (SIV Nef Mutant 3 amino acid sequence)
MGSSNSKRQQQGLLKLWRGLRGKPGADWVLLSDPLIGQSSTVQEECGKALKKSWGKGKMTPD
GRRLQEGDTFDEWDDDEEEVGFPVQPRVPLRQMTYKLAVDFSHFLKSKGGLDGIYYSEAAEKIL
NLYALNEWGIIDDWQAYSPGPGIRYPRVFGFCFKLVPVDLHEEHHNCERHCAAHPAQMGAAPDGI
DHGEVLVWKFDPKLAVEYRPDMFKDMHEHAKR
SEQ ID NO: 21 (SIV Nef Mutant 4 amino acid sequence)
MGSSNSKRQQQGLLKLWRGLRGKPGADWVLLSDPLIGQSSTVQEECGKALKKSWGKGKMTPD
GRRLQEGDTFDEWDDDEEEVGFPVQPRVPLRQMTYKLAVDFSHFLKSKGGLDGIYYSERREKIL
NLYALNEWGIIDDWQAYSPGPGIRYPRVFGFCFKLVPVDLHEEHHNCERHCAAHPAQMGAAPDGI
DHGEVLVWKFDPKLAVEYRPDMFKDMHEHAKR
SEQ ID NO: 22 (SIV Nef Mutant 5 amino acid sequence)
MGSSNSKRQQQGLLKLWRGLRGKPGADWVLLSDPLIGQSSTVQEECGKALKKSWGKGKMTPD
GRRLQEGDTFDEWDDDEEEVGFPVQPRVPLRQMTYKLAVDFSHFLKSKGGLDGIYYSERREKIL
NLYALNEWGIIDDWQAYSPGPGIRYPRVFGFCFKLVPVDLHEEARNCERHCAAHPAQMGAAPDGI
DHGEVLVWKFDPKLAVEYRPDMFKDMHEHAKR
SEQ ID NO: 23 (gRNA nucleic acid sequence)
GAGAATCAAAATCGGTGAAT
SEQ ID NO: 24 (SIV Nef-P2A-LNGFR nucleic acid sequence; SIV Nef sequence is bolded, P2A
sequence is underlined, LNGFR sequence is italicized, restriction sites are in lower case)
gaattcATGGGCTCCAGCAACTCCAAGAGGCAGCAACAGGGCTTGCTCAAGCTCTGGCGAG
GGCTGCGAGGGAAGCCTGGGGCAGACTGGGTGCTATTGTCCGATCCGCTTATCGGGCA
GTCATCAACAGTCCAAGAAGAGTGCGGCAAGGCCTTGAAAAAGTCCTGGGGTAAAGGT
AAAATGACTCCAGACGGCCGCCGCCTGCAAGAAGGAGACACCTTTGATGAGTGGGATG
ATGATGAAGAAGAAGTAGGCTTCCCTGTGCAACCTCGAGTCCCCTTAAGACAGATGACC
TATAAATTAGCAGTGGACTTTTCCCACTTTTTAAAATCAAAGGGGGGACTGGATGGGATA
TATTACTCTGAAAGAAGAGAAAAGATCCTGAATTTGTATGCCTTGAACGAGTGGGGAAT
AATAGATGATTGGCAAGCTTACTCACCAGGCCCGGGGATAAGGTACCCGAGAGTCTTTG
GCTTCTGCTTTAAGCTAGTCCCAGTGGACCTGCATGAGGAGGCACGCAACTGTGAGAG
ACACTGTCTGATGCATCCAGCACAGATGGGGGAAGATCCTGATGGAATAGATCATGGAG
AAGTCTTGGTCTGGAAGTTTGACCCGAAGTTGGCGGTGGAGTACCGCCCGGACATGTT
TAAGGACATGCACGAACATGCAAAGCGCacgcgtGGAAGCGGAGCTACTAACTTCAGCCTGCT
GAAGCAGGCTGGAGACGTGGAGGAGAACCCTGGACCTggatccATGGGGGCAGGTGCCACCGG
CCGCGCCATGGACGGGCCGCGCCTGCTGCTGTTGCTGCTTCTGGGGGTGTCCCTTGGAGGTGCC
AAGGAGGCATGCCCCACAGGCCTGTACACACACAGCGGTGAGTGCTGCAAAGCCTGCAACCTGGG
CGAGGGTGTGGCCCAGCCTTGTGGAGCCAACCAGACCGTGTGTGAGCCCTGCCTGGACAGCGTG
ACGTTCTCCGACGTGGTGAGCGCGACCGAGCCGTGCAAGCCGTGCACCGAGTGCGTGGGGCTCC
AGAGCATGTCGGCGCCGTGCGTGGAGGCCGACGACGCCGTGTGCCGCTGCGCCTACGGCTACTA
CCAGGATGAGACGACTGGGCGCTGCGAGGCGTGCCGCGTGTGCGAGGCGGGCTCGGGCCTCGT
GTTCTCCTGCCAGGACAAGCAGAACACCGTGTGCGAGGAGTGCCCCGACGGCACGTATTCCGACG
AGGCCAACCACGTGGACCCGTGCCTGCCCTGCACCGTGTGCGAGGACACCGAGCGCCAGCTCCG
CGAGTGCACACGCTGGGCCGACGCCGAGTGCGAGGAGATCCCTGGCCGTTGGATTACACGGTCC
ACACCCCCAGAGGGCTCGGACAGCACAGCCCCCAGCACCAGGAGCCTGAGGCACCTCCAGAAC
AAGACCTCATAGCCAGCACGGTGGCAGGTGTGGTGACCACAGTGATGGGCAGCTCCCAGCCCGTG
GTGACCCGAGGCACCACCGACAACCTCATCCCTGTCTATTGCTCCATCCTGGCTGCTGTGGTTGTG
GGCCTTGTGGCCTACATAGCCTTCtgatctaga
SEQ ID NO: 25 (HIV1 Nef-T2A-Puro nucleic acid sequence; HIV1 Nef sequence is bolded, T2A
sequence is underlined, Puro sequence is italicized restriction sites are in lower case)
gaattcATGGGTGGCAAGTGGTCAAAAAGTAGTGTGATTGGATGGCCTACTGTAAGGGAAA
GAATGAGACGAGCTGAGCCAGCAGCAGATAGGGTGGGAGCAGCATCTCGAGACCTGGA
AAAACATGGAGCAATCACAAGTAGCAATACAGCAGCTACCAATGCTGCTTGTGCCTGGC
TAGAAGCACAAGAGGAGGAGGAGGTGGGTTTTCCAGTCACACCTCAGGTACCTTTAAG
ACCAATGACTTACAAGGCAGCTGTAGATCTTAGCCACTTTTTAAAAGAAAGGGGGGAC
TGGAAGGGCTAATTCACTCCCAAAGAAGACAAGATATCCTTGATCTGTGGATCTACCAC
ACACAAGGCTACTTCCCTGATTgGCAGAACTACACACCAGGGCCAGGGGTCAGATATCC
ACTGACCTTTGGATGGTGCTACAAGCTAGTACCAGTTGAGCCAGATAAGATAGAAGAGG
CCAATAAAGGAGAGAACACCAGCTTGTTACACCCTGTGAGCCTGCATGGGATGGATGAC
CCGGAGAGAGAAGTGTTAGAGTGGAGGTTTGACAGCCGCCTAGCATTTCATCACGTGG
CCCGAGAGCTGCATCCGGAGTACTTCAAGAACTGCactagtGGCAGTGGAGAGGGCAGAGG
AAGTCTGCTAACATGCGGTGACGTCGAGGAGAATCCTGGCCCAacgcgtATGACCGAGTACAAGC
CCACGGTGCGCCTCGCCACCCGCGACGACGTCCCCAGGGCCGTACGCACCCTCGCCGCCGCGTT
CGCCGACTACCCCGCCACGCGCCACACCGTCGATCCGGACCGCCACATCGAGCGGGTCACCGAG
CTGCAAGAACTCTTCCTCACGCGCGTCGGGCTCGACATCGGCAAGGTGTGGGTCGCGGACGACG
GCGCCGCGGTGGCGGTCTGGACCACGCCGGAGAGCGTCGAAGCGGGGGCGGTGTTCGCCGAGA
TCGGCCCGCGCATGGCCGAGTTGAGCGGTTCCCGGCTGGCCGCGCAGCAACAGATGGAAGGCCT
CCTGGCGCCGCACCGGCCCAAGGAGCCCGCGTGGTTCCTGGCCACCGTCGGCGTCTCGCCCGA
CCACCAGGGCAAGGGTCTGGGCAGCGCCGTCGTGCTCCCCGGAGTGGAGGCGGCCGAGCGCGC
CGGGGTGCCCGCCTTCCTGGAGACCTCCGCGCCCCGCAACCTCCCCTTCTACGAGCGGCTCGGC
TTCACCGTCACCGCCGACGTCGAGGTGCCCGAAGGACCGCGCACCTGGTGCATGACCCGCAAGC
CCGGTGCCTGAggatcc
SEQ ID NO: 26 (HIV2 Nef-T2A-Puro nucleic acid sequence; HIV1 Nef sequence is bolded, T2A
sequence is underlined, Puro sequence is italicized, restriction sites are in lower case)
gaattcATGGGTGCGAGTGGATCCAAGAAGCTTTCCAAGCATTCGCGAGGACTACGAGAGA
GACTCTTGCGGGCGCGTGGGGATGGTTATGGGAAGCAGCGCGACGCATCGGGAGGGG
AATACTCGCAGTTCCAAGAAGAATCAGGCAGGGAGCAGAACTCGCCCTCCTGTGAGGG
ACAGCAGTATCAGCAGGGAGAGTACATGAACAGCCCATGGAGAAACCCAGCAACAGAA
AGACAGAAAGATTTGTATAGGCAGCAAAATATGGATGATGTAGATTCTGATGATGATGAC
CTAATAGGAGTTCCTGTTACACCAAGAGTACCACGGAGAGAAATGACCTATAAATTGGC
AATAGATATGTCACATTTTATAAAAGAAAAAGGGGGACTGCAAGGGATGTTTTACAGTAG
GAGGAGACATAGAATCCTAGACATATACCTAGAAAAAGAGGAAGGGATAATACCAGATT
GGCAGAATTATACTCATGGGCCAGGAGTAAGGTACCCAATGTACTTCGGGTGGCTGTGG
AAGCTAGTATCAGTAGAACTCTCACAAGAGGCAGAGGAAGATGAGGCCAACTGCTTAGT
ACACCCAGCACAAACAAGCAGACATGATGATGAGCATGGGGAGACATTAGTGTGGCAG
TTTGACTCCATGCTGGCCTATAACTACAAGGCCTTCACTCTGTACCCAGAAGAGTTTGG
GCACAAGTCAGGATTGCCAGAGAAAGAATGGAAGGCAAAACTGAAAGCAAGAGGGATA
CCATATAGTGAAactagtGGCAGTGGAGAGGGCAGAGGAAGTCTGCTAACATGCGGTGACGTCG
AGGAGAATCCTGGCCCAacgcgtATGACCGAGTACAAGCCCACGGTGCGCCTCGCCACCCCGCGAC
GACGTCCCCAGGGCCGTACGCACCCTCGCCGCCGCGTTCGCCGACTACCCCGCCACGCGCCACA
CCGTCGATCCGGACCGCCACATCGAGCGGGTCACCGAGCTGCAAGAACTCTTCCTCACGCGCGTC
GGGCTCGACATCGGCAAGGTGTGGGTCGCGGACGACGGCGCCGCGGTGGCGGTCTGGACCACG
CCGGAGAGCGTCGAAGCGGGGGCGGTGTTCGCCGAGATCGGCCCGCGCATGGCCGAGTTGAGC
GGTTCCCGGCTGGCCGCGCAGCAACAGATGGAAGGCCTCCTGGCGCCGCACCGGCCCAAGGAG
CCCGCGTGGTTCCTGGCCACCGTCGGCGTCTCGCCCGACCACCAGGGCAAGGGTCTGGGCAGC
GCCGTCGTGCTCCCCGGAGTGGAGGCGGCCGAGCGCGCCGGGGTGCCCGCCTTCCTGGAGACC
TCCGCGCCCCGCAACCTCCCCTTCTACGAGCGGCTCGGCTTCACCGTCACCGCCGACGTCGAGGT
GCCCGAAGGACCGCGCACCTGGTGCATGACCCGCAAGCCCGGTGCCTGAggatcc
SEQ ID NO: 27 (SIV Nef-P2A-LNGFR amino acid sequence; SIV Nef sequence is bolded, P2A
sequence is underlined, LNGFR sequence is italicized, restriction sites are squared)

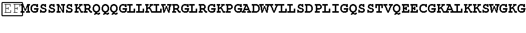

KMTPDGRRLQEGDTFDEWDDDEEEVGFPVQPRVPLRQMTYKLAVDFSHFLKSKGGLDGIY
YSERREKILNLYALNEWGIIDDWQAYSPGPGIRYPRVFGFCFKLVPVDLHEEARNCERHCLM

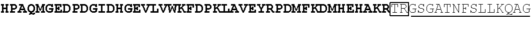

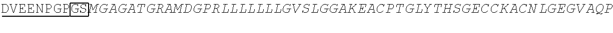

CGANQTVCEPCLDSVTFSDVVSATEPCKPCTECVGLQSMSAPCVEADDAVCRCAYGYYQDETTGRCEA
CRVCEAGSGLVFSCQDKQNTVCEECPDGTYSDEANHVDPCLPCTVCEDTERQLRECTRWADAECEEI
PGRWITRSTPPEGSDSTAPSTQEPEAPPEQDLIASTVAGVVTTVMGSSQPVVTRGTTDNLIPVYCSILAAV

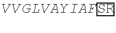

SEQ ID NO: 28 (HIV1 Nef-T2A-Puro amino acid sequence; HIV1 Nef sequence is bolded, T2A
sequence is underlined, Puro sequence is italicized, restriction sites are squared)

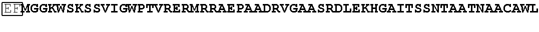

EAQEEEEVGFPVTPQVPLRPMTYKAAVDLSHFLKEKGGLEGLIHSQRRQDILDLWIYHTQG
YFPDWQNYTPGPGVRYPLTFGWCYKLVPVEPDKIEEANKGENTSLLHPVSLHGMDDPERE

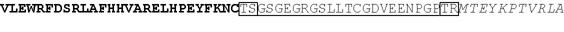

TRDDVPRAVRTLAAAFADYPATRHTVDPDRHIERVTELQELFLTRVGLDIGKVWVADDGAAVAVWTTP
ESVEAGAVFAEIGPRMAELSGSRLAAQQQMEGLLAPHRPKEPAWFLATVGVSPDHQGKGLGSAVVLP

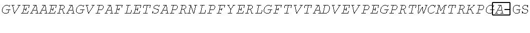

SEQ ID NO: 29 (HIV2 Nef-T2A-Puro amino acid sequence; HIV2 Nef sequence is bolded, T2A
sequence is underlined, Puro sequence is italicized, restriction sites are squared)

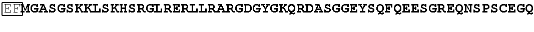

QYQQGEYMNSPWRNPATERQKDLYRQQNMDDVDSDDDDLIGVPVTPRVPRREMTYKLAI
DMSHFIKEKGGLQGMFYSRRRHRILDIYLEKEEGIIPDWQNYTHGPGVRYPMYFGWLWKL
VSVELSQEAEEDEANCLVHPAQTSRHDDEHGETLVWQFDSMLAYNYKAFTLYPEEFGHKS

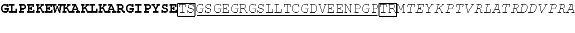

VRTLAAAFADYPATRHTVDPDRHIERVTELQELFLTRVGLDIGKVWVADDGAAVAVWTTPESVEAGAN
FAEIGPRMAELSGSRLAAQQQMEGLLAPHRPKEPAWFLATVGVSPDHQGKGLGSAVVLPGVEAAERA

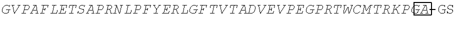

SEQ ID NO: 30 (P2A nucleic acid sequence)
GGAAGCGGAGCTACTAACTTCAGCCTGCTGAAGCAGGCTGGAGACGTGGAGGAGAACCCTG
GACCT
SEQ ID NO: 31 (T2A nucleic acid sequence)
GGCAGTGGAGAGGGCAGAGGAAGTCTGCTAACATGCGGTGACGTCGAGGAGAATCCTGGCC
CA
SEQ ID NO: 32 (E2A nucleic acid sequence)
GGAAGCGGACAGTGTACTAATTATGCTCTCTTGAAATTGGCTGGAGATGTTGAGAGCAACCC
TGGACCT
SEQ ID NO: 33 (F2A nucleic acid sequence)
GGAAGCGGAGTGAAACAGACTTTGAATTTTGACCTTCTCAAGTTGGCGGGAGACGTGGAGT
CCAACCCTGGACCT
SEQ ID NO: 34 (IRES nucleic acid sequence)
GCCCCTCTCCCCTCCCCCCCCCCTAACGTTACTGGCCGAAGCCGCTTGGAATAAGGCCGGTGT
GCGTTTGTCTATATGTTATTTTCCACCATATTGCCGTCTTTTGGCAATGTGAGGGCCCGGAAA
CCTGGCCCTGTCTTCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGAAGACAAACAACGTC
TGTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGTTTTCCCTTT
GAAAAACACGATGATAATATGGCCACA
SEQ ID NO: 35 (PGK nucleic acid sequence)
GGGTAGGGGAGGCGCTTTTCCCAAGGCAGTCTGGAGCATGCGCTTTAGCAGCCCCGCTGGGC
ACTTGGCGCTACACAAGTGGCCTCTGGCCTCGCACACATTCCACATCCACCGGTAGGCGCCA
ACCGGCTCCGTTCTTTGGTGGCCCCTTCGCGCCACCTTCTACTCCTCCCCTAGTCAGGAAGTT
CCCCCCCGCCCCGCAGCTCGCGTCGTGCAGGACGTGACAAATGGAAGTAGCACGTCTCACTA
GTCTCGTGCAGATGGACAGCACCGCTGAGCAATGGAAGCGGGTAGGCCTTTGGGGCAGCGG
CCAATAGCAGCTTTGCTCCTTCGCTTTCTGGGCTCAGAGGCTGGGAAGGGGTGGGTCCGGGG
GCGGGCTCAGGGGCGGGCTCAGGGGCGGGGCGGGCGCCCGAAGGTCCTCCGGAGGCCCGGC
ATTCTGCACGCTTCAAAAGCGCACGTCTGCCGCGCTGTTCTCCTCTTCCTCATCTCCGGGCCT
TTCGACCTGCAGCCCAAGCTTACC
SEQ ID NO: 36 (P2A amino acid sequence)
GSGATNFSLLKQAGDVEENPGP
SEQ ID NO: 37 (T2A amino acid sequence)
GSGEGRGSLLTCGDVEENPGP
SEQ ID NO: 38 (E2A amino acid sequence)
GSGQCNYALLKLAGDVESNPGP
SEQ ID NO: 39 (F2A amino acid sequence)
GSGVKQTLNFDLLKLAGDVESNPGP
SEQ ID NO: 40 (linker amino acid sequence)
GGGGS
SEQ ID NO: 41 (linker amino acid sequence)
(GGGGS)₂
SEQ ID NO: 42 (linker amino acid sequence)
(GGGS)₃
SEQ ID NO: 43 (linker amino acid sequence)
(GGGS)₄
SEQ ID NO: 44 (linker amino acid sequence)
GGGGSGGGGSGGGGGGSGSGGGGS
SEQ ID NO: 45 (linker amino acid sequence)
GGGGSGGGGSGGGGGGSGSGGGGSGGGGSGGGGS
SEQ ID NO: 46 (linker amino acid sequence)
(GGGGS)₃
SEQ ID NO: 47 (linker amino acid sequence)
(GGGGS)₄
SEQ ID NO: 48 (M167, SIV Nef-IRES-CD20 scFv (Rituximab) CAR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCTCTCCCTCCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGTTTTCCTTT
GAAAAACACGATGATAATATGGCCACAggatccgccgccaccATGGCCCTGCCAGTGACCGCCTTGC
TCCTTCCCCTGGCTCTTCTGCTGCACGCTGCTAGACCTCAGGTGCAGCTTCAGCAGCCTGGCG
CTGAGCTGGTGAAGCCCGGAGCTAGCGTGAAGATGTCCTGCAAGGCCAGCGGCTATACCTTC
ACCTCATACAACATGCACTGGGTGAAGCAGACCCCTGGAAGAGGCCTCGAGTGGATTGGAG
CTATCTACCCTGGAAACGGAGACACCAGCTATAACCAGAAGTTCAAGGGAAAGGCTACCCT
GACCGCTGACAAGAGCAGCAGCACCGCTTACATGCAGCTGAGCAGCCTTACAAGCGAGGAC
TCTGCCGTGTACTACTGCGCCAGAAGCACCTATTACGGCGGCGACTGGTACTTCAACGTGTG
GGGAGCTGGAACCACCGTGACCGTTAGCGCCGGCGGCGGAGGCTCTGGCGGCGGAGGAAGC
GGCGGCGGCGGCTCCCAGATCGTGCTGTCTCAGAGCCCCGCTATCTTGAGCGCCTCCCCTGG
AGAGAAGGTGACCATGACTTGCAGAGCTAGCAGCAGCGTGAGCTACATCCACTGGTTCCAA
CAGAAGCCAGGCAGCTCCCCTAAGCCTTGGATCTACGCTACCAGCAACCTTGCCTCAGGCGT
TCCCGTGAGATTCTCTGGATCTGGAAGCGGCACATCCTACTCCCTGACCATCTCCCGGGTCG
AGGCTGAGGACGCTGCTACTTACTACTGCCAGCAGTGGACTAGCAACCCCCCCACATTTGGC
GGCGGCACCAAACTGGAGATCAAGactagtaccacgacgccagcgccgcgaccaccaacaccggcgcccaccatcgcgtcg
cagcccctgtccctgcgcccagaggcgtgccggccagcggcggggggcgcagtgcacacgagggggctggacttcgcctgtgatatctacatctgg
gcgcccttggccgggacttgtggggtccttctcctgtcactggttatcaccctttactgcaaacggggcagaaagaaactcctgtatatattcaaacaaccat
ttatgagaccagtacaaactactcaagaggaagatggctgtagctgccgatttccagaagaagaagaaggaggatgtgaactgagagtgaagttcagca
ggagcgcagacgcccccgcgtaccagcagggccagaaccagctctataacgagctcaatctaggacgaagagaggagtacgatgttttggacaaga
gacgtggccgggaccctgagatggggggaaagccgagaaggaagaaccctcaggaaggcctgtacaatgaactgcagaaagataagatggcggag
gcctacagtgagattgggatgaaaggcgagcgccggaggggcaaggggcacgatggcctttaccagggtctcagtacagccaccaaggacacctac
gacgcccttcacatgcaggccctgccccctcgctaa
SEQ ID NO: 49 (M168, SIV Nef-IRES-CD20 scFv (Leu-16) CAR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCCTCTCCCTCCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGTTTTCCTTT
GAAAAACACGATGATAATATGGCCACAggatccgccgccaccATGGCCCTGCCAGTGACCGCCTTGC
TCCTTCCCCTGGCTCTTCTGCTGCACGCTGCTAGACCTGAGGTGCAGCTGCAGCAGAGCGGA
GCTGAGCTGGTGAAGCCTGGCGCTAGCGTGAAGATGAGCTGCAAGGCCAGCGGCTACACCT
TCACCAGCTATAACATGCACTGGGTGAAGCAGACCCCTGGACAGGGACTGGAGTGGATCGG
AGCTATCTACCCTGGAAACGGAGACACCTCATACAACCAGAAGTTCAAGGGAAAGGCTACC
CTGACCGCTGACAAGAGCAGCAGCACCGCTTACATGCAGCTGAGCTCACTGACCAGCGAGG
ACTCCGCCGACTACTACTGCGCCAGAAGCAACTACTACGGAAGCAGCTACTGGTTCTTCGAC
GTGTGGGGAGCTGGAACCACCGTGACCGTGTCAAGCGGCGGCGGAGGcTCCGGAGGCGGAG
GATCTGGCGGCGGCGGCAGCGACATCGTGCTGACCCAGAGCCCTGCTATCCTGTCTGCCAGC
CCTGGAGAGAAGGTGACCATGACCTGCAGAGCTAGCAGCAGCGTGAACTACATGGACTGGT
ATCAGAAAAAGCCCGGCAGCTCACCTAAGCCTTGGATCTACGCTACCAGCAACTTAGCCAGC
GGCGTGCCTGCTAGATTCTCCGGAAGCGGCTCTGGAACCAGCTACTCCCTTACCATCAGCAG
AGTGGAGGCTGAGGACGCTGCTACCTACTACTGCCAGCAGTGGAGCTTCAACCCTCCTACCT
TCGGAGGAGGAACCAAGCTGGAGATCAAGactagtaccacgacgccagcgccgcgaccaccaacaccggcgcccacca
tcgcgtcgcagcccctgtccctgcgcccagaggcgtgccggccagcggcggggggcgcagtgcacacgagggggctggacttcgcctgtgatatct
acatctgggcgcccttggccgggacttgtggggtccttctcctgtcactggttatcaccctttactgcaaacggggcagaaagaaactcctgtatatattcaa
acaaccatttatgagaccagtacaaactactcaagaggaagatggctgtagctgccgatttccagaagaagaagaaggaggatgtgaactgagagtgaa
gttcagcaggagcgcagacgcccccgcgtaccagcagggccagaaccagctctataacgagctcaatctaggacgaagagaggagtacgatgttttg
gacaagagacgtggccgggaccctgagatggggggaaagccgagaaggaagaaccctcaggaaggcctgtacaatgacctgcagaaagataagat
ggcggaggcctacagtgagattgggatgaaaggcgagcgccggaggggcaaggggcacgatggcctttaccagggtctcagtacagccaccaagg
acacctacgacgcccttcacatgcaggccctgccccctcgctaa
SEQ ID NO: 50 (M169, SIV Nef-IRES-CD19 × CD20 scFv CAR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtgG
CCCCTCTCCCTCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCCAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGTTTTCCTTT
GAAAAACACGATGATAATATGGCCACAggatccgccgccaccATGGCCCTGCCTGTCACCGCCCTGC
TGCTGCCCCTGGCTCTGCTGCTGCACGCCGCAAGACCTGAAGTCCAGCTGCAGCAGTCCGGG
GCAGAGCTGGTGAAGCCAGGAGCCTCCGTGAAGATGTCTTGTAAGGCCAGCGGCTACACCT
TCACATCCTATAACATGCACTGGGTGAAGCAGACCCCTGGACAGGGCCTGGAGTGGATCGG
AGCAATCTACCCAGGCAACGGCGACACAAGCTATAATCAGAAGTTTAAGGGCAAGGCCACC
CTGACAGCCGATAAGAGCTCCTCTACCGCCTACATGCAGCTGAGCTCCCTGACAAGCGAGGA
CTCCGCCGATTACTATTGCGCCCGGTCCAATTACTATGGCTCTAGCTACTGGTTCTTTGACGT
GTGGGGAGCAGGAACCACAGTGACCGTGTCCTCTGGAGGAGGAGGAAGCGGAGGAGGAGG
ATCTGGCGGCGGCGGCTCTGATATCGTGCTGACACAGAGCCCAGCAATCCTGTCCGCCTCTC
CAGGAGAGAAGGTGACCATGACATGTCGGGCCAGCTCCTCTGTGAACTACATGGACTGGTA
TCAGAAGAAGCCCGGCAGCTCCCCTAAGCCATGGATCTACGCCACCTCCAATCTGGCATCTG
GAGTGCCTGCAAGGTTCAGCGGcTCCGGATCTGGCACCAGCTATTCCCTGACAATCTCTCGC
GTGGAGGCAGAGGATGCAGCAACCTACTATTGCCAGCAGTGGAGCTTCAACCCCCCTACCTT
TGGCGGCGGCACAAAGCTGGAGATCAAGGGCGGCGGCGGCTCCGGCGGCGGCGGGAGCGG
CGGCGGCGGCTCTGGCGGCGGCGGCAGCGGCGGCGGCGGCTCCGACATCCAGATGACCCAG
ACCACATCTAGCCTGTCTGCCAGCCTGGGCGACAGGGTGACAATCAGCTGTCGCGCCTCCCA
GGATATCTCTAAGTACCTGAATTGGTATCAGCAGAAGCCAGATGGCACCGTGAAGCTGCTGA
TCTACCACACAAGCCGGCTGCACTCCGGAGTGCCAAGCCGGTTCAGCGGCTCTGGCAGCGGC
ACCGACTATAGCCTGACAATCTCCAACCTGGAGCAGGAGGATATCGCCACCTACTTCTGCCA
GCAGGGCAATACCCTGCCTTATACATTTGGCGGAGGAACAAAGCTGGAGATCACCGGCTCC
ACATCTGGAAGCGGCAAGCCAGGATCTGGAGAGGGAAGCACCAAGGGAGAGGTGAAGCTG
CAGGAGTCCGGACCAGGCCTGGTGGCACCTTCCCAGTCTCTGAGCGTGACCTGTACAGTGTC
TGGCGTGAGCCTGCCTGACTACGGCGTGTCCTGGATCAGGCAGCCACCAAGAAAGGGCCTG
GAGTGGCTGGGCGTGATCTGGGGCAGCGAGACAACATACTATAACTCCGCCCTGAAGAGCC
GGCTGACCATCATCAAGGATAACTCCAAGTCTCAGGTGTTCCTGAAGATGAATAGCCTGCAG
ACCGACGATACAGCCATCTACTATTGCGCCAAGCACTACTATTATGGAGGCAGTTATGCTAT
GGACTATTGGGGGCAGGGCACAAGCGTCACCGTCTCATCAactagtaccacgacgccagcgccgcgaccacca
acaccggcgcccaccatcgcgtcgcagcccctgtccctgcgcccagaggcgtgccggccagcggcggggggcgcagtgcacacgagggggctgg
acttcgcctgtgatatctacatctgggcgcccttggccgggacttgtggggtccttctcctgtcactggttatcaccctttactgcaaacggggcagaaaga
aactcctgtatatattcaaacaaccatttatgagaccagtacaaactactcaagaggaagatggctgtagctgccgatttccagaagaagaagaaggagg
atgtgaactgagagtgaagttcagcaggagcgcagacgcccccgcgtaccagcagggccagaaccagctctataacgagctcaatctaggacgaaga
gaggagtacgatgttttggacaagagacgtggccgggaccctgagatggggggaaagccgagaaggaagaaccctcaggaagcctgtacaatgaa
ctgcagaaagataagatggcggaggcctacagtgagattgggatgaaaggcgagcgccggaggggcaaggggcacgatggcctttaccagggtctc
agtacagccaccaaggacacctacgacgcccttcacatgcaggccctgccccctcgctaa
SEQ ID NO: 51 (M170, SIV Nef-IRES-CD19 scFv CAR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCTCTCCCTCCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGACCACGGGGACGTGGTTTTCCTTT
GAAAAACACGATGATAATATGGCCACAggatccgccgccaccATGGCCCTGCCTGTGACCGCCCTGC
TGCTGCCCCTGGCCCTGCTGCTGCACGCTGCTAGACCTGATATTCAGATGACCCAGACCACT
AGCTCCCTGTCCGCCTCTCTGGGCGACAGAGTGACAATCAGCTGCAGGGCCTCCCAGGATAT
CTCTAAGTATCTGAACTGGTACCAGCAGAAGCCAGACGGCACAGTGAAGCTGCTGATCTATC
ACACCAGCCGCCTGCACTCCGGAGTGCCATCTCGGTTCAGCGGcTCCGGATCTGGCACAGAC
TACAGCCTGACCATCTCCAACCTGGAGCAGGAGGATATCGCCACCTATTTCTGCCAGCAGGG
CAATACACTGCCCTACACCTTTGGCGGCGGCACAAAGCTGGAGATCACCGGAGGAGGAGGA
AGCGGCGGAGGAGGCTCCGGCGGCGGCGGCTCTGAGGTGAAGCTGCAGGAGTCCGGACCTG
GCCTGGTGGCACCAAGCCAGTCCCTGTCTGTGACATGTACCGTGTCCGGCGTGTCTCTGCCT
GATTACGGCGTGTCTTGGATCAGGCAGCCACCTAGGAAGGGCCTGGAGTGGCTGGGCGTGA
TCTGGGGCAGCGAGACAACATACTATAATTCTGCCCTGAAGAGCAGACTGACCATCATCAA
GGACAACAGCAAGTCCCAGGTGTTCCTGAAGATGAATAGCCTGCAGACAGACGATACCGCC
ATCTACTATTGCGCCAAGCACTACTATTACGGCGGCAGCTATGCCATGGATTACTGGGGCCA
GGGCACATCCGTGACCGTGTCTAGCactagtaccacgacgccagcgccgcgaccaccaacaccggcgcccaccatcgcgtcg
cagcccctgtccctgcgcccagaggcgtgccggccagcggcggggggcgcagtgcacacgagggggctggacttcgcctgtgatatctacatctgg
gcgcccttggccgggacttgtggggtccttctcctgtcactggttatcaccctttactgcaaacggggcagaaagaaactcctgtatatattcaaacaaccat
ttatgagaccagtacaaactactcaagaggaagatggctgtagctgccgatttccagaagaagaagaaggaggatgtgaactgagagtgaagttcagca
ggagcgcagacgcccccgcgtaccagcagggccagaaccagctctataacgagctcaatctaggacgaagagaggagtacgatgttttggacaaaga
gacgtggccgggaccctgagatggggggaaagccgagaaggaagaaccctcaggaaggcctgtacaatgaactgcagaaagataagatggcggag
gcctacagtgagattgggatgaaaggcgagcgccggaggggcaaggggcacgatggcctttaccagggtctcagtacagccaccaaggacacctac
gacgcccttcacatgcaggccctgccccctcgctaa
SEQ ID NO: 52 (M171, SIV Nef-IRES-BCMA BiVHH CAR1 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCTCTCCCTCCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCGAACCACGGGGACGTGGTTTTCCTTT
GAAAAACACGATGATAATATGGCCACAggatccgccgccaccatggctctgcccgtcaccgctctgctgctgcctctggctct
gctgctgcacgctgctcgccctcaggtcaaactggaagaatctggcggaggcctggtgcaggcaggacggagcctgcgcctgagctgcgcagcatcc
gagcacaccttcagctcccacgtgatgggctggtttcggcaggccccaggcaaggagagagagagcgtggccgtgatcggctggagggacatctcca
catcttacgccgattccgtgaagggccggttcaccatcagccgggacaacgccaagaagacactgtatctgcagatgaacagcctgaagcccgaggac
accgccgtgtactattgcgcagcaaggagaatcgacgcagcagactttgattcctggggccagggcacccaggtgacagtgtctagcggaggaggag
gatctgaggtgcagctggtggagagcggaggcggcctggtgcaggccggaggctctctgaggctgagctgtgcagcatccggaagaaccttcacaat
gggctggtttaggcaggcaccaggaaaggagagggagttcgtggcagcaatcagcctgtcccctaccctggcctactatgccgagagcgtgaagggc
aggtttaccatctcccgcgataacgccaagaatacagtggtgctgcagatgaactccctgaaacctgaggacacagccctgtactattgtgccgccgatc
ggaagagcgtgatgagcattagaccagactattgggggcagggaacacaggtgaccgtgagcagcactagtaccacgacgccagcgccgcgacca
ccaacaccggcgcccaccatcgcgtcgcagcccctgtccctgcgcccagaggcgtgccggccagcggcggggggcgcagtgcacacgagggggc
tggacttcgcctgtgatatctacatctgggcgcccttggccgggacttgtggggtccttctcctgtcactggttatcaccctttactgcaaacggggcagaaa
gaaactcctgtatatattcaaacaaccatttatgagaccagtacaaactactcaagaggaagatggctgtagctgccgatttccagaagaagaagaagga
ggatgtgaactgagagtgaagttcagcaggagcgcagacgcccccgcgtaccagcagggccagaaccagctctataacgagctcaatctaggacgaa
gagaggagtacgatgttttggacaagagacgtggccgggaccctgagatggggggaaagccgagaaggaagaaccctcaggaaggcctgtacaatg
aactgcagaaagataagatggcggaggcctacagtgagattgggatgaaaggcgagcgccggaggggcaaggggcacgatggcctttaccagggt
ctcagtacagccaccaaggacacctacgacgcccttcacatgcaggccctgccccctcgctaa
SEQ ID NO: 43 (M172, SIV Nef-IRES-BCMA BiVHH CAR2 nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCTCTCCCTCCCCCCCCCCTAACGTTACTGGCCGAAGCCGCTTGGAATAAGGCCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAAACGTCTAGGCCCCCCGAACCACGGGGACGTGGTTTTCCTTT
GAAAAACACGATGATAATATGGCCACAggatccgccgccaccATGGCTCTGCCCGTCACCGCACTGC
TGCTGCCTCTGGCTCTGCTGCTGCACGCTGCTCGCCCTCAGGTCAAACTGGAAGAATCTGGC
GGAGGCCTGGTGCAGGCAGGCAGGTCCCTGAGGCTGTCTTGCGCAGCAAGCGAGCACACCT
TTAGCTCCCACGTGATGGGATGGTTCAGGCAGGCACCAGGCAAGGAGAGAGAGTCCGTGGC
CGTGATCGGCTGGAGGGACATCTCCACATCTTACGCCGATTCTGTGAAGGGCCGGTTTACCA
TCAGCAGAGACAACGCCAAGAAGACACTGTATCTGCAGATGAATAGCCTGAAGCCTGAGGA
CACCGCCGTGTACTATTGCGCAGCAAGGAGAATCGATGCAGCAGACTTCGATTCCTGGGGAC
AGGGAACCCAGGTGACAGTGTCTAGCGGAGGAGGAGGAAGCGCCGTGCAGCTGGTGGAGTC
CGGCGGCGGCCTGGTGCAGGCCGGCGATTCTCTGCGGCTGACCTGTACAGCCTCCGGCAGAG
CCTTCTCTACCTACTTTATGGCCTGGTTTAGACAGGCCCCTGGCAAGGAGAGGGAGTTTGTG
GCAGGAATCGCATGGAGCGGAGGcTCCACAGCATACGCCGACTCCGTGAAGGGCAGGTTCA
CCATCTCTCGCGATAACGCCAAGAATACAGTGTATCTGCAGATGAACTCTCTGAAGAGCGAG
GACACAGCCGTGTACTATTGTGCCAGCCGGGGAATCGAGGTGGAGGAATTTGGGGCTTGGG
GGCAGGGAACTCAGGTGACCGTCTCATCAactagtaccacgacgccagcgccgcgaccaccaacaccggcgcccaccat
cgcgtcgcagcccctgtccctgcgcccagaggcgtgccggccagcggcggggggcgcagtgcacacgagggggctggacttcgcctgtgatatcta
catctgggcgcccttggccgggacttgtggggtccttctcctgtcactggttatcaccctttactgcaaacggggcagaaagaaactcctgtatatattcaaa
caaccatttatgagaccagtacaaactactcaagaggaagatggctgtagctgccgatttccagaagaagaagaaggaggatgtgaactgagagtgaag
ttcagcaggagcgcagacgcccccgcgtaccagcagggccagaaccagctctataacgagctcaatctaggacgaagagaggagtacgatgttttgg
acaagagacgtggccgggaccctgagatggggggaaagccgagaaggaagaaccctcaggaaggcctgtacaatgaactgcagaaagataagatg
gcggaggcctacagtgagattgggatgaaaggcgagcgccggaggggcaaggggcacgatggcctttaccagggtctcagtacagccaccaagga
cacctacgacgcccttcacatgcaggccctgccccctcgctaa
SEQ ID NO: 54 (M173, SIV Nef-IRES-BCMA mono-VHH CAR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTCTGATGCATCCAGCACAGATGGG
GGAAGATCCTGATGGAATAGATCATGGAGAAGTCTTGGTCTGGAAGTTTGACCCGAAGTTG
GCGGTGGAGTACCGCCCGGACATGTTTAAGGACATGCACGAACATGCAAAGCGCtgaacgcgtG
CCCCTCTCCCTCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAAC
CTGGCCCTGTCTTCTTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCTCTGCGGGCCAAA
AGCCACGTGTATAAGATACACCTGCAAAGGCGGCACAACCCCAGTGCCACGTTGTGAGTTG
GATAGTTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTTACATG
TGTTTAGTCGAGGTTAAAAAACGTCTAGGCCCCCCGAACCACGGGGACGTGGTTTTCCTTT
GAAAAACACGATGATAATATGGCCACAggatccgccgccaccatggctctgcccgtcaccgctctgctgctgcctctggctct
gctgctgcacgctgctcgccctcaggtcaaactggaagaatctggcggaggcctggtgcaggcaggacggagcctgcgcctgagctgcgcagcatt
gagcacaccttcagctcccacgtgatgggctggtttcggcaggccccaggcaaggagagagagagcgtggccgtgatcggctggagggacatctcca
catcttacgccgattccgtgaagggccggttcaccatcagccgggacaacgccaagaagacactgtatctgcagatgaacagcctgaagcccgaggac
accgccgtgtactattgcgcagcaaggagaatcgacgcagcagactttgattcctggggccagggcacccaggtgacagtgctagcactagtaccac
gacgccagcgccgcgaccaccaacaccggcgcccaccatcgcgtcgcagcccctgtccctgcgcccagaggcgtgccggccagcggcggggggc
gcagtgcacacgagggggctggacttcgcctgtgatatctacatctgggcgcccttggccgggacttgtggggtccttctcctgtcactggttatcaccctt
tactgcaaacggggcagaaagaaactcctgtatatattcaaacaaccatttatgagaccagtacaaactactcaagaggaagatggctgtagctgccgatt
tccagaagaagaagaaggaggatgtgaactgagagtgaagttcagcaggagcgcagacgcccccgcgtaccagcagggccagaaccagctctataa
cgagctcaatctaggacgaagagaggagtacgatgttttggacaagagacgtggccgggaccctgagatggggggaaagccgagaaggaagaacc
ctcaggaaggcctgtacaatgaactgcagaaagataagatggcggaggcctacagtgagattgggatgaaaggcgagcgccggaggggcaagggg
cacgatggcctttaccagggtctcagtacagccaccaaggacacctacgacgcccttcacatgcaggccctgccccctcgctaa
SEQ ID NO: 55 (anti-CD20 scFv (Rituximab) CAR amino acid sequence)
MALPVTALLLPLALLLHAARPQVQLQQPGAELVKPGASVKMSCKASGYTFTSYNMHWVKQTP
GRGLEWIGAIYPGNGDTSYNQKFKGKATLTADKSSSTAYMQLSSLTSEDSAVYYCARSTYYGGD
WYFNVWGAGTTVTVSAGGGGSGGGGSGGGGSQIVLSQSPAILSASPGEKVTMTCRASSSVSYIH
WFQQKPGSSPKPWIYATSNLASGVPVRFSGSGSGTSYSLTISRVEAEDAATYYCQQWTSNPPTFG
GGTKLEIKTSTTTPAPRPPTPAPTIASQPLSLRPEACRPAAGGAVHTRGLDFACIYIWAPLAGTC
GVLLLSLVITLYCKRGRKKLLYIFKQPFMRPVQTTQEEDGCSCRFPEEEEEGGCELRVKFSRSADAP
AYQQGQNQLYNELNLGRREEYDVLDKRRGRDPEMGGKPRRKNPQEGLYNELQKDKMAEAYSE
IGMKGERRRGKGHDGLYQGLSTATKDTYDALHMQALPPR
SEQ ID NO: 56 (anti-CD20 scFv (Leu-16) CAR amino acid sequence)
MALPVTALLLPLALLLHAARPEVQLQQSGAELVKPGASVKMSCKASGYTFTSYNMHWVKQTPG
QGLEWIGAIYPGNGDTSYNQKFKGKATLTADKSSSTAYMQLSSLTSEDSADYYCARSNYYGSSY
WFFDVWGAGTTVTVSSGGGGSGGGGSGGGGSDIVLTQSPAILSASPGEKVTMTCRASSSVNYM
DWYQKKPGSSPKPWIYATSNLASGVPARFSGSGSGTSYSLTISRVEAEDAATYYCQQWSFNPPTF
GGGTKLEIKTSTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 57 (C19 × CD20 scFv CAR amino acid sequence)
MALPVTALLLPLALLLHAARPEVQLQQSGAELVKPGASVKMSCKASGYTFTSYNMHWVKKQTPG
QGLEWIGAIYPGNGDTSYNQKFKGKATLTADKSSSTAYMQLSSLTSEDSADYYCARSNYYGSSY
WFFDVWGAGTTVTVSSGGGGSGGGGSGGGGSDIVLTQSPAILSASPGEKVTMTCRASSSVNYM
DWYQKKPGSSPKPWIYATSNLASGVPARFSGSGSGTSYSLTISRVEAEDAATYYCQQWSFNPPTF
GGGTKLEIKGGGGSGGGGSGGGGSGGGGSGGGGSDIQMTQTTSSLSASLGDRVTISCRASQDISK
YLNWYQQKPDGTVKLLIYHTSRLHSGVPSRFSGSGSGTDYSLTISNLEQEDIATYFCQQGNTLPY
TFGGGTKLEITGSTSGSGKPGSGEGSTKGEVKLQESGPGLVAPSQSLSVTCTVSGVSLPDYGVSWI
RQPPRKGLEWLGVIWGSETTYYNSALKSRLTIIKDNSKSQVFLKMNSLQTDDTAIYYCAKHYYY
GGSYAMDYWGQGTSVTVSSTSTTTPAPRPPTPAPTIASQPLSLRPEACRPAAGGAVHTRGLDFAC
DIYIWAPLAGTCGVLLLSLVITLYCKRGRKKLLYIFKQPFMRPVQTTQEEDGCSCRFPEEEEGGCE
LRVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 58 (anti-CD19 scFv CAR amino acid sequence)
MALPVTALLLPLALLLHAARPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGGGG
SGGGGSGGGGSEVKLQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGGSYAMDYWGQGTSV
TVSSTSTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 59 (anti-BCMA BiVHH CAR1 amino acid sequence)
MALPVTALLLPLALLLHAARPQVKLEESGGGLVQAGRSLRLSCAASEHTFSSHVMGWFRQAPG
KERESVAVIGWRDISTSYADSVKGRFTISRDNAKKTLYLQMNSLKPEDTAVYYCAARRIDAADF
DSWGQGTQVTVSSGGGGSEVQLVESGGGLVQAGGSLRLSCAASGRTFTMGWFRQAPGKEREFV
AAISLSPTLAYYYAESVKGRFTISRDNAKNTVVLQMNSLKPEDTALYYCAADRKSVMSIRPDYWG
QGTQVTVSSTSTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRCCDPEMGGKPRRKNPQEGLYNELQKDKMAEA
YSEIGMKGERRRGKGHDGLYQGLSTATKDTYDALHMQALPPR
SEQ ID NO: 60 (anti-BCMA BiVHH CAR2 amino acid sequence)
MALPVTALLLPLALLLHAARPQVKLEESGGGLVQAGRSLRLSCAASEHTFSSHVMGWFRQAPG
KERESVAVIGWRDISTSYADSVKGRFTISRDNAKKTLYLQMNSLKPEDTAVYYCAARRIDAADF
DSWGQGTQVTVSSGGGGSAVQLVESGGGLVQAGDSLRLTCTASGRAFSTYFMAWFRQAPGKE
REFVAGIAWSGGSTAYADSVKGRFTISRDNAKNTVYLQMNSLKSEDTAVYYCASRGIEVEEFGA
WGQGTQVTVSSTSTTTPAPRPPTPAPTIASQPLSLRPEACRPAAGGAVHTRGLDFACIYIWAPLA
GTCGVLLLSLVITLYCKRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 61 (anti-BCMA mono-VHH CAR amino acid sequence)
MALPVTALLLPLALLLHAARPQVKLEESGGGLVQAGRSLRLSCAASEHTFSSHVMGWFRQAPG
KERESVAVIGWRDISTSYADSVKGRFTISRDNAKKTLYLQMNSLKPEDTAVYYCAARRIDAADF
DSWGQGTQVTVSSTSTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 62 (M133, BCMA BiVHH CAR1-IRES-SIV Nef M116 nucleic acid sequence)
gccgccaccatggctctgcccgtcaccgctctgctgctgcctctggctctgctgctgcacgctgctcgccctcaggtcaaactggaagaatctggcggag
gcctggtgcaggcaggacggagcctgcgcctgagctgcgcagcatccgagcacaccttcagctcccacgtgatgggctggtttcggcaggccccagg
caaggagagagagagcgtggccgtgatcggctggagggacatctccacatcttacgccgattccgtgaagggccggttcaccatcagccgggacaac
gccaagaagacactgtatctgcagatgaacagcctgaagcccgaggacaccgccgtgtactattgcgcagcaaggagaatcgacgcagcagacttga
ttcctggggccagggcacccaggtgacagtgtctagcggaggaggaggatctgaggtgcagctggtggagagcggaggcggcctggtgcaggccg
gaggctctctgaggctgagctgtgcagcatccggaagaaccttcacaatgggctggtttaggcaggcaccaggaaaggagagggagttcgtggcagc
aatcagcctgtcccctaccctggcctactatgccgagagcgtgaagggcaggtttaccatctcccgcgataacgccaagaatacagtggtgctgcagatg
aactccctgaaacctgaggacacgccctgtactattgtgccgccgatcggaagagcgtgatgagcattagaccagactattgggggcagggaacaca
ggtgaccgtgagcagcactagtaccacgacgccagcgccgcgaccaccaacaccggcgcccaccatcgcgtcgcagcccctgtccctgcgcccaga
ggcgtgccggccagcggcggggggcgcagtgcacacgagggggctggacttcgcctgtgatatctacatctgggcgcccttggccgggacttgtgg
ggtccttctcctgtcactggttatcaccctttactgcaaacggggcagaaagaaactcctgtatatattcaaacaaccatttatgagaccagtacaaactactc
aagaggaagatggctgtagctgccgatttccagaagaagaagaaggaggatgtgaactggttaacagagtgaagttcagcaggagcgcagacgcccc
cgcgtaccagcagggccagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcctacagtgagattgg
gatgaaaggcgagcgccggaggggcaaggggcacgatggcctttaccagggtctcagtacagccaccaaggacacctacgacgcccttcacatgca
ggccctgccccctcgctaatgaacgcgtGCCCCTCTCCCTCCCCCCCCCCTAACGTTACTGGCCGAAGCCGCT
TGGAATAAGGCCGGTGTGCGTTTGTCTATATGTTATTTTCCACCATATTGCCGTCTTTTGGCA
ATGTGAGGGCCCGGAAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCCTCTGGAAGCTTCT
TGAAGACAAACAACGTCTGTAGCGACCCTTTGCAGGCAGCGGAACCCCCCACCTGGCGACA
GGTGCCTCTGCGGCCAAAAGCCACGTGTATAAGATACACCTGCAAAGGCGGCACAACCCCA
GTGCCACGTTGTGAGTTGGATAGTTGTGGAAAGAGTCAAATGGCTCTCCTCAAGCGTATTCA
ACAAGGGGCTGAAGGATGCCCAGAAGGTACCCCATTGTATGGGATCTGATCTGGGGCCTCG
GTGCACATGCTTTACATGTGTTTAGTCGAGGTTAAAAAAACGTCTAGGCCCCCCGAACCACG
GGGACGTGGTTTTCCTTTGAAAAACACGATGATAATATGGCCACAggatccgccaccATGGGCTCC
AGCAACTCCAAGAGGCAGCAACAGGGCTTGCTCAAGCTCTGGCGAGGGCTGCGAGGGAAGC
CTGGGGCAGACTGGGTGCTATTGTCCGATCCGCTTATCGGGCAGTCATCAACAGTCCAAGAA
GAGTGCGGCAAGGCCTTGAAAAAGTCCTGGGGTAAAGGTAAAATGACTCCAGACGGCCGCC
GCCTGCAAGAAGGAGACACCTTTGATGAGTGGGATGATGATGAAGAAGAAGTAGGCTTCCC
TGTGCAACCTCGAGTCCCCTTAAGACAGATGACCTATAAATTAGCAGTGGACTTTTCCCACT
TTTTAAAATCAAAGGGGGGACTGGATGGGATATATTACTCTGAAAGAAGAGAAAAGATCCT
GAATTTGTATGCCTTGAACGAGTGGGGAATAATAGATGATTGGCAAGCTTACTCACCAGGCC
CGGGGATAAGGTACCCGAGAGTCTTTGGCTTCTGCTTTAAGCTAGTCCCAGTGGACCTGCAT
GAGGAGGCACGCAACTGTGAGAGACACTGTgctgcaCATCCAGCACAGATGGGGGAAGATCCT
GATGGAATAGATCATGGAGAAGTCTTGGTCTGGAAGTTTGACCCGAAGTTGGCGGTGGAGT
ACCGCCCGGACATGTTTAAGGACATGCACGAACATGCAAAGCGCtaa
SEQ ID NO: 63 (M572, SIV Nef M116-IRES-CD20 chimeric TCR nucleic acid sequence)
ATGGGCTCCAGCAACTCCAAGAGGCAGCAACAGGGCTTGCTCAAGCTCTGGCGAGGGCTGC
GAGGGAAGCCTGGGGCAGACTGGGTGCTATTGTCCGATCCGCTTATCGGGCAGTCATCAACA
GTCCAAGAAGAGTGCGGCAAGGCCTTGAAAAAGTCCTGGGGTAAAGGTAAATGACTCCAG
ACGGCCGCCGCCTGCAAGAAGGAGACACCTTTGATGAGTGGGATGATGATGAAGAAGAAGT
AGGCTTCCCTGTGCAACCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTgctgcaCATCCAGCACAGATGGGGG
AAGATCCTGATGGAATAGATCATGGAGAAGTCTTGGTCTGGAAGTTTGACCCGAAGTTGGCG
GTGGAGTACCGCCCGGACATGTTTAAGGACATGCACGAACATGCAAAGCGCtgaacgcgtGCCCC
TCTCCCTCCCCCCCCCCTAACGTTACTGGCCGAAGCCGCTTGGAATAAGGCCGGTGTGCGTTT
GTCTATATGTTATTTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAACAACGTCTGTAGC
GACCCTTTGCAGGCAGCGGAACCCCCCACCTGGCGACAGGTGCCTCTGCGGCCAAAAGCCA
CGTGTATAAGATACACCTGCAAAGGCGGCACAACCCCAGTGCCACGTTGTGAGTTGGATAGT
TGTGGAAAGAGTCAAATGGCTCTCCTCAAGCGTATTCAACAAGGGGCTGAAGGATGCCCAG
AAGGTACCCCATTGTATGGGATCTGATCTGGGGCCTCGGTGCACATGCTTTACATGTGTTTA
GTCGAGGTTAAAAAAACGTCTAGGCCCCCCGAACCACGGGGACGTGGTTTTCCTTTGAAAAA
CACGATGATAATATGGCCACAGgatccgccgccaccatgcagtctggaacccactggagggtgctgggactgtgcctgctgagcg
tgggcgtgtggggacagGAGGTGCAGCTGCAGCAGAGCGGAGCTGAGCTGGTGAAGCCTGGCGCTAG
CGTGAAGATGAGCTGCAAGGCCAGCGGCTACACCTTCACCAGCTATAACATGCACTGGGTG
AAGCAGACCCCTGGACAGGGACTGGAGTGGATCGGAGCTATCTACCCTGGAAACGGAGACA
CCTCATACAACCAGAAGTTCAAGGGAAAGGCTACCCTGACCGCTGACAAGAGCAGCAGCAC
CGCTTACATGCAGCTGAGCTCACTGACCAGCGAGGACTCCGCCGACTACTACTGCGCCAGAA
GCAACTACTACGGAAGCAGCTACTGGTTCTTCGACGTGTGGGGAGCTGGAACCACCGTGACC
GTGTCAAGCGGCGGCGGAGGcTCCGGAGGCGGAGGATCTGGCGGCGGCGGCAGCGACATCG
TGCTGACCCAGAGCCCTGCTATCCTGTCTGCCAGCCCTGGAGAGAAGGTGACCATGACCTGC
AGAGCTAGCAGCAGCGTGAACTACATGGACTGGTATCAGAAAAAGCCCGGCAGCTCACCTA
AGCCTTGGATCTACGCTACCAGCAACTTAGCCAGCGGCGTGCCTGCTAGATTCTCCGGAAGC
GGCTCTGGAACCAGCTACTCCCTTACCATCAGCAGAGTGGAGGCTGAGGACGCTGCTACCTA
CTACTGCCAGCAGTGGAGCTTCAACCCTCCTACCTTCGGAGGAGGAACCAAGCTGGAGATCA
AGactagtGGCGGCGGAGGCTCTGGCGGCGGAGGAAGCGGCGGCGGCGGCTCCgatggcaacgaggag
atgggcggcatcacccagacaccctacaaggtgtccatctctggcaccacagtgatcctgacctgtccacagtatcccggctctgagatcctgtggcagc
acaacgacaagaatatcggcggcgatgaggacgataagaatatcggcagcgacgaggatcacctgtctctgaaggagttcagcgagctggagcagtc
cggctactacgtgtgctaccctcggggctccaagccagaggacgccaacttttacctgtatctgcgggccagagtgtgcgagaattgtatggagatggac
gtgatgtccgtggccaccatcgtgatcgtggatatctgtatcacaggcggcctgctgctgctggtgtactattggagcaagaaccggaaggccaaggcca
agcctgtgaccagaggagcaggagcaggaggcaggcagaggggacagaacaaggagaggccacctccagtgcccaatcctgactacgagccaat
caggaagggccagcgcgatctgtatagcggcctgaatcagaggcgcatctgataa
SEQ ID NO: 64 (anti-CD20 chimeric TCR amino acid sequence; CD3ϵ ([no signal peptide]
extracellular-helical-cytoplasmic) amino acid sequence is underlined)
MQSGTHWRVLGLCLLSVGVWGQEVQLQQSGAELVKPGASVKMSCKASGYTFTSYNMHWVKQ
TPGQGLEWIGAIYPGNGDTSYNQKFKGKATLTADKSSSTAYMQLSSLTSEDSADYYCARSNYYG
SSYWFFDVWGAGTTVTVSSGGGGSGGGGSGGGGSDIVLTQSPAILSASPGEKVTMTCRASSSVN
YMDWYQKKPGSSPKPWIYATSNLASGVPARFSGSGSGTSYSLTISRVEAEDAATYYCQQWSFNP
PTFGGGTKLEIKTSGGGGSGGGGSGGGGSDGNEEMGGITQTPYKVSISGTTVILTCPQYPGSEILW
QHNDKNIGGDEDDKNIGSDEDHLSLKEFSELEQSGYYVCYPRGSKPEDANFYLYLRARVCENCM
EMDVMSVATIVIVDICITGGLLLLVYYWSKNRKAKAKPVTRGAGAGGRQRGQNKERPPPVPNP
DYEPIRKGQRDLYSGLNQRRI
SEQ ID NO: 65 (M574, SIV Nef M116-IRES-CD20 TAC nucleic acid sequence)
ATGGGCTCCAGCAACTCCAAGAGGCAGCAACAGGGCTTGCTCAAGCTCTGGCGAGGGCTG
GAGGGAAGCCTGGGGCAGACTGGGTGCTATTGTCCGATCCGCTTATCGGGCAGTCATCAACA
GTCCAAGAAGAGTGCGGCAAGGCCTTGAAAAGTCCTGGGGTAAAGGTAAAATGACTCCAG
ACGGCCGCCGCCTGCAAGAAGGAGACACCTTTGATGAGTGGGATGATGATGAAGAAGAAGT
AGGCTTCCCTGTGCAACCCTCGAGTCCCCTTAAGACAGATGACCTATAAATTAGCAGTGGACT
TTTCCCACTTTTTAAAATCAAAGGGGGGACTGGATGGGATATATTACTCTGAAAGAAGAGAA
AAGATCCTGAATTTGTATGCCTTGAACGAGTGGGGAATAATAGATGATTGGCAAGCTTACTC
ACCAGGCCCGGGGATAAGGTACCCGAGAGTCTTTGGCTTCTGCTTTAAGCTAGTCCCAGTGG
ACCTGCATGAGGAGGCACGCAACTGTGAGAGACACTGTgctgcaCATCCAGCACAGATGGGGG
AAGATCCTGATGGAATAGATCATGGAGAAGTCTTGGTCTGGAAGTTTGACCCGAAGTTGGCG
GTGGAGTACCGCCCGGACATGTTTAAGGACATGCACGAACATGCAAAGCGCtgaacgcgtGCCCC
TCTCCCTCCCCCCCCCCTAACGTTACTGGCCGAAGCCGCTTGGAATAAGGCCGGTGTGCGTTT
GTCTATATGTTATTTTCCACCATATTGCCGTCTTTTGGCAATGTGAGGGCCCGGAAACCTGGC
CCTGTCTTCTTGACGAGCATTCCTAGGGGTCTTTCCCCCTCTCGCCAAAGGAATGCAAGGTCTG
TTGAATGTCGTGAAGGAAGCAGTTCCTCTGGAAGCTTCTTGAAGACAAACAACGTCTGTAGC
GACCCTTTGCAGGCAGCGGAACCCCCCACCTGGCGACAGGTGCCTCTGCGGCCAAAAGCCA
CGTGTATAAGATACACCTGCAAAGGCGGCACAACCCCAGTGCCACGTTGTGAGTTGGATAGT
TGTGGAAAGAGTCAAATGGCTCTCCTCAAGCGTATTCAACAAGGGGCTGAAGGATGCCCAG
AAGGTACCCCATTGTATGGGATCTGATCTGGGGCCTCGGTGCACATGCTTTACATGTGTTTA
GTCGAGGTTAAAAAAACGTCTAGGCCCCCCGAACCACGGGGACGTGGTTTTCCTTTGAAAAA
CACGATGATAATATGGCCACAGGATCCgccgccaccatgcagtctggaacccactggagggtgctgggactgtgcctgctga
gcgtgggcgtgtgggggacagGAGGTGCAGCTGCAGCAGAGCGGAGCTGAGCTGGTGAAGCCTGGCGCT
AGCGTGAAGATGAGCTGCAAGGCCAGCGGCTACACCTTCACCAGCTATAACATGCACTGGG
TGAAGCAGACCCCTGGACAGGGACTGGAGTGGATCGGAGCTATCTACCCTGGAAACGGAGA
CACCTCATACAACCAGAAGTTCAAGGGAAAGGCTACCCTGACCGCTGACAAGAGCAGCAGC
ACCGCTTACATGCAGCTGAGCTCACTGACCAGCGAGGACTCCGCCGACTACTACTGCGCCAG
AAGCAACTACTACGGAAGCAGCTACTGGTTCTTCGACGTGTGGGGAGCTGGAACCACCGTG
ACCGTGTCAAGCGGCGGCGGAGGcTCCGGAGGCGGAGGATCTGGCGGCGGCGGCAGCGACA
TCGTGCTGACCCAGAGCCCTGCTATCCTGTCTGCCAGCCCTGGAGAGAAGGTGACCATGACC
TGCAGAGCTAGCAGCAGCGTGAACTACATGGACTGGTATCAGAAAAAGCCCGGCAGCTCAC
CTAAGCCTTGGATCTACGCTACCAGCAACTTAGCCAGCGGCGTGCCTGCTAGATTCTCCGGA
AGCGGCTCTGGAACCAGCTACTCCCTTACCATCAGCAGAGTGGAGGCTGAGGACGCTGCTAC
CTACTACTGCCAGCAGTGGAGCTTCAACCCTCCTACCTTCGGAGGAGGAACCAAGCTGGAGA
TCAAGactagtggcggcggcggctctggaggaggaggcagcggcggcggaggctccggcggcggcggctctatggacattcagatgacccag
tccccaagctccctgtctgccagcgtgggagacagagtgaccatcacatgcagggccagccaggatatccgcaactatctgaattggtatcagcagaaa
cccggcaaggcccctaagctgctgatctattacaccagcaggctggagtccggagtgccatcaagattctccggctctggcagcggaaccgactacac
cctgacaatctctagcctgcagccagaggatttcgccacatattactgccagcagggcaacaccctgccctggacatttggccagggcaccaaggtgga
gatcaagggaggaggaggcagcgggggcggcggctccggaggaggcggctctgaggtgcagctggtggagagcggaggaggactggtgcagcc
tggaggcagcctgcggctgtcctgtgccgccagcggctattccttcaccggctacacaatgaattgggtcagacaggcaccaggaaagggactggagt
gggtggccctgatcaaccctaccaagggcgtgtccacatataatcagaagttcaaggacaggtttaccatctctgtggataagagcaagaacacagccta
cctgcagatgaatagcctgagggccgaggacaccgccgtgtattactgcgcacgcagcggatattacggagactccgattggtactttgacgtgtgggg
ccagggcaccctggtgacagtgtcctccggcggaggaggcagctccggacaggtgctgctggagtccaatatcaaggtgctgccaacctggtctacac
ctgtgcagccaatggcactgatcgtgctgggaggagtggcaggactgctgctgttcatcggactgggcatcttcttttgcgtgcgctgtaggcaccggag
aaggcaggcagagaggatgtctcagatcaagagactgctgagcgagaagaagacctgccagtgtcctcaccgctttcagaagacatgtagcccaatct
gataa
SEQ ID NO: 66 (anti-CD20 TAC amino acid sequence; CD4 (partial extracellular-helical-
cytoplasmic) amino acid sequence is underlined)
MQSGTHWWRVLGLCLLSVGVWGQEVQLQQSGAELVKPGASVKMSCKASGYTFTSYNMHWVKQ
TPGQGLEWIGAIYPGNGDTSYNQKFKGKATLTADKSSSTAYMQLSSLTSEDSADYYCARSNYYG
SSYWFFDVWGAGTTVTVSSGGGGSGGGGSGGGGSDIVLTQSPAILSASPGEKVTMTCRASSSVN
YMDWYQKKPGSSPKPWIYATSNLASGVPARFSGSGSGTSYSLTISRVEAEDAATYYCQQWSFNP
PTFGGGTKLEIKTSGGGGSGGGGSGGGGSGGGGSMDIQMTQSPSSLSASVGDRVTITCRASQDIR
NYLNWYQQKPGKAPKLLIYYTSRLESGVPSRFSGSGSGTDYTLTISSLQPEDFATYYCQQGNTLP
WTFGQGTKVEIKGGGGSGGGGSGGGGSEVQLVESGGGLVQPGGSLRLSCAASGYSFTGYTMN
WVRQAPGKGLEWVALINPTKGVSTYNQKFKDRFTISVDKSKNTAYLQMNSLRAEDTAVYYCAR
SGYYGDSDWYFDVWGQGTLVTVSSGGGGSSGQVLLEESNIKVLPTWSTPVQPMALIVLGGVAGL
LLFIGLGIFFCVRCRHRRRQAERMSQIKRLLSEKKTCQCPHRFQKTCSPI

1-66. (canceled)

67. A method of producing a modified T cell, comprising: introducing into a precursor T cell a first nucleic acid encoding a Negative Regulatory Factor (Nef) protein, wherein the Nef protein upon expression results in down-modulation of the endogenous T cell receptor (TCR) in the modified T cell.

68. The method of claim 67, wherein the down-modulation of the endogenous TCR comprises down-regulating cell surface expression of endogenous TCR by at least about 50%.

69. The method of claim 67, wherein the Nef protein upon expression does not down-modulate endogenous CD3ζ or down-modulates endogenous CD3ζ by at most about 50%.

70. The method of claim 67, wherein the modified T cell expressing the Nef protein comprises a modified endogenous TCR locus.

71. The method of claim 67, wherein the Nef protein is selected from the group consisting of SIV Nef, HIV1 Nef, HIV2 Nef, and Nef homologous protein.

72. The method of claim 67, wherein the Nef protein is a wildtype Nef protein.

73. The method of claim 67, wherein the Nef protein is a mutant Nef protein.

74. The method of claim 73, wherein the mutant Nef protein comprises an amino acid sequence of any one of SEQ ID NOs: 18-22.

75. The method of claim 67, wherein the precursor T cell comprises a second nucleic acid encoding a functional exogenous receptor comprising an extracellular ligand binding domain and optionally an intracellular signaling domain.

76. The method of claim 67, further comprising introducing into the precursor T cell a second nucleic acid encoding a functional exogenous receptor comprising an extracellular ligand binding domain and optionally an intracellular signaling domain.

77. The method of claim 76, wherein the first nucleic acid and the second nucleic acid are on separate vectors.

78. The method of claim 76, wherein the first nucleic acid and the second nucleic acid are on the same vector.

79. The method of claim 78, wherein the first nucleic acid and the second nucleic acid are operably linked to the same promoter.

80. The method of claim 79, wherein the first nucleic acid is upstream of the second nucleic acid.

81. The method of claim 79, wherein the first nucleic acid and the second nucleic acid are connected via a linking sequence.

82. The method of claim 76, further comprising isolating or enriching modified T cells comprising the first and/or the second nucleic acid.

83. The method of claim 67, further comprising isolating or enriching TCR-negative T cells from the modified T cells expressing the Nef protein.

84. The method of claim 76, wherein the functional exogenous receptor is a chimeric TCR (cTCR), a T cell antigen coupler (TAC), a TAC-like chimeric receptor, or a chimeric antigen receptor (CAR).

85. A modified T cell obtained by the method of claim 84.

86. A method of treating a disease in an individual, comprising administering to the individual an effective amount of the modified T cell of claim 85.

87. A non-naturally occurring Nef protein, comprising one or more mutations in myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof, or one or more mutations at any of amino acid residues listed in Table 11.


SEQUENCE LISTING (66 sequences)

SEQ ID NO: 1 - DNA, 669 bp, Simian Immunodeficiency Virus
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgc

SEQ ID NO: 2 - DNA, 618 bp, Human Immunodeficiency Virus
atgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgc

SEQ ID NO: 3 - DNA, 771 bp, Human Immunodeficiency Virus
atgggtgcgagtggatccaagaagctttccaagcattcgcgaggactacgagagagactcttgcgggcgcgtggggatggttatgggaagcagcgcgacgcatcgggaggggaatactcgcagttccaagaagaatcaggcagggagcagaactcgccctcctgtgagggacagcagtatcagcagggagagtacatgaacagcccatggagaaacccagcaacagaaagacagaaagatttgtataggcagcaaaatatggatgatgtagattctgatgatgatgacctaataggagttcctgttacaccaagagtaccacggagagaaatgacctataaattggcaatagatatgtcacattttataaaagaaaaagggggactgcaagggatgttttacagtaggaggagacatagaatcctagacatatacctagaaaaagaggaagggataataccagattggcagaattatactcatgggccaggagtaaggtacccaatgtacttcgggtggctgtggaagctagtatcagtagaactctcacaagaggcagaggaagatgaggccaactgcttagtacacccagcacaaacaagcagacatgatgatgagcatggggagacattagtgtggcagtttgactccatgctggcctataactacaaggccttcactctgtacccagaagagtttgggcacaagtcaggattgccagagaaagaatggaaggcaaaactgaaagcaagagggataccatatagtgaa

SEQ ID NO: 4 - DNA, 630 bp, Human Immunodeficiency Virus
atgggtggcaagtggtcaaaatgcagcatagttggatggcctgatataagagagagaatgagacgaactgagccagcagcagagccagcagcagaaggagtaggagcagcgtctcaagacttagataaacatggagcacttacaagtagcaacacaaacaccactaatgctgattgtgcttggccggaagcgcaagaggatgaaggagaagtaggctttgcagtcagacctcagagtcctttaagaccaatgacttataagggagcatttgatctcggcttctttttaaaagaagggggactggaagggttaatttactctaagaaaaggcaagagatccttgatttgtgggtctatcatacacaaggctacttccctgattggcaaaactacacaccgggaccaggggtcagatacccactgacttttgggtggtgcttcaagctggtaccagttgacccaaaggcagtagaagaggccaacgaaggagaagacaactgtctgctacacccagtgtgccagcatggaatggaggatgaacacagagaagtattaatgtggaagtttgacagtcaactagcacgcagacacatggcccgagagctacatccggagttctacaaagactgc

SEQ ID NO: 5 - DNA, 387 bp, Human Immunodeficiency Virus
atgggtggcaagtggtcaaaatgcagcataggtggatggcctcagataagagagagaatgagacgaactgagccagcagtagagccagcagcagagccagcagcagaaggagtaggagcagcgtggctacttcctgattggcaaaactacacaccgggaccaggagtcagatacccactgacttttgggtggtgcttcaagctggtaccagtagacccaggggcagtagaagaggccaacgaaggagaaaacaactgtttgctacacccggtgtgccagcatggaatggaggatgagcaaagagaagtattagtgtgcaagtttgacagtctactagcacgcagacacatggcccgcgagctacatccggagttctacaaagactgc

SEQ ID NO: 6 - DNA, 540 bp, Human Immunodeficiency Virus
atgggtgcgagtggatccaagaagcgttccaagcccttgcaaggactacaagagagactcttgcaggcgcggggagagacttgtggagggcgctgcaacgaatcgggagggggatacttgcagtcccacgaaggatcaggcagggagcagaactcgccctcctgtgagggacagcgatatcagcagggagattttgtaaataccccatggagaaccccagcagcagaaagggagaaagaattgtacaaacagcaaaatatggatgatgtagatctagatgatgatgaccaagtaggattccctgtcacaccaagagtaccattaagaccaatgacattcaaattggcagtagatatgtctcattttataaaagaaaaagggggactggaagggctgttttatagtcagagaagacatagaatcttagacttatacttagacaaggcttttactctgtacccagaggaatttgggcataattcaggactgccagagaaagagtggaaggcgagactgaaagcaaggggaataccatttagt

SEQ ID NO: 7 - DNA, 669 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgc

SEQ ID NO: 8 - DNA, 669 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaagctgcagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgc

SEQ ID NO: 9 - DNA, 669 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaagctgcagaaaagatcctgaatttgtatgccttgaacgagtggggaataatagatgattggcaagcttactcaccaggcccggggataaggtacccgagagtctttggcttctgctttaagctagtcccagtggacctgcatgaggagcatcacaactgtgagagacactgtgctgcacatccagcacagatgggggcggcacctgatggaatagatcatggagaagtcttggtctggaagtttgacccgaagttggcggtggagtaccgcccggacatgtttaaggacatgcacgaacatgcaaagcgc

SEQ ID NO: 10 - DNA, 669 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggagcatcacaactgtgagagacactgtgctgcacatccagcacagatgggggcggcacctgatggaatagatcatggagaagtcttggtctggaagtttgacccgaagttggcggtggagtaccgcccggacatgtttaaggacatgcacgaacatgcaaagcgc

SEQ ID NO: 11 - DNA, 669 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggcggcacctgatggaatagatcatggagaagtcttggtctggaagtttgacccgaagttggcggtggagtaccgcccggacatgtttaaggacatgcacgaacatgcaaagcgc

SEQ ID NO: 12 - protein, 223 aa, Simian Immunodeficiency Virus
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCLMHPAQMGEDPDGIDHGEVLVWKFDPKLAVEYRPDMFKDMHEHAKR

SEQ ID NO: 13 - protein, 206 aa, Human Immunodeficiency Virus
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 14 - protein, 257 aa, Human Immunodeficiency Virus
MGASGSKKLSKHSRGLRERLLRARGDGYGKQRDASGGEYSQFQEESGREQNSPSCEGQQYQQGEYMNSPWRNPATERQKDLYRQQNMDDVDSDDDDLIGVPVTPRVPRREMTYKLAIDMSHFIKEKGGLQGMFYSRRRHRILDIYLEKEEGIIPDWQNYTHGPGVRYPMYFGWLWKLVSVELSQEAEEDEANCLVHPAQTSRHDDEHGETLVWQFDSMLAYNYKAFTLYPEEFGHKSGLPEKEWKAKLKARGIPYSE

SEQ ID NO: 15 - protein, 210 aa, Human Immunodeficiency Virus
MGGKWSKCSIVGWPDIRERMRRTEPAAEPAAEGVGAASQDLDKHGALTSSNTNTTNADCAWPEAQEDEGEVGFAVRPQSPLRPMTYKGAFDLGFFLKEGGLEGLIYSKKRQEILDLWVYHTQGYFPDWQNYTPGPGVRYPLTFGWCFKLVPVDPKAVEEANEGEDNCLLHPVCQHGMEDEHREVLMWKFDSQLARRHMARELHPEFYKDC

SEQ ID NO: 16 - protein, 129 aa, Human Immunodeficiency Virus
MGGKWSKCSIGGWPQIRERMRRTEPAVEPAAEPAAEGVGAAWLLPDWQNYTPGPGVRYPLTFGWCFKLVPVDPGAVEEANEGENNCLLHPVCQHGMEDEQREVLVCKFDSLLARRHMARELHPEFYKDC

SEQ ID NO: 17 - protein, 180 aa, Human Immunodeficiency Virus
MGASGSKKRSKPLQGLQERLLQARGETCGGRCNESGGGYLQSHEGSGREQNSPSCEGQRYQQGDFVNTPWRTPAAEREKELYKQQNMDDVDLDDDDQVGFPVTPRVPLRPMTFKLAVDMSHFIKEKGGLEGLFYSQRRHRILDLYLDKAFTLYPEEFGHNSGLPEKEWKARLKARGIPFS

SEQ ID NO: 18 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCAAHPAQMGEDPDGIDHGEVLVWKFDPKLAVEYRPDMFKDMHEHAKR

SEQ ID NO: 19 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSEAAEKILNLYALNEWGIIDDWQAYSPGPGIRYPRVFGFCFKLVPVDLHEEARNCERHCAAHPAQMGEDPDGIDHGEVLVWKFDPKLAVEYRPDMFKDMHEHAKR

SEQ ID NO: 20 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSEAAEKILNLYALNEWGIIDDWQAYSPGPGIRYPRVFGFCFKLVPVDLHEEHHNCERHCAAHPAQMGAAPDGIDHGEVLVWKFDPKLAVEYRPDMFKDMHEHAKR

SEQ ID NO: 21 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEHHNCERHCAAHPAQMGAAPDGIDHGEVLVWKFDPKLAVEYRPDMFKDMHEHAKR

SEQ ID NO: 22 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCAAHPAQMGAAPDGIDHGEVLVWKFDPKLAVEYRPDMFKDMHEHAKR

SEQ ID NO: 23 - DNA, 20 bp, Artificial Sequence
  Synthetic Construct
gagaatcaaaatcggtgaat

SEQ ID NO: 24 - DNA, 1578 bp, Artificial Sequence
  Synthetic Construct
gaattcatgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgcacgcgtggaagcggagctactaacttcagcctgctgaagcaggctggagacgtggaggagaaccctggacctggatccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttctgatctaga

SEQ ID NO: 25 - DNA, 1305 bp, Artificial Sequence
  Synthetic Construct
gaattcatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgcactagtggcagtggagagggcagaggaagtctgctaacatgcggtgacgtcgaggagaatcctggcccaacgcgtatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatcc

SEQ ID NO: 26 - DNA, 1458 bp, Artificial Sequence
  Synthetic Construct
gaattcatgggtgcgagtggatccaagaagctttccaagcattcgcgaggactacgagagagactcttgcgggcgcgtggggatggttatgggaagcagcgcgacgcatcgggaggggaatactcgcagttccaagaagaatcaggcagggagcagaactcgccctcctgtgagggacagcagtatcagcagggagagtacatgaacagcccatggagaaacccagcaacagaaagacagaaagatttgtataggcagcaaaatatggatgatgtagattctgatgatgatgacctaataggagttcctgttacaccaagagtaccacggagagaaatgacctataaattggcaatagatatgtcacattttataaaagaaaaagggggactgcaagggatgttttacagtaggaggagacatagaatcctagacatatacctagaaaaagaggaagggataataccagattggcagaattatactcatgggccaggagtaaggtacccaatgtacttcgggtggctgtggaagctagtatcagtagaactctcacaagaggcagaggaagatgaggccaactgcttagtacacccagcacaaacaagcagacatgatgatgagcatggggagacattagtgtggcagtttgactccatgctggcctataactacaaggccttcactctgtacccagaagagtttgggcacaagtcaggattgccagagaaagaatggaaggcaaaactgaaagcaagagggataccatatagtgaaactagtggcagtggagagggcagaggaagtctgctaacatgcggtgacgtcgaggagaatcctggcccaacgcgtatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatcc

SEQ ID NO: 27 - protein, 525 aa, Artificial Sequence
  Synthetic Construct
EFMGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCLMHPAQMGEDPDGIDHGEVLVWKFDPKLAVEYRPDMFKDMHEHAKRTRGSGATNFSLLKQAGDVEENPGPGSMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFSR

SEQ ID NO: 28 - protein, 434 aa, Artificial Sequence
  Synthetic Construct
EFMGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNCTSGSGEGRGSLLTCGDVEENPGPTRMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGS

SEQ ID NO: 29 - protein, 485 aa, Artificial Sequence
  Synthetic Construct
EFMGASGSKKLSKHSRGLRERLLRARGDGYGKQRDASGGEYSQFQEESGREQNSPSCEGQQYQQGEYMNSPWRNPATERQKDLYRQQNMDDVDSDDDDLIGVPVTPRVPRREMTYKLAIDMSHFIKEKGGLQGMFYSRRRHRILDIYLEKEEGIIPDWQNYTHGPGVRYPMYFGWLWKLVSVELSQEAEEDEANCLVHPAQTSRHDDEHGETLVWQFDSMLAYNYKAFTLYPEEFGHKSGLPEKEWKAKLKARGIPYSETSGSGEGRGSLLTCGDVEENPGPTRMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGS

SEQ ID NO: 30 - DNA, 66 bp, Artificial Sequence
  Synthetic Construct
ggaagcggagctactaacttcagcctgctgaagcaggctggagacgtggaggagaaccctggacct

SEQ ID NO: 31 - DNA, 63 bp, Artificial Sequence
  Synthetic Construct
ggcagtggagagggcagaggaagtctgctaacatgcggtgacgtcgaggagaatcctggccca

SEQ ID NO: 32 - DNA, 69 bp, Artificial Sequence
  Synthetic Construct
ggaagcggacagtgtactaattatgctctcttgaaattggctggagatgttgagagcaaccctggacct

SEQ ID NO: 33 - DNA, 75 bp, Artificial Sequence
  Synthetic Construct
ggaagcggagtgaaacagactttgaattttgaccttctcaagttggcgggagacgtggagtccaaccctggacct

SEQ ID NO: 34 - DNA, 585 bp, Artificial Sequence
  Synthetic Construct
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 35 - DNA, 520 bp, Artificial Sequence
  Synthetic Construct
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttacc

SEQ ID NO: 36 - protein, 22 aa, Artificial Sequence
  Synthetic Construct
GSGATNFSLLKQAGDVEENPGP

SEQ ID NO: 37 - protein, 21 aa, Artificial Sequence
  Synthetic Construct
GSGEGRGSLLTCGDVEENPGP

SEQ ID NO: 38 - protein, 23 aa, Artificial Sequence
  Synthetic Construct
GSGQCTNYALLKLAGDVESNPGP

SEQ ID NO: 39 - protein, 25 aa, Artificial Sequence
  Synthetic Construct
GSGVKQTLNFDLLKLAGDVESNPGP

SEQ ID NO: 40 - protein, 5 aa, Artificial Sequence
  Synthetic Construct
GGGGS

SEQ ID NO: 41 - protein, 10 aa, Artificial Sequence
  Synthetic Construct
GGGGSGGGGS

SEQ ID NO: 42 - protein, 12 aa, Artificial Sequence
  Synthetic Construct
GGGSGGGSGGGS

SEQ ID NO: 43 - protein, 16 aa, Artificial Sequence
  Synthetic Construct
GGGSGGGSGGGSGGGS

SEQ ID NO: 44 - protein, 24 aa, Artificial Sequence
  Synthetic Construct
GGGGSGGGGSGGGGGGSGSGGGGS

SEQ ID NO: 45 - protein, 34 aa, Artificial Sequence
  Synthetic Construct
GGGGSGGGGSGGGGGGSGSGGGGSGGGGSGGGGS

SEQ ID NO: 46 - protein, 15 aa, Artificial Sequence
  Synthetic Construct
GGGGSGGGGSGGGGS

SEQ ID NO: 47 - protein, 20 aa, Artificial Sequence
  Synthetic Construct
GGGGSGGGGSGGGGSGGGGS

SEQ ID NO: 48 - DNA, 2745 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgccagtgaccgccttgctccttcccctggctcttctgctgcacgctgctagacctcaggtgcagcttcagcagcctggcgctgagctggtgaagcccggagctagcgtgaagatgtcctgcaaggccagcggctataccttcacctcatacaacatgcactgggtgaagcagacccctggaagaggcctcgagtggattggagctatctaccctggaaacggagacaccagctataaccagaagttcaagggaaaggctaccctgaccgctgacaagagcagcagcaccgcttacatgcagctgagcagccttacaagcgaggactctgccgtgtactactgcgccagaagcacctattacggcggcgactggtacttcaacgtgtggggagctggaaccaccgtgaccgttagcgccggcggcggaggctctggcggcggaggaagcggcggcggcggctcccagatcgtgctgtctcagagccccgctatcttgagcgcctcccctggagagaaggtgaccatgacttgcagagctagcagcagcgtgagctacatccactggttccaacagaagccaggcagctcccctaagccttggatctacgctaccagcaaccttgcctcaggcgttcccgtgagattctctggatctggaagcggcacatcctactccctgaccatctcccgggtcgaggctgaggacgctgctacttactactgccagcagtggactagcaacccccccacatttggcggcggcaccaaactggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - DNA, 2748 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 50 - DNA, 3558 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgcctgtcaccgccctgctgctgcccctggctctgctgctgcacgccgcaagacctgaagtccagctgcagcagtccggggcagagctggtgaagccaggagcctccgtgaagatgtcttgtaaggccagcggctacaccttcacatcctataacatgcactgggtgaagcagacccctggacagggcctggagtggatcggagcaatctacccaggcaacggcgacacaagctataatcagaagtttaagggcaaggccaccctgacagccgataagagctcctctaccgcctacatgcagctgagctccctgacaagcgaggactccgccgattactattgcgcccggtccaattactatggctctagctactggttctttgacgtgtggggagcaggaaccacagtgaccgtgtcctctggaggaggaggaagcggaggaggaggatctggcggcggcggctctgatatcgtgctgacacagagcccagcaatcctgtccgcctctccaggagagaaggtgaccatgacatgtcgggccagctcctctgtgaactacatggactggtatcagaagaagcccggcagctcccctaagccatggatctacgccacctccaatctggcatctggagtgcctgcaaggttcagcggctccggatctggcaccagctattccctgacaatctctcgcgtggaggcagaggatgcagcaacctactattgccagcagtggagcttcaacccccctacctttggcggcggcacaaagctggagatcaagggcggcggcggctccggcggcggcgggagcggcggcggcggctctggcggcggcggcagcggcggcggcggctccgacatccagatgacccagaccacatctagcctgtctgccagcctgggcgacagggtgacaatcagctgtcgcgcctcccaggatatctctaagtacctgaattggtatcagcagaagccagatggcaccgtgaagctgctgatctaccacacaagccggctgcactccggagtgccaagccggttcagcggctctggcagcggcaccgactatagcctgacaatctccaacctggagcaggaggatatcgccacctacttctgccagcagggcaataccctgccttatacatttggcggaggaacaaagctggagatcaccggctccacatctggaagcggcaagccaggatctggagagggaagcaccaagggagaggtgaagctgcaggagtccggaccaggcctggtggcaccttcccagtctctgagcgtgacctgtacagtgtctggcgtgagcctgcctgactacggcgtgtcctggatcaggcagccaccaagaaagggcctggagtggctgggcgtgatctggggcagcgagacaacatactataactccgccctgaagagccggctgaccatcatcaaggataactccaagtctcaggtgttcctgaagatgaatagcctgcagaccgacgatacagccatctactattgcgccaagcactactattatggaggcagttatgctatggactattgggggcagggcacaagcgtcaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - DNA, 2745 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgcctgtgaccgccctgctgctgcccctggccctgctgctgcacgctgctagacctgatattcagatgacccagaccactagctccctgtccgcctctctgggcgacagagtgacaatcagctgcagggcctcccaggatatctctaagtatctgaactggtaccagcagaagccagacggcacagtgaagctgctgatctatcacaccagccgcctgcactccggagtgccatctcggttcagcggctccggatctggcacagactacagcctgaccatctccaacctggagcaggaggatatcgccacctatttctgccagcagggcaatacactgccctacacctttggcggcggcacaaagctggagatcaccggaggaggaggaagcggcggaggaggctccggcggcggcggctctgaggtgaagctgcaggagtccggacctggcctggtggcaccaagccagtccctgtctgtgacatgtaccgtgtccggcgtgtctctgcctgattacggcgtgtcttggatcaggcagccacctaggaagggcctggagtggctgggcgtgatctggggcagcgagacaacatactataattctgccctgaagagcagactgaccatcatcaaggacaacagcaagtcccaggtgttcctgaagatgaatagcctgcagacagacgataccgccatctactattgcgccaagcactactattacggcggcagctatgccatggattactggggccagggcacatccgtgaccgtgtctagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 52 - DNA, 2743 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggctctgcccgtcaccgctctgctgctgcctctggctctgctgctgcacgctgctcgccctcaggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagcggaggaggaggatctgaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctcgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 53 - DNA, 2748 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggctctgcccgtcaccgcactgctgctgcctctggctctgctgctgcacgctgctcgccctcaggtcaaactggaagaatctggcggaggcctggtgcaggcaggcaggtccctgaggctgtcttgcgcagcaagcgagcacacctttagctcccacgtgatgggatggttcaggcaggcaccaggcaaggagagagagtccgtggccgtgatcggctggagggacatctccacatcttacgccgattctgtgaagggccggtttaccatcagcagagacaacgccaagaagacactgtatctgcagatgaatagcctgaagcctgaggacaccgccgtgtactattgcgcagcaaggagaatcgatgcagcagacttcgattcctggggacagggaacccaggtgacagtgtctagcggaggaggaggaagcgccgtgcagctggtggagtccggcggcggcctggtgcaggccggcgattctctgcggctgacctgtacagcctccggcagagccttctctacctactttatggcctggtttagacaggcccctggcaaggagagggagtttgtggcaggaatcgcatggagcggaggctccacagcatacgccgactccgtgaagggcaggttcaccatctctcgcgataacgccaagaatacagtgtatctgcagatgaactctctgaagagcgaggacacagccgtgtactattgtgccagccggggaatcgaggtggaggaatttggggcttgggggcagggaactcaggtgaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 54 - DNA, 2376 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggctctgcccgtcaccgctctgctgctgcctctggctctgctgctgcacgctgctcgccctcaggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 55 - protein, 488 aa, Artificial Sequence
  Synthetic Construct
MALPVTALLLPLALLLHAARPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 489 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 759 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 488 aa, Artificial Sequence
  Synthetic Construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 488 aa, Artificial Sequence
  Synthetic Construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 489 aa, Artificial Sequence
  Synthetic Construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 365 aa, Artificial Sequence
  Synthetic Construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 2760 bp, Artificial Sequence
  Synthetic Construct
gccgccaccatggctctgcccgtcaccgctctgctgctgcctctggctctgctgctgcacgctgctcgccctcaggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagcggaggaggaggatctgaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactggttaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccaccatgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctaa

SEQ ID NO: 63 - DNA, 2685 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatgcagtctggaacccactggagggtgctgggactgtgcctgctgagcgtgggcgtgtggggacaggaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtggcggcggaggctctggcggcggaggaagcggcggcggcggctccgatggcaacgaggagatgggcggcatcacccagacaccctacaaggtgtccatctctggcaccacagtgatcctgacctgtccacagtatcccggctctgagatcctgtggcagcacaacgacaagaatatcggcggcgatgaggacgataagaatatcggcagcgacgaggatcacctgtctctgaaggagttcagcgagctggagcagtccggctactacgtgtgctaccctcggggctccaagccagaggacgccaacttttacctgtatctgcgggccagagtgtgcgagaattgtatggagatggacgtgatgtccgtggccaccatcgtgatcgtggatatctgtatcacaggcggcctgctgctgctggtgtactattggagcaagaaccggaaggccaaggccaagcctgtgaccagaggagcaggagcaggaggcaggcagaggggacagaacaaggagaggccacctccagtgcccaatcctgactacgagccaatcaggaagggccagcgcgatctgtatagcggcctgaatcagaggcgcatctgataa

SEQ ID NO: 64 - protein, 467 aa, Artificial Sequence
  Synthetic Construct
MQSGTHWRVLGLCLLSVGVWGQEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 65 - DNA, 3147 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatgcagtctggaacccactggagggtgctgggactgtgcctgctgagcgtgggcgtgtggggacaggaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtggcggcggcggctctggaggaggaggcagcggcggcggaggctccggcggcggcggctctatggacattcagatgacccagtccccaagctccctgtctgccagcgtgggagacagagtgaccatcacatgcagggccagccaggatatccgcaactatctgaattggtatcagcagaaacccggcaaggcccctaagctgctgatctattacaccagcaggctggagtccggagtgccatcaagattctccggctctggcagcggaaccgactacaccctgacaatctctagcctgcagccagaggatttcgccacatattactgccagcagggcaacaccctgccctggacatttggccagggcaccaaggtggagatcaagggaggaggaggcagcgggggcggcggctccggaggaggcggctctgaggtgcagctggtggagagcggaggaggactggtgcagcctggaggcagcctgcggctgtcctgtgccgccagcggctattccttcaccggctacacaatgaattgggtcagacaggcaccaggaaagggactggagtgggtggccctgatcaaccctaccaagggcgtgtccacatataatcagaagttcaaggacaggtttaccatctctgtggataagagcaagaacacagcctacctgcagatgaatagcctgagggccgaggacaccgccgtgtattactgcgcacgcagcggatattacggagactccgattggtactttgacgtgtggggccagggcaccctggtgacagtgtcctccggcggaggaggcagctccggacaggtgctgctggagtccaatatcaaggtgctgccaacctggtctacacctgtgcagccaatggcactgatcgtgctgggaggagtggcaggactgctgctgttcatcggactgggcatcttcttttgcgtgcgctgtaggcaccggagaaggcaggcagagaggatgtctcagatcaagagactgctgagcgagaagaagacctgccagtgtcctcaccgctttcagaagacatgtagcccaatctgataa

SEQ ID NO: 66 - protein, 621 aa, Artificial Sequence
  Synthetic Construct
MQSGTHWRVLGLCLLSVGVWGQEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSGGGGSGGGGSGGGGSGGGGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSGGGGSSGQVLLESNIKVLPTWSTPVQPMALIVLGGVAGLLLFIGLGIFFCVRCRHRRRQAERMSQIKRLLSEKKTCQCPHRFQKTCSPI